SEQUENCE LISTING (236 sequences)

SEQ ID NO: 1 - protein, 106 aa, Mus musculus
DIVLTQSPVSLAVSLGQRATISCRASESVDDYGISFMHWYQQKPGQPPKLLIYRASDLESGIPARFSGSGSGTDFTLTINPVETDDVAIYYCQQTSKDPWTFGGGT

SEQ ID NO: 2 - protein, 122 aa, Mus musculus
EVQLQESGPSLVKPSQTLSLTCSVTGDSITRGYWNWIRKFPGNKLEFMGYINYSGTTYYNPSLKSRISITRDTSKNQYYLQLNSVTTEDTATYYCATPHTYGAMDYWGQGTTLTVSAASTKG

SEQ ID NO: 3 - protein, 112 aa, Artificial Sequence
  Synthetic polypeptide
DIQMTQSPSSLSASVGDRVTITCRASESVDDYGISFMHWYQQKPGKAPKLLIYRASDLESGVPSRFSGSGSGTDFTLTISSLQPEDFATYYCQQTSKDPWTFGQGTKVEIKR

SEQ ID NO: 4 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISADTSKNTAYLQMNSLRAEDTAVYYCATPHTYGAMDYWGQGTLVTVSS

SEQ ID NO: 5 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATPHTYGAMDYWGQGTLVTVSS

SEQ ID NO: 6 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATPHTYGAMDYWGQGTLVTVSS

SEQ ID NO: 7 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATTLPYGAMDYWGQGTLVTVSS

SEQ ID NO: 8 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCANSNFYGAMDYWGQGTLVTVSS

SEQ ID NO: 9 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCANLNYYGAMDYWGQGTLVTVSS

SEQ ID NO: 10 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCANANYYGAMDYWGQGTLVTVSS

SEQ ID NO: 11 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATSHNTGEMDYWGQGTLVTVSS

SEQ ID NO: 12 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATTLPYGAMDYWGQGTLVTVSS

SEQ ID NO: 13 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCANSNFYGAMDYWGQGTLVTVSS

SEQ ID NO: 14 - protein, 237 aa, Artificial Sequence
  Synthetic polypeptide
MGWSCIILFLVATATGVHSDIQMTQSPSSLSASVGDRVTITCRASESVDDYGISFMHWYQQKPGKAPKLLIYRASDLESGVPSRFSGSGSGTDFTLTISSLQPEDFATYYCQQTSKDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 441 aa, Artificial Sequence
  Synthetic polypeptide
ESGGGLVQPGGSLRLSCAASGDSITRGYWNWVRQAPGKGLEWVGYINYSGTTYYNPSLKSRFTISRDTSKNTFYLQMNSLRAEDTAVYYCANSNFYGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATNHLYGAMDYWGQGTLVTVSS

SEQ ID NO: 17 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCARPHNYGAMDYWGQGTLVTVSS

SEQ ID NO: 18 - protein, 117 aa, Artificial Sequence; identical to SEQ ID NO: 17
  Synthetic polypeptide

SEQ ID NO: 19 - protein, 108 aa, Mus musculus
DIVMSQSPSSLAVSVGEKVTMSCKSSQSLLYSSNQNNYLAWYQQKPGQSPKLLIYWASTRESGVPDRFTGSGSGTDFTLTISSVKAEDLAVYYCQQYYTYPYTFGGGT

SEQ ID NO: 20 - protein, 123 aa, Mus musculus
EVKLVESGGGLVQPGGSLRLSCAISGFTVTAYYMSWVRQPPGKALEWLGFIRDKANGYTTEYNPSVKGRFTISRDNSQSIFYLQMNTLRAEDSATYYCAQVRRALDYWGQGTTVTVSAASTKG

SEQ ID NO: 21 - protein, 114 aa, Artificial Sequence
  Synthetic polypeptide
DIQMTQSPSSLSASVGDRVTITCKSSQSLLYSSNQNNYLAWYQQKPGKAPKLLIYWASTRESGVPSRFSGSGSGTDFTLTISSLQPEDFATYYCQQYYTYPYTFGQGTKVEIKR

SEQ ID NO: 22 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 23 - protein, 114 aa, Artificial Sequence
  Synthetic polypeptide
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGQGTKVEIKR

SEQ ID NO: 24 - protein, 118 aa, Artificial Sequence
  Synthetic polypeptide
EVQLVESGGGLVQPGGSLRLSCAASGLPMAGFYTSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 25 - protein, 114 aa, Artificial Sequence
  Synthetic polypeptide
DIQMTQSPSSLSASVGDRVTITCKSSQSLLYSSNQNNYLAWYQQKPGKAPKLLIYWASIRDSGVPSRFSGSGSGTDFTLTISSLQPEDFATYYCQQYYTYPYTFGQGTKVEIKR

SEQ ID NO: 26 - protein, 118 aa, Artificial Sequence
  Synthetic polypeptide
EVQLVESGGGLVQPGGSLRLSCAASDSPRSGYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 27 - protein, 118 aa, Artificial Sequence
  Synthetic polypeptide
EVQLVESGGGLVQPGGSLRLSCAASAWPVTGYYMSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 28 - protein, 118 aa, Artificial Sequence
  Synthetic polypeptide
EVQLVESGGGLVQPGGSLRLSCAASGSTVSSYYFSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 29 - protein, 118 aa, Artificial Sequence
  Synthetic polypeptide
EVQLVESGGGLVQPGGSLRLSCAASSPAVAPHYWSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 30 - protein, 118 aa, Artificial Sequence
  Synthetic polypeptide
EVQLVESGGGLVQPGGSLRLSCAASGFPYTSYYITWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 31 - protein, 118 aa, Artificial Sequence
  Synthetic polypeptide
EVQLVESGGGLVQPGGSLRLSCAASGFTGGSYYIGWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 32 - protein, 118 aa, Artificial Sequence
  Synthetic polypeptide
EVQLVESGGGLVQPGGSLRLSCAASGFTESAYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 33 - protein, 118 aa, Artificial Sequence
  Synthetic polypeptide
EVQLVESGGGLVQPGGSLRLSCAASGATAAAYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 34 - protein, 118 aa, Artificial Sequence
  Synthetic polypeptide
EVQLVESGGGLVQPGGSLRLSCAASGATGIGYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 35 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSS

SEQ ID NO: 36 - protein, 118 aa, Artificial Sequence
  Synthetic polypeptide
EVQLVESGGGLVQPGGSLRLSCAASGWTEHGHYMSWVRQAPGKGLEWVGFIRDKANGYTTEYNPSVKGRFTISRDTSKNTFYLQMNSLRAEDTAVYYCAQVRRALDYWGQGTLVTVSS

SEQ ID NO: 37 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 38 - protein, 118 aa, Artificial Sequence
  Synthetic polypeptide
EVQLVESGGGLVQPGGSLRLSCAASGRPRRGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 39 - protein, 118 aa, Artificial Sequence
  Synthetic polypeptide
EVQLVESGGGLVQPGGSLRLSCAASGVTGGSFYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 40 - protein, 118 aa, Artificial Sequence
  Synthetic polypeptide
EVQLVESGGGLVQPGGSLRLSCAASGLTGTGYYTSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 41 - protein, 118 aa, Artificial Sequence
  Synthetic polypeptide
EVQLVESGGGLVQPGGSLRLSCAASGFPVTGSYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 42 - protein, 118 aa, Artificial Sequence
  Synthetic polypeptide
EVQLVESGGGLVQPGGSLRLSCAASGFTTTARYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 43 - protein, 118 aa, Artificial Sequence
  Synthetic polypeptide
EVQLVESGGGLVQPGGSLRLSCAASGFTVTASYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 44 - protein, 118 aa, Artificial Sequence
  Synthetic polypeptide
EVQLVESGGGLVQPGGSLRLSCAASGITVTASYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 45 - protein, 118 aa, Artificial Sequence
  Synthetic polypeptide
EVQLVESGGGLVQPGGSLRLSCAASGFALRGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 46 - protein, 118 aa, Artificial Sequence
  Synthetic polypeptide
EVQLVESGGGLVQPGGSLRLSCAASEFAVTGSYIAWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 47 - protein, 118 aa, Artificial Sequence
  Synthetic polypeptide
EVQLVESGGGLVQPGGSLRLSCAASTRAVTGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 48 - protein, 118 aa, Artificial Sequence
  Synthetic polypeptide
EVQLVESGGGLVQPGGSLRLSCAASGYIATGHYIAWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 49 - protein, 118 aa, Artificial Sequence
  Synthetic polypeptide
EVQLVESGGGLVQPGGSLRLSCAASVDKLTGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 50 - protein, 118 aa, Artificial Sequence
  Synthetic polypeptide
EVQLVESGGGLVQPGGSLRLSCAASGFLGPGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 51 - protein, 118 aa, Artificial Sequence
  Synthetic polypeptide
EVQLVESGGGLVQPGGSLRLSCAASGLQATGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 52 - protein, 118 aa, Artificial Sequence
  Synthetic polypeptide
EVQLVESGGGLVQPGGSLRLSCAASGLSMTGVYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 53 - protein, 118 aa, Artificial Sequence
  Synthetic polypeptide
EVQLVESGGGLVQPGGSLRLSCAASGSSLTGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 54 - protein, 118 aa, Artificial Sequence
  Synthetic polypeptide
EVQLVESGGGLVQPGGSLRLSCAASGFTLAGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 55 - protein, 118 aa, Artificial Sequence
  Synthetic polypeptide
EVQLVESGGGLVQPGGSLRLSCAASGFTVNGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 56 - protein, 118 aa, Artificial Sequence; identical to SEQ ID NO: 55
  Synthetic polypeptide

SEQ ID NO: 57 - protein, 118 aa, Artificial Sequence
  Synthetic polypeptide
EVQLVESGGGLVQPGGSLRLSCAASGFTWTGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 58 - protein, 118 aa, Artificial Sequence
  Synthetic polypeptide
EVQLVESGGGLVQPGGSLRLSCAASGLTVTGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 59 - protein, 118 aa, Artificial Sequence
  Synthetic polypeptide
EVQLVESGGGLVQPGGSLRLSCAASGFTVSPYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 60 - protein, 118 aa, Artificial Sequence
  Synthetic polypeptide
EVQLVESGGGLVQPGGSLRLSCAASGDSLDTSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 61 - protein, 118 aa, Artificial Sequence
  Synthetic polypeptide
EVQLVESGGGLVQPGGSLRLSCAASGDTDGTYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 62 - protein, 118 aa, Artificial Sequence
  Synthetic polypeptide
EVQLVESGGGLVQPGGSLRLSCAASGFPVTGSYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 63 - protein, 118 aa, Artificial Sequence
  Synthetic polypeptide
EVQLVESGGGLVQPGGSLRLSCAASGFSDTGHYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 64 - protein, 118 aa, Artificial Sequence
  Synthetic polypeptide
EVQLVESGGGLVQPGGSLRLSCAASGISLNGYYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 65 - protein, 118 aa, Artificial Sequence
  Synthetic polypeptide
EVQLVESGGGLVQPGGSLRLSCAASGFTDYGNYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 66 - protein, 118 aa, Artificial Sequence
  Synthetic polypeptide
EVQLVESGGGLVQPGGSLRLSCAASGFPGTGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 67 - protein, 118 aa, Artificial Sequence
  Synthetic polypeptide
EVQLVESGGGLVQPGGSLRLSCAASGFPLTGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 68 - protein, 118 aa, Artificial Sequence
  Synthetic polypeptide
EVQLVESGGGLVQPGGSLRLSCAASGFSLIGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 69 - protein, 118 aa, Artificial Sequence
  Synthetic polypeptide
EVQLVESGGGLVQPGGSLRLSCAASGFTLSAHYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 70 - protein, 118 aa, Artificial Sequence
  Synthetic polypeptide
EVQLVESGGGLVQPGGSLRLSCAASSYTENGYYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 71 - protein, 118 aa, Artificial Sequence
  Synthetic polypeptide
EVQLVESGGGLVQPGGSLRLSCAASGFTEGGFYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 72 - protein, 118 aa, Artificial Sequence
  Synthetic polypeptide
EVQLVESGGGLVQPGGSLRLSCAASGFIEDSYYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 73 - protein, 118 aa, Artificial Sequence
  Synthetic polypeptide
EVQLVESGGGLVQPGGSLRLSCAASGFNGGTFYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 74 - protein, 220 aa, Artificial Sequence
  Synthetic polypeptide
DIQMTQSPSSLSASVGDRVTITCKSSQSLLYSSNQNNYLAWYQQKPGKAPKLLIYWASTRESGVPSRFSGSGSGTDFTLTISSLQPEDFATYYCQQYYTYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 447 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 447 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHXHYTQKSLSLSPG

SEQ ID NO: 77 - protein, 108 aa, Mus musculus
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPFTFGSGT

SEQ ID NO: 78 - protein, 123 aa, Mus musculus
DVQLQESGPSLVKPSQTLSLTCSVTGDSITSGYWNWIRKFPGNKLEYMGYISYSGSTYYNPSLKSRISITRDTSKNQFYLQLLSVTTEDTATYYCAGLDGLYWYFDVWGAGTLVTVSSASTKG

SEQ ID NO: 79 - protein, 114 aa, Artificial Sequence
  Synthetic polypeptide
DIQMTQSPSSLSASVGDRVTITCRSSQSLVHSNGNTYLHWYQQKPGKAPKLLIYKVSNRFSGVPSRFSGSGSGTDFTLTISSLQPEDFATYYCSQSTHVPPFTFGQGTKVEIKR

SEQ ID NO: 80 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISADTSKNTAYLQMNSLRAEDTAVYYCAGLDGLYWYFDVWGQGTLVTVSS

SEQ ID NO: 81 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISRDTSKNTFYLQMNSLRAEDTAVYYCAGLDGLYWYFDVWGQGTLVTVSS

SEQ ID NO: 82 - protein, 118 aa, Artificial Sequence
  Synthetic polypeptide
EVQLVESGGGLVQPGGSLRLSCAASGDSINFGYWNWVRQAPGKGLEWVGYISYSGSTYYNPSLKSRFTISRDTSKNTFYLQMNSLRAEDTAVYYCAALNDLFLYFDVWGQGTLVTVSS

SEQ ID NO: 83 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISRDTSKNTFYLQMNSLRAEDTAVYYCAGLNDLYLYFDVWGQGTLVTVSS

SEQ ID NO: 84 - protein, 118 aa, Artificial Sequence
  Synthetic polypeptide
EVQLVESGGGLVQPGGSLRLSCAASGDSITSGYWNWVRQAPGKGLEWVGNISYSGSTYYNPSLKSRFTISRDTSKNTFYLQMNSLRAEDTAVYYCAGLDGLYWYFDVWGQGTLVTVSS

SEQ ID NO: 85 - protein, 118 aa, Artificial Sequence
  Synthetic polypeptide
EVQLVESGGGLVQPGGSLRLSCAASGDSITSGYWNWVRQAPGKGLEWVGNIGYSGSTYYNPSLKSRFTISRDTSKNTFYLQMNSLRAEDTAVYYCAGLDGLYWYFDVWGQGTLVTVSS

SEQ ID NO: 86 - protein, 214 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 232 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYSSVRGCAGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 88 - protein, 119 aa, Artificial Sequence
  Synthetic polypeptide
EVQLVESGGGLVQPGGSLRLSCAASGFTISGSSIHWVRQAPGKGLEWVGTIYPYGGNTYYADSVKGRFTISADTSKNTAYLQMNSLRAEDTAVYYCARRAFVMSGMDYWGQGTLVTVSS

SEQ ID NO: 89 - protein, 117 aa, Artificial Sequence
  Synthetic polypeptide
EVQLVESGGGLVQPGGSLRLSCAASGFTITGSYIHWVRQAPGKGLEWVAWIYPDGGYTDYADSVKGRFTISADTSKNTAYLQMNSLRAEDTAVYYCASKPAGPFGYWGQGTLVTVSS

SEQ ID NO: 90 - protein, 119 aa, Artificial Sequence
  Synthetic polypeptide
EVQLVESGGGLVQPGGSLRLSCAASGFTITGYGIHWVRQAPGKGLEWVAGITPANGYTDYADSVKGRFTISADTSKNTAYLQMNSLRAEDTAVYYCAKSFPFHYNFDYWGQGTLVTVSS

SEQ ID NO: 91 - protein, 120 aa, Artificial Sequence
  Synthetic polypeptide
EVQLVESGGGLVQPGGSLRLSCAASGFTINSSAIHWVRQAPGKGLEWVGYITPASGYTDYADSVKGRFTISADTSKNTAYLQMNSLRAEDTAVYYCAKGFHWYRGFFDYWGQGTLVTVSS

SEQ ID NO: 92 - protein, 116 aa, Artificial Sequence
  Synthetic polypeptide
EVQLVESGGGLVQPGGSLRLSCAASGFTITGSYIHWVRQAPGKGLEWVAWIYPDGGYTDYADSVKGRFTISADTSKNTAYLQMNSLRAEDTAVYYCASKPAGFGYWGQGTLVTVSS

SEQ ID NO: 93 - protein, 125 aa, Artificial Sequence
  Synthetic polypeptide
EVQLVESGGGLVQPGGSLRLSCAASGFTISSTGIHWVRQAPGKGLEWVGGISPSSGSTNYADSVKGRFTISADTSKNTAYLQMNSLRAEDTAVYYCARRKVVSSHVTNKYVMDYWGQGTLVTVSS

SEQ ID NO: 94 - protein, 121 aa, Artificial Sequence
  Synthetic polypeptide
EVQLVESGGGLVQPGGSLRLSCAASGFTINGSWIHWVRQAPGKGLEWVAWITPSNGSTDYADSVKGRFTISADTSKNTAYLQMNSLRAEDTAVYYCARLSRRPWLWGMDYWGQGTLVTVSS

SEQ ID NO: 95 - protein, 120 aa, Artificial Sequence
  Synthetic polypeptide
EVQLVESGGGLVQPGGSLRLSCAASGFTIXXXXIHWVRQAPGKGLEWVGWISPXXGNTXYADSVKGRFTISADTSKNTAYLQMNSLRAEDTAVYYCARXXXXXXAAMDYWGQGTLVTVSS

SEQ ID NO: 96 - protein, 120 aa, Artificial Sequence
  Synthetic polypeptide
EVQLVESGGGLVQPGGSLRLSCAASGFTIXXXXIHWVRQAPGKGLEWVGXISPXXGDTYYADSVKGRFTISADTSKNTAYLQMNSLRAEDTAVYYCARALCAPXXAMDYWGQGTLVTVSS

SEQ ID NO: 97 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRITPPTFGQGTKVEIKR

SEQ ID NO: 98 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNRLGVCAGGMDYWGQGTLVTVSS

SEQ ID NO: 99 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQTSTSPPTFGQGTKVEIKR

SEQ ID NO: 100 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNNLGVCAGAMDYWGQGTLVTVSS

SEQ ID NO: 101 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYSTPPTFGQGTKVEIKR

SEQ ID NO: 102 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYDRARVCAGAMDYWGQGTLVTVSS

SEQ ID NO: 103 - protein, 108 aa, Artificial Sequence
  Synthetic polypeptide
DIQMTQSPSSLSASVGDRVTITCRASQDVSTAVAWYQQKPGKAPKLLIYGASNLYSGVPSRFSGSGSGTDFTLTISSLQPEDFATYYCQQSHASPPTFGQGTKVEIKR

SEQ ID NO: 104 - protein, 125 aa, Artificial Sequence
  Synthetic polypeptide
EVQLVESGGGLVQPGGSLRLSCAASGFTISRRSIHWVRQAPGKGLEWVAWITPSDGNTDYADSVKGRFTISADTSKNTAYLQMNSLRAEDTAVYYCARRVCYSSVRGCAGAMDYWGQGTLVTVSS

SEQ ID NO: 105 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRISPPTFGQGTKVEIKR

SEQ ID NO: 106 - protein, 125 aa, Artificial Sequence
  Synthetic polypeptide
EVQLVESGGGLVQPGGSLRLSCAASGFTISSNSIHWVRQAPGKGLEWVAWVTPSGGSTDYADSVKGRFTISADTSKNTAYLQMNSLRAEDTAVYYCARRVCYNRLGVCAGGMDYWGQGTLVTVSS

SEQ ID NO: 107 - protein, 125 aa, Artificial Sequence
  Synthetic polypeptide
EVQLVESGGGLVQPGGSLRLSCAASGFTISSSSIHWVRQAPGKGLEWVAWITPGHGSTDYADSVKGRFTISADTSKNTAYLQMNSLRAEDTAVYYCARRVCYNRLGVCAGGMDYWGQGTLVTVSS

SEQ ID NO: 108 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSHNTPPTFGQGTKVEIKR

SEQ ID NO: 109 - protein, 125 aa, Artificial Sequence
  Synthetic polypeptide
EVQLVESGGGLVQPGGSLRLSCAASGFSISSNSIHWVRQAPGKGLEWVAWITPTHGSTDYADSVKGRFTISADTSKNTAYLQMNSLRAEDTAVYYCARRVCYNRLGVCAGGMDYWGQGTLVTVSS

SEQ ID NO: 110 - protein, 125 aa, Artificial Sequence
  Synthetic polypeptide
EVQLVESGGGLVQPGGSLRLSCAASGFSIARSSIHWVRQAPGKGLEWVAWILPSAGSTDYADSVKGRFTISADTSKNTAYLQMNSLRAEDTAVYYCARRVCYNRLGVCAGGMDYWGQGTLVTVSS

SEQ ID NO: 111 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSLITPPTFGQGTKVEIKR

SEQ ID NO: 112 - protein, 125 aa, Artificial Sequence
  Synthetic polypeptide
EVQLVESGGGLVQPGGSLRLSCAASGFSIRSISIHWVRQAPGKGLEWVAWITPFNGTTDYADSVKGRFTISADTSKNTAYLQMNSLRAEDTAVYYCARRVCYNRLGVCAGGMDYWGQGTLVTVSS

SEQ ID NO: 113 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRMSPPTFGQGTKVEIKR

SEQ ID NO: 114 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNHLGVCAGGMDYWGQGTLVTVSS

SEQ ID NO: 115 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRTTPPTFGQGTKVEIKR

SEQ ID NO: 116 - protein, 125 aa, Artificial Sequence
  Synthetic polypeptide
EVQLVESGGGLVQPGGSLRLSCAASGFSISNHSIHWVRQAPGKGLEWVAWVTPSYGITDYADSVKGRFTISADTSKNTAYLQMNSLRAEDTAVYYCARRVCYNRLGVCAGGMDYWGQGTLVTVSS

SEQ ID NO: 117 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSLMTPPTFGQGTKVEIKR

SEQ ID NO: 118 - protein, 125 aa, Artificial Sequence
  Synthetic polypeptide
EVQLVESGGGLVQPGGSLRLSCAASGFTISSNSIHWVRQAPGKGLEWVAWVTPGVGSTDYADSVKGRFTISADTSKNTAYLQMNSLRAEDTAVYYCARRVCYNRLGVCAGGMDYWGQGTLVTVSS

SEQ ID NO: 119 - protein, 108 aa, Artificial Sequence; identical to SEQ ID NO: 105
  Synthetic polypeptide

SEQ ID NO: 120 - protein, 125 aa, Artificial Sequence
  Synthetic polypeptide
EVQLVESGGGLVQPGGSLRLSCAASGFSISRRSIHWVRQAPGKGLEWVAWITPLYGSTHYADSVKGRFTISADTSKNTAYLQMNSLRAEDTAVYYCARRVCYNRLGVCAGGMDYWGQGTLVTVSS

SEQ ID NO: 121 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSGISPPTFGQGTKVEIKR

SEQ ID NO: 122 - protein, 125 aa, Artificial Sequence
  Synthetic polypeptide
EVQLVESGGGLVQPGGSLRLSCAASGFSIRNNSIHWVRQAPGKGLEWVAWVLPSNGVTDYADSVKGRFTISADTSKNTAYLQMNSLRAEDTAVYYCARRVCYNRLGVCAGGMDYWGQGTLVTVSS

SEQ ID NO: 123 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGQGTKVEIKR

SEQ ID NO: 124 - protein, 125 aa, Artificial Sequence
  Synthetic polypeptide
EVQLVESGGGLVQPGGSLRLSCAASGFTISNSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 125 - protein, 125 aa, Artificial Sequence
  Synthetic polypeptide
EVQLVESGGGLVQPGGSLRLSCAASGFTISASSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 126 - protein, 125 aa, Artificial Sequence
  Synthetic polypeptide
EVQLVESGGGLVQPGGSLRLSCAASGFTISQSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 127 - protein, 125 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 128 - protein, 214 aa, Artificial Sequence [sequence omitted]

SEQ ID NO: 129 - protein, 232 aa, Artificial Sequence [sequence omitted]

SEQ ID NO: 130 - protein, 454 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 454 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHXHYTQKSLSLSPG

SEQ ID NO: 132 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 217 aa, Artificial Sequence
  Synthetic polypeptide
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALXNHYTQKSLSLSPG

SEQ ID NO: 135 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 137 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 138 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWZSNGQPENNYKTTPPVLDSDGSFFLYSRLTVDKSRWQEGNVFSCSVMHEALHNHYTQKSLSLSLGK

SEQ ID NO: 139 - protein, 215 aa, Mus musculus
TVPEVSSVFIFPPKPKDVLTITLTPKVTCVVVDISKDDPEVQFSWFVDDVEVHTAQTQPREEQFNSTFRSVSELPIMHQDCLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 140 - protein, 218 aa, Mus musculus
PAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 141 - protein, 218 aa, Mus musculus
PAPNLEGGPSVFIFPPNIKDVLMISLTPKVTCVVVDVSEDDPDVQISWFVNNVEVHTAQTQTHREDYNSTIRVVSHLPIQHQDWMSGKEFKCKVNNKDLPSPIERTISKPKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 142 - protein, 217 aa, Mus musculus
PPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPG

SEQ ID NO: 143 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 144 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 145 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 146 - protein, 185 aa, Homo sapiens
MRRGPRSLRGRDAPAPTPCVPAECFDLLVRHCVACGLLRTPRPKPAGAASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 147 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 148 - protein, 175 aa, Rattus norvegicus
MGVRRLRVRSRRSRDSPVSTQCNQTECFDPLVRNCVSCELFYTPETRHASSLEPGTALQPQEGSGLRPDVALLFGAPALLGLVLALTLVGLVSLVGWRWRQQRRTASLDTSEGVQQESLENVFVPPSETLHASAPNWPPFKEDADNILSCHSIPVPATELGSTELVTTKTAGPEQ

SEQ ID NO: 149 - protein, 183 aa, Macaca mulatta
MKRGPRSLRGRDAPAPTPCVPAECFDLLVRHCVACGLLRTPRPKPAAPASSPAPRTALQPQESVGAGAGEAALSLPGLLFGAPALLGLALVLALVLVGLVSWRRRQRRLRGASSAEAPDGDKDKDEPLDKVIILSPGISDAAAPAWPPPGEDPGTTPPGHSVPVPATELGSTELVTTKTAGPE

SEQ ID NO: 150 - protein, 23 aa, Artificial Sequence
  Synthetic polypeptide
TPCVPAECFDLLVRHCVACGLLR

SEQ ID NO: 151 - protein, 61 aa, Homo sapiens
MRRGPRSLRGRDAPAPTPCVPAECFDLLVRHCVACGLLRTPRPKPAGASSPAPRTALQPQE

SEQ ID NO: 152 - protein, 64 aa, Mus musculus
MGARRLRVRSQRSRDSSVPTQCNQTECFDPLVRNCVSCELFHTPDTGHTSSLEPGTALQPQEGS

SEQ ID NO: 153 - protein, 314 aa, Artificial Sequence
  Synthetic polypeptide
MSALLILALVGAAVASTGARRLRVRSQRSRDSSVPTQCNQTECFDPLVRNCVSCELFHTPDTGHTSSLEPGTALQPQEGQVTGDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 154 - DNA, 36 bp, Artificial Sequence
  Synthetic nucleotide sequence
tcttgtgacaaaactcacagtggcggtggctctggt

SEQ ID NO: 155 - DNA, 48 bp, Artificial Sequence
  Synthetic nucleotide sequence
tattactgtcagcaacattaataaaggccttaacctcccacgttcgga

SEQ ID NO: 156 - DNA, 55 bp, Artificial Sequence
  Synthetic nucleotide sequence
acctgccgtgccagtcagrdtrktrvwanwthtgtagcctggtatcaacagaaac

SEQ ID NO: 157 - DNA, 55 bp, Artificial Sequence
  Synthetic nucleotide sequence
acctgccgtgccagtcagrdtrktrvwanwthtctggcctggtatcaacagaaac

SEQ ID NO: 158 - DNA, 48 bp, Artificial Sequence
  Synthetic nucleotide sequence
ccgaagcctctgatttackbggcatccavcctctactctggagtccct

SEQ ID NO: 159 - DNA, 54 bp, Artificial Sequence
  Synthetic nucleotide sequence
ccgaagcttctgatttackbggcatccavcctcgmatctggagtcccttctcgc

SEQ ID NO: 160 - DNA, 57 bp, Artificial Sequence
  Synthetic nucleotide sequence
gcaacttattactgtcagcaatmtdmcrvtnhtcctykgacgttcggacagggtacc

SEQ ID NO: 161 - DNA, 57 bp, Artificial Sequence
  Synthetic nucleotide sequence
gcaacttattactgtcagcaatmtdmcrvtnhtccttwtacgttcggacagggtacc

SEQ ID NO: 162 - DNA, 57 bp, Artificial Sequence
  Synthetic nucleotide sequence
gcaacttattactgtcagcaasrtdmcrvtnhtcctykgacgttcggacagggtacc

SEQ ID NO: 163 - DNA, 57 bp, Artificial Sequence
  Synthetic nucleotide sequence
gcaacttattactgtcagcaasrtdmcrvtnhtccttwtacgttcggacagggtacc

SEQ ID NO: 164 - DNA, 57 bp, Artificial Sequence
  Synthetic nucleotide sequence
gcaacttattactgtcagcaannnnnnnnnnnnccgnnnacgttcggacagggtacc

SEQ ID NO: 165 - DNA, 57 bp, Artificial Sequence
  Synthetic nucleotide sequence
tgtgcagcttctggcttcwccnttnnnnnnnnnnnnnnnnnntgggtgcgtcaggcc

SEQ ID NO: 166 - DNA, 69 bp, Artificial Sequence
  Synthetic nucleotide sequence
aagggcctggaatgggttgstnnnatcnnnnnnnnnnnnnnnnnnnnnnnntatgccgatagcgtcaag

SEQ ID NO: 167 - DNA, 79 bp, Artificial Sequence
  Synthetic nucleotide sequence
gccgtctattattgtgctcgtnnnnnntgcnnnnnnnnnnnnnnnnnntgcnnnnnnnnnatggactactggggtcaag

SEQ ID NO: 168 - DNA, 79 bp, Artificial Sequence
  Synthetic nucleotide sequence
gccgtctattattgtgctcgtnnnnnnnnnnnnnnnnnnnnnnnnnnnnnnnnnnnnnnnatggactactggggtcaag

SEQ ID NO: 169 - DNA, 63 bp, Artificial Sequence
  Synthetic nucleotide sequence
gccgtctattattgtgctnnnnnnnnntgcnnnnnnnnnnnnnnnggctgcgcgggggcaatg

SEQ ID NO: 170 - DNA, 63 bp, Artificial Sequence
  Synthetic nucleotide sequence
gctcgtcgggtctgctacnnnnnnnnnnnnnnntgcnnnnnnnnnatggactactggggtcaa

SEQ ID NO: 171 - DNA, 28 bp, Artificial Sequence
  Synthetic nucleotide sequence
gctcggttgccgccgggcgttttttatg

SEQ ID NO: 172 - DNA, 51 bp, Artificial Sequence
  Synthetic nucleotide sequence
acttattactgtcagcaannnnnnnnnnnnccgnnnacgttcggacagggt

SEQ ID NO: 173 - DNA, 54 bp, Artificial Sequence
  Synthetic nucleotide sequence
acttattactgtcagcaannnnnnnnnnnnnnnccgnnnacgttcggacagggt

SEQ ID NO: 174 - DNA, 48 bp, Artificial Sequence
  Synthetic nucleotide sequence
acttattactgtcagcaannknnknnkccgcccacgttcggacagggt

SEQ ID NO: 175 - DNA, 49 bp, Artificial Sequence
  Synthetic nucleotide sequence
gcagcttctggcttcwccattnnnnnnnnnnnnatacactgggtgcgtc

SEQ ID NO: 176 - DNA, 63 bp, Artificial Sequence
  Synthetic nucleotide sequence
ctggaatgggttgcttggrttnnncctnnnnnnggtnnnactnnntatgccgatagcgtcaag

SEQ ID NO: 177 - DNA, 60 bp, Artificial Sequence
  Synthetic nucleotide sequence
gtctattattgtgctcgtnnnnnntgcnnnnnnnnnnnnnnnnnntgcgctggtgggatg

SEQ ID NO: 178 - DNA, 75 bp, Artificial Sequence
  Synthetic nucleotide sequence
gtctattattgtgctcgtnnnnnntgcnnnnnnnnncttggtgtttgcnnnnnnnnnatggactactggggtcaa

SEQ ID NO: 179 - DNA, 72 bp, Artificial Sequence
  Synthetic nucleotide sequence
gtctattattgtgctcgtnnnnnnrstnnnnnnnnnnnnnnnnnnrstgstgstgsgatggactactggggt

SEQ ID NO: 180 - DNA, 70 bp, Artificial Sequence
  Synthetic nucleotide sequence
tattattgtgctcgtcggnnnrstnnnnnnnnnnnnnnnnnnrstnnnnnnnnnatggactactggggtc

SEQ ID NO: 181 - DNA, 55 bp, Artificial Sequence
  Synthetic nucleotide sequence
acctgccgtgccagtsaagamrttkccasckctgtagcctggtatcaacagaaac

SEQ ID NO: 182 - DNA, 54 bp, Artificial Sequence
  Synthetic nucleotide sequence
ccgaagcttctgatttwckccgcatcctwcctctwctctggagtcccttctcgc

SEQ ID NO: 183 - DNA, 57 bp, Artificial Sequence
  Synthetic nucleotide sequence
gcaacttattactgtcagcaskccsaarttkccccgscaacgttcggacagggtacc

SEQ ID NO: 184 - DNA, 51 bp, Artificial Sequence
  Synthetic nucleotide sequence
gcagcttctggcttcaccattkcckcckcckccatacactgggtgcgtcag

SEQ ID NO: 185 - DNA, 51 bp, Artificial Sequence
  Synthetic nucleotide sequence
gcagcttctggcttcaccattagtkccagckccatacactgggtgcgtcag

SEQ ID NO: 186 - DNA, 51 bp, Artificial Sequence
  Synthetic nucleotide sequence
gcagcttctggcttcaccattkccagckcctctatacactgggtgcgtcag

SEQ ID NO: 187 - DNA, 72 bp, Artificial Sequence
  Synthetic nucleotide sequence
aagggcctggaatgggttgcatkgrttmtcscakccrttgsttwcascgamtatgccgatagcgtcaagggc

SEQ ID NO: 188 - DNA, 72 bp, Artificial Sequence
  Synthetic nucleotide sequence
aagggcctggaatgggttgcttggrttcttscatctrttggttwcactgamtatgccgatagcgtcaagggc

SEQ ID NO: 189 - DNA, 72 bp, Artificial Sequence
  Synthetic nucleotide sequence
aagggcctggaatgggttgcttkggttmtccctkccgtggsttttascgactatgccgatagcgtcaagggc

SEQ ID NO: 190 - DNA, 81 bp, Artificial Sequence
  Synthetic nucleotide sequence
actgccgtctattattgtgcaaraarartttgctwcracaramtcgstrtttgckctgstgstatggactactggggtcaa

SEQ ID NO: 191 - DNA, 81 bp, Artificial Sequence
  Synthetic nucleotide sequence
actgccgtctattattgtgctcgtaragtctgctwcaacaracttgstgtttgckctggtgstatggactactggggtcaa

SEQ ID NO: 192 - DNA, 81 bp, Artificial Sequence
  Synthetic nucleotide sequence
actgccgtctattattgtgctaracggrtttgctacraccgcmtcggtrtttgcgctgstggtatggactactggggtcaa

SEQ ID NO: 193 - protein, 125 aa, Artificial Sequence
  Synthetic
EVQLVESGGGLVQPGGSLRLSCAASGFTIASSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 194 - protein, 107 aa, Artificial Sequence
  Synthetic
DIQMTQSPSSLSASVGDRVTITCRASEDIATSVAWYQQKPGKAPKLLIFAASFLYSGVPSRFSGSGSGTDFTLTISSLQPEDFATYYCQQSQISPPTFGQGTKVEIK

SEQ ID NO: 195 - protein, 107 aa, Artificial Sequence
  Synthetic
DIQMTQSPSSLSASVGDRVTITCRASEDISSSVAWYQQKPGKAPKLLIFSASFLFSGVPSRFSGSGSGTDFTLTISSLQPEDFATYYCQQSEVSPPTFGQGTKVEIK

SEQ ID NO: 196 - protein, 107 aa, Artificial Sequence
  Synthetic
DIQMTQSPSSLSASVGDRVTITCRASEDISTAVAWYQQKPGKAPKLLIFAASYLYSGVPSRFSGSGSGTDFTLTISSLQPEDFATYYCQQSQISPPTFGQGTKVEIK

SEQ ID NO: 197 - protein, 107 aa, Artificial Sequence
  Synthetic
DIQMTQSPSSLSASVGDRVTITCRASEDISSAVAWYQQKPGKAPKLLIFAASYLFSGVPSRFSGSGSGTDFTLTISSLQPEDFATYYCQQSQVSPPTFGQGTKVEIK

SEQ ID NO: 198 - protein, 107 aa, Artificial Sequence
  Synthetic
DIQMTQSPSSLSASVGDRVTITCRASEDISSAVAWYQQKPGKAPKLLIFAASFLYSGVPSRFSGSGSGTDFTLTISSLQPEDFATYYCQQSQISPPTFGQGTKVEIK

SEQ ID NO: 199 - protein, 107 aa, Artificial Sequence
  Synthetic
DIQMTQSPSSLSASVGDRVTITCRASEEIATAVAWYQQKPGKAPKLLIFAASYLFSGVPSRFSGSGSGTDFTLTISSLQPEDFATYYCQQSQVAPPTFGQGTKVEIK

SEQ ID NO: 200 - protein, 107 aa, Artificial Sequence
  Synthetic peptide
DIQMTQSPSSLSASVGDRVTITCRASEEIATAVAWYQQKPGKAPKLLIFAASFLYSGVPSRFSGSGSGTDFTLTISSLQPEDFATYYCQQSEVSPPTFGQGTKVEIK

SEQ ID NO: 201 - protein, 107 aa, Artificial Sequence
  Synthetic polypeptide
DIQMTQSPSSLSASVGDRVTITCRASQDIATSVAWYQQKPGKAPKLLIFAASYLFSGVPSRFSGSGSGTDFTLTISSLQPEDFATYYCQQSQVSPPTFGQGTKVEIK

SEQ ID NO: 202 - protein, 107 aa, Artificial Sequence
  Synthetic polypeptide
DIQMTQSPSSLSASVGDRVTITCRASEEISTAVAWYQQKPGKAPKLLIFSASYLYSGVPSRFSGSGSGTDFTLTISSLQPEDFATYYCQQSEVSPPTFGQGTKVEIK

SEQ ID NO: 203 - protein, 107 aa, Artificial Sequence
  Synthetic polypeptide
DIQMTQSPSSLSASVGDRVTITCRASEDISSAVAWYQQKPGKAPKLLIFSASFLFSGVPSRFSGSGSGTDFTLTISSLQPEDFATYYCQQSQVSPPTFGQGTKVEIK

SEQ ID NO: 204 - protein, 107 aa, Artificial Sequence
  Synthetic polypeptide
DIQMTQSPSSLSASVGDRVTITCRASQDVATAVAWYQQKPGKAPKLLIFAASFLYSGVPSRFSGSGSGTDFTLTISSLQPEDFATYYCQQSQISPPTFGQGTKVEIK

SEQ ID NO: 205 - protein, 107 aa, Artificial Sequence
  Synthetic polypeptide
DIQMTQSPSSLSASVGDRVTITCRASEEIATSVAWYQQKPGKAPKLLIFSASYLYSGVPSRFSGSGSGTDFTLTISSLQPEDFATYYCQQSQVSPPTFGQGTKVEIK

SEQ ID NO: 206 - protein, 107 aa, Artificial Sequence
  Synthetic polypeptide
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGQGTKVEIK

SEQ ID NO: 207 - protein, 107 aa, Artificial Sequence
  Synthetic polypeptide
DIQMTQSPSSLSASVGDRVTITCRASEDVSSAVAWYQQKPGKAPKLLIFAASYLFSGVPSRFSGSGSGTDFTLTISSLQPEDFATYYCQHSQVSPPTFGQGTKVEIK

SEQ ID NO: 208 - protein, 8 aa, Artificial Sequence
  Synthetic polypeptide
RDNSKNTL

SEQ ID NO: 209 - protein, 8 aa, Artificial Sequence
  Synthetic polypeptide
RDTSKNTA

SEQ ID NO: 210 - protein, 8 aa, Artificial Sequence
  Synthetic polypeptide
RDTSKNTF

SEQ ID NO: 211 - protein, 8 aa, Artificial Sequence
  Synthetic polypeptide
RDTSKNTL

SEQ ID NO: 212 - protein, 8 aa, Mus musculus
QVRRALDY

SEQ ID NO: 213 - protein, 20 aa, Mus musculus
GFIRDKANGYTTEYNPSVKG

SEQ ID NO: 214 - protein, 10 aa, Mus musculus
GFTVTAYYMS

SEQ ID NO: 215 - protein, 7 aa, Artificial Sequence
  Synthetic polypeptide
WAXXXXS

SEQ ID NO: 216 - protein, 10 aa, Artificial Sequence
  Synthetic polypeptide
XXXXXXXYXX

SEQ ID NO: 217 - protein, 10 aa, Artificial Sequence
  Synthetic polypeptide
XXXXXGAMDY

SEQ ID NO: 218 - protein, 10 aa, Artificial Sequence
  Synthetic polypeptide
XXXXXGXMDY

SEQ ID NO: 219 - protein, 10 aa, Artificial Sequence
  Synthetic polypeptide
NSNFYGAMDY

SEQ ID NO: 220 - protein, 16 aa, Artificial Sequence
  Synthetic polypeptide
RVCYNXLGVCAGGMDY

SEQ ID NO: 221 - protein, 16 aa, Artificial Sequence
  Synthetic polypeptide
RVCYNRLGVCAGGMDY

SEQ ID NO: 222 - protein, 10 aa, Artificial Sequence
  Synthetic polypeptide
GFTISSNSIH

SEQ ID NO: 223 - protein, 11 aa, Artificial Sequence
  Synthetic polypeptide
AWITPSDGNTD

SEQ ID NO: 224 - protein, 10 aa, Artificial Sequence
  Synthetic polypeptide
GFTISSSSIH

SEQ ID NO: 225 - protein, 11 aa, Artificial Sequence
  Synthetic polypeptide
AWVLPSVGFTD

SEQ ID NO: 226 - protein, 11 aa, Artificial Sequence
  Synthetic polypeptide
RASXXXXXXVA

SEQ ID NO: 227 - protein, 8 aa, Artificial Sequence
  Synthetic polypeptide
XXASXLXS

SEQ ID NO: 228 - protein, 9 aa, Artificial Sequence
  Synthetic polypeptide
QXSXXXPPT

SEQ ID NO: 229 - protein, 11 aa, Artificial Sequence
  Synthetic polypeptide
RASEDISTAVA

SEQ ID NO: 230 - protein, 8 aa, Artificial Sequence
  Synthetic polypeptide
YAASFLYS

SEQ ID NO: 231 - protein, 9 aa, Artificial Sequence
  Synthetic polypeptide
QQSQISPPT

SEQ ID NO: 232 - protein, 17 aa, Mus musculus
KSSQSLLYSSNQNNYLA

SEQ ID NO: 233 - protein, 7 aa, Mus musculus
WASTRES

SEQ ID NO: 234 - protein, 9 aa, Mus musculus
QQYYTYPYT

SEQ ID NO: 235 - protein, 10 aa, Mus musculus
TPHTYGAMDY

SEQ ID NO: 236 - protein, 17 aa, Mus musculus
KSSQSLLYSSNQNNYLA

(19) United States
(12) Patent Application Publication
(10) Pub. No.: US 2010/0280227 A1
(43) Pub. Date: Nov. 4, 2010

(54) POLYPEPTIDES THAT BIND BR3 AND USES THEREOF

(75) Inventors: CHRISTINE M. AMBROSE, READING, MA (US); MERCEDESZ BALAZS, HAYWARD, CA (US); LAURA DEFORGE, PACIFICA, CA (US); MARK S. DENNIS, SAN CARLOS, CA (US); GERMAINE FUH, PACIFICA, CA (US); STEPHEN D. HURST, SAN FRANCISCO, CA (US); CHINGWEI V. LEE, FOSTER CITY, CA (US); HENRY B. LOWMAN, EL GRANADA, CA (US); FLAVIUS MARTIN, HAYWARD, CA (US); GERALD R. NAKAMURA, SAN FRANCISCO, CA (US); DHAYA SESHASAYEE, UNION CITY, CA (US); MELISSA A. STAROVASNIK, SAN FRANCISCO, CA (US); JEFFREY S. THOMPSON, STONEHAM, MA (US)

Correspondence Address: CLARK & ELBING LLP, 101 FEDERAL STREET, BOSTON, MA, 02110, US

(73) Assignee: GENENTECH, INC., SOUTH SAN FRANCISCO, CA (US)

(21) Appl. No.: 12/835,570

(22) Filed: Jul. 13, 2010

Related U.S. Application Data
(63) Continuation of application No. 12/693,324, filed on Jan. 25, 2010, now abandoned.
Application No. 12/693,324 is a continuation of application No. 11/793,946, filed on Mar. 27, 2008, now abandoned.
(60) Provisional application No. 60/640,323, filed on Dec. 31, 2004.

(51) Int. Cl. C07K 16/18

(52) U.S. Cl. 530/387.3; 530/391.1; 530/391.7

(57) ABSTRACT

The present invention relates to novel BR3 binding antibodies and polypeptides, including antagonist and agonist polypeptides. The present invention also relates to the use of the BR3 binding antibodies and polypeptides in, e.g., methods of treatment, screening methods, diagnostic methods, assays and protein purification methods.

### CROSS REFERENCE TO RELATED APPLICATIONS

This application is a continuation of U.S. patent application Ser. No. 12/693,324, filed Jan. 25, 2010 which is a continuation of U.S. patent application Ser. No. 11/793,946, filed Mar. 27, 2008, which is a National Stage Application PCT/US2005/047072, filed Dec. 23, 2005, which claims the benefit of U.S. Provisional Application No. 60/640,323, filed Dec. 31, 2004.

### FIELD OF THE INVENTION

The invention relates to antibodies and polypeptides that bind BR3 and uses thereof.

### BACKGROUND OF THE INVENTION

BAFF (also known as BLyS, TALL-1, THANK, TNFSF13B, or zTNF4) is a member of the TNF ligand superfamily that is essential for B cell survival and maturation (reviewed in Mackay & Browning (2002) Nature Rev. Immunol. 2, 465-475). BAFF overexpression in transgenic mice leads to B cell hyperplasia and development of severe autoimmune disease (Mackay, et al. (1999) J. Exp. Med. 190, 1697-1710; Gross, et al. (2000) Nature 404, 995-999; Khare, et al. (2000) Proc. Natl. Acad. Sci. U.S.A. 97, 3370-33752-4). BAFF levels are elevated in human patients with a variety of autoimmune disorders, such as systemic lupus erythematosus, rheumatoid arthritis, Wegener's granulomatosis and Sjogren's syndrome (Cheema, G. S, et al., (2001) Arthritis Rheum. 44, 1313-1319; Groom, J., et al, (2002) J. Clin. Invest. 109, 59-68; Zhang, J., et al., (2001) J. Immuno. 166, 6-10; Krumbholz et al., ANCA Workshop, Prague, Czech Republic, 2003). Furthermore, BAFF levels correlate with disease severity, suggesting that BAFF may play a direct role in the pathogenesis of these illnesses. BAFF blockade in animal models of collagen-induced arthritis (CIA), lupus (e.g., BWF1 mice), multiple sclerosis (e.g., experimental autoimmune encephalomyelitis (EAE)) resulted in an alleviation of the disease. BR3:Fc treatment in a chronic graft-versus-host disease (cGVHD) model significantly inhibited splenomegaly associated with cGVHD, not by preventing B cell activation, but by inhibiting B cell survival (Kalled, S L et al. (2005) Curr Dir Autoimmun. 8:206-42). Thus, it is likely that BAFF blockade will provide efficacy in other animal models of autoimmunity with a strong B cell component.

In addition, there have been reports that both CD4⁺ and CD8⁺ T cells can be costimulated by recombinant BAFF to produce Type I and Type II cytokines and increase CD25 expression (Ng, L G, et al. 2004. J Immunol 173:807). Further, BAFF-R:Fc reportedly blocked BAFF-mediated human T cell proliferation (Huard, B, et al., (2000) J Immunol 167:6225). Still further, some patients with B-lymphoid malignancies have elevated levels of BAFF (Kern, C et al., (2004) Blood 103(2):679-88). According to one report, adding soluble BAFF or APRIL protected B-CLL cells against spontaneous and drug-induced apoptosis and stimulated NF-kappaB activation. Conversely, adding soluble BCMA-Fc or anti-BAFF and anti-APRIL antibodies enhanced B-CLL apoptosis (Kern, C et al., supra). BAFF may act as an essential autocrine survival factor for malignant B cells (Mackay F, et al., (2004) Curr Opin Pharmacol. 4(4):347-54). Thus, BAFF has been linked to a variety of disease states.

BAFF binds to three members of the TNF receptor superfamily, TACI, BCMA, and BR3 (also known as BAFF-R) (Gross, et al., supra; 8. Thompson, J. S., et al., (2001) Science 293, 2108-2111. Yan, M., et al.; (2001) Curr. Biol. 11, 1547-1552; Yan, M., et al., (2000) Nat. Immunol. 1, 37-41. Schiemann, B., et al., (2001) Science 293, 2111-2114). Of the three, only BR3 is specific for BAFF; the other two also bind the related TNF family member, APRIL. Comparison of the phenotypes of BAFF and receptor knockout or mutant mice indicates that signaling through BR3 mediates the B cell survival functions of BAFF (Thompson, et al., supra; Yan, (2002), supra; Schiemann, supra). In contrast, TACI appears to act as an inhibitory receptor (Yan, M., (2001) Nat. Immunol. 2, 638-643), while the role of BCMA is less clear (Schiemann, supra).

BR3 is a 184-residue type III transmembrane protein expressed on the surface of B cells (Thompson, et al., supra; Yan, (2002), supra). The intracellular region bears no sequence similarity to known structural domains or protein-protein interaction motifs. Several lines of investigation have provided strong evidence that BR3 is the primary receptor through which B cells receive a BAFF-mediated survival signal (reviewed in Kalled, S., et al., Curr Dir Autoimmun. 2005; 8:206-42). This has been confirmed by the recent generation of BAFF-R knockout mice wherein these BAFF-R^{-/-} mice (Shulga-Morkskaya, S. et al., (2004) J Immunol. 15; 173(4):2331-41). BR3 is expressed in a variety of disease tissue including multiple myeloma and non-Hodgkin's Lymphoma (Novak, A J (2004) Blood 104:2247-2253; Novak, A J (2004) Blood 103:689-694).

### SUMMARY OF THE INVENTION

The present invention provides novel BR3-binding polypeptides, including BR3 binding immunoadhesins, antibodies and peptides lacking an Fc region, and their unexpected and beneficial properties in the methods of this invention, including for example, their use as potent agents for depleting B cells, for stimulating B cell proliferation and survival, for therapeutic use or for diagnostic or research use.

The present invention provides BR3 binding polypeptides that comprise any one, any combination or all of the following properties: (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a mouse BR3 extracellular domain sequence with an apparent Kd value of 500 nM or less, 100 nM or less, 50 nM or less, 10 nM or less, 5 nM or less or 1 nM or less; (3) has a functional epitope on human BR3 comprising a specific residue(s); (4) inhibits the binding of human BR3 to human BAFF; (5) has antibody dependent cellular cytotoxicity (ADCC) in the presence of human effector cells or has increased ADCC in the presence of human effector cells compared to wild-type IgG or has decreased ADCC in the presence of human effector cells compared to wild-type IgG or native sequence IgG Fc; (6) is derived from any one of antibodies disclosed herein and (7) binds the human Fc neonatal receptor (FcRn) with a higher affinity than a polypeptide or parent polypeptide having wild type or native sequence IgG Fc; and (8) kills or depletes B cells in vitro or in vivo, preferably by at least 20% when compared to the baseline level or appropriate negative control which is not treated with such antibody. BR3 binding polypeptides include peptides that bind BR3 (e.g., derived from phage display) that are fused to Fc domains (e.g., peptibodies).

In one embodiment, compared to treatment with a control antibody that does not bind a B cell surface antigen or as compared to the baseline level before treatment, an antibody of this invention can deplete at least 20% of the B cells in any one, any combination or all of following population of cells in mice: (1) B cells in blood, (2) B cells in the lymph nodes, (3) follicular B cells in the spleen and (4) marginal zone B cells in the spleen. In other embodiments, B cell depletion is 25%, 30%, 40%, 50%, 60%, 70%, 80% or greater.

The present invention also provides agonistic BR3 binding polypeptides that comprise any one, any combination or all of the following properties: (1) binds to a human BR3 extracellular domain sequence with an apparent Kd value of 500 nM or less, 100 nM or less, 50 nM or less, 10 nM or less, 5 nM or less or 1 nM or less; (2) has a functional epitope on human BR3 specific residues; (3) stimulates B cell proliferation in vitro; (4) inhibits the binding of human BR3 to human BAFF; (5) is derived from any one of antibodies disclosed herein; (6) binds the human Fc neonatal receptor (FcRn) with a higher affinity than a polypeptide or parent polypeptide having wild type or native sequence IgG Fc and (7) stimulates B cell proliferation or B cell survival in vivo. According to one embodiment, the agonistic antibody has less or no ADCC function compared to a wild-type IgG1 or native IgG1 Fc sequence or the 9.1RF antibody. According to one embodiment, the agonistic antibody of this invention has at least the following substitutions D265A/N297A (EU numbering system) in the Fc region. According to one embodiment, the agonistic antibody has an IgG Fc sequence of human IgG4.

According to one embodiment, the BR3 binding polypeptides of this invention have a functional epitope on human BR3 comprising residues F25, V33 and A34, wherein the monoclonal antibody is not the 9.1 antibody or the 2.1 antibody. According to a further embodiment, the functional epitope further comprises residue R30. According to one embodiment, the BR3 binding polypeptides of this invention have a functional epitope on human BR3 comprising residues P21 and A22. According to one embodiment, the BR3 binding polypeptides of this invention have a functional epitope on human BR3 comprising residues L38 and R39, wherein the antibody is not the 9.1 antibody. According to one embodiment, the BR3 binding polypeptides have a functional epitope on human BR3 comprising residue G36, wherein the antibody is not the 2.1 antibody. According to one embodiment, the BR3 binding polypeptides of this invention have a functional epitope on human BR3 comprising residues V29 and L28. According to yet another embodiment, the functional epitope further comprises L28 and V29 According to one embodiment, the anti-BR3 antibody that has a functional epitope on human BR3 that comprises any one, any combination or all of L38, R39, P21 and A22 is an antagonistic BR3 binding antibody. According to another embodiment, the anti-BR3 antibody that has a functional epitope on human BR3 that comprises G36 is an agonistic BR3 binding antibody.

The present invention provides the antibodies of Table 2, BR3 binding antibodies derived from those antibodies and antibodies that bind BR3 and have an H1, H2, H3, L1, L2 or L3 regions with at least 70% homology to any one of the underlined portions of the antibodies sequences described in the Figures or to the CDRs or hypervariable regions described in the Sequence Listing. According to one embodiment, an antibody of this invention binds BR3 and has H1, H2 and H3 regions with at least 70% homology to the H1, H2 and H3 region, respectively, of any one of the antibodies of Table 2. According to one embodiment, an antibody of this invention binds BR3 and has L1, L2 and L3 regions with at least 70% homology to the L1, L2 and L3 region, respectively, of any one of the antibodies of Table 2. According to one embodiment, the antibodies bind BR3 and have a VH domain with at least 70% homology to a VH domain of any one of the antibodies of Table 2.

The present invention provides humanized anti-BR3 antibodies comprising an H3 hypervariable region (HVR3) comprising the residues QVRRALDY (SEQ ID NO:212). According to another embodiment, a BR3 binding antibody comprises: (1) an H3 hypervariable region (HVR3) comprising the residues QVRRALDY (SEQ ID NO:212); and (2) a heavy chain framework 3 region (HC-FR3) comprising the residues RDTSKNTF (SEQ ID NO:210). In one embodiment, the BR3 binding antibody further comprises an HVR1 comprising residues numbered 26-35 and an HVR2 comprising residues 49-65 (Kabat numbering) of an antibody sequence of any one of SEQ ID NOs: 35-36. In another embodiment, the anti-BR3 antibody further comprises residues GFTVTAYYMS (SEQ ID NO:214) in the H1 hypervariable region (HVR1) and residues GFIRDKANGYTTEYNPSVKG (SEQ ID NO: 213) in the H2 hypervariable region (HVR2). According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3.

According to another embodiment, an anti-BR3 binding antibody comprises: (1) an H3 hypervariable region (HVR3) comprising QVRRALDY (SEQ ID NO:212); and (2) a heavy chain framework 3 region (HC-FR3) comprising RDTSKNTL (SEQ ID NO:211). In one embodiment, the BR3 binding antibody comprises residues numbered 26-35 and 49-65 (Kabat numbering) of any one of the antibody sequences of SEQ ID NOs:37-73. According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3.

According to another embodiment, an anti-BR3 binding antibody comprises a L2 hypervariable region (LVR2) comprising Formula I:

| | |
|---|---|
| W-A-X3-X4-X5-X6-S (Formula I), | (SEQ ID NO: 215) |

wherein X3 is Q or S; X4 is H, I or T; X5 is L or R and X6 is D or E and wherein Formula I is not WASTRES (SEQ ID NO:233). According to one embodiment, the anti-BR3 antibody further comprises an H3 hypervariable region (HVR3) comprising QVRRALDY (SEQ ID NO:212). According to one embodiment, the LVR2 comprises residues numbered 50-56 (Kabat numbering) of the antibody sequence selected from the group consisting of SEQ ID NOs:23 and 25. According to one embodiment, the antibody further comprises residues GFTVTAYYMS (SEQ ID NO:214) in the HVR1 and residues GFIRDKANGYTTEYNPSVKG (SEQ ID NO:213) in the HVR2. According to one embodiment, the antibody further comprises residues KSSQSLLYSSNQNNYLA (SEQ ID NO:232) in the LVR1 and residues QQSQISPPT (SEQ ID NO:231) in the LVR3.

According to another embodiment, an anti-BR3 binding antibody comprises: a H1 hypervariable region (HVR1) comprising Formula II:

(SEQ ID NO: 216)
X1-X2-X3-X4-X5-X6-X7-Y-X9-X10 (Formula II),

wherein X1 is G or D, S, A, V, E or T; X2 is L, S, W, P, F, A, V, I, R, Y or D; X3 is P, T, A, N, S, I, K, L or Q; X4 is M, R, V, Y, G, E, A, T, L, W or D; X5 is A, S, T, G, I, R, P, N, D, Y or H; X6 is G, A, S, P or T; X7 is F, H, Y, R, S, V or N; X9 is T, I, M, F, W or V; X10 is T, G, S or A and wherein Formula II is not GFTVTAYYMS (SEQ ID NO:214). According to one embodiment, the antibody further comprises an H3 hypervariable region (HVR3) comprising QVRRALDY (SEQ ID NO:212). According to one embodiment, the HVR1 comprises residues numbered 26-35 (Kabat numbering) of the antibody sequence selected from the group consisting of SEQ ID NOs:24, 26-34, 36 and 38-73. According to one embodiment, the antibody further comprises residues WASTRES (SEQ ID NO:233) in the LVR2. According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3. According to one embodiment, the antibody further comprises residues GFIRDKANGYTTEYNPSVKG (SEQ ID NO:213) in the HVR2.

According to another embodiment, a BR3 binding antibody of this invention is an antibody that comprises: (1) an H3 hypervariable region (HVR3) comprising QVRRALDY (SEQ ID NO:212) and (2) residues numbered 50-56 of the LVR2 and residues numbered 26-35 of the HVR1 of an antibody selected from the group consisting of Hu9.1-73, Hu9.1-70, Hu9.1-56, Hu9.1-51, Hu9.1-59, Hu9.1-61, Hu9.1-A, Hu9.1-B and Hu9.1-C. According to one embodiment, the antibody further comprises residues GFIRDKANGYTTEYNPSVKG (SEQ ID NO:213) in the HVR2. According to one embodiment, the antibody further comprises residues KSSQSLLYSSNQNNYLA (SEQ ID NO:232) in the LVR1 and residues QQSQISPPT (SEQ ID NO:231) in the LVR3.

The present invention also provides anti-BR3 antibodies comprising an HVR3 comprising residues numbered 94-102 (Kabat numbering) of the antibody sequence selected from the group consisting of SEQ ID NOS:7-13 and 16-18. According to one embodiment, the antibody further comprises an HVR1 and HVR2 comprising residues 26-35 and residues 49-65 (Kabat numbering), respectively, of the antibody sequence of any one of SEQ ID NOS:7-13 and 16-18. According to one embodiment, the LVR1, LVR2 and LVR3 of the antibody comprises residues 24-34, residues 50-56 and residues 89-97 (Kabat numbering), respectively, of the antibody sequence of SEQ ID NO:3.

According to one embodiment, the anti-BR3 comprises a variable heavy chain domain comprising the variable heavy chain sequence of any one of SEQ ID NOs:22, 24 and 26-73. According to one embodiment, the anti-BR3 comprises a variable light chain domain comprising the variable light chain sequence of any one of SEQ ID NOs:21, 23 and 25. According to another embodiment, the antibody comprises the sequence of SEQ ID NO:74. According to another embodiment, the antibody comprises the sequence of SEQ ID NO:76, wherein X is A, W, H, Y, S or F. According to one specific embodiment, the antibody comprises the sequence of SEQ ID NO:75.

The present invention provides an anti-BR3 antibody comprising an HVR3 comprising Formula III:

(SEQ ID NO: 218)

X1-X2-X3-X4-X5-G-X7-MDY (Formula III),

wherein X1 is N, T or R; X2 is A, S, T, L, N or P; X3 is N, H or L; X4 is P, Y, F, N, T or L; X5 is Y, T or D; and X7 is A or E. According to one embodiment, Formula III is not TPHTYGAMDY (SEQ ID NO:235). According to one embodiment, Formula III is NSNFYGAMDY (SEQ ID NO:219). According to one embodiment, the antibody further comprises an HC-FR3 comprising residues RDTSKNTF (SEQ ID NO:210) or RDTSKNTL (SEQ ID NO:211). According to one embodiment, the LVR1, LVR2 and LVR3 of the antibody comprise residues 24-34, residues 50-56 and residues 89-97 (Kabat numbering), respectively, of the antibody sequence of SEQ ID NO:3. According to one embodiment, the HVR1 and HVR2 of the antibody comprise residues 26-35 and residues 49-65 (Kabat numbering), respectively, of the antibody sequence of SEQ ID NO:4.

Alternatively, the present invention provides an anti-BR3 antibody comprising an HVR3 comprising Formula III:

(SEQ ID NO: 218)

X1-X2-X3-X4-X5-G-X7-MDY (Formula III),

wherein X1 is N, T or R; X2 is A, S, T, L, N or P; X3 is N, H or L; X4 is P, Y, F, N, T or L; X5 is Y, T or D; and X7 is A or E and wherein the antibody further comprises an HC-FR3 comprising residues RDTSKNTF (SEQ ID NO:210) or RDTSKNTL (SEQ ID NO:211). According to one embodiment, when the HC-FR3 comprises RDTSKNTF (SEQ ID NO:210), then HVR3 of the antibody comprises residues 94-102 (Kabat numbering) of the antibody sequence of any one of SEQ ID NOs:6-9 and 16-17. According to one embodiment, when the HC-FR3 comprises RDTSKNTL (SEQ ID NO:211), then the HVR3 of the antibody comprises residues 94-102 (Kabat numbering) of the antibody sequence of any one of SEQ ID NOs:5 and 10-13. According to one embodiment, the LVR1, LVR2 and LVR3 of the antibody comprise residues 24-34, residues 50-56 and residues 89-97 (Kabat numbering), respectively, of the antibody sequence of SEQ ID NO:3. According to one embodiment, the HVR1 and HVR2 of the antibody comprise residues 26-35 and residues 49-65 (Kabat numbering) of the antibody sequence of SEQ ID NO:4, respectively.

In one embodiment, the sequence of Formula III is Formula IV:

| | |
|---|---|
| X1-X2-X3-X4-X5-GAMDY (Formula IV), | (SEQ ID NO: 218) |

wherein X1 is N, T or R; X2 is S, T, L, N or P; X3 is N or L; X4 is P, Y, F, N or L; X5 is Y or D.

According to one embodiment, the anti-BR3 antibody comprises an HVR3 comprising the sequence of Formula IV and a HC-FR3 comprising the sequence of SEQ ID NO:210. In a further embodiment, the antibody comprises the light chain sequence of SEQ ID NO:14. An a further embodiment, the antibody comprises an Fc region having D265A/N297A (EU numbering) mutations.

According to one embodiment, the anti-BR3 comprises a variable heavy chain domain comprising the variable heavy chain sequence of any one of SEQ ID NOs:4-13 and 16-18. According to one embodiment, the anti-BR3 comprises a variable light chain domain comprising the variable light chain sequence of SEQ ID NO:3. According to another embodiment, the antibody comprises the sequence of SEQ ID NO:14. According to another embodiment, the antibody comprises the sequence of SEQ ID NO:15.

The present invention provides an anti-BR3 antibody comprising the variable light chain sequence SEQ ID NO:77 and the variable heavy chain sequence SEQ ID NO:78, and variants thereof. According to one embodiment, an anti-BR3 antibody comprises the variable light chain sequence of SEQ ID NO:79. According to another embodiment, an anti-BR3 antibody comprises the variable heavy chain sequence of any one of SEQ ID NOs:80-85. According to one embodiment, an anti-BR3 antibody comprises an HVR1 comprising residues numbered 26-35 (Kabat numbering) of the antibody sequence of any one of SEQ ID NOs:80 or 82. According to one embodiment, an anti-BR3 antibody comprises an HVR2 comprising residues numbered 49-65 (Kabat numbering) of the antibody sequence of any one of SEQ ID NOs:80, 84 or 85. According to one embodiment, an anti-BR3 antibody comprises an HVR3 comprising residues numbered 94-102 (Kabat numbering) of the antibody sequence of any one of SEQ ID NOs:80, 82 or 83. In another embodiment, the anti-BR3 antibody comprises (1) an HVR3 comprising residues 94-102 (Kabat numbering) of the antibody sequence of any one of SEQ ID NOs: 81-85 and (2) a heavy chain framework 3 region (HC-FR3) comprising RDTSKNTF (SEQ ID NO:210). According to one embodiment, an anti-BR3 antibody comprises residues numbered 26-35, 49-65 and 94-102 of the antibody sequence of any one of SEQ ID NOs:80-85. According to one embodiment, the anti-BR3 antibody comprises an LVR1 comprising residues numbered 24-34 (Kabat numbering) of the antibody sequence SEQ ID NO:79. According to one embodiment, the anti-BR3 antibody comprises an LVR2 comprising residues numbered 50-56 (Kabat numbering) of the antibody sequence SEQ ID NO:79. According to one embodiment, the anti-BR3 antibody comprises an LVR3 comprising residues numbered 89-97 (Kabat numbering) of the antibody sequence SEQ ID NO:79. According to another embodiment, the LVR1, LVR2 and LVR3 of an anti-BR3 antibody comprises residues numbered 24-34, 50-56 and 89-97 (Kabat numbering), respectively, of SEQ ID NO:79.

According to one embodiment, the anti-BR3 comprises a variable heavy chain domain comprising the variable heavy chain sequence of any one of SEQ ID NOs78 and 80-85. According to one embodiment, the anti-BR3 comprises a variable light chain domain comprising the variable light chain sequence of SEQ ID NO:77 and 79.

The present invention provides is an anti-BR3 antibody comprising an HVR3 comprising residues numbered 95-102 of the antibody sequence of any one of SEQ ID NOs:87-94. The present invention provides an anti-BR3 antibody comprising an HVR2 comprising residues numbered 49-58 of the antibody sequence of any one of SEQ ID NOs87-94, 98, 100, 102, 104, 106, 107, 109-110, 112, 114, 116, 118, 120, 122, 124-127, 129 and 193. The present invention provides an anti-BR3 antibody comprising an HVR1 comprising residues numbered 24-34 of the antibody sequence of any one of SEQ ID NOs:87-94, 98, 100, 102, 104, 106, 107, 109-110, 112, 114, 116, 118, 120, 122, 124-127, 129 and 193. The present invention provides an anti-BR3 antibody comprising an LVR1 comprising residues numbered 24-34 of the antibody sequence of any one of SEQ ID NOs:86, 97, 99, 101, 103, 105, 108, 111, 113, 115, 117, 119, 121, 123, 128 and 194-207. The present invention provides an anti-BR3 antibody comprising an LVR2 comprising residues numbered 50-56 of the antibody sequence of any one of SEQ ID NOs:86, 97, 99, 101, 103, 105, 108, 111, 113, 115, 117, 119, 121, 123, 128 and 194-207. The present invention provides an anti-BR3 antibody comprising an LVR3 comprising residues numbered 89-97 of the antibody sequence of any one of SEQ ID NOs:86, 97, 99, 101, 103, 105, 108, 111, 113, 115, 117, 119, 121, 123, 128 and 194-207. According to one embodiment, the LVR1, LVR2 and LVR3 comprises residues numbered 24-34, 50-56 and 89-97 of the antibody sequence of any one of SEQ ID NOs:86, 97, 99, 101, 103, 105, 108, 111, 113, 115, 117, 119, 121, 123, 128 and 194-207. According to one embodiment, the HVR1, HVR2 and HVR3 comprises residues numbered 24-34, 49-58 and 95-102 of the antibody sequence of any one of SEQ ID NOs87-94, 98, 100, 102, 104, 106, 107, 109-110, 112, 114, 116, 118, 120, 122, 124-127, 129 and 193. In one embodiment, the anti-BR3 antibody comprises a variable heavy chain domain comprising the VH sequence of any one of SEQ ID NOs87-94, 98, 100, 102, 104, 106, 107, 109-110, 112, 114, 116, 118, 120, 122, 124-127, 129 and 193. In one embodiment, the anti-BR3 antibody comprises a variable light chain domain comprising the VL sequence of any one of SEQ ID NOs:86, 97, 99, 101, 103, 105, 108, 111, 113, 115, 117, 119, 121, 123, 128 and 194-207.

The present invention provides an anti-BR3 antibody comprising HVR3 comprising RVCYN-X6-LGVCAGGMDY (SEQ ID NO:220) (Formula V), wherein X6 is R or H.

The present invention provides an anti-BR3 antibody comprising an LVR1 comprising the Formula VI:

RAS-X4-X5-X6-X7-X8-X9-VA (Formula VI),

wherein X4 is Q or E; X5 is D or E; X6 is I or E; X7 is S or A, X8 is S or T and X9 is A or S.

The present invention provides an anti-BR3 antibody comprising an LVR2 comprising the Formula VII:

X1-X2-A-S—X5-L-X7-S (Formula VII),

### Wherein X1 is Y or F; X2 is S, A or G; X5 is N, F or Y; and X7 is F or Y.

The present invention provides an anti-BR3 antibody comprising an LVR3 comprising the Formula VIII:

Q-X2-S—X4-X5-X6-PPT (Formula VIII),

wherein X2 is Q or H; X4 is G, L, R, H, Y, Q or E; X5 is N, T, M, S, A, T, I or V; and X6 is T or S. According to one embodiment, the anti-BR3 antibody comprises a light chain comprising the sequences of Formula I, II and III. According to another embodiment, the anti-BR3 antibody comprises a light chain comprising the sequences of Formula I, II and III and comprises a HVR3 comprising the sequence of Formula V or SEQ ID NO:220.

The present invention provides anti-BR3 binding antibody comprises an H3 comprising RVCYNRLGVCAGGMDY (SEQ ID NO:221); an H1 comprising residues SGFTISSNSIH (SEQ ID NO:222) and an H2 comprising AWITPSDGNTD (SEQ ID NO: 223). In another embodiment, the anti-BR3 binding antibody comprises an H3 comprising RVCYNRLGVCAGGMDY (SEQ ID NO:221); an H1 comprising residues SGFTISSSSIH (SEQ ID NO:224) and an H2 comprising AWVLPSVGFTD (SEQ ID NO: 225).

According to one embodiment, the anti-BR3 comprises a variable heavy chain comprising the variable heavy chain sequence of any one of SEQ ID NOs87-96, 98, 100, 102, 104, 106, 107, 109-110, 112, 114, 116, 118, 120, 122, 124-127, 129 and 193. According to one embodiment, the anti-BR3 comprises a variable light chain comprising the variable light chain sequence of any one of SEQ ID NOs:86, 97, 99, 101, 103, 105, 108, 111, 113, 115, 117, 119, 121, 123, 128 and 194-207.

In one embodiment, the BR3 binding antibody can competitively inhibit the binding of an antibody produced by the hybridoma deposited as 3.1 (ATCC Deposit PTA-6622) or 12B12.1 (ATCC Deposit PTA-6624) to the human BR3 extracellular domain. In a further embodiment, the antibody comprises the variable region sequence of the antibody produced by the hybridoma deposited as 3.1 (ATCC Deposit PTA-6622) or 12B12.1 (ATCC Deposit PTA-6624) to the human BR3 extracellular domain. In another embodiment, the antibody comprises the hypervariable region sequence of the antibody produced by the hybridoma deposited as 3.1 (ATCC Deposit PTA-6622) or 12B12.1 (ATCC Deposit PTA-6624). In another embodiment, antibody is a humanized form of the antibody produced by the hybridoma deposited as 3.1 (ATCC Deposit PTA-6622) or 12B12.1 (ATCC Deposit PTA-6624).

In one embodiment, the BR3 binding antibody can competitively inhibit the binding of an antibody produced by the hybridoma deposited as 3.1 (ATCC Deposit PTA-6622) or 12B12.1 (ATCC Deposit PTA-6624) to human BR3. In a further embodiment, the antibody comprises the variable region sequence of the antibody produced by the hybridoma deposited as 3.1 (ATCC Deposit PTA-6622) or 12B12.1 (ATCC Deposit PTA-6624) to human BR3. In another embodiment, the antibody comprises the hypervariable region sequence of the antibody produced by the hybridoma deposited as 3.1 (ATCC Deposit PTA-6622) or 12B12.1 (ATCC Deposit PTA-6624). In another embodiment, antibody is a humanized form of the antibody produced by the hybridoma deposited as 3.1 (ATCC Deposit PTA-6622) or 12B12.1 (ATCC Deposit PTA-6624).

In one embodiment, the antibody of this invention binds to the same epitope as any one of the antibodies specifically described herein. In another embodiment, the antibody of this invention comprises the sequence of the deposited antibodies.

The present invention provides BR3 binding antibodies and immunoadhesins with altered Fc effector function, such as ADCC, CDC and FcRn binding. In one embodiment, antibodies and immunoadhesins with increased ADCC activity compared to a wild-type human IgG1 is contemplated. According to another embodiment, antibodies and immunoadhesins and other BR3 binding polypeptides with decreased ADCC activity compared to a wild-type human IgG1 is contemplated. According to yet another embodiment, antibodies and immunoadhesins with increased FcRn binding affinity compared to a wild-type human IgG1 is contemplated. According to one embodiment, the antibody or immunoadhesin has at least one substitution in the Fc region selected from the group consisting of: 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 and 439 of the Fc region, wherein the numbering of the residues in the Fc region is according to the EU numbering system. According to one embodiment, residue 434 is a residue selected from the group consisting of A, W, Y, F and H. According to another embodiment, the antibody or immunadhesin has the following substitutions S298A/E333A/K334A. According to another embodiment, the antibody or immunadhesin has the following substitution K322A. According to another embodiment, the antibody or immunadhesin comprises the sequence of SEQ ID NO:134, wherein X is any amino acid selected from the group consisting of A, W, H, Y and F. According to another embodiment, the antibody or immunadhesin has any one or any combination of the following substitutions K246H, H268D, E283L, S324G, S239D and I332E. According to yet another embodiment, an antibody or immunadhesin of this invention has at least the following substitutions D265A/N297A.

According to one embodiment of the invention, the BR3 binding polypeptide is conjugated to a cytotoxic agent or a chemotherapeutic agent.

According to another embodiment, the antibody is a monoclonal antibody. According to another embodiment, the antibody is a humanized antibody. According to another embodiment, the antibody is a human antibody. According to another embodiment, the antibody is a chimeric antibody. According to another embodiment, the antibody is selected from the group consisting of a Fab, Fab′, a F(ab)′₂, single-chain Fv (scFv), an Fv fragment; a diabody and a linear antibody. According to another embodiment, the antibody is a multi-specific antibody such as a bispecific antibody.

Also provided is a composition comprising an antibody or polypeptide of any one of the preceding embodiments, and a carrier. In one embodiment, the carrier is a pharmaceutically acceptable carrier. These compositions can be provided in an article of manufacture or a kit.

The invention also provided a liquid formulation comprising an anti-BR3 antibody in a histidine buffer. According to one embodiment, the buffer is a histidine sulfate buffer. According to another embodiment, a formulation or composition of this invention is packaged as a pre-filled syringe.

The invention also provides an isolated nucleic acid that encodes any of the antibody sequences disclosed herein, including an expression vector for expressing the antibody.

Another aspect of the invention are host cells comprising the preceding nucleic acids, and host cells that produce the antibody. In one preferred embodiment of the latter, the host cell is a CHO cell. A method of producing these antibodies is provided, the method comprising culturing the host cell that produces the antibody and recovering the antibody from the cell culture.

Yet another aspect of the invention is an article of manufacture comprising a container and a composition contained therein and a package insert, wherein the composition comprises an antibody of any of the preceding embodiments. According to one embodiment, the article of manufacture is a diagnostic kit comprising a BR3-binding antibody of this invention.

The invention also provides methods of treating the diseases disclosed herein by administration of a BR3 binding antibody, polypeptide or functional fragment thereof, to a mammal such as a human patient having a bone marrow transplant and a human patient suffering from the disease such as an autoimmune disease, a cancer, a B cell neoplasm, a BR3 positive cancer or an immunodeficiency disease. According to one preferred embodiment for treating an autoimmune disease, B cell neoplasm or a BR3 positive cancer, the BR3 binding polypeptide or antibody to be administered is preferably an antagonist BR3-binding antibody or polypeptide or is not an agonist BR3 binding antibody or polypeptide. According to one preferred embodiment for treating an immunodeficiency disease, the BR3 binding antibody or polypeptide to be used is an agonist BR3-binding antibody or polypeptide of this invention. According to one embodiment, the cancers to be treated according to this invention is selected from the group consisting of non-Hodgkin's lymphoma, chronic lymphocytic leukemia, multiple myeloma, (including follicular lymphoma, diffuse large B cell lymphoma, marginal zone lymphoma and mantle cell lymphoma).

In one embodiment of the methods for treating an autoimmune disease, cancer, B cell neoplasm or a BR3 positive cancer, the antibody is a BR3-binding antibody that has increased ability to bind FcRn at pH 6.0 compared to a 9.1RF antibody of this invention. In one embodiment of the methods for treating an autoimmune disease, B cell neoplasm or a BR3 positive cancer, the BR3 binding antibody is a BR3-binding antibody that has increased ADCC effector function in the presence of human effector cells compared to a 9.1RF antibody.

In one embodiment, the BR3 positive cancer is a B cell lymphoma or leukemia including non-Hodgkin's lymphoma (NHL) or lymphocyte predominant Hodgkin's disease (LPHD), chronic lymphocytic leukemia (CLL), acute lymphocytic leukemia (ALL) or small lymphocytic lymphoma (SLL). According to another embodiment, the BR3 positive cancer is multiple myeloma. In additional embodiments, the treatment method further comprises administering to the patient at least one chemotherapeutic agent, wherein for non-Hodgkin's lymphoma (NHL), the chemotherapeutic agent is selected from the group consisting of doxorubicin, cyclophosphamide, vincristine and prednisolone.

Also provided is a method of treating an autoimmune disease, comprising administering to a patient suffering from the autoimmune disease, a therapeutically effective amount of a BR3 binding antibody or polypeptide of this invention. According to one embodiment, the autoimmune disease is selected from the group consisting of rheumatoid arthritis, juvenile rheumatoid arthritis, lupus including systemic lupus erythematosus (SLE), Wegener's disease, inflammatory bowel disease including Crohn's Disease and ulcerative colitis, idiopathic thrombocytopenic purpura (ITP), thrombotic thrombocytopenic purpura (TTP), autoimmune thrombocytopenia, multiple sclerosis, psoriasis, Ig neuropathies including IgA nephropathy, IgM polyneuropathies, and IgG neuropathy, myasthenia gravis, vasculitis including ANCA-associated vasculitis, diabetes mellitus, Reynaud's syndrome, Sjorgen's syndrome, neuromyelitis optica (NMO), pemphigus including paraneoplastic pemphigus, pemphigus vulgaris and pemphigus foliaceus, polymyositis/dermatomyositis and glomerulonephritis. Where the autoimmune disease is rheumatoid arthritis, the antibody can be administered in conjunction with a second therapeutic agent. According to one embodiment, the second therapeutic agent is methotrexate.

In these treatment methods for autoimmune diseases, B cell neoplasms, BR3 positive cancers, the BR3 binding antibodies can be administered alone or in conjunction with a second therapeutic agent such as a second antibody, another B cell depleting agent, a chemotherapeutic agent, an immunosuppressive agent or another biologic that modulates human immune responses (e.g., a biologic response modifier). The second antibody can be one that binds CD20 or a different B cell antigen, or a NK or T cell antigen. In one embodiment, the anti-CD20 antibody is selected from the group consisting of rituximab (RITUXAN®), m2H7 (murine 2H7), hu2H7 (humanized 2H7) and all its functional variants, hu2H7.v16 (v stands for version), v31, v96, v114 and v115, (e.g., see, WO 2004/056312). In one embodiment, the second antibody is a radiolabeled anti-CD20 antibody. In other embodiments, the CD20 binding antibody is conjugated to a cytotoxic agent including a toxin or a radioactive isotope. In another embodiment, the second therapeutic agent is selected from the group consisting of an interleukin (e.g., IL-2, IL-12), an interferon, fludarabine, cyclophosphamide, an antibody that targets TNF-alpha (e.g., Enbrel®, Remicade®, and Humira®), a colony-stimulating factors (e.g., CSF, GM-CSF, G-CSF). In another embodiment, the second antibody or biologic can be another BAFF antagonist (e.g., a BR3 antibody, anti-BAFF antibody, TACI-Fc, BCMA-Fc and BR3-Fc). According to one embodiment, the BAFF antagonist that is being administered as a second therapeutic for autoimmune diseases or cancer does not have ADCC activity. In another embodiment, the second therapeutic is selected from the group consisting of an anti-VEGF antibody (e.x., the Avastin™ antibody), anti-CD64 antibody, an anti-C32a antibody, an anti-CD 16 antibody, anti-INFalpha antibody, anti-CD79a antibody, an anti-CD70b antibody, an anti-CD52 antibody, anti-CD40 antibody, CTLA4-Ig, anti-CD22 antibody, anti-CD23 antibody, anti-CD80 antibody, anti-HLA-DR antibody, anti-MHCII (IA) antibody, anti-IL-7 antibody, anti-IL-2 antibody, anti-IL-4 antibody, an anti-IL-21 antibody and anti-IL-10 antibody. Specific examples of B cell depletion agents include, but are not limited to, the aforementioned anti-CD20 antibodies, Alemtuzumab (anti-CD52 antibody), and Epratuzumab or CMC-544 (Wyeth) (anti-CD22 antibodies). In another embodiment, the second therapeutic is a small molecule that depletes B cells or an IAP inhibitor.

In another aspect, the invention provides a method of treating an autoimmune disease selected from the group consisting of Dermatomyositis, Wegner's granulomatosis, ANCA-associated vasculitis (AAV), Aplastic anemia, Autoimmune hemolytic anemia (AIHA), factor VIII deficiency, hemophilia A, Autoimmune neutropenia, Castleman's syndrome, Goodpasture's syndrome, solid organ transplant rejection, graft versus host disease (GVHD), IgM mediated, thrombotic thrombocytopenic purpura (TTP), Hashimoto's Thyroiditis, autoimmune hepatitis, lymphoid interstitial pneumonitis (LIP), bronchiolitis obliterans (non-transplant) vs. NSIP, Guillain-Barre Syndrome, large vessel vasculitis, giant cell (Takayasu's) arteritis, medium vessel vasculitis, Kawasaki's Disease, polyarteritis nodosa, comprising administering to a patient suffering from the disease, a therapeutically effective amount of a BR3 binding antibody.

The present invention also provides a method for treating an immunodeficiency disease in a mammal comprising the step of administering a therapeutically effective amount of an agonist BR3 binding antibody or polypeptide of this invention.

The present invention provides a method for isolating BR3 using the antibodies of the invention. The present invention also provides a method for screening inhibitors of B cell proliferation comprising the steps of: (a) stimulating the B cell with a BR3 agonist antibody; (b) administering a candidate compound; and (c) detecting BR3 activity such as B cell proliferation. The present invention also provides a method for identifying and monitoring downstream markers of BR3 pathway comprising the steps of: (a) stimulating the B cell with a BR3 agonist antibody and (b) detecting alterations in gene expression and/or protein activity of the cell.

The present invention also provides a method for diagnosing an autoimmune disease or a cancer to be treated with a BR3 binding therapy antagonist which comprises: (a) contacting a biological sample from a test subject with a BR3 binding antibody or polypeptide of this invention; (b) assaying the level of BR3 polypeptide in the biological sample; and (c) comparing the level of BR3 polypeptide in the biological sample in the biological sample with a standard level of BR3 protein; whereby the presence or an increase in the level of BR3 protein compared to the standard level of BR3 protein is indicative of an autoimmune disease or cancer to be treated with a BR3 binding therapy.

The present invention also provides a method of detecting BR3 polypeptide comprising the steps of binding the anti-BR3 antibody or immunoadhesin of this invention in a test sample or a subject and comparing the antibody or immunoadhesin bound compared to a control antibody or immunoadhesin. In one embodiment, the antibody or immunoadhesin is used in an assay selected from the group consisting of a FACS analysis, an immunohistochemistry assay (1HC) and an ELISA assay. Non-BAFF blocking anti-BR3 antibodies have the advantage of detecting BR3 whether it is bound to ligand or not and can be useful in measuring free and bound BR3.

### BRIEF DESCRIPTION OF THE DRAWINGS

FIG. 1 shows variable domain sequences of 2.1 grafted anti-BR3 antibody numbered according to the Kabat numbering system. Bolded letters indicate R71A, N73T, and L78A changes compared to human consensus III sequence. The underlined portions refer to regions comprising CDR sequence (H1, H2, H3, L1, L2 and L3).

FIG. 2 shows variable domain sequences of 9.1 grafted anti-BR3 antibody numbered according to the Kabat numbering system. Bolded letters indicate R71A, N73T, and L78A changes compared to human consensus III sequence. The underlined portions refer to regions comprising CDR sequence (H1, H2, H3, L1, L2 and L3).

FIG. 3 shows variable domain sequences of 11G9 grafted anti-BR3 antibody numbered according to the Kabat numbering system. The underlined portions refer to regions comprising CDR sequence (H1, H2, H3, L1, L2 and L3). Bolded letters indicate R71A, N73T, and L78A changes compared to human consensus III sequence.

FIG. 4 shows the results of soft randomizing the CDR regions of 9.1 grafted anti-BR3 antibody and selection. The variable domains of the listed antibodies are the same as the 9.1 grafted variable domain sequence except for the residues changes in the L2 and H1 regions shown.

FIG. 5 shows a comparison of the mouse VH framework region and the human “RF” and “RL” framework sequences.

FIG. 6 shows antigen binding by grafted Fabs with modified frameworks.

FIG. 7 shows selected sequences from the 2.1-RL and 2.1-RF CDR Repair libraries at round 5. The variable domains of the listed antibodies are the same as the 2.1-RF or 2.1-RL variable domain sequences except for the residues changes in the H3 regions shown.

FIG. 8 shows selected sequences from the 9.1-RL and 9.1-RF CDR Repair libraries at round 5. The variable domains of the listed antibodies are the same as the 9.1-RF or 9.1-RL variable domain sequences except for the residues changes in the H1 regions shown.

FIG. 9 shows selected sequences from the 11G9-RF CDR Repair library at round 5. The variable domains of the listed antibodies are the same as the 11G9-RF variable domain sequence except for the residues changes in the H1, H2 and H3 regions shown.

FIG. 10 shows a BIAcore analysis of selected anti-BR3 humanized MAbs.

FIG. 11 shows the results of a solution binding competition ELISA for selected F(ab)′2 phage clones bound in solution with increasing amounts of (A) a polypeptide having the mouse BR3 ECD or (B) human BR3 ECD.

FIG. 12 shows amino acid sequences from phage-derived anti-BR3 antibodies numbered according to the Kabat numbering system. “LN” refers to the number of residues between and including residues numbered 95-102. “#” refers the number of times the clone was selected during screening. “Clone” refers to the assigned phage clone number. Residues 151, P52a, G55, T57 of CDR-H2 not shown. The remaining residues comprising each antibody (1-23, 35-49, 57-88 and 98-107) are as described for V3 in FIG. 15. “X” indicates that the sequence is unknown.

FIG. 13 shows the IC50 values of selected F(ab)′₂ phage using solution binding competition ELISA and percentage of F(ab)′2 phage bound to the extracellular domain of mBR3 or hBR3 in the presence of BAFF.

FIG. 14 shows an ELISA assay that shows the inhibition of F(ab)′2 phage binding to mBR3-Fc coated wells in the presence of increased BAFF concentrations.

FIG. 15 shows variable domain sequences of phage-derived V3 anti-BR3 antibody numbered according to the Kabat numbering system.

FIG. 16 shows (A) sequences from V3-derived clones and (B) the IC50 values of the F(ab)′₂ phage and blocked binding to BR3 with hybrid mBAFF. Residues 51(A), 52(S) and 54(L) of the LC-CDR2 not shown.

FIG. 17 shows residues from the V3-1 derived clones and their IC50 values.

FIG. 18 shows affinity improved V3-46s phage clones and their phage IC50 values for binding to mouse and human BR3. Amino acids shown are residues numbered 27-32 (“L1”), 49-55 (“L2”) and 88-94 (“L3”) of SEQ ID NOS: 194-207 according to the Kabat numbering system.

FIG. 19 shows competitive and direct binding of anti-BR3 mAbs to BJAB Cells. (A) BAFF Competitive Binding Assay. (B) Direct Binding Assay. Isotype controls showed no binding, and the detection antibody bound equivalently to mouse IgG1, IgG2a, and IgG2b.

FIG. 20 shows the results of competitive and direct binding assays with V3-1m and B9C11 binding to BJAB Cells (Human BR3) (panels A and B, respectively) and BHK Cells (Murine BR3) (panels C and D, respectively).

FIG. 21 shows competition ELISAs for anti-human BR3 mAb characterization. The mAbs were incubated at the indicated concentrations with a constant amount of biotinylated mAb 9.1 (panel A), 2.1 (panel B), 11G9 (panel C), or 1E9 (panel D).

FIG. 22 shows the competitive binding of V3-1m, B9C11, and P1B8 to Murine BR3. Competition ELISAs were performed using biotinylated V3-1m (panel A) and biotinylated B9C11 (panel B).

FIG. 23 shows antibodies 2.1, 11G9 and 9.1 inhibit the proliferation of B cells from two different donors (panels A and B, respectively).

FIG. 24 shows antibody V3-1m inhibits the proliferation of B cells stimulated by: (A) anti-IgM (5 ug/ml) plus BAFF (2 ng/ml) or (B) anti-IgM (5 ug/ml) plus BAFF (10 ng/ml).

FIG. 25 shows that 9.1-RF blocks BAFF-dependent human B cell proliferation and does not agonize. (A) Human primary B cells treated with anti-IgM+BAFF+9.1-RF. (B) Human primary B cells treated with anti-IgM+9.1-RF.

FIG. 26 shows that 2.1-46 stimulates B cell proliferation. (A) Cells treated with anti-IgM+BAFF+2.1-46. (B) Cells treated with anti-IgM+2.1-46.

FIG. 27 shows a schematic of various points of interaction between BR3 and antibodies 11G9, 2.1, 9.1 and V3-1 based on shotgun ala-scanning results. The circled residues indicate potential sites of O-linked glycosylation.

FIG. 28 shows B cell populations in the peripheral blood of a chronic lymphocytic leukemia (CLL) patient using antibodies against B cell markers. Panels A, C and D show FACS analyses using anti-CD 19 and either anti-CD27 antibodies, anti-CD20 antibodies or anti-CD5 antibodies. Panel B is a histogram showing BR3 expression in malignant populations. The boxes indicate the malignant populations.

FIG. 29 shows the results of an ADCC activity assay with humanized anti-BR3 antibodies and (A) BJAB cells, (B) Ramos cells or (C) WIL2s cells.

FIG. 30 shows a flow cytometry analysis of mouse B cells in the blood (panels A-C), lymph nodes (panels D-F) and spleen (panels G-I) after 7 days of treatment with V3-1, BR3-Fc or a control antibody.

FIG. 31 shows (A) the absolute number of mouse B cells contained in 1 ml of blood; (B) the % of B cells in lymph nodes; (C) the absolute numbers of follicular B cells (FO—CD21+CD23+) or (D) marginal zone B cells (MZ—CD21high CD23low) in the spleen at days 1, 3, 7 and 15 post-treatment with V3-1, BR3-Fc or a control antibody.

FIG. 32 shows B cell populations in mice at day 15 after treatment with a control antibody, BR3-Fc or V3-1. (A-1 to A-6) FACS analysis of B cell populations in the spleen or Peyer's Patches of mice after treatment; (B) histogram of plasmablasts in the spleen after treatment; and (C) histogram of germinal center cells in Peyer's Patches after treatment.

FIG. 33 shows the reduction of B cells in the blood (panel A) and the spleen (panel B) in BALB/c mice at day 6 post-treatment using anti-BR3 antibody having ADCC activity and BAFF blocking ability, a non-blocking anti-BR3 antibody, an Fc-defective mutant anti-BR3 antibody or BR3-Fc.

FIG. 34 shows the results of treating NZBxW F1 mice (lupus nephritis model) with anti-BR3 antibody, mV3-1, mBR3-Fc and control antibody. (A) shows the reduction in time to progression of anti-BR3 antibody treated mice and BR3-Fc treated mice compared to control mice. (B) shows numbers of B cells per ml of blood in mice treated with BR3-Fc (p<0.01), control (p<0.03) and mV3-1 (p<0.001). (C) shows the number of total B cells per spleen of mice treated with BR3-Fc, control and mCB1 (p<0.00001). The horizontal lines in (B) and (C) indicate the mean level of the group. Data is expressed as individual mouse data points (n=25).

FIG. 35 shows B cell depletion in SCID model mice treated with human PBMC and antiBR3 antibodies or mBR3-Fc as indicated (day 4). (A) percentage of activated/GC B cells (CD19hi/CD38int), (B) number of activated/GC B cells, (C) percentage of plasmablasts (CD19lo/CD38hi/CD139neg), (D) number of plasmablasts and (E) percentage of activated/GC cells (CD19hi/CD38+).

FIG. 36 shows the binding of 9.1RF (panel A), 9.1RF N434A (panel B) and 9.1RF N434W (panel C) antibodies to human or cyno FcRn at equilibrium (pH 6.0 and pH 7.4). R_{eq} is the number of response units from the chip at equilibrium.

FIG. 37 shows ELISA assays with Fc gamma receptor binding to anti-BR3 antibodies or the Herceptin® antibody (positive control). Panel A: FcγRI. Panel B: FcγRIIA. Panel C:FcγRIIB. Panel D: FcγRIII (F158). Panel E: FcγRIII (V158).

FIG. 38 shows an analysis of B cell levels post treatment with anti-BR3 antibodies (V3-1) versus anti-CD20 antibodies (2H7) in the blood (panel A) and lymph nodes (panel B) at 1 hour, 1 Day, 8 days or 15 days.

FIG. 39 shows an analysis of B cell levels post treatment with anti-BR3 antibodies versus anti-CD20 antibodies in the follicular B cells (panel A) and marginal zone B cells (panel B) at 1 day, 8 days and 15 days.

FIG. 40 shows B cell depletion in blood (panel A) and tissue (panel B) from cyno monkeys treated with 9.1RF. Data is from ATA-monkeys (5 cynos treated with 20 mg/kg; 3 cynos treated with 2 mg/kg).

FIG. 41 shows the levels of B cell populations in the blood of cyno monkeys treated with 9.1RF or 9.1RF N434W over time: (A) CD20+/CD21+ cells, (B) CD21+/CD27+ cells and (C) CD21+/CD27-cells.

### DETAILED DESCRIPTION OF THE INVENTION

The terms “BAFF,” “BAFF polypeptide,” “TALL-1” or “TALL-1 polypeptide,” “BLyS” when used herein encompass “native sequence BAFF polypeptides” and “BAFF variants”. “BAFF” is a designation given to those polypeptides which are encoded by any one of the amino acid sequences of SEQ ID NO:143 or SEQ ID NO:144 and homologs and fragments and variants thereof, which have the biological activity of the native sequence BAFF. A biological activity of BAFF can be selected from the group consisting of promoting B cell survival, promoting B cell maturation and binding to BR3, BCMA or TACI. Variants of BAFF will preferably have at least 80% or any successive integer up to 100% including, more preferably, at least 90%, and even more preferably, at least 95% amino acid sequence identity with a native sequence of a BAFF polypeptide. A “native sequence” BAFF polypeptide comprises a polypeptide having the same amino acid sequence as the corresponding BAFF polypeptide derived from nature. For example, BAFF, exists in a soluble form following cleavage from the cell surface by furin-type proteases. Such native sequence BAFF polypeptides can be isolated from nature or can be produced by recombinant and/or synthetic means. The term “native sequence BAFF polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “BAFF” includes those polypeptides described in Shu et al., J. Leukocyte Biol., 65:680 (1999); GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., Science, 285:260-263 (1999); Schneider et al., J. Exp. Med., 189:1747-1756 (1999); Mukhopadhyay et al., J. Biol. Chem., 274:15978-15981 (1999).

The term “BAFF antagonist” as used herein is used in the broadest sense, and includes any molecule that (1) binds a native sequence BAFF polypeptide or binds a native sequence BR3 polypeptide to partially or fully block BR3 interaction with BAFF polypeptide, and (2) partially or fully blocks, inhibits, or neutralizes native sequence BAFF signaling. Native sequence BAFF polypeptide signaling promotes, among other things, B cell survival and B cell maturation. The inhibition, blockage or neutralization of BAFF signaling results in, among other things, a reduction in the number of B cells. A BAFF antagonist according to this invention will partially or fully block, inhibit, or neutralize one or more biological activities of a BAFF polypeptide, in vitro or in vivo. In one embodiment, a biologically active BAFF potentiates any one or any combination of the following events in vitro or in vivo: an increased survival of B cells, an increased level of IgG and/or IgM production, or stimulated B cell proliferation.

The term “TACI antagonist” as used herein is used in the broadest sense, and includes any molecule that (1) binds a native sequence BAFF polypeptide or binds a native sequence TACI polypeptide to partially or fully block TACI interaction with BAFF polypeptide, and (2) partially or fully blocks, inhibits, or neutralizes native sequence BAFF signaling.

The term “BCMA antagonist” as used herein is used in the broadest sense, and includes any molecule that (1) binds a native sequence BAFF polypeptide or binds a native sequence BCMA polypeptide to partially or fully block BCMA interaction with BAFF polypeptide, and (2) partially or fully blocks, inhibits, or neutralizes native sequence BAFF signaling.

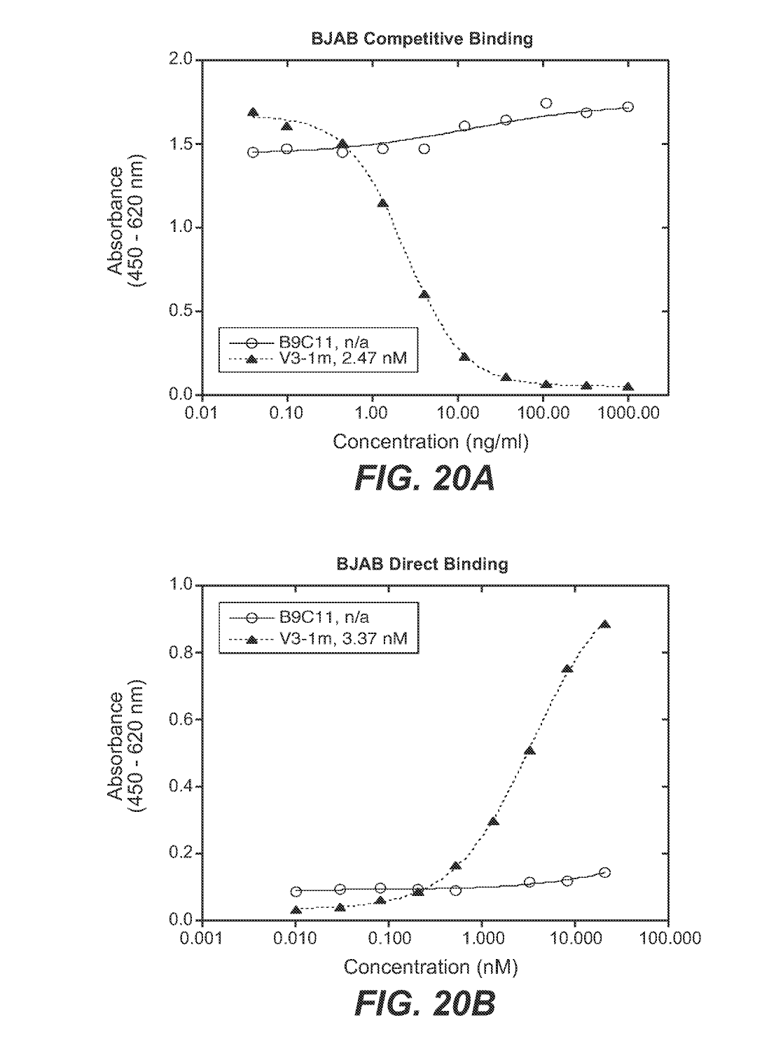

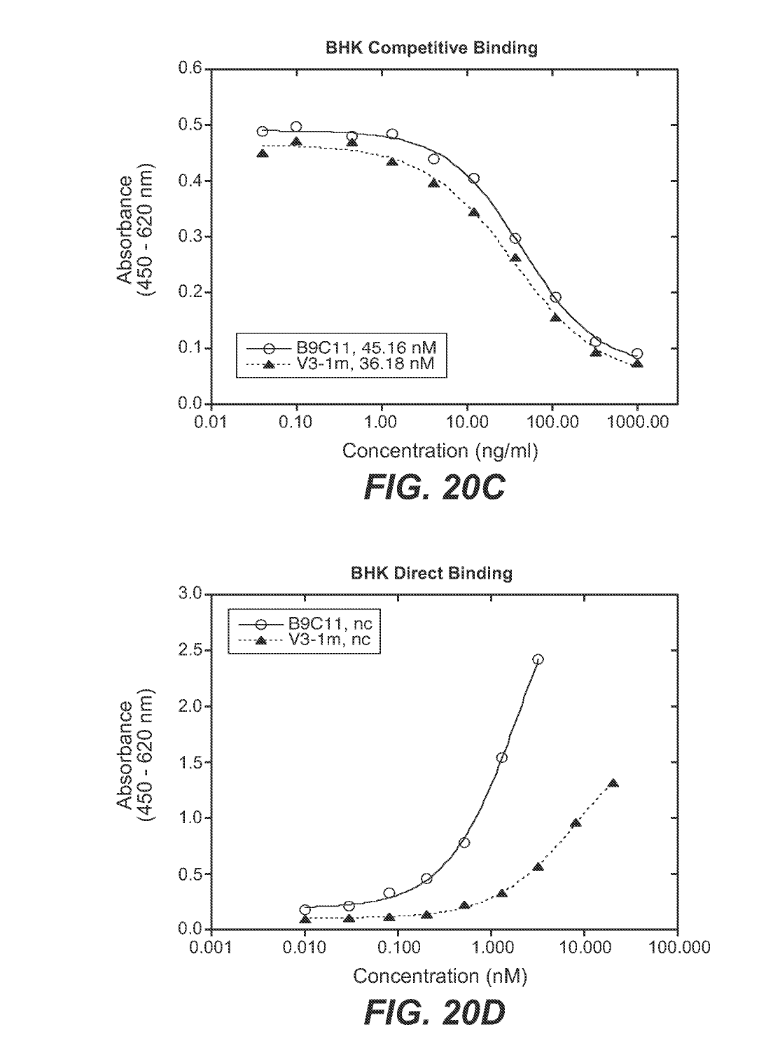

As mentioned above, a BAFF antagonist can function in a direct or indirect manner to partially or fully block, inhibit or neutralize BAFF signaling, in vitro or in vivo. For instance, the BAFF antagonist can directly bind BAFF. For example, anti-BAFF antibodies that bind within a region of human BAFF comprising residues 162-275 and/or a neighboring residue of a residue selected from the group consisting of 162, 163, 206, 211, 231, 233, 264 and 265 of human BAFF such that the antibody sterically hinders BAFF binding to BR3 is contemplated. In another example, a direct binder is a polypeptide comprising the extracellular domain of a BAFF receptor such as TACI, BR3 and BCMA, or comprising the boxed minimal region of the ECDs (corresponding to residues 19-35 of human BR3). Alternatively, the BAFF antagonist can bind an extracellular domain of a native sequence BR3 at its BAFF binding region to partially or fully block, inhibit or neutralize BAFF binding to BR3 in vitro, in situ, or in vivo. For example, such indirect antagonist is an anti-BR3 antibody that binds in a region of BR3 comprising residues 23-38 of human BR3 or a neighboring region of those residues such that binding of human BR3 to BAFF is sterically hindered. Other examples of BAFF binding Fc proteins that can be BAFF antagonists can be found in WO 02/66516, WO 00/40716, WO 01/87979, WO 03/024991, WO 02/16412, WO 02/38766, WO 02/092620 and WO 01/12812. BAFF antagonists include BAFF-binding sequences listed in FIG. 20 of WO 02/24909 and those described in WO 2003/024991, WO 02/092620, fragments of those sequences that bind BAFF, and fusion proteins comprising those sequences (e.g., Fc fusion proteins).

The terms “BR3”, “BR3 polypeptide” or “BR3 receptor” when used herein encompass “native sequence BR3 polypeptides” and “BR3 variants” (which are further defined herein). “BR3” is a designation given to those polypeptides comprising any one of SEQ ID NOs:145-149 and variants or fragments thereof. The BR3 polypeptides of the invention can be isolated from a variety of sources, such as from human tissue types or from another source, or prepared by recombinant and/or synthetic methods. The term BR3, includes the BR3 polypeptides described in WO 02/24909 and WO 03/14294.

A “native sequence” BR3 polypeptide comprises a polypeptide having the same amino acid sequence as the corresponding BR3 polypeptide derived from nature. Such native sequence BR3 polypeptides can be isolated from nature or can be produced by recombinant and/or synthetic means. The term “native sequence BR3 polypeptide” specifically encompasses naturally-occurring truncated, soluble or secreted forms (e.g., an extracellular domain sequence), naturally-occurring variant forms (e.g., alternatively spliced forms) and naturally-occurring allelic variants of the polypeptide. The BR3 polypeptides of the invention include the BR3 polypeptide comprising or consisting of the contiguous sequence of amino acid residues 1 to 184 of a human BR3.

A BR3 “extracellular domain” or “ECD” refers to a form of the BR3 polypeptide which is essentially free of the transmembrane and cytoplasmic domains. ECD forms of BR3 include those comprising any one of amino acids 1 to 77, 2 to 62, 2-71, 1-61, 8-71, 17-42, 19-35 or 2-63 of BR3.

“BR3 variant” means a BR3 polypeptide having at least about 60% amino acid sequence identity with the residues 19-35 of BR3 ECD and binds a native sequence BAFF polypeptide. See Gordon, N. C., et al., (2003) Biochemistry 42:5977-5983) Optionally, the BR3 variant includes a single cysteine rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native sequence BAFF polypeptide are also contemplated. According to an embodiment, a BR3 variant polypeptide will have at least about 65% amino acid sequence identity, at least about 70% amino acid sequence identity, at least about 75% amino acid sequence identity, at least about 80% amino acid sequence identity, at least about 80% amino acid sequence identity, at least about 85% amino acid sequence identity, at least about 90% amino acid sequence identity, at least about 95% amino acid sequence identity, at least about 98% amino acid sequence identity or at least about 99% amino acid sequence identity in that portion corresponding to residues 19-35 of human BR3.

Of residues human BR3 polypeptide or a specified fragment thereof, BR3 variant polypeptides do not encompass the native BR3 polypeptide sequence. Ordinarily, BR3 variant polypeptides are at least about 17 amino acids in length, or more.

The term “antibody” is used in the broadest sense and specifically covers, for example, monoclonal antibodies, polyclonal antibodies, antibodies with polyepitopic specificity, single chain antibodies, multi-specific antibodies and fragments of antibodies. According to some embodiments, a polypeptide of this invention is fused into an antibody framework, for example, in the variable domain or in a CDR such that the antibody can bind to and inhibit BAFF binding to BR3 or BAFF signaling. The antibodies comprising a polypeptide of this invention can be chimeric, humanized, or human. The antibodies comprising a polypeptide of this invention can be an antibody fragment. Such antibodies and methods of generating them are described in more detail below. Alternatively, an antibody of this invention can be produced by immunizing an animal with a polypeptide of this invention. Thus, an antibody directed against a polypeptide of this invention is contemplated.

As used herein, the terms “anti-BR3” and “BR3 binding” are used interchangeably and indicate that the antibody or polypeptide binds a BR3 polypeptide. Preferrably, the anti-BR3 antibody binds to an epitope on a BR3 polypeptide having an amino acid sequence selected from the group consisting of SEQ ID NO:145-149 and does not bind to human TACI or human BCMA. Preferably, the anti-BR3 antibody binds a human BR3 extracellular domain sequence with an apparent Kd value of 500 nM or less, 100 nM or less, 50 nM or less, 10 nM or less, 5 nM or less or 1 nM or less as a Fab in a BIAcore Assay at 25° C. According to one embodiment, the antibody or polypeptide binds to BR3 with an apparent Kd between 0.001 pM and 500 nM.

“Antagonistic anti-BR3 antibodies” according to this invention refer to antibodies that bind a BR3 polypeptide and inhibit BR3 signalling (e.g, inhibit BR3 related B cell proliferation, B cell survival or both B cell proliferation and survival).

“Agonistic anti-BR3 antibodies” according to this invention refer to antibodies that bind a BR3 polypeptide and stimulate BR3 signalling (e.g., BR3-related B cell proliferation, B cell survival or both B cell proliferation and survival).

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

CD20 binding antibody and anti-CD20 antibody are used interchangeably herein and encompass all antibodies that bind CD20 with sufficient affinity such that the antibody is useful as a therapeutic agent in targeting a cell expressing the antigen, and do not significantly cross-react with other proteins such as a negative control protein in the assays described below. Bispecific antibodies wherein one arm of the antibody binds CD20 are also contemplated. Also encompassed by this definition of CD20 binding antibody are functional fragments of the preceding antibodies. The CD20 binding antibody will bind CD20 with a Kd of <10 nM. In preferred embodiments, the binding is at a Kd of <7.5 nM, more preferably <5 nM, even more preferably at between 1-5 nM, most preferably, <1 nM.

Examples of antibodies which bind the CD20 antigen include: “C2B8” which is now called “Rituximab” (“RITUXAN®”) (U.S. Pat. No. 5,736,137, expressly incorporated herein by reference); the yttrium-[90]-labeled 2B8 murine antibody designated “Y2B8” or “Ibritumomab Tiuxetan” ZEVALIN® (U.S. Pat. No. 5,736,137, expressly incorporated herein by reference); murine IgG2a “B1,” also called “Tositumomab,” (Beckman Coulter) optionally labeled with ¹³¹I to generate the “131I-B1” antibody (iodine I131 tositumomab, BEXXAR™) (U.S. Pat. No. 5,595,721, expressly incorporated herein by reference); murine monoclonal antibody “1F5” (Press et al. Blood 69(2):584-591 (1987) and variants thereof including “framework patched” or humanized 1F5 (WO03/002607, Leung, S.); ATCC deposit HB-96450); murine 2H7 and chimeric 2H7 antibody (U.S. Pat. No. 5,677,180, expressly incorporated herein by reference); humanized 2H7; huMax-CD20 (Genmab, Denmark); AME-133 (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

The terms “rituximab” or “RITUXAN®” herein refer to the genetically engineered chimeric murine/human monoclonal antibody directed against the CD20 antigen and designated “C2B8” in U.S. Pat. No. 5,736,137 expressly incorporated herein by reference, including fragments thereof which retain the ability to bind CD20.

In a specific embodiment, the anti-CD20 antibodies bind human and primate CD20. In specific embodiments, the antibodies that bind CD20 are humanized or chimeric. CD20 binding antibodies include rituximab (RITUXAN®), m2H7 (murine 2H7), hu2H7 (humanized 2H7) and all its functional variants, including without limitation, hu2H7.v16 (v stands for version), v31, v73, v75, as well as fucose deficient variants, and other 2H7 variants described in WO2004/056312. Unless indicated, the sequences disclosed herein of the humanized 2H7v.16 and variants thereof are of the mature polypeptide, i.e., without the leader sequence.

Patents and patent publications concerning CD20 antibodies include U.S. Pat. Nos. 5,776,456, 5,736,137, 5,843,439, 6,399,061, and 6,682,734, as well as US patent appln nos. US 2002/0197255A1, US 2003/0021781A1, US 2003/0082172 A1, US 2003/0095963 A1, US 2003/0147885 A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US2001/0018041A1, US2003/0180292A1, WO01/34194 (Hanna and Hariharan); US appln no. US2002/0006404 and WO02/04021 (Hanna and Hariharan); US appln no. US2002/0012665 A1 and WO01/74388 (Hanna, N.); US appln no. US 2002/0058029 A1 (Hanna, N.); US appln no. US 2003/0103971 A1 (Hariharan and Hanna); US appln no. US2002/0009444A1, and WO01/80884 (Grillo-Lopez, A.); WO01/97858 (White, C.); US appln no. US2002/0128488A1 and WO02/34790 (Reff, M.); WO02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US appln no. US 2002/0004587A1 and WO01/77342 (Miller and Presta); US appln no. US2002/0197256 (Grewal, I.); US Appln no. US 2003/0157108 A1 (Presta, L.); U.S. Pat. Nos. 6,565,827B1, 6,090,365B1, 6,287,537B1, 6,015,542, 5,843,398, and 5,595,721, (Kaminski et al.); U.S. Pat. Nos. 5,500,362, 5,677,180, 5,721,108, 6,120,767, 6,652,852B1 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); US Appl No. US 2003/0133930 A1 and WO00/74718 (Goldenberg and Hansen); WO00/76542 (Golay et al.); WO01/72333 (Wolin and Rosenblatt); U.S. Pat. No. 6,368,596B1 (Ghetie et al.); U.S. Pat. No. 6,306,393 and US Appln no. US2002/0041847 A1, (Goldenberg, D.); US Appln no. US2003/0026801A1 (Weiner and Hartmann); WO02/102312 (Engleman, E.); US Patent Application No. 2003/0068664 (Albitar et al.); WO03/002607 (Leung, S.); WO 03/049694, US2002/0009427A1, and US 2003/0185796 A1 (Wolin et al.); WO03/061694 (Sing and Siegall); US 2003/0219818 A1 (Bohen et al.); US 2003/0219433 A1 and WO 03/068821 (Hansen et al.); US2003/0219818A1 (Bohen et al.); US2002/0136719A1 (Shenoy et al.); WO2004/032828 (Wahl et al.), each of which is expressly incorporated herein by reference. See, also, U.S. Pat. No. 5,849,898 and EP appln no. 330,191 (Seed et al.); U.S. Pat. No. 4,861,579 and EP332,865A2 (Meyer and Weiss); U.S. Pat. No. 4,861,579 (Meyer et al.); WO95/03770 (Bhat et al.); US 2003/0219433 A1 (Hansen et al.).

The CD20 antibodies can be naked antibody or conjugated to a cytotoxic compound such as a radioisotope, or a toxin. Such antibodies include the antibody Zevalin™ which is linked to the radioisotope, Yttrium-90 (IDEC Pharmaceuticals, San Diego, Calif.), and Bexxar™ which is conjugated to 1-131 (Corixa, Wash.). The humanized 2H7 variants include those that have amino acid substitutions in the FR and affinity maturation variants with changes in the grafted CDRs. The substituted amino acids in the CDR or FR are not limited to those present in the donor or acceptor antibody. In other embodiments, the anti-CD20 antibodies of the invention further comprise changes in amino acid residues in the Fc region that lead to improved effector function including enhanced CDC and/or ADCC function and B-cell killing (also referred to herein as B-cell depletion). In particular, three mutations have been identified for improving CDC and ADCC activity: S298A/E333A/K334A (also referred to herein as a triple Ala mutant or variant; numbering in the Fc region is according to the EU numbering system; Kabat et al., supra) as described (Idusogie et al., supra (2001); Shields et al., supra).

Other anti-CD20 antibodies of the invention include those having specific changes that improve stability. In one embodiment, the chimeric anti-CD20 antibody has murine V regions and human C region. One such specific chimeric anti-CD20 antibody is Rituxan® (Rituximab®; Genentech, Inc.). Rituximab and hu2H7 can mediate lysis of B-cells through both complement-dependent cytotoxicity (CDC) and antibody-dependent cellular cytotoxicity (ADCC). Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Inhibitors of Apoptosis (IAP) refers to a family of proteins that inhibit apoptosis (Deveraux, et al., (1999) Genes Dev 13(3):239-252). Examples of IAPs includes melanoma IAP (ML-IAP) and human X-chromosome linked IAP (XIAP) cellular IAP 1 (cIAP-1), and cellular IAP 2 (cIAP-2), which inhibit caspase 3, caspase 7 and caspase 9 activity (Deveraux et al., J Clin Immunol (1999), 19:388-398; Deveraux et al., (1998) EMBO J. 17, 2215-2223; Vucic et al., (2000) Current Bio 10:1359-1366).

Examples of inhibitors of LAP (IAP inhibitors) includes antisense oligonucleotides directed against XIAP, cIAP-1, cIAP-2 or ML-IAP, Smac/DIABLO-derived peptides or other molecules that block the interaction between IAPs and their caspases, and molecules that inhibit IAP-mediated suppression of caspase activity (Sasaki et al, Cancer Res., 2000, 60(20):5659; Lin et al, Biochem J., 2001, 353:299; Hu et al, Clin. Cancer Res., 2003, 9(7):2826; Arnt et al, J. Biol. Chem., 2002, 277(46):44236; Fulda et al, Nature Med., 2002, 8(8):808; Guo et al, Blood, 2002, 99(9):3419; Vucic et al, J. Biol. Chem., 2002, 277(14):12275; Yang et al, Cancer Res., 2003, 63(4):831); WO 2005/097791, WO 2005/094818, US 2005/0197403 and U.S. Pat. No. 6,673,917).

A “B cell surface marker” or “B cell surface antigen” herein is an antigen expressed on the surface of a B cell which can be targeted with an antagonist which binds thereto. Exemplary B cell surface markers include, but are not limited to, CD10, CD19, CD20, CD21, CD22, CD23, CD24, CD37, CD40, CD52, D53, CD72, CD73, CD74, CDw75, CDw76, CD77, CDw78, CD79a, CD79b, CD80, CD81, CD82, CD83, CDw84, CD85, CD86, CD180 (RP105), FcRH2 (IRTA4), CD79A, C79B, CR2, CCR6, CD72, P2×5, HLA-DOB, CXCR5 (BLR1), FCER2, BR3 (aka BAFF-R), TACI, BTLA, NAG14 (aka LRRC4), SLGC16270 (ala LOC283663), FcRH1 (IRTA5), FcRH5 (IRTA2), ATWD578 (aka MGC15619), FcRH3 (IRTA3), FcRH4 (IRTA1), FcRH6 (aka LOC343413) and BCMA (aka TNFRSF17), HLA-DO, HLA-Dr10 and MHC Class II.

According to a preferred embodiment, the antibodies of this invention do not include the 9.1 antibody and the 2.1 antibody deposited and described in WO 02/24909.

According to one preferred embodiment, the “apparent Kd” or “apparent Kd value” as used herein is in one preferred embodiment is measured by surface plasmon resonance such as by performing a BIAcore® assay. In one preferred embodiment, an apparent Kd value for a BR3-binding antibody of this invention is measured by performing surface plasmon resonance wherein either a BR3 ECD is immobilized on a sensor chip and an anti-BR3 antibody in Fab form is flowed over the BR3 ECD-immobilized chip or an anti-BR3 antibody in IgG form is immobilized on a sensor chip and a BR3 ECD is flowed over the IgG-immobilized sensor chip, e.g., as described in Example 8 herein. According to one preferred embodiment, the sensor chips are immobilized with protein such that there is approximately 10 response units (RU) of coupled protein on a chip. In another preferred embodiment, an apparent Kd value for an FcRn-binding antibody of this invention is measured by performing surface plasmon resonance wherein a FcRn polypeptide is immobilized to a sensor chip and an antibody is flowed over the chip, e.g., as described in Example 16.

A “functional epitope” according to this invention refers to amino acid residues of an antigen that contribute energetically to the binding of an antibody. Mutation of any one of the energetically contributing residues of the antigen (for example, mutation of wild-type BR3 by alanine or homolog mutation) will disrupt the binding of the antibody to the antigen. In one preferred embodiment of this invention, a residue that is comprised within the functional epitope on an anti-BR3 antibody can be determined by shot-gun alanine scanning using phage displaying ala mutants of BR3 or a portion thereof (e.g, the extracellular domain or residues 17-42 if desired region of study). According to one preferred embodiment, the functional epitope is determined according to the procedure described in Example 9.

The term “variable” refers to the fact that certain segments of the variable domains differ extensively in sequence among antibodies. The V regions mediate antigen binding and define specificity of a particular antibody for its particular antigen. However, the variability is not evenly distributed across the 110-amino acid span of the variable domains. Instead, the V domains consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

Hypervariable regions may comprise “extended hypervariable regions” as follows: 24-36 or 24-34 (L1), 46-56 or 50-56 (L2) and 89-97 (L3) in the VL and 26-35 (H1), 50-65 or 49-65 (H2) and 93-102, 94-102 or 95-102 (H3) in the VH. The variable domain residues are numbered according to Kabat et al., supra for each of these definitions.

“Framework” or “FR” residues are those variable domain residues other than the hypervariable region residues as herein defined. For example, light chain framework 1 (LC-FR1), framework 2 (LC-FR2), framework 3 (LC-FR3) and framework 4 (LC-FR4) region comprise residues numbered 1-23, 35-49, 57-88 and 98-107 of an antibody (Kabat numbering system), respectively. In another example, heavy chain framework 1 (HC-FR1), heavy chain framework 2 (HC-FR2), heavy chain framework 3 (HC-FR3) and heavy chain framework 4 (HC-FR4) comprise residues 1-25, 36-48, 66-92 and 103-113, respectively, of an antibody (Kabat numbering system).

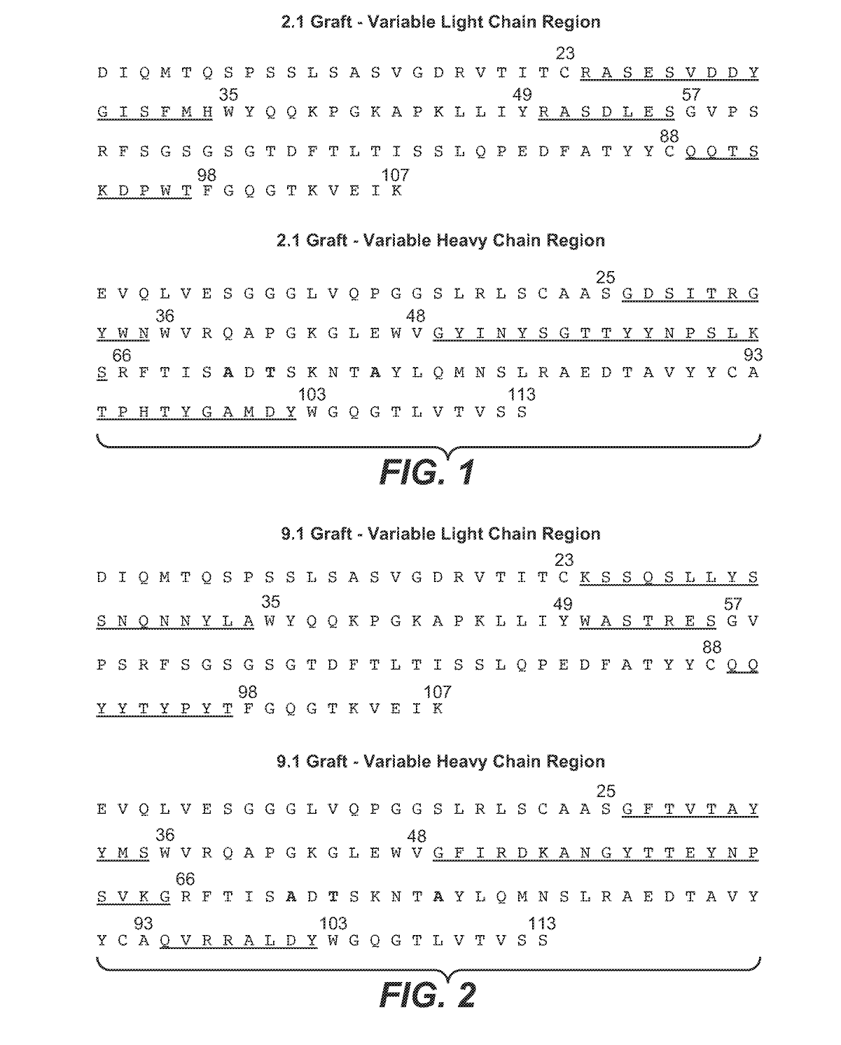

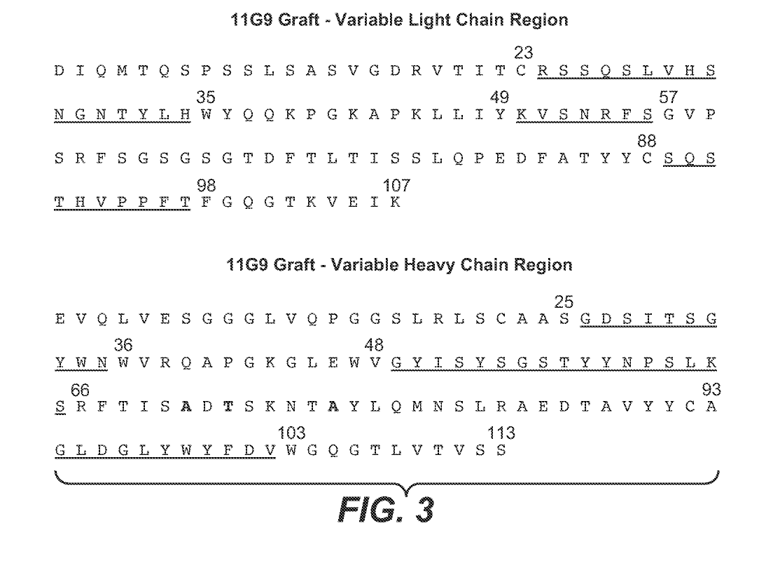

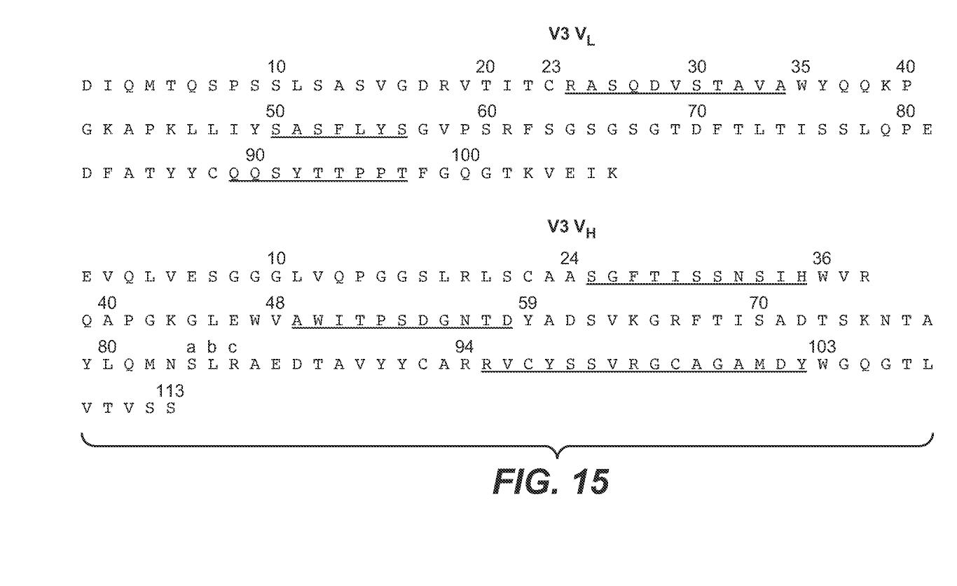

According to one embodiment, the residues corresponding to the majority of the residues in the CDR regions of the light chain of antibodies derived from the 9.1, 2.1, and 11G9 antibodies are underlined in FIGS. 1-3. According to another embodiment, the residues corresponding to the majority of the residues of the CDR regions of the heavy and the light chain of antibodies derived from the V3 antibodies are underlined in FIG. 15.

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human and the human H chain subgroup III V domains were prepared. The consensus V sequence does not have any known antibody binding specificity or affinity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34): 12467-12472 (2004); and Lee et al. J. Immunol. Methods 284(1-2): 119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while portions of the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. In some embodiments, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are generally made to further refine and maximize antibody performance. Typically, the humanized antibody will comprise substantially all of at least one variable domain, in which all or substantially all of the hypervariable loops derived from a non-human immunoglobulin and all or substantially all of the FR regions are derived from a human immunoglobulin sequence although the FR regions may include one or more amino acid substitutions to, e.g., improve binding affinity. In some embodiments, the number of these amino acid substitutions in the FR are typically no more than 6 in the H chain, and in the L chain, no more than 3. In one preferred embodiment, the humanized antibody will also comprise at least a portion of an immunoglobulin constant region (Fc), typically that of a human immunoglobulin or a human consensus constant sequence. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by, e.g., immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991). See also, Lonberg and Huszar, Int. Rev. Immunol. 13:65-93 (1995). PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598.

“Antibody fragments” comprise a portion of a full length antibody, generally the antigen binding or variable region thereof. Examples of antibody fragments include Fab, Fab′, F(ab′)2, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Functional fragments” of the BR3 binding antibodies of the invention are those fragments that retain binding to BR3 with substantially the same affinity as the intact full chain molecule from which they are derived and are active in at least one assay selected from the group consisting of depletion of B cells, inhibition of B cell proliferation or inhibition of BAFF binding to BR3 as measured by in vitro or in vivo assays such as those described herein.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation. A “native sequence Fc region” comprises an amino acid sequence identical to the amino acid sequence of an Fc region found in nature. Examples of Fc sequences are described in SEQ ID NOs:. 133, 135-141. and include a native sequence human IgG1 Fc region (non-A and A allotypes, SEQ ID NO:133 and 135, respectively); native sequence human IgG2 Fc region (SEQ ID NO:136); native sequence human IgG3 Fc region (SEQ ID NO:137); and native sequence human IgG4 Fc region (SEQ ID NO:138) as well as naturally occurring variants thereof. Examples of native sequence murine Fc regions are described in SEQ ID NOs:139-142 (IgG1, IgG2a, IgG2b, IgG3, respectively).

A “variant Fc region” comprises an amino acid sequence which differs from that of a native sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. In one embodiment, the variant Fc region herein will possess at least about 80% homology, at least about 85% homology, at least about 90% homology, at least about 95% homology or at least about 99% homology with a native sequence Fc region (e.g., SEQ ID NO: 133). According to another embodiment, the variant Fc region herein will possess at least about 80% homology, at least about 85% homology, at least about 90% homology, at least about 95% homology or at least about 99% homology with an Fc region of a parent polypeptide.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The term “Fc region-comprising polypeptide” refers to a polypeptide, such as an antibody or immunoadhesin (see definitions below), which comprises an Fc region. The C-terminal lysine (residue 447 according to the EU numbering system) of the Fc region may be removed, for example, during purification of the polypeptide or by recombinantly engineering the nucleic acid encoding the polypeptide. Accordingly, a composition comprising polypeptides, including antibodies, having an Fc region according to this invention can comprise polypeptides populations with all K447 residues removed, polypeptide populations with no K447 residues removed or polypeptide populations having a mixture of polypeptides with and without the K447 residue.

Throughout the present specification and claims, the Kabat numbering system is generally used when referring to a residue in the variable domain (approximately, residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

The terms “Fc receptor” or “FcR” are used to describe a receptor that binds to the Fc region of an antibody. In one embodiment, an FcR of this invention is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

WO00/42072 (Presta) and Shields et al. J. Biol. Chem. 9(2): 6591-6604 (2001) describe antibody variants with improved or diminished binding to FcRs. The contents of those publications are specifically incorporated herein by reference.

The “CH1 domain” of a human IgG Fc region (also referred to as “C1” of “H1” domain) usually extends from about amino acid 118 to about amino acid 215 (EU numbering system).

“Hinge region” is generally defined as stretching from Glu216 to Pro230 of human IgG1 (Burton, Molec. Immunol. 1.22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. In previous reports, FcR binding was generally attributed to amino acid residues in the lower hinge region of an IgG Fc region.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” (also referred to as “C2” or “H3” domain) comprises the stretch of residues C-terminal to a CH2 domain in an Fc region (i.e. from about amino acid residue 341 to the C-terminal end of an antibody sequence, typically at amino acid residue 446 or 447 of an IgG)

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “binding domain” refers to the region of a polypeptide that binds to another molecule. In the case of an FcR, the binding domain can comprise a portion of a polypeptide chain thereof (e.g. the alpha chain thereof) which is responsible for binding an Fc region. One useful binding domain is the extracellular domain of an FcR alpha chain.

A polypeptide with a variant IgG Fc with “altered” FcR binding affinity or ADCC activity is one which has either enhanced or diminished FcR binding activity (e.g, FcγR or FcRn) and/or ADCC activity compared to a parent polypeptide or to a polypeptide comprising a native sequence Fc region.

The variant Fc which “exhibits increased binding” to an FcR binds at least one FcR with higher affinity (e.g., lower apparent Kd or IC50 value) than the parent polypeptide or a native sequence IgG Fc. According to some embodiments, the improvement in binding compared to a parent polypeptide is about 3 fold, preferably about 5, 10, 25, 50, 60, 100, 150, 200, up to 500 fold, or about 25% to 1000% improvement in binding. The polypeptide variant which “exhibits decreased binding” to an FcR, binds at least one FcR with lower affinity (e.g, higher apparent Kd or higher IC50 value) than a parent polypeptide. The decrease in binding compared to a parent polypeptide may be about 40% or more decrease in binding. In one embodiment, Fc variants which display decreased binding to an FcR possess little or no appreciable binding to an FcR, e.g., 0-20% binding to the FcR compared to a native sequence IgG Fc region, e.g. as determined in the Examples herein.

“Antibody-dependent cell-mediated cytotoxicity” or “ADCC” refers to a form of cytotoxicity in which secreted Ig bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or in the Examples below may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using the in vitro ADCC assay as herein disclosed, but other assays or methods for determining ADCC activity, e.g. in an animal model etc, are contemplated. In one embodiment, the preferred variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Human effector cells” are leukocytes which express one or more FcRs and perform effector functions. According to one embodiment, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

Methods of measuring binding to FcRn are known (see, e.g., Ghetie 1997, Hinton 2004) as well as described in the Examples below. Binding to human FcRn in vivo and serum half life of human FcRn high affinity binding polypeptides can be assayed, e.g, in transgenic mice or transfected human cell lines expressing human FcRn, or in primates administered with the Fc variant polypeptides. In one embodiment, the polypeptide and specifically the antibodies of the invention having a variant IgG Fc exhibits increased binding affinity for human FcRn over a polypeptide having wild-type IgG Fc, by at least 2 fold, at least 5 fold, at least 10 fold, at least 50 fold, at least 60 fold, at least 70 fold, at least 80 fold, at least 100 fold, at least 125 fold, at least 150 fold. In a specific embodiment, the binding affinity for human FcRn is increased about 170 fold.

For binding affinity to FcRn, in one embodiment, the EC50 or apparent Kd (at pH 6.0) of the polypeptide is less than 1 uM, more preferably less than or equal to 100 nM, more preferably less than or equal to 10 nM. In one embodiment, for increased binding affinity to FcγRIII (F158; i.e. low-affinity isotype) the EC50 or apparent Kd less is than or equal to 10 nM, and for FcγRIII (V158; high-affinity isotype) the EC50 or apparent Kd is less than or equal to 3 nM. According to another embodiment, a reduction in binding of an antibody to a Fc receptor relative to a control antibody (e.g., the Herceptin® antibody) may be considered significant relative to the control antibody if the ratio of the values of the absorbances at the midpoints of the test antibody and control antibody binding curves (e.g, A_{450 nm(antibody)}/A_{450 nm(control Ab)}) is less than or equal to 40%. According to another embodiment, an increase in binding of an antibody to a Fc receptor relative to a control antibody (e.g., the Herceptin® antibody) may be considered significant relative to the control antibody if the ratio of the values of the absorbances at the midpoints of the test antibody and control antibody binding curves (e.g, A_{450 nm(antibody)}/A_{450 nm(control Ab)}) is greater than or equal to 125%. See, e.g., Example 16.

A “parent polypeptide” or “parent antibody” is a polypeptide or antibody comprising an amino acid sequence from which the variant polypeptide or antibody arose and against which the variant polypeptide or antibody is being compared. Typically the parent polypeptide or parent antibody lacks one or more of the Fc region modifications disclosed herein and differs in effector function compared to a polypeptide variant as herein disclosed. The parent polypeptide may comprise a native sequence Fc region or an Fc region with pre-existing amino acid sequence modifications (such as additions, deletions and/or substitutions).

A “fusion protein” and a “fusion polypeptide” refer to a polypeptide having two portions of a polypeptide sequence covalently linked together. In most embodiments, each of the portions are polypeptide sequences not typically associated with each other in nature and/or have different properties. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions will be linked in reading frame with each other.

An “isolated” antibody or polypeptide is one which has been identified and separated and/or recovered from a component of the environment from which it was produced. Contaminant components can be, e.g., materials which would interfere with diagnostic or therapeutic uses for the antibody, and may include enzymes, hormones, and other proteinaceous or nonproteinaceous solutes. In one preferred embodiment, the antibody or polypeptide will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody or polypeptide includes the antibody or polypeptide in situ within recombinant cells since at least one component of the antibody's natural environment will not be present. Ordinarily, however, isolated antibody or polypeptide will be prepared by at least one purification step.

An “isolated” polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

The cell that produces a BR3 binding antibody of the invention will include the bacterial and eukaryotic host cells into which nucleic acid encoding the antibodies have been introduced. Suitable host cells are disclosed below.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 C; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42 C, with a 10 minute wash at 42 C in 0.2×SSC (sodium chloride/sodium citrate) followed by a 10 minute high-stringency wash consisting of 0.1×SSC containing EDTA at 55 C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50 C. The skilled artisan will recognize how to adjust the temperature, ionic strength, etc. as necessary to accommodate factors such as probe length and the like.

The term “epitope tagged” when used herein refers to a chimeric polypeptide comprising a polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues). Polypeptides and antibodies of this invention that are epitope-tagged are contemplated.

“Biologically active” and “biological activity” and “biological characteristics” with respect to an anti-BR3 polypeptide or antibody of this invention means the antibody or polypeptide binds BR3. According to one preferred embodiment, the antibody binds human BR3 polypeptide.

In a further embodiment, an anti-BR3 polypeptide or antibody of this invention also has any one, any combination or all of the following activities: (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a rodent BR3 extracellular domain sequence with an apparent Kd value of 500 nM or less, 100 nM or less, 50 nM or less, 10 nM or less, 5 nM or less or 1 nM or less; and (3) inhibits human BR3 binding to human BAFF. Depending on the desired use for the antibody, the antibody can further comprise the any one of the following activities (1) has antibody dependent cellular cytotoxicity (ADCC) in the presence of human effector cells compared to wild-type or native sequence IgG Fc; (2) has increased ADCC in the presence of human effector cells compared to wild-type or native sequence IgG Fc or (3) has decreased ADCC in the presence of human effector cells compared to wild-type or native sequence IgG Fc. According to another embodiment, an antibody of this invention binds the human Fc neonatal receptor (FcRn) with a higher affinity than a polypeptide or parent polypeptide having wild type or native sequence IgG Fc.

“Biologically active” and “biological activity” and “biological characteristics” with respect to an antagonist anti-BR3 polypeptide or antibody of this invention means the antibody or polypeptide has any one, any combination or all of the following activities: (1) inhibits B cell proliferation; (2) inhibits B cell survival; (3) kills or depletes B cells in vivo. According to one embodiment, the depletion of B cells when compared to the baseline level or appropriate negative control which is not treated with such anti-BR3 antibody or polypeptide is at least 20%. According to another embodiment, the antagonistic antibody has antibody dependent cellular cytotoxicity (ADCC) in the presence of human effector cells compared to wild-type or native sequence IgG Fc or has increased ADCC in the presence of human effector cells compared to wild-type or native sequence IgG Fc.

“Biologically active” and “biological activity” and “biological characteristics” with respect to an agonist anti-BR3 polypeptide or antibody of this invention means the antibody or polypeptide has one or both of the following activities: (1) stimulates B cell proliferation and (2) stimulates B cell survival. According to one embodiment, the agonistic antibody has decreased ADCC in the presence of human effector cells compared to wild-type or native sequence IgG Fc.

The amino acid sequences specifically disclosed herein are contiguous amino acid sequences unless otherwise specified.

Variations in polypeptides of this invention described herein, can be made, for example, using any of the techniques and guidelines for conservative and non-conservative mutations. Variations can be a substitution, deletion or insertion of one or more codons encoding the polypeptide that results in a change in the amino acid sequence of the polypeptide. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature native sequence.

The term “conservative” amino acid substitution as used within this invention is meant to refer to amino acid substitutions which substitute functionally equivalent amino acids. Conservative amino acid changes result in minimal change in the amino acid structure or function of the resulting peptide. For example, one or more amino acids of a similar polarity act as functional equivalents and result in a silent alteration within the amino acid sequence of the peptide. In general, substitutions within a group can be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain can constitute a critical feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

Conservative substitutions include the following specific substitutions based on the similarities in side chains and exemplary substitutions and preferred substitutions listed below. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Non-conservative substitutions will entail exchanging a member of one of these classes for another class. Such substituted residues also may be introduced into the conservative substitution sites or, more preferably, into the remaining (non-conserved) sites.

The term “amino acid” within the scope of the present invention is used in its broadest sense and is meant to include the naturally occurring L alpha-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio (The Peptides: Analysis, Synthesis, Biology,) Eds. Gross and Meiehofer, Vol. 5 p 341, Academic Press, Inc, N.Y. 1983, which is incorporated herein by reference.

Peptides synthesized by the standard solid phase synthesis techniques described here, for example, are not limited to amino acids encoded by genes for substitutions involving the amino acids. Commonly encountered amino acids which are not encoded by the genetic code, include, for example, those described in International Publication No. WO 90/01940, as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparigine (EtAsn) for Asn, and Gln; Hydroxyllysine (Hyl) for Lys; allohydroxyllysine (AHyl) for Lys; 3-(and 4)hydroxyproline (3Hyp, 4Hyp) for Pro, Ser, and Thr; allo-isoleucine (AIle) for Ile, Leu, and Val; -amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn or Or) for Lys, Arg and His; Citrulline (Cit) and methionine sulfoxide (MSO) for Thr, Asn and Gln; -methylphenylalanine (MePhe), trimethylphenylalanine, halo (F, Cl, Br, and I)phenylalanine, triflourylphenylalanine, for Phe.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol, 150:1 (1976)]. If alanine substitution does not yield adequate amounts of variant, an isoteric amino acid can be used.

The term “detecting” is intended to include determining the presence or absence of a molecule or determining qualitatively or quantitatively the amount of a molecule. The term thus refers to the use of the materials, compositions, and methods of the present invention for qualitative and quantitative determinations. In general, the particular technique used for detection is not critical for practice of the invention.

For example, “detecting” according to the invention may include detecting: the presence or absence of a molecule, number of cells expressing the polypeptide, a change in the levels of the molecule or amount of the molecule bound to a target or target bound to the molecule; a change in biological function/activity of a molecule (e.g., ligand or receptor binding activity, intracellular signaling (such as NF-kB activation), tumor cell proliferation, B cell proliferation, or survival, etc.), e.g., using methods that are known in the art. In some embodiments, “detecting” may include detecting wild type levels of the molecule (e.g., mRNA or polypeptide levels). Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, or more e.g., 100-fold. Thus, for example, referral to a BR3 molecule can refer to its mRNA or protein, etc.

As used herein a “BR3 molecule” as used herein refers to a molecule substantially identical to: a BR3 polypeptide; a nucleic acid molecule encoding a BR3 polypeptide; as well as isoforms, fragments, analogs, or variants of the polypeptide or the nucleic acid molecule. For example, a BR3 molecule can include an isoform, fragment, analog, or variant of a BR3 polypeptide derived from a mammal, which BR3 molecule has the ability to bind BAFF.

As used herein a “BAFF molecule” as used herein refers to a molecule substantially identical to: a BAFF polypeptide; a nucleic acid molecule encoding a BAFF polypeptide; as well as isoforms, fragments, analogs, or variants of the polypeptide or the nucleic acid molecule. For example, a BAFF molecule can include an isoform, fragment, analog, or variant of a BAFF polypeptide derived from a mammal, which BAFF molecule that has the ability to bind BR3.

As used herein, a subject to be treated is a mammal (e.g., human, non-human primate, rat, mouse, cow, horse, pig, sheep, goat, dog, cat, etc.). The subject may be a clinical patient, a clinical trial volunteer, an experimental animal, etc. The subject may be suspected of having or at risk for having a cancer or immune disease, be diagnosed with a cancer or immune disease, or be a control subject that is confirmed to not have a cancer. Many diagnostic methods for cancer and immune disease and the clinical delineation of cancer or immune diagnoses are known in the art. According to one preferred embodiment, the subject to be treated according to this invention is a human.

“Treating” or “treatment” or “alleviation” refers to measures, wherein the object is to prevent or slow down (lessen) the targeted pathologic condition or disorder or relieve some of the symptoms of the disorder. Those in need of treatment include can include those already with the disorder as well as those prone to have the disorder or those in whom the disorder is to be prevented. A subject or mammal is successfully “treated” for a cancer if, after receiving a therapeutic amount of a polypeptide or an antibody of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the polypeptides of this invention can prevent growth and/or kill existing cancer cells, it can be cytostatic and/or cytotoxic. Reduction of these signs or symptoms may also be felt by the patient.

The term “therapeutically effective amount” refers to an amount of a polypeptide of this invention effective to “alleviate” or “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM. For example, useful immunoadhesins according to this invention can be polypeptides that complise the BAFF binding portions of a polypeptide or BR3 binding portions of a polypeptide (e.g., a portion of a BAFF receptor excluding the transmembrane or cytoplasmic sequences fused to an Fc region, TACI receptor extracellular domain-Fc or BCMA extracellular domain-Fc or BR3 extracellular domain-Fc). In one embodiment, a polypeptide sequence of this invention is fused to a constant domain of an immunoglobulin sequence.

An “immunodeficiency disease” is a disorder or condition where the immune response is reduced (e.g., severe combined immunodeficiency (SCID)-X linked, SCID-autosomal, adenosine deaminase deficiency (ADA deficiency), X-linked agammaglobulinemia (XLA). Bruton's disease, congenital agammaglobulinemia, X-linked infantile agammaglobulinemia, acquired agammaglobulinemia, adult onset agammaglobulinemia, late-onset agammaglobulinemia, dysgammaglobulinemia, hypogammaglobulinemia, transient hypogammaglobulinemia of infancy, unspecified hypogammaglobulinemia, agammaglobulinemia, common variable immunodeficiency (CVID) (acquired), Wiskott-Aldrich Syndrome (WAS), X-linked immunodeficiency with hyper IgM, non X-linked immunodeficiency with hyper IgM, selective IgA deficiency, IgG subclass deficiency (with or without IgA deficiency), antibody deficiency with normal or elevated Igs, immunodeficiency with thymoma, Ig heavy chain deletions, kappa chain deficiency, B cell lymphoproliferative disorder (BLPD), selective IgM immunodeficiency, recessive agammaglobulinemia (Swiss type), reticular dysgenesis, neonatal neutropenia, severe congenital leukopenia, thymic alymphoplasia-aplasia or dysplasia with immunodeficiency, ataxia-telangiectasia telangiectasia (cerebellar ataxia, oculocutaneous telangiectasia and immunodeficiency), short limbed dwarfism, X-linked lymphoproliferative syndrome (XLP), Nezelof syndrome-cumbined immunodeficiency with Igs, purine nucleotide phosphorylase deficiency (PNP), MHC Class II deficiency (Bare Lymphocyte Syndrome) and severe combined immunodeficiency,) or conditions associated with an immunodeficiency, Janus Associated Kinase 3 (JAK3) deficiency, DiGeorge's syndrome (isolated T cell deficiency) and Associated syndromes e.g., Down syndrome, chronic mucocutaneous candidiasis, hyper-IgE syndrome, chronic granulomatous disease, partial albinism and WHIM syndrome (warts, hypogammaglobulinemia, infection, and myelokathexis [retention of leukocytes in a hypercellular marrow]).

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP) and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

As used herein, “B cell depletion” refers to a reduction in B cell levels in an animal or human after drug or antibody treatment, as compared to the B cell level before treatment. B cell levels are measurable using well known assays such as those described in the Experimental Examples. B cell depletion can be complete or partial. In one embodiment, the depletion of BR3 expressing B cells is at least 25%. Not to be limited by any one mechanism, possible mechanisms of B-cell depletion include ADCC, CDC, apoptosis, modulation of calcium flux or a combination of two or more of the preceding.

A “B cell surface marker” or “B cell surface antigen” herein is an antigen expressed on the surface of a B cells.

“B cell depletion agents” refers to agents that reduce peripheral B cells by at least 25%. In another embodiment, the depletion of peripheral B cells is at least 30%, 40%, 50%, 60%, 70%, 80% or 90%. In one preferred embodiment, the B cell depletion agent specifically binds to a white blood cell and not other cells types. In another embodiment, the B cell depletion agent specifically binds to a B cell and not other cell types. In one embodiment, the B cell depletion agent is an antibody. In one preferred embodiment, the antibody is a monoclonal antibody. In another embodiment, the antibody is conjugated to a chemotherapeutic agent or a cytotoxic agent. Specific examples of B cell depletion agents include, but are not limited to, the aforementioned anti-CD20 antibodies.

The B cell neoplasms include Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia and BR3-positive neoplasms. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment and can be characterized by BR3-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

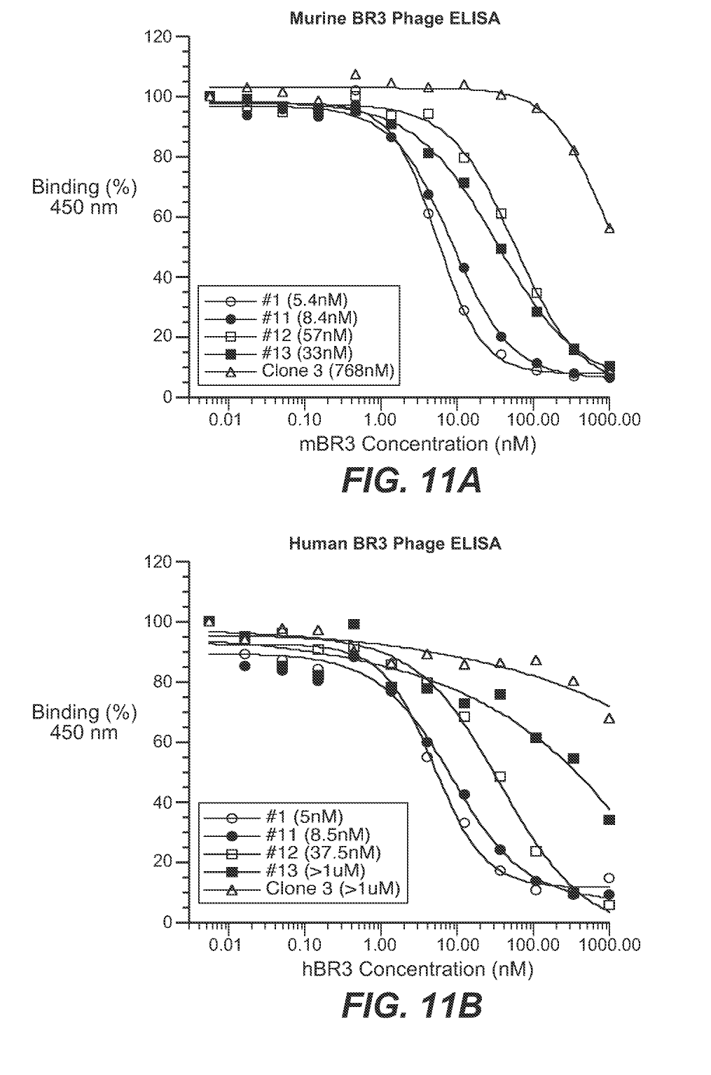

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology, Third Edition; A. Victor Hoffbrand and John E. Pettit (eds.) (Harcourt Publishers Limited 2000) (see, in particular FIG. 11.57, 11.58 and/or 11.59). More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphacytic leukemia and/or prolymphocytic leukemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukemia and/or lymphoma, adult T-cell lymphoma and/or leukemia, T cell chronic lymphocytic leukemia and/or prolymphacytic leukemia, large granular lymphocytic leukemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD). According to one preferred embodiment, the cancer comprises a tumor that expresses a BR3 polypeptide on its surface (BR3-positive). According to another embodiment, the BR3-expressing cancer is a CLL cancer.

In specific embodiments, the anti-BR3 antibodies and polypeptides of this invention are used to treat any one or more of the diseases selected from the group consisting of non-Hodgkin's lymphoma (NHL), lymphocyte predominant Hodgkin's disease (LPHD), chronic lymphocytic leukemia (CLL), acute lymphocytic leukemia (ALL), small lymphocytic lymphoma (SLL), diffuse large B cell lymphoma (DLBCL), follicular lymphoma, which are types of non-Hodgkin's lymphoma (NHL), rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome, glomerulonephritis and multiple myeloma.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², Bi²¹³, P³² and radioactive isotopes of Lu), chemotherapeutic agents, and toxins such as small molecule toxins or enzymatically active toxins of bacterial, fungal, plant or animal origin, including fragments and/or variants thereof. According to one embodiment, the cytotoxic agent is capable of being internalized. According to another embodiment, the active portion of the cytotoxic agent is 1100 kD or less. According to one embodiment the chemotherapeutic agent is selected from the group consisting of methotrexate, adriamicin, vinca alkaloids (vincristine, vinblastine, etoposide), doxorubicin, melphalan, mitomycin C, chlorambucil, daunorubicin, or other intercalating agents, enzymes and fragments thereof such as nucleolytic enzymes, antibiotics, and toxins such as small molecule toxins or enzymatically active toxins of bacterial, fungal, plant or animal origin, (e.g., monomethylauristatin (MMAE) including fragments and/or variants thereof, and the various antitumor or anticancer agents or grow inhibitory agents disclosed below. Other cytotoxic agents are described below.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, actinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-oxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAX™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON. toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL® paclitaxel, and topo II inhibitors such as doxorubicin, epirubicin, daunorubicin, etoposide, and bleomycin. Those agents that arrest G1 also spill over into S-phase arrest, for example, DNA alkylating agents such as tanoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antieioplastic drugs” by Murakaini et al. (W B Saunders: Philadelphia, 1995), especially p. 13.

An antibody that “induces cell death” is one that causes a viable cell to become nonviable. The cell is generally one that expresses the antigen to which the antibody binds, especially where the cell overexpresses the antigen. Preferably, the cell is a cancer cell, e.g., a breast, ovarian, stomach, endometrial, salivary gland, lung, kidney, colon, thyroid, pancreatic or bladder cell. In vitro, the cell may be a SKBR3, BT474, Calu 3, MDA-MB-453, MDA-MB-361 or SKOV3 cell. Cell death in vitro may be determined in the absence of complement and immune effector cells to distinguish cell death induced by antibody dependent cell-mediated cytotoxicity (ADCC) or complement dependent cytotoxicity (CDC). Thus, the assay for cell death may be performed using heat inactivated serum (i.e. in the absence of complement) and in the absence of immune effector cells. To determine whether the antibody is able to induce cell death, loss of membrane integrity as evaluated by uptake of propidium iodide (PI), trypan blue (see Moore et al. Cytotechnology, 17: 1-11 (1995)) or 7AAD can be assessed relative to untreated cells.

An antibody that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is one which expresses the antigen to which the antibody binds and may be one that overexpresses the antigen. The cell may be a tumor cell, e.g., a breast, ovarian, stomach, endometrial, salivary gland, lung, kidney, colon, thyroid, pancreatic or bladder cell. In vitro, the cell may be a SKBR3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering as disclosed in the example herein; and nuclear/chromatin condensation along with DNA fragmentation can be evaluated by any increase in hypodiploid cells. Preferably, the antibody that induces apoptosis is one which results in about 2 to 50 fold, preferably about 5 to 50 fold, and most preferably about 10 to 50 fold, induction of annexin binding relative to untreated cell in an annexin binding assay using cells expressing the antigen to which the antibody binds.

Examples of antibodies that induce apoptosis include the anti-DR5 antibodies 3F1 1.39.7 (ATCC HB-12456); 3H3.14.5 (ATCC HB-12534); 3D5.1.10 (ATCC HB-12536); and 3H3.14.5 (ATCC HB-12534), including humanized and/or affinity-matured variants thereof; the human anti-DR5 receptor antibodies 16E2 and 20E6, including affinity-matured variants thereof (WO98/5 1793, expressly incorporated herein by reference); the anti-DR4 antibodies 4E7.24.3 (ATCC HB-12454); 4H6.17.8 (ATCC HB-12455); 1H5.25.9 (ATCC HB-12695); 4G7.18.8 (ATCC PTA-99); and 5G I 1.17.1 (ATCC HB-12694), including humanized and/or affinity-matured variants thereof.

In order to screen for antibodies which bind to an epitope on an antigen bound by an antibody of interest, a routine cross-blocking assay such as that described in Antibodies, A Laboratory Manual, eds. Harlow and Lane (New York: Cold Spring Harbor Laboratory, 1988) can be performed.

A “conjugate” refers to any hybrid molecule, including fusion proteins and as well as molecules that contain both amino acid or protein portions and non-protein portions (e.g., toxin-antibody conjugates, or pegylated-antibody conjugates). Conjugates may be synthesized or engineered by a variety of techniques known in the art including, for example, recombinant DNA techniques, solid phase synthesis, solution phase synthesis, organic chemical synthetic techniques or a combination of these techniques. The choice of synthesis will depend upon the particular molecule to be generated. For example, a hybrid molecule not entirely “protein” in nature may be synthesized by a combination of recombinant techniques and solution phase techniques.

According to one embodiment, the conjugate is an antibody or polypeptide of interest covalently linked to a salvage receptor binding epitope (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is useful for increasing the in vivo serum half-life of the IgG molecule (e.g., Ghetie, V et al., (2000) Ann. Rev. Immunol. 18:739-766, Table 1).

In another embodiment, the conjugate can be formed, by linkage (especially an antibody fragment) to serum albumin or a portion of serum albumin that binds to the FcRn receptor or a serum albumin-binding peptide or to a non-protein polymer (e.g., a polyethylene glycol moiety). Such polypeptide sequences are disclosed, for example, in WO01/45746. In one preferred embodiment, the serum albumin peptide to be attached comprises an amino acid sequence of DICLPRWGCLW. In another embodiment, the half-life of a Fab according to this invention is increased by these methods. See also, Dennis, M. S., et al., (2002) JBC 277(38):35035-35043 for serum albumin binding peptide sequences.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

### A. Compositions and Methods of the Invention

The invention provides antibodies that bind human BR3, and optionally other primate BR3 as well. According to one embodiment, the H chain has at least one, two or all of the H chain CDRs of a non-human species anti-human BR3 antibody (donor antibody), and substantially all of the framework residues of a human consensus antibody as the recipient antibody. The donor antibody can be from various non-human species including mouse, rat, guinea pig, goat, rabbit, horse, primate but typically will be a murine antibody. “Substantially all” in this context is meant that the recipient FR regions in the humanized antibody may include one or more amino acid substitutions not originally present in the human consensus FR sequence. These FR changes may comprise residues not found in the recipient or the donor antibody.

In one embodiment, the donor antibody is the murine 9.1 antibody, the V region including the CDR and FR sequences of each of the VH and VL chains of which are shown in SEQ ID NO:19 and SEQ ID NO:20. In one embodiment, the residues for the human Fab framework correspond to or were derived from the consensus sequence of a human Vκ subgroup I and of a V_{H} subgroup III. According to one embodiment, a humanized BR3 antibody of the invention has at least one of the CDRs in the H chain of the murine donor antibody. In one embodiment, the humanized BR3 antibody that binds human BR3 comprises the heavy chain CDRs of the H chain of the donor antibody.

In a full length antibody, the humanized BR3 binding antibody of the invention will comprise a V domain joined to a C domain of a human immunoglobulin, e.g., SEQ ID NO:132. In a preferred embodiment, the H chain C region is from human IgG, such as IgG1 or IgG3. According to one embodiment, the L chain C domain is from a human K chain. According to another embodiment, the Fc sequence of a full length BR3 binding antibody is SEQ ID NO:134, wherein X is selected from the group consisting of N, A, Y, F and H.

The BR3 binding antibodies will bind at least human BR3. According to one embodiment, the BR3-binding antibody will bind other primate BR3 such as that of monkeys including cynomolgus and rhesus monkeys, and chimpanzees. According to another embodiment, the BR3 binding antibody or polypeptide binds a rodent BR3 protein and a human BR3 protein. In another embodiment, the BR3 polypeptide binds a mouse BR3 polypeptide sequence and a human BR3 polypeptide sequence.

According to one embodiment, the biological activity of an antagonist BR3 binding antibodies is any one, any combination or all of the activities selected from the group consisting of: (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a mouse BR3 extracellular domain sequence with an apparent Kd value of 500 nM or less, 100 nM or less, 50 nM or less, 10 nM or less, 5 nM or less or 1 nM or less; (3) has a functional epitope on human BR3 comprising residues F25, V33 and A34, wherein the monoclonal antibody; (4) inhibits human BAFF and human BR3 binding; (5) has antibody dependent cellular cytotoxicity (ADCC) in the presence of human effector cells or has increased ADCC in the presence of human effector cells; (6) binds the human Fc neonatal receptor (FcRn) with a higher affinity than a polypeptide or parent polypeptide having wild type or native sequence IgG Fc; (9) kills or depletes B cells in vitro or in vivo, preferably by at least 20% when compared to the baseline level or appropriate negative control which is not treated with such antibody; (10) inhibits B cell proliferation in vitro or in vivo and (11) inhibits B cell survival in vitro or in vivo. According to one embodiment of the polypeptides or antibodies of this invention, the functional epitope further comprises residue R30. According to yet another embodiment of this invention, the functional epitope further comprises residues L28 and V29.

In one embodiment, compared to treatment with a control antibody that does not bind a B cell surface antigen or as compared to the baseline level before treatment, the variable domain of an antibody of this invention fused to an Fc region of an mIgG2A can deplete at least 20% of the B cells in any one, any combination or all of following population of cells in mice: (1) B cells in blood, (2) B cells in the lymph nodes, (3) follicular B cells in the spleen and (4) marginal zone B cells in the spleen. In other embodiments, B cell depletion is 25%, 30%, 40%, 50%, 60%, 70%, 80% or greater. In one preferred embodiment, the depletion is measured at day 15 post treatment with antibody. In another preferred embodiment, the depletion assay is carried out as described in Example 18 or 19 herein. In another preferred embodiment, the depletion is measured by the population of peripheral B cells in a mouse day 15 post-treatment.

According to another embodiment the biological activity of an agonist BR3 binding antibody of this invention is any one, any combination or all of the activities selected from the group consisting of: (1) binds to a human BR3 extracellular domain sequence with an apparent Kd value of 500 nM or less, 100 nM or less, 50 nM or less, 10 nM or less, 5 nM or less or 1 nM or less; (2) has a functional epitope on human BR3 comprising residues F25, V33 and A34, wherein the monoclonal antibody is not the 9.1 antibody or the 2.1 antibody; (3) stimulates B cell proliferation or survival in vitro; (4) inhibits human BAFF and human BR3 binding; (5) stimulates B cell proliferation or survival in vivo; (6) binds the human Fc neonatal receptor (FcRn) with a higher affinity than a polypeptide or parent polypeptide having wild type or native sequence IgG Fc.

The desired level of B cell depletion will depend on the disease. For the treatment of a BR3 positive cancer, it may be desirable to maximize the depletion of the B cells which are the target of the anti-BR3 antibodies and polypeptides of the invention. Thus, for the treatment of a BR3 positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. According to one preferred embodiment, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

For treatment of an autoimmune disease, it can be desirable to modulate the extent of B cell depletion depending on the disease and/or the severity of the condition in the individual patient, by adjusting the dosage of BR3 binding antibody or polypeptide. Thus, B cell depletion can but does not have to be complete. Total B cell depletion may be desired in initial treatment but in subsequent treatments, the dosage may be adjusted to achieve only partial depletion. In one embodiment, the B cell depletion is at least 20%, i.e., 80% or less of BR3 positive B cells remain as compared to the baseline level before treatment. In other embodiments, B cell depletion is 25%, 30%, 40%, 50%, 60%, 70%, 80% or greater. According to one preferred embodiment, the B cell depletion is sufficient to halt progression of the disease, more preferably to alleviate the signs and symptoms of the particular disease under treatment, even more preferably to cure the disease.

The invention also provides bispecific BR3 binding antibodies wherein one arm of the antibody has a humanized H and L chain of the BR3 binding antibody of the invention, and the other arm has V region binding specificity for a second antigen. In specific embodiments, the second antigen is selected from the group consisting of CD3, CD64, CD32A, CD16, NKG2D or other NK activating ligands.

Any cysteine residue not involved in maintaining the proper conformation of the anti-BR3 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human BR3. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-BR3 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-BR3 antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement mediated lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Screening for Antibodies with the Desired Properties

Antibodies with certain biological characteristics may be selected as described in the Experimental Examples. For example, antibodies that bind BR3 can be selected by binding to BR3 in ELISA assays or, more preferably, by binding to BR3 expressed on the surface of cells (e.g., BJAB cell line). See, e.g., Example 5.

The growth inhibitory effects of an anti-BR3 antibody of the invention may be assessed by the Examples or methods known in the art, e.g., using cells which express BR3 either endogenously or following transfection with the BR3 gene. For example, in one preferred embodiment, primary B cells expressing BR3 can be used in proliferation and survival assays (e.g., Example 7). In another example, tumor cell lines and BR3-transfected cells may treated with an anti-BR3 monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-BR3 antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line.

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. BR3-expressing tumor cells are incubated with medium alone or medium containing of the appropriate monoclonal antibody at e.g, about 10 μg/ml. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson). Those antibodies which induce statistically significant levels of cell death as determined by PI uptake may be selected as cell death-inducing antibodies.

To screen for antibodies which bind to an epitope on BR3 bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-BR3 antibody of the invention. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of BR3 can be used in competition assays with the test antibodies or with a test antibody and an antibody with a characterized or known epitope.

### Examples of Specific Anti-BR3Antibodies

Antibodies of this invention specifically include antibodies comprising the variable heavy chain sequence of any one of the antibodies disclosed in Table 2 (below), and BR3-binding fragments thereof that has not been produced by a hybridoma cell. Antibodies of this invention specifically include antibodies comprising a variable heavy chain sequence comprising the sequence of any one of SEQ ID NO: 4-13, 15-18, 22, 24, 26-73, 75-76, 78, 80-85, 87-96, 98, 100, 102, 104, 106-107, 109-110, 112, 114, 116, 118, 120, 122, 124, 126 and 127, and BR3-binding fragments thereof. According to a further embodiment, an antibody of this invention comprises the variable heavy and the variable light chain region of any one of the antibodies disclosed in Table 2, and BR3-binding fragments thereof. According to one embodiment, the antibody further comprises an Fc region comprising the sequence of SEQ ID NO:134, wherein X is an amino acid selected from the group consisting of N, A, W, Y, F and H. According to another embodiment, the antibody comprises the sequence of SEQ ID NO:76 or SEQ ID NO:131, wherein X is an amino acid selected from the group consisting of N, A, W, Y, F and H.

TABLE 2
Examples of Antibody Sequences

| ANTIBODY | SEQ ID NO: | | SEQ ID NO: | | FRAMEWORK |
|---|---|---|---|---|---|
| 2.1 | 1 | (VL) | 2 | (VH) | Mouse |
| hu2.1-Graft | 3 | (VL) | 4 | (VH) | R71A/N73T/L78A |
| Hu2.1-RL | 3 | (VL) | 5 | (VH) | RL |
| Hu2.1-RF | 3 | (VL) | 6 | (VH) | RF |
| Hu2.1-40 | 3 | (VL) | 7 | (VH) | RF |
| Hu2.1-46 | 3 | (VL) | 8 | (VH) | RF |
| Hu2.1-30 | 3 | (VL) | 9 | (VH) | RF |
| Hu2.1-93 | 3 | (VL) | 10 | (VH) | RL |
| Hu2.1-94 | 3 | (VL) | 11 | (VH) | RL |
| Hu2.1-40L | 3 | (VL) | 12 | (VH) | RL |
| Hu2.1-89 | 3 | (VL) | 13 | (VH) | RL |
| Hu2.1-46.DANA-IgG | 14 | (LC) | 15 | (HC) | RF |
| Hu2.1-27 | 3 | (VL) | 16 | (VH) | RF |
| Hu2.1-36 | 3 | (VL) | 17 | (VH) | RF |
| Hu2.1-31 | 3 | (VL) | 18 | (VH) | RF |
| 9.1 | 19 | (VL) | 20 | (VH) | Mouse |
| Hu9.1-graft | 21 | (VL) | 22 | (VH) | R71A/N73T/L78A |
| Hu9.1-73 | 23 | (VL) | 24 | (VH) | R71A/N73T/L78A |
| Hu9.1-70 | 25 | (VL) | 26 | (VH) | R71A/N73T/L78A |
| Hu9.1-56 | 21 | (VL) | 27 | (VH) | R71A/N73T/L78A |
| Hu9.1-51 | 21 | (VL) | 28 | (VH) | R71A/N73T/L78A |
| Hu9.1-59 | 21 | (VL) | 29 | (VH) | R71A/N73T/L78A |
| Hu9.1-61 | 21 | (VL) | 30 | (VH) | R71A/N73T/L78A |
| Hu9.1-A | 21 | (VL) | 31 | (VH) | R71A/N73T/L78A |
| Hu9.1-B | 21 | (VL) | 32 | (VH) | R71A/N73T/L78A |
| Hu9.1-C | 21 | (VL) | 33 | (VH) | R71A/N73T/L78A |
| Hu9.1-66 | 21 | (VL) | 34 | (VH) | R71A/N73T/L78A |
| Hu9.1-RF | 21 | (VL) | 35 | (VH) | RF |
| Hu9.1-48 | 21 | (VL) | 36 | (VH) | RF |
| Hu9.1-RL | 21 | (VL) | 37 | (VH) | RL |
| Hu9.1-91 | 21 | (VL) | 38 | (VH) | RL |
| Hu9.1-90 | 21 | (VL) | 39 | (VH) | RL |
| Hu9.1-75 | 21 | (VL) | 40 | (VH) | RL |
| Hu9.1-88 | 21 | (VL) | 41 | (VH) | RL |
| Hu9.1RL-9 | 21 | (VL) | 42 | (VH) | RL |
| Hu9.1RL-44 | 21 | (VL) | 43 | (VH) | RL |
| Hu9.1RL-13 | 21 | (VL) | 44 | (VH) | RL |
| Hu9.1RL-47 | 21 | (VL) | 45 | (VH) | RL |
| Hu9.1RL-28 | 21 | (VL) | 46 | (VH) | RL |
| Hu9.1RL-43 | 21 | (VL) | 47 | (VH) | RL |
| Hu9.1RL-16 | 21 | (VL) | 48 | (VH) | RL |
| Hu9.1RL-70 | 21 | (VL) | 49 | (VH) | RL |
| Hu9.1RL-30 | 21 | (VL) | 50 | (VH) | RL |
| Hu9.1RL-32 | 21 | (VL) | 51 | (VH) | RL |
| Hu9.1RL-37 | 21 | (VL) | 52 | (VH) | RL |
| Hu9.1RL-29 | 21 | (VL) | 53 | (VH) | RL |
| Hu9.1RL-10 | 21 | (VL) | 54 | (VH) | RL |
| Hu9.1RL-24 | 21 | (VL) | 55 | (VH) | RL |
| Hu9.1RL-39 | 21 | (VL) | 56 | (VH) | RL |
| Hu9.1RL-31 | 21 | (VL) | 57 | (VH) | RL |
| Hu9.1RL-18 | 21 | (VL) | 58 | (VH) | RL |
| Hu9.1RL-23 | 21 | (VL) | 59 | (VH) | RL |
| Hu9.1RL-41 | 21 | (VL) | 60 | (VH) | RL |
| Hu9.1RL-95 | 21 | (VL) | 61 | (VH) | RL |
| Hu9.1RL-14 | 21 | (VL) | 62 | (VH) | RL |
| Hu9.1RL-57 | 21 | (VL) | 63 | (VH) | RL |
| Hu9.1RL-15 | 21 | (VL) | 64 | (VH) | RL |
| Hu9.1RL-54 | 21 | (VL) | 65 | (VH) | RL |
| Hu9.1RL-12 | 21 | (VL) | 66 | (VH) | RL |
| Hu9.1RL-34 | 21 | (VL) | 67 | (VH) | RL |
| Hu9.1RL-25 | 21 | (VL) | 68 | (VH) | RL |
| Hu9.1RL-71 | 21 | (VL) | 69 | (VH) | RL |
| Hu9.1RL-5 | 21 | (VL) | 70 | (VH) | RL |
| Hu9.1RL-79 | 21 | (VL) | 71 | (VH) | RL |
| Hu9.1RL-66 | 21 | (VL) | 72 | (VH) | RL |
| Hu9.1RL-69 | 21 | (VL) | 73 | (VH) | RL |
| 9.1RF-IgG | 74 | (LC) | 75 | (HC) | RF |
| 9.1RF-IgG (N434X) | 74 | (LC) | 76 | (HC) | RF |
| 11G9 | 77 | (VL) | 78 | (VH) | Mouse |
| Hu11G9-graft | 79 | (VL) | 80 | (VH) | R71A/N73T/L78A |
| Hu11G9-RF | 79 | (VL) | 81 | (VH) | RF |
| Hu11G9-36 | 79 | (VL) | 82 | (VH) | RF |
| Hu11G9-46 | 79 | (VL) | 83 | (VH) | RF |
| Hu11G9-35 | 79 | (VL) | 84 | (VH) | RF |
| Hu11G9-29 | 79 | (VL) | 85 | (VH) | RF |
| V3-Fab | 86 | (LC) | 87 | (HC) | |
| V24 | 86 | (VL) | 88 | (VH) | |
| V44 | 86 | (VL) | 89 | (VH) | |
| V89 | 86 | (VL) | 90 | (VH) | |
| V96 | 86 | (VL) | 91 | (VH) | |
| V46 | 86 | (VL) | 92 | (VH) | |
| V51 | 86 | (VL) | 93 | (VH) | |
| V75 | 86 | (VL) | 94 | (VH) | |
| V58 | 86 | (VL) | 95 | (VH) | |
| V60 | 86 | (VL) | 96 | (VH) | |
| V3-1 | 97 | (VL) | 98 | (VH) | |
| V3-11 | 99 | (VL) | 100 | (VH) | |
| V3-12 | 101 | (VL) | 102 | (VH) | |
| V3-13 | 103 | (VL) | 104 | (VH) | |
| V3-3 | 105 | (VL) | 106 | (VH) | |
| V3-5 | 97 | (VL) | 107 | (VH) | |
| V3-9 | 108 | (VL) | 98 | (VH) | |
| V3-16 | 97 | (VL) | 109 | (VH) | |
| V3-19 | 97 | (VL) | 110 | (VH) | |
| V3-24 | 111 | (VL) | 112 | (VH) | |
| V3-27 | 113 | (VL) | 114 | (VH) | |
| V3-34 | 115 | (VL) | 116 | (VH) | |
| V3-35 | 117 | (VL) | 118 | (VH) | |
| V3-37 | 119 | (VL) | 120 | (VH) | |
| V3-41 | 121 | (VL) | 122 | (VH) | |
| V3-46 | 123 | (VL) | 124 | (VH) | |
| V3-46a | 123 | (VL) | 125 | (VH) | |
| V3-46q | 123 | (VL) | 126 | (VH) | |
| V3-46s | 123 | (VL) | 127 | (VH) | |
| V3-46sFab | 128 | (LC) | 129 | (HC) | |
| V3-46s IgG | 128 | (LC) | 130 | (HC) | |
| V3-46s IgG (N434X) | 128 | (LC) | 131 | (HC) | |
| V3-46s-1 | 194 | (LC) | 127 | (VH) | |
| V3-46s-7 | 195 | (LC) | 127 | (VH) | |
| V3-46s-9 | 196 | (LC) | 127 | (VH) | |
| V3-46s-10 | 197 | (LC) | 127 | (VH) | |
| V3-46s-12 | 198 | (LC) | 193 | (VH) | |
| V3-46s-13 | 199 | (LC) | 127 | (VH) | |
| V3-46s-29 | 200 | (LC) | 127 | (VH) | |
| V3-46s-31 | 201 | (LC) | 127 | (VH) | |
| V3-46s-33 | 202 | (LC) | 127 | (VH) | |
| V3-46s-34 | 203 | (LC) | 127 | (VH) | |
| V3-46s-37 | 204 | (LC) | 127 | (VH) | |
| V3-46s-40 | 205 | (LC) | 127 | (VH) | |
| V3-46s-42 | 206 | (LC) | 127 | (VH) | |
| V3-46s-45 | 207 | (LC) | 127 | (VH) | |

Antibodies of this invention include BR3-binding antibodies having an H3 sequence that is at least about 70% amino acid sequence identity, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identity, to the H3 sequence of any one of the sequences of SEQ ID NO:s: 4-13, 15-18, 22, 24, 26-73, 75-76, 78, 80-85, 87-96, 98, 100, 102, 104, 106-107, 109-110, 112, 114, 116, 118, 120, 122, 124, 126 and 127, and BR3 binding fragments of those antibodies.

Antibodies of this invention include BR3-binding antibodies having H1, H2 and H3 sequences that are at least 70% identical to the underlined portions of any one of the antibodies sequences described in the Figures or to the CDRs of hypervariable regions described in the Sequence Listing, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include BR3-binding antibodies having L1, L2 and L3 sequences that are at least 70% identical to the underlined portions of any one of the antibodies sequences described in the Figures or to the CDRs or hypervariable regions described in the Sequence Listing, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include BR3-binding antibodies having a VH domain with at least 70% homology to a VH domain of any one of the antibodies of Table 2, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include any BR3-binding antibody comprising a heavy chain CDR3 sequence of an antibody sequence of Table 2 that has not been produced by a hybridoma cell. Antibodies of this invention include any BR3-binding antibody comprising a heavy chain CDR3 sequence of any one of SEQ ID NO:s:7-13, 15-18, 36, 38-73, 78, 82-85, 87-96, 98, 100, 102, 104, 106-107, 109-110, 112, 114, 116, 118, 120, 122, 124, 126 and 127, or comprising a H3 sequence that is derived a H3 sequence of any one of SEQ ID NO:s:7-13, 15-18, 36, 38-73, 78, 82-85, 87-96, 98, 100, 102, 104, 106-107, 109-110, 112, 114, 116, 118, 120, 122, 124, 126 and 127. In another embodiment, an antibody of this invention includes any BR3-binding antibody comprising a CDR-H1, CDR-H2 and CDR-H3 of any one of the sequences selected from the group consisting of SEQ ID NOs:7-13, 15-18, 36, 38-73, 78, 82-85, 87-96, 98, 100, 102, 104, 106-107, 109-110, 112, 114, 116, 118, 120, 122, 124, 126 and 127 or is derived from an antibody comprising the CDR-H1, CDR-H2 and CDR-H3 sequences. Antibodies of this invention include any BR3-binding antibody comprising a heavy chain H1, H2 and H3 sequence of an antibody of Table 2 that has not been produced by a hybridoma cell.

Antibodies of this invention include the antibodies comprising a polypeptide sequence encoded by the Hu9.1-RF-H-IgG nucleic acid sequence deposited as ATCC deposit number PTA-6315 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to any one of the variable regions sequence of the Hu9.1-RF-H-IgG polypeptide sequence. Antibodies of this invention include the antibodies comprising a polypeptide sequence encoded by the Hu9.1-RF-L-IgG nucleic acid sequence deposited as ATCC deposit number PTA-6316 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu9.1-RF-L-IgG polypeptide sequence.

Antibodies of this invention include the antibodies comprising a polypeptide sequence encoded by the Hu2.1-46.DANA-H-IgG nucleic acid sequence deposited as ATCC deposit number PTA-6313 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu2.1-46.DANA-H-IgG polypeptide sequence. Antibodies of this invention include the antibodies comprising a polypeptide sequence encoded by the Hu2.1-46.DANA-L-IgG nucleic acid sequence deposited as ATCC deposit number PTA-6314 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu2.1-46.DANA-L-IgG polypeptide sequence.

Antibodies of this invention include the antibodies comprising a polypeptide sequence encoded by the HuV3-46s-H-IgG nucleic acid sequence deposited as ATCC deposit number PTA-6317 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the HuV3-46s-H-IgG polypeptide sequence. Antibodies of this invention include the antibodies comprising a polypeptide sequence encoded by the HuV3-46s-L-IgG nucleic acid sequence deposited as ATCC deposit number PTA-6318 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the HuV3-46s-L-IgG polypeptide sequence.

Antibodies of this invention include the Hu9.1-RF-IgG antibody comprising the heavy chain sequence of ATCC deposit no. PTA-6315 and the light chain sequence of ATCC deposit no. PTA-6316. Antibodies of this invention include the Hu2.1-46.DANA-IgG antibody comprising the heavy sequence of ATCC deposit no. PTA-6313 and the light chain sequence of ATCC deposit no. PTA-6314. Antibodies of this invention include the HuV3-46s-IgG antibody comprising the heavy sequence of ATCC deposit no. PTA-6317 and the light chain sequence of ATCC deposit no. PTA-6318.

According to one preferred embodiment, the antibodies of this invention specifically bind to a sequence of a native human BR3 polypeptide. According to yet another embodiment, an antibody of this invention has improved binding to the FcRn receptor at pH 6.0 compared to the antibody known as 9.1-RF Ig. According to yet another embodiment, an antibody of this invention has improved ADCC function in the presence of human effector cells compared to the antibody known as 9.1-RF Ig. According to yet another embodiment, an antibody of this invention has decreased ADCC function in the presence of human effector cells compared to the antibody known as 9.1-RF Ig.

It is understood that all antibodies of this invention include antibodies lacking a signal sequence and antibodies lacking the K447 residue of the Fc region.

### Vectors, Host Cells and Recombinant Methods

The invention also provides an isolated nucleic acid encoding a BR3 binding antibody or BR3 binding polypeptide, vectors and host cells comprising the nucleic acid, and recombinant techniques for the production of the antibody.

For recombinant production of the BR3 binding antibodies and polypeptides, the nucleic acid encoding it is isolated and inserted into a replicable vector for further cloning (amplification of the DNA) or for expression. DNA encoding the monoclonal antibody or polypeptide is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The antibody or polypeptide of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native BR3 binding antibody signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, α factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the BR3 binding antibody.

(ii) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the BR3 binding antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding BR3 binding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(iv) Promoter Component

Expression and cloning vectors usually contain a promoter that is recognized by the host organism and is operably linked to the nucleic acid encoding the BR3 binding antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the BR3 binding antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), or from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of a DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibody. One useful transcription termination component is the bovine growth hormone polyadenylation region. See WO94/11026 and the expression vector disclosed therein.

(vii) Selection and transformation of host cells Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Sallmonella, e.g., Sallmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g, in CHO cells.

In addition to prokaryotes, eukaryotic microbes such as filamentous fungi or yeast are suitable cloning or expression hosts for BR3 binding antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated BR3 binding antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Antibody Conjugates

The antibody may be conjugated to a cytotoxic agent such as a toxin or a radioactive isotope. In certain embodiments, the toxin is calicheamicin, a maytansinoid, a dolastatin, auristatin E and analogs or derivatives thereof, are preferable.

Preferred drugs/toxins include DNA damaging agents, inhibitors of microtubule polymerization or depolymerization and antimetabolites. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, N-(5,5-diacetoxypentyl)doxorubicin, morpholino-doxorubicin, 1-(2-choroehthyl)-1,2-dimethanesulfonyl hydrazide, N⁸-acetyl spermidine, aminopterin methopterin, esperamicin, mitomycin C, mitomycin A, actinomycin, bleomycin, caminomycin, aminopterin, tallysomycin, podophyllotoxin and podophyllotoxin derivatives such as etoposide or etoposide phosphate, vinblastine, vincristine, vindesine, taxol, taxotere, retinoic acid, butyric acid, N⁸-acetyl spermiidine, camptothecin, calicheamicin, bryostatins, cephalostatins, ansamitocin, actosin, maytansinoids such as DM-1, maytansine, maytansinol, N-desmethyl-4,5-desepoxymaytansinol, C-19-dechloromaytansinol, C-20-hydroxymaytansinol, C-20-demethoxymaytansinol, C-9-SH maytansinol, C-14-alkoxymethylmaytansinol, C-14-hydroxy or acetyloxymethlmaytansinol, C-15-hydroxy/acetyloxymaytansinol, C-15-methoxymaytansinol, C-18-N-demethylmaytansinol and 4,5-deoxymaytansinol, auristatins such as auristatin E, M, PHE and PE; dolostatins such as dolostatin A, dolostatin B, dolostatin C, dolostatin D, dolostatin E (20-epi and 11-epi), dolostatin G, dolostatin H, dolostatin I, dolostatin 1, dolostatin 2, dolostatin 3, dolostatin 4, dolostatin 5, dolostatin 6, dolostatin 7, dolostatin 8, dolostatin 9, dolostatin 10, deo-dolostatin 10, dolostatin 11, dolostatin 12, dolostatin 13, dolostatin 14, dolostatin 15, dolostatin 16, dolostatin 17, and dolostatin 18; cephalostatins such as cephalostatin 1, cephalostatin 2, cephalostatin 3, cephalostatin 4, cephalostatin 5, cephalostatin 6, cephalostatin 7, 25′-epi-cephalostatin 7,20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene.

There are many linking groups known in the art for making antibody-maytansinoid conjugates, including, for example, those disclosed in U.S. Pat. No. 5,208,020 or EP Patent 0 425 235 B1, and Chari et al. Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an BR3 binding antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, γx₂^{I}, γ₃^{I}, N-acetyl-γ₁^{I}, PSAG and θ^{I}₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Radioactive Isotopes

For selective destruction of the tumor, the antibody may comprise a highly radioactive atom. A variety of radioactive isotopes are available for the production of radioconjugated anti-BR3 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I₁₂₃, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

### Therapeutic Uses of the BR3 Binding Antibodies

The BR3 binding antibodies of the invention are useful to treat a number of malignant and non-malignant diseases including autoimmune diseases and related conditions, and BR3 positive cancers including B cell lymphomas and leukemias. Stem cells (B-cell progenitors) in bone marrow lack the BR3 antigen, allowing healthy B-cells to regenerate after treatment and return to normal levels within several months.

Autoimmune diseases or autoimmune related conditions include arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliatis), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, Lymphoid interstitial pneumonitis (HIV), bronchiolitis obliterans (non-transplant) vs NSIP, Guillain-Barre' Syndrome, Large Vessel Vasculitis (including Polymyalgia Rheumatica and Giant Cell (Takayasu's) Arteritis), Medium Vessel Vasculitis (including Kawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, coronary artery disease.

BR3 positive cancers are those comprising abnormal proliferation of cells that express BR3 on the cell surface. The BR3 positive B cell neoplasms include BR3-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia. The non-Hodgkins lymphoma include low grade/follicular non-Hodgkin's lymphoma (NHL), small lymphocytic lymphoma (SLL), intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment and is characterized by BR3-positive malignant cells. CLL is one of four major types of leukemia. A cancer of mature B-cells called lymphocytes, CLL is manifested by progressive accumulation of cells in blood, bone marrow and lymphatic tissues.

In specific embodiments, the BR3 binding antibodies and functional fragments thereof are used to treat non-Hodgkin's lymphoma (NHL), lymphocyte predominant Hodgkin's disease (LPHD), small lymphocytic lymphoma (SLL), chronic lymphocytic leukemia, rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

The BR3 binding antibodies or functional fragments thereof are useful as a single-agent treatment in, e.g., for relapsed or refractory low-grade or follicular, BR3-positive, B-cell NHL, or can be administered to patients in conjunction with other drugs in a multi drug regimen.

Indolent lymphoma is a slow-growing, incurable disease in which the average patient survives between six and 10 years following numerous periods of remission and relapse. In one embodiment, the humanized BR3 binding antibodies or functional fragments thereof are used to treat indolent NHL.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission, stable disease.

The following references describe lymphomas and CLL, their diagnoses, treatment and standard medical procedures for measuring treatment efficacy.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D:Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, the antibodies of the invention are useful to treat rheumatoid arthritis. RA is characterized by inflammation of multiple joints, cartilage loss and bone erosion that leads to joint destruction and ultimately reduced joint function. Additionally, since RA is a systemic disease, it can have effects in other tissues such as the lungs, eyes and bone marrow. Fewer than 50 percent of patients who have had RA for more than 10 years can continue to work or function normally on a day-to-day basis.

The antibodies can be used as first-line therapy in patients with early RA (i.e., methotrexate (MTX) naive) and as monotherapy, or in combination with, e.g., MTX or cyclophosphamide. Or, the antibodies can be used in treatment as second-line therapy for patients who were DMARD and/or MTX refractory, and as monotherapy or in combination with, e.g., MTX. The humanized BR3 binding antibodies are useful to prevent and control joint damage, delay structural damage, decrease pain associated with inflammation in RA, and generally reduce the signs and symptoms in moderate to severe RA. The RA patient can be treated with the humanized BR3 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized BR3 binding antibody of the invention. In one embodiment of this treatment, the patients are in a 17-day treatment regimen receiving humanized BR3 binding antibody alone (1 g iv infusions on days 1 and 15); BR3 binding antibody plus cyclophosphamide (750 mg iv infusion days 3 and 17); or BR3 binding antibody plus methotrexate.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g, baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.
   The ACR 50 and 70 are defined analogously. Preferably, the patient is administered an amount of a BR3 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized BR3 binding antibodies of the invention can be used to prevent the joint damage as well as reduce disease signs and symptoms of the disorder.

Yet another aspect of the invention is a method of treating Lupus or SLE by administering to the patient suffering from SLE, a therapeutically effective amount of a BR3 binding antibody of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, soriatic arthritis and Crohn's disease. Treatment success can be determined by validated patient and physician global assessment measuring tools.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. These therapies have a combination of safety concerns, time consuming regimens, or inconvenient processes of treatment. Furthermore, some require expensive equipment and dedicated space in the office setting. Systemic medications can produce serious side effects, including hypertension, hyperlipidemia, bone marrow suppression, liver disease, kidney disease and gastrointestinal upset. Also, the use of phototherapy can increase the incidence of skin cancers. In addition to the inconvenience and discomfort associated with the use of topical therapies, phototherapy and systemic treatments require cycling patients on and off therapy and monitoring lifetime exposure due to their side effects.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. Thus, another aspect of the invention is a method of treating the diseases disclosed by administering a BR3 binding antibody wherein the antibody has reduced or no complement dependent cytotoxicity.

Dosage

Depending on the indication to be treated and factors relevant to the dosing that a physician of skill in the field would be familiar with, the antibodies of the invention will be administered at a dosage that is efficacious for the treatment of that indication while minimizing toxicity and side effects. For the treatment of a cancer, an autoimmune disease or an immunodeficiency disease, the therapeutically effective dosage can be in the range of 50 mg/dose to 2.5 g/m². In one embodiment, the dosage administered is about 250 mg/m² to about 400 mg/m² or 500 mg/m². In another embodiment, the dosage is about 250-375 mg/m². In yet another embodiment, the dosage range is 275-375 mg/m².

In one embodiment of the treatment of a BR3 positive B cell neoplasm described herein (e.g., chronic lymphocytic leukemia (CLL), non-Hodgkins lymphoma (NHL), follicular lymphoma (FL) or multiple myeloma), the antibody is administered at a range of 50 mg/dose to 2.5 g/m². For the treatment of patients suffering from B-cell lymphoma such as non-Hodgkins lymphoma, in a specific embodiment, the anti-BR3 antibodies and humanized anti-BR3 antibodies of the invention will be administered to a human patient at a dosage of 10 mg/kg or 375 mg/m². For treating NHL, one dosing regimen would be to administer one dose of the antibody composition a dosage of 10 mg/kg in the first week of treatment, followed by a 2 week interval, then a second dose of the same amount of antibody is administered. Generally, NHL patients can receive such treatment once during a year but upon recurrence of the lymphoma, such treatment can be repeated. In another dosing regimen, patients treated with low-grade NHL receive four weeks of an anti-BR3 antibody (375 mg/m2 weekly) followed at week five by three additional courses of the antibody plus standard CHOP (cyclophosphamide, doxorubicin, vincristine and prednisone) or CVP (cyclophosphamide, vincristine, prednisone) chemotherapy, which was given every three weeks for three cycles.

For treating rheumatoid arthritis, in one embodiment, the dosage range for the anti-BR3 antibody is 125 mg/m² (equivalent to about 200 mg/dose) to 600 mg/m², given in two doses, e.g., the first dose of 200 mg is administered on day one followed by a second dose of 200 mg on day 15. In different embodiments, the dosage is selected from the group consisting of 250 mg/dose, 275 mg/dose, 300 mg/dose, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose and 600 mg/dose.

In treating disease, the BR3 binding antibodies of the invention can be administered to the patient chronically or intermittently, as determined by the physician of skill in the disease.

A patient administered a drug by intravenous infusion or subcutaneously may experience adverse events such as fever, chills, burning sensation, asthenia and headache. To alleviate or minimize such adverse events, the patient may receive an initial conditioning dose(s) of the antibody followed by a therapeutic dose. The conditioning dose(s) will be lower than the therapeutic dose to condition the patient to tolerate higher dosages.

It is contemplated that BR3 binding antibodies of this invention that (1) lack ADCC function or have reduced ADCC function compared to an antibody comprising a wild type human IgG Fc; (2) lack the ability to partially or fully inhibit BAFF binding to BR3 or (3) lack ADCC function or have reduced ADCC function compared to an antibody comprising a wild type human IgG Fc and lack the ability to partially or fully inhibit BAFF binding to BR3 will be useful, for example, as in a replacement therapy, alternative therapy or a maintenance therapy for patients that have or are expected to have significantly adverse responses to therapies with anti-BR3 antibodies that inhibit BAFF and BR3 binding and have ADCC function. For example, it is contemplated that a patient can be first treated with anti-BR3 antibodies that inhibit BAFF and BR3 binding and have ADCC function followed by treatments with anti-BR3 antibodies that (1) lack ADCC function or have reduced ADCC function compared to antibodies comprising wild type human IgG Fc; (2) lack the ability to partially or fully inhibit BAFF binding to BR3 or (3) lack ADCC function or have reduced ADCC function compared to antibodies comprising wild type human IgG Fc and lack the ability to partially or fully inhibit BAFF binding to BR3.

Route of Administration

The BR3 binding antibodies are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes, generally by intravenous or subcutaneous administration.

In on embodiment, the anti-BR3 antibody is administered by intravenous infusion with 0.9% sodium chloride solution as an infusion vehicle. In another embodiment, the anti-BR3 antibodies are administered with a pre-filled syringe.

Combination Therapy

The BR3-binding antibodies or polypeptides of this invention can be used in combination with a second therapeutic agent to treat the disease. It should be understood that the term second therapeutic agent does not preclude treating the subjects other additional therapies. The reference to a second therapeutic agent is meant to differentiate the agent from the specific BR3-binding antibody or polypeptide also being used. In one embodiment, a patient to be treated with the BR3 binding antibodies or polypeptides for an autoimmune disease or a cancer can be treated concurrently, sequentially (before or after), or alternatingly with a biologic response modifier (BRM) to stimulate or restore the ability of the immune system to fight disease and/or infection in a multidrug regimen. BRMs can include monoclonal antibodies, such as antibodies that target TNF-alpha or IL-1 (e.g., Enbrel®, Remicade®, and Humira®), interferon, interleukins (e.g, IL-2, IL-12) and various types of colony-stimulating factors (CSF, GM-CSF, G-CSF). For example, the BRMs may interfere with inflammatory activity, ultimately decreasing joint damage.

In one embodiment, the second therapeutic is an IAP inhibitor.

In another embodiment, a patient to be treated with the BR3 binding antibodies or polypeptides for an autoimmune disease or a cancer can be treated concurrently, sequentially (before or after), or alternatingly with a B cell depleting agent.

In one embodiment, a patient to be treated with the BR3 binding antibodies for an autoimmune disease or a cancer can be treated concurrently, sequentially (before or after), or alternatingly with a BAFF antagonist.

In another embodiment, the cancers and neoplasms described above, the patient can be treated with the BR3 binding antibodies of the present invention in conjunction with one or more therapeutic agents such as a chemotherapeutic agent in a multidrug regimen. The BR3 binding antibody can be administered concurrently, sequentially (before or after), or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the BR3 binding antibody is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with an anti-BR3 antibody of the present invention in conjunction with CHOP (cyclophosphamide, doxorubicin, vincristine and prednisone) therapy. In another embodiment, a cancer or neoplasm in a patient can be treated with a BR3 binding antibody of the invention in combination with CVP (cyclophosphamide, vincristine, and prednisone) chemotherapy. In a specific embodiment, the patient suffering from BR3-positive NHL is treated with humanized anti-BR3 antibody in conjunction with CVP. In a specific embodiment of the treatment of chronic lymphocytic leukemia (CLL,) the BR3 binding antibody is administered in conjunction with chemotherapy with one or more nucleoside analogs, such as fludarabine, Cladribine (2-chlorodeoxyadenosine, 2-CdA[Leustatin]), pentostatin (Nipent), with cyclophosphamide.

In treating the autoimmune diseases or autoimmune related conditions described above, the patient can be treated with the BR3 binding antibodies of the present invention in conjunction with a second therapeutic agent, such as an immunosuppressive agent, such as in a multi drug regimen. The BR3 binding antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-L-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a BR3 antibody of the invention in conjunction with any one or more of the following drugs: DMARDS (disease-modifying anti-rheumatic drugs (e.g., methotrexate), NSAI or NSAID (non-steroidal anti-inflammatory drugs), HUMIRA® (adalimumab; Abbott Laboratories), ARAVA® (leflunomide), REMICADE® (infliximab; Centocor Inc., of Malvern, Pa.), ENBREL® (etanercept; Immunex, WA), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNF. Infliximab is a chimeric monoclonal antibody that binds to TNF. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., “Guidelines for the management of rheumatoid arthritis” Arthritis & Rheumatism 46(2): 328-346 (February, 2002). In a specific embodiment, the RA patient is treated with a BR3 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with a BR3 binding antibody of the invention in conjunction with, for example, Remicade® (infliximab; from Centocor Inc., of Malvern, Pa.), ENBREL® (etanercept; Immunex, WA).

Treatments for SLE include high-dose corticosteroids and/or cyclophosphamide (HDCC).

For the treatment of psoriasis, patients can be administered a BR3 binding antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with the BR3 binding antibody sequentially or concurrently with cyclosporine.

### Pharmaceutical Formulations

Therapeutic formulations of the BR3-binding antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-BR3 antibody formulations are described in WO98/56418, expressly incorporated herein by reference. Another formulation is a liquid multidose formulation comprising the anti-BR3 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-BR3 formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

One formulation for the humanized anti-BR3 antibody is antibody at 12-14 mg/mL in 10 mM histidine, 6% sucrose, 0.02% polysorbate 20, pH 5.8.

In a specific embodiment, anti-BR3 antibody and in particular 9.1RF, 9.1RF (N434 mutants), or V3-46s is formulated at 20 mg/mL antibody in 10 mM histidine sulfate, 60 mg/ml sucrose., 0.2 mg/ml polysorbate 20, and Sterile Water for Injection, at pH5.8.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Articles of Manufacture and Kits

Another embodiment of the invention is an article of manufacture containing materials useful for the treatment of autoimmune diseases and related conditions and BR3 positive cancers such as non-Hodgkin's lymphoma. Yet another embodiment of the invention is an article of manufacture containing materials useful for the treatment of immunodeficiency diseases. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a BR3 binding antibody of the invention. The label or package insert indicates that the composition is used for treating the particular condition. The label or package insert will further comprise instructions for administering the antibody composition to the patient. Articles of manufacture and kits comprising combinatorial therapies described herein are also contemplated.

Package insert refers to instructions customarily included in commercial packages of therapeutic products, that contain information about the indications, usage, dosage, administration, contraindications and/or warnings concerning the use of such therapeutic products. In one embodiment, the package insert indicates that the composition is used for treating non-Hodgkins' lymphoma.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for B-cell killing assays, as a positive control for apoptosis assays, for purification or immunoprecipitation of BR3 from cells. For isolation and purification of BR3, the kit can contain an anti-BR3 antibody coupled to beads (e.g., sepharose beads). Kits can be provided which contain the antibodies for detection and quantitation of BR3 in vitro, e.g. in an ELISA or a Western blot. As with the article of manufacture, the kit comprises a container and a label or package insert on or associated with the container. The container holds a composition comprising at least one anti-BR3 antibody of the invention. Additional containers may be included that contain, e.g., diluents and buffers, control antibodies. The label or package insert may provide a description of the composition as well as instructions for the intended in vitro or diagnostic use.

### Monoclonal-Antibodies

Anti-BR3 antibodies can be monoclonal antibodies. Monoclonal antibodies can be prepared, e.g., using hybridoma methods, such as those described by Kohler and Milstein, Nature, 256:495 (1975) or can be made by recombinant DNA methods (U.S. Pat. No. 4,816,567) or can be produced by the methods described herein in the Example section. In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the BR3 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the BR3 polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using techniques known in the art.

### Human and Humanized Antibodies

The anti-BR3 antibodies can further comprise humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that typically contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al., Nature, 321: 522-525 (1986); Riechmann et al., Nature, 332: 323-329 (1988); Presta, Curr. Op. Struct. Biol., 2:593-596 (1992).

Some methods for humanizing non-human antibodies are described in the art and below in the Examples. Generally, a humanized antibody has one or more amino acid residues introduced into it from a source that is non-human. These non-human amino acid residues are often referred to as “import” residues, which are typically taken from an “import” variable domain. According to one embodiment, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to one embodiment of this technique, antibody V domain sequences are cloned in-frame into either a major or minor coat protein gene of a filamentous bacteriophage, such as M13 or fd, and displayed as functional antibody fragments on the surface of the phage particle. Phage display can be performed in a variety of formats, e.g., as described below in the Examples section or as reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human antibodies can also be produced using various techniques known in the art, including phage display libraries. Hoogenboom and Winter, J. Mol. Biol., 227: 381 (1991); Marks et al., J. Mol. Biol., 222: 581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Multi-Specific Anti-BR3 Antibodies

Multi-specific antibodies are monoclonal, preferably human or humanized, antibodies that have binding specificities for two or more different antigens (e.g., bispecific antibodies have binding specificities for at least two antigens). For example, one of the binding specificities can be for the BR3 polypeptide, the other one can be for any other antigen. According to one preferred embodiment, the other antigen is a cell-surface protein or receptor or receptor subunit. For example, the cell-surface protein can be a natural killer (NK) cell receptor. Thus, according to one embodiment, a bispecific antibody of this invention can bind BR3 and bind a NK cell and, optionally, activate the NK cell.

Examples of methods for making bispecific antibodies have been described. Traditionally, the recombinant production of bispecific antibodies is based on the co-expression of two immunoglobulin heavy-chain/light-chain pairs, where the two heavy chains have different specificities. Milstein and Cuello, Nature, 305: 537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10: 3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121:210 (1986).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered that has dual Fc regions and can thereby have enhanced complement lysis and ADCC capabilities. See, Stevenson et al., Anti-Cancer Drug Design, 3: 219-230 (1989).

Mutations or alterations in the Fc region sequences can be made to improve FcR binding (e.g., FcgammaR, FcRn). According to one embodiment, an antibody of this invention has at least one altered effector function selected from the group consisting of ADCC, CDC, and improved FcRn binding compared to a native IgG or a parent antibody. Examples of several useful specific mutations are described in, e.g., Shields, R L et al. (2001) JBC 276(6)6591-6604; Presta, L. G., (2002) Biochemical Society Transactions 30(4):487-490; and WO publication WO00/42072.

According to one embodiment, the Fc receptor mutation is a substitution at least one position selected from the group consisting of: 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 or 439 of the Fc region, wherein the numbering of the residues in the Fc region is according to the EU numbering system. According to one specific embodiment, the substitution is a 434 residue substitution selected from the group consisting of N434A, N434F, N4343Y and N434H. According to another embodiment, the substitutions are a D265A/N297A mutation. According to another embodiment, the substitutions are S298A/E333A/K334A or S298A/K326A/E333A/K334A. According to another embodiment, the substitution is K322A.

Examples of native sequence human IgG Fc region sequences, humIgG1 (non-A and A allotypes) (SEQ ID NOs:133 and 135, respectively), humIgG2 (SEQ ID NO:136), humIgG3 (SEQ ID NO:137) and humIgG4 (SEQ ID NO:138) have been described previously. Examples of native sequence murine IgG Fc region sequences, murIgG1 (SEQ ID NO:139), murIgG2A (SEQ ID NO:140), murIgG2B (SEQ ID NO:141) and murIgG3 (SEQ ID NO:142), have also been described previously.

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody can be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

### Pharmaceutical Compositions of Antibodies and Polypeptides

Antibodies specifically binding a BR3 polypeptide identified herein, as well as other molecules identified by the screening assays disclosed hereinbefore, can be administered for the treatment of various disorders as noted above and below in the form of pharmaceutical compositions.

Lipofectins or liposomes can be used to deliver the polypeptides and antibodies or compositions of this invention into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

### Diagnostic Use and Imaging

Labeled antibodies, and derivatives and analogs thereof, which specifically bind to a BR3 can be used for diagnostic purposes to detect, diagnose, or monitor diseases and/or disorders associated with the expression, aberrant expression and/or activity of a polypeptide of the invention. According to one preferred embodiment, the anti-BR3 antibodies used in diagnostic assays or imaging assays that involve injection of the anti-BR3 antibody into the subject are antibodies that do not block the interaction between BAFF and BR3 or only partially blocks the interaction between BAFF and BR3. The invention provides for the detection of aberrant expression of a BR3 polypeptide, comprising (a) assaying the expression of the BR3 polypeptide in cells or body fluid of an individual using one or more antibodies of this invention and (b) comparing the level of gene expression with a standard gene expression level, whereby an increase or decrease in the assayed gene expression level compared to the standard expression level is indicative of aberrant expression.

The invention provides a diagnostic assay for diagnosing a disorder to be treated with an anti-BR3 antibody or polypeptide of this invention, comprising (a) assaying the expression of BR3 polypeptide in cells or body fluid of an individual using an antibody of this invention, (b) assaying the expression of BAFF polypeptide in cells or body fluid of the individual and (c) comparing the level of BAFF gene expression with a standard gene expression level, whereby an increase or decrease in the assayed BAFF gene expression level compared to the standard expression level and the presence of BR3 polypeptide in the fluid or diseased tissue is indicative of a disorder to be treated with an anti-BR3 antibody or polypeptide. With respect to cancer, the presence of BR3 or a relatively high amount of BR3 transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru; luminol; and fluorescent labels, such as fluorescein and rhodamine, and biotin.

Techniques known in the art may be applied to label antibodies of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

Diagnosis of a disease or disorder associated with expression or aberrant expression of a BR3 molecule in an animal, preferably a mammal and most preferably a human can comprise the step of detecting BR3 molecules in the mammal. In one embodiment, diagnosis comprises: (a) administering (for example, parenterally, subcutaneously, or intraperitoneally) to a mammal an effective amount of a labeled anti-BR3 antibody or polypeptide which specifically binds to the BR3 molecule, respectively; (b) waiting for a time interval following the administering for permitting the labeled molecule to preferentially concentrate at sites in the subject where the BR3 molecule is expressed (and for unbound labeled molecule to be cleared to background level); (c) determining background level; and (d) detecting the labeled molecule in the subject, such that detection of labeled molecule above the background level indicates that the subject has a particular disease or disorder associated with expression or aberrant expression of BR3. Background level can be determined by various methods including, comparing the amount of labeled molecule detected to a standard value previously determined for a particular system. According to specific embodiments, the antibodies of the invention are used to quantitate or qualitate concentrations of cells of B cell lineage or cells of monocytic lineage.

According to one specific embodiment, BR3 polypeptide expression or overexpression is determined in a diagnostic or prognostic assay by evaluating levels of BR3 present on the surface of a cell, or secreted by the cell (e.g., via an immunohistochemistry assay using anti-BR3 antibodies or anti-BAFF antibodies; FACS analysis, etc.). Alternatively, or additionally, one can measure levels of BR3 polypeptide-encoding nucleic acid or mRNA in the cell, e.g., via fluorescent in situ hybridization using a nucleic acid based probe corresponding to a BR3-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). One can also study BR3 molecules or BAFF molecules overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

### Assays

The agonist anti-BR3 antibodies of this invention are used for directly stimulating the BR3 biological pathway and not the TACI or the BCMA receptor pathways (i.e., “BR3-specific”). Such agonist antibodies can be used to identify downstream markers of the BR3-specific signaling pathway. Accordingly, an assay for identifying downstream markers of the BR3 pathway can comprise the steps of administering an agonist BR3 binding, BR3-specific antibody or polypeptide to a cell expressing BR3 on its cell surface and detecting changes in gene expression (e.g, microarray or ELISA assay) or protein activity of the cell. According to another embodiment of this invention, the agonist antibody can be used to screen for BR3 pathway specific inhibitors. Said method of screening can, e.g., comprise the steps of administering a BR3 binding, BR3-specific antibody or polypeptide to a cell expressing BR3 on its cell surface, administering a candidate compound to the cell and determining whether the candidate compound inhibited proliferation of the cell or survival of the cell or both.

All publications (including patents and patent applications) cited herein are hereby incorporated in their entirety by reference, including U.S. Provisional Application No. 60/640,323, filed Dec. 31, 2004.

The following DNA sequences were deposited under the terms of the Budapest Treaty with the American Type Culture Collection (ATCC), 10801 University Blvd., Manassas, Va. 20110-2209, USA as described below:

| Material | Deposit No. | Deposit Date |
|---|---|---|
| Hu9.1-RF-H-IgG | PTA-6315 | Nov. 17, 2004 |
| Hu9.1-RF-L-IgG | PTA-6316 | Nov. 17, 2004 |
| Hu2.1-46.DANA-H-IgG | PTA-6313 | Nov. 17, 2004 |
| Hu2.1-46.DANA-L-IgG | PTA-6314 | Nov. 17, 2004 |
| HuV3-46s-H-IgG | PTA-6317 | Nov. 17, 2004 |
| HuV3-46s-L-IgG | PTA-6318 | Nov. 17, 2004 |
| Murine B Cells: 12B12.1 | PTA-6624 | Apr. 8, 2005 |
| Murine B Cells: 3.1 | PTA-6622 | Apr. 8, 2005 |

The deposits herein were made under the provisions of the Budapest Treaty on the International Recognition of the Deposit of Microorganisms for the Purpose of Patent Procedure and the Regulations thereunder (Budapest Treaty). This assures maintenance of a viable culture of the deposits for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposits to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. 122 and the Commissioner's rules pursuant to thereto (including 37 C.F.R. 1.14 with particular reference to 8860G 638).

The assignee of the present application has agreed that if a culture of the materials on deposits should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, Va. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Materials

Murine monoclonal antibodies that bind to BR3 were generated from mice immunized with aggregated human BR3-Fc. Those antibodies include those produced from hybridomas referred to as 11G9, 8G4, 7B2, 1E9, 12B12, 1E9, 1A11, 8E4, 10E2 and 12B12. Hybridomas producing murine monoclonal antibodies referred to as 2.1 and 9.1, have been previously described (International Patent Application PCT/US01/28006 (WO 02/24909)) and deposited in the American Type Culture Collection (ATCC) as ATCC NO. 3689 and ATCC NO. 3688, respectively (10801 University Blvd., Manassas, Va. 20110-2209, USA). The B9C11 antibody, a hamster anti-mouse BR3 antibody that is specific for murine BR3 and does not bind human BR3, as well as the antibodies from hybridoma 3.1, were obtained from Biogen Idec, Inc.

MiniBR3 peptide (TPCVPAECFDLLVRHCVACGLLR (SEQ ID NO:150) was synthesized as a C-terminal amide on a Pioneer peptide synthesizer (PE Biosystems) using standard Fmoc chemistry. Peptides were cleaved from resin by treatment with 5% triisopropyl silane in TFA for 1.5-4 hr at room temperature. After removal of TFA by rotary evaporation, peptides were precipitated by addition of ethyl ether, then purified by reversed-phase HPLC (acetonitrile/H₂O/0.1% TFA). Peptide identity was confirmed by electrospray mass spectrometry. After lyophilization, the oxidized peptide was purified by HPLC. HPLC fractions containing reduced miniBR3 were adjusted to a pH of ˜9 with NH₄OH; the disulfide between cysteines 24 and 35 was then formed by addition of a small excess of K₃Fe(CN)₆, and the oxidized peptide purified by HPLC. Acm groups were removed (with concomitant formation of the second disulfide) by treatment of the HPLC eluate with a small excess of I₂ over ˜4 h. The progress of the oxidation was monitored by analytical HPLC, and the final product was again purified by HPLC. MiniBR3 was amino-terminally biotinylated while on resin, then cleaved and purified exactly as described above for the unmodified peptide.

The human BR3 extracellular domain (hBR3-ECD) and the mouse BR3 extracellular domain (mBR3-ECD) constructs were produced in bacteria by subcloning their sequences into the pET32a expression vector (Novagen), creating a fusion with an N-terminal thioredoxin (TRX)-His-tag followed by an enterokinase protease site. E. coli BL21(DE3) cells (Novagen) were grown at 30° C. and protein expression was induced with IPTG. TRX-BR3 was purified over a Ni-NTA column (Qiagen), eluted with an imidazole gradient, and cleaved with enterokinase (Novagen). BR3 was then purified over an S-Sepharose column, refolded overnight in PBS, pH 7.8, in the presence of 3 mM oxidized and 1 mM reduced glutathione, dialyzed against PBS, repurified over a MonoS column, concentrated, and dialyzed into PBS. The human BR3 extracellular sequence used:

(SEQ ID NO: 151)
MRRGPRSLRGRDAPAPTPCVPAECFDLLVRHCVACGLLRTPRPKPAGASS
PAPRTALQPQE.

The mouse extracellular sequence:

(SEQ ID NO: 152)
MGARRLRVRS QRSRDSSVPTQCNQTECFDP LVRNCVSCELFHTPDTGH
TSSLEPGTALQPQEGS.

The human and mouse BR3-Fc proteins were produced in chinese hamster ovary cells (CHO cells) as described previously (Pelletier, M., et al., (2003) J. Biol. Chem. 278, 33127-33133). The mouse BR3-Fc sequence (mBR3-Fc) was described originally in the Yan et al., (2001) Current Biology 11, 1547-1552. The murine BR3-Fc sequence is as follows:

(SEQ ID NO: 153)
MSALLILALVGAAVASTGARRLRVRSQRSRDSSVPTQCNQTECFDPLVR
NCVSCELFHTPDTGHTSSLEPGTALQPQEGQVTGDKKIVPRDCGCKPCIC
TVPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQWN
GQPAENYKNTQPIMNTNGSYFVYSKLNVQKSNWEAGNTFTCSVLHEGLHN
HHTEKSLSHSPGK.

Variant human BR3-Fc fusion (vBR3-Fc) generally relates to an Fc fusion protein comprising a variant sequence of the ECD sequence of the naturally occurring human BR3 sequence, which variant also binds BAFF and has tends to aggregate less than native human BR3 sequence.

Human BAFF as used herein can be expressed and purified as previously described (Gordon, N. C., et al., (2003) Biochemistry 42, 5977-5983). A DNA fragment encoding BAFF residues 82-285 was cloned into the pET15b (Novagen) expression vector, creating a fusion with an N-terminal His-tag followed by a thrombin cleavage site. E. coli BL21(DE3) (Novagen) cultures were grown to mid-log phase at 37° C. in LB medium with 50 mg/L carbenicillin and then cooled to 16° C. prior to induction with 1.0 mM IPTG. Cells were harvested by centrifugation after 12 h of further growth and stored at −80° C. The cell pellet was resuspended in 50 mM Tris, pH 8.0, and 500 mM NaCl and sonicated on ice. After centrifugation, the supernatant was loaded onto a Ni-NTA agarose column (Qiagen). The column was washed with 50 mM Tris, pH 8.0, 500 mM NaCl, and 20 mM imidazole and then eluted with a step gradient in the same buffer with 250 mM imidazole. BAFF-containing fractions were pooled, thrombin was added, and the sample was dialyzed overnight against 20 mM Tris, pH 8.0, and 5 mM CaCl2 at 4° C. The protein was further purified on a monoQ (Pharmacia) column and finally on an S-200 size exclusion column in 20 mM Tris, 150 mM NaCl, and 5 mM MgCl₂.

In some experiments, a hybrid BAFF molecule was used. The hybrid BAFF molecule comprised residues 82-134 of human BAFF recombinantly fused to the N-terminal of residues 128-309 of mouse BAFF. The recombinant protein was expressed in bacteria and purified as described above. The addition of the human sequence aided in the expression of the mBAFF protein. In other experiments, human BAFF expressed in CHO cells were used in B cell proliferation assays.

### Example 2

### Competitive Elisa Assay

A competitive ELISA assay was used to measure the relative affinity of anti-BR3 antibodies for the extracellular domain of human BR3 and miniBR3. In these experiments the binding of biotinylated BR3-ECD to antibody adsorbed on microtiter plate (Nunc MaxiSorp) wells was competed with unlabeled BR3-ECD or miniBR3. BR3-ECD was biotinylated by reaction with a 10-fold molar excess of sulfo-NHS-biotin (Pierce) at ambient temperature for 2 hours. Antibodies were coated at 5 μg/mL in coating buffer (50 mM sodium carbonate pH 9.6) for 2 hours at room temperature followed by blocking with PBS/0.05% Tween-20/2.5% (wt/vol) powdered skim milk for 1 hour. The amount of biotin-BR3-ECD required to produce an absorbance at 492 nm of about 1.0 after detection with streptavidin-HRP was determined. For Mabs 3.1 and 12B12 the concentration of biotin-BR3-ECD required was 5 nM, for 8G4 and 11G9 it was 2 nM, and for 2.1 and 9.1 the biotin-BR3-ECD concentration was 200 pM. Solutions containing these concentrations of biotin-BR3-ECD and a varied concentration of unlabeled BR3-ECD or mini-BR3 were prepared and added to individual wells of a microtiter plate coated with antibody. After incubation for 2 hours with shaking the solutions were decanted and the wells were rinsed 6× with PBS/0.05% Tween-20. Streptavidin-HRP (0.5 μg/mL) was added, incubated with shaking for 30 minutes, and then the wells were emptied and rinsed as above. The bound HRP was detected by adding a solution containing PBS, 0.01% hydrogen peroxide, and 0.8 mg/mL O-phenylenediamine. Color was allowed to develop for 20 minutes and then the reaction was quenched by adding an equal volume of 1 M phosphoric acid. Absorbance at 492 nm was measured on a plate reader (Thermo LabSystems). The absorbance as a function of competitor concentration was analyzed by using a four-parameter equation (1) to determine the IC50 for inhibition of biotin-BR3-ECD binding:

((m1−m4)/(1+(m0/m3)̂m2))+m4 (1)

where m1 is the absorbance with no competitor, m4 is the absorbance at infinite inhibitor concentration, m0 is the competitor concentration, and m3 is the IC50 value.

TABLE 3

IC50 (nM)

| Antibody | BR3-ECD | mini-BR3 |
|---|---|---|
| 2.1 | 9 | 9 |
| 9.1 | 9 | 16 |
| 8G4 | 8 | 22 |
| 11G9 | 10 | 6 |
| 3.1 | 330 | >1000 |
| 12B12 | 60 | >1000 |

2.1, 9.1, 8.G4 and 11G9 bound the 26-residue miniBR3 with an affinity similar to that of the full-length BR3 extracellular domain (Table 3). As shown below, those antibodies also blocked BR3 binding to BAFF. The 3.1 and 12B12 antibodies, which did not bind as well to miniBR3 also did not block BAFF-BR3 interaction.

### Example 3

### Humanized Antibodies

(a) Materials and Methods

The residue numbers referred to below were designated according to Kabat (Kabat et al., Sequences of proteins of immunological interest, 5th Ed., Public Health Service, National Institutes of Health, Bethesda, Md. (1991)). Single letter amino acid abbreviations are used. DNA degeneracies are represented using the IUB code (N=A/C/G/T, D=A/G/T, V=A/C/G, B=C/G/T, H=A/C/T, K=G/T, M=A/C, R=A/G, S=G/C, W=A/T, Y=C/T).

Direct Hypervariable Region Grafts onto the Acceptor Human Consensus Framework—

The VL and VH domains from murine 2.1, 11G9 and 9.1 were aligned with the human consensus kappa I (huKI) and human subgroup III consensus VH (huIII) domains. To make the CDR grafts, huKI and the acceptor VH framework, which differs from the human subgroup III consensus VH domain at 3 positions: R71A, N73T, and L78A (Carter et al., Proc. Natl. Acad. Sci. USA 89:4285 (1992)) were used. See bolded letters in FIGS. 1-3. Hypervariable regions from murine 2.1 (mu2.1), 11G9 (mu11G9) and 9.1 (mu9.1) antibodies were engineered into the acceptor human consensus framework to generate a direct CDR-graft (2.1graft, 11G9graft and 9.1graft) (FIGS. 1-3). In the VL domain, the following regions were grafted to the human consensus acceptor: positions 24-34 (L1), 50-56 (L2) and 89-97 (L3) (Kabat numbering system). In the VH domain, positions 26-35 (H1), 49-65 (H2) and 94-102 (H3) (Kabat numbering system) were grafted (FIGS. 1-3). MacCallum et al. (MacCallum et al. J. Mol. Biol. 262: 732-745 (1996)) have analyzed antibody and antigen complex crystal structures and found positions 93 and 94 of the heavy chain are part of the contact region thus it seems reasonable to include these positions in the definition of CDR-H3 when humanizing antibodies. The nucleic acid sequences encoding the grafted CDR-human framework sequences were contained in a phagemid. The phagemid was a monovalent Fab-g3 display vector and included 2 open reading frames under control of the phoA promoter. The first open reading frame consisted of the stII signal sequence fused to the VL and CH1 domains of the acceptor light chain and the second consisted of the stII signal sequence fused to the VH and CH1 domains of the acceptor heavy chain followed by the minor phage coat protein P3.

The direct-graft variants were generated by Kunkel mutagenesis using a separate oligonucleotide for each hypervariable region. Correct clones were assessed by DNA sequencing.

Soft randomization of the hypervariable regions—For each grafted antibody, sequence diversity was introduced into each hypervariable region using a soft randomization strategy that maintains a bias towards the murine hypervariable region sequence. This was accomplished using a poisoned oligonucleotide synthesis strategy first described by Gallop et al., J. Med. Chem. 37:1233-1251 (1994). For a given position within a hypervariable region to be mutated, the codon encoding the wild-type amino acid is poisoned with a 70-10-10-10 mixture of nucleotides resulting in an average 50 percent mutation rate at each position.

Soft randomized oligonucleotides were patterned after the murine hypervariable region sequences and encompassed the same regions defined by the direct hypervariable region grafts. The amino acid position at the beginning of H2 (position 49) in the VH domain, was limited in sequence diversity to A, G, S or T by using the codon RGC.

Generation of phage libraries—Randomized oligonucleotide pools designed for each hypervariable region were phosphorylated separately in six 20 μl reactions containing 660 ng of oligonucleotide, 50 mM Tris pH 7.5, 10 mM MgCl₂, 1 mM ATP, 20 mM DTT, and 5 U polynucleotide kinase for 1 h at 37° C. The six phosphorylated oligonucleotide pools were then combined with 20 μg of Kunkel template in 50 mM Tris pH 7.5, 10 mM MgCl₂ in a final volume of 500 μl resulting in an oligonucleotide to template ratio of 3. The mixture was annealed at 90° C. for 4 min, 50° C. for 5 min and then cooled on ice. Excess, unannealed oligonucleotide was removed with a QIAQUICK PCR purification kit (Qiagen kit 28106) using a modified protocol to prevent excessive denaturation of the annealed DNA. To the 500 μl of annealed mixture, 150 μl of PB was added, and the mixture was split between 2 silica columns. Following a wash of each column with 750 μl of PE and an extra spin to dry the columns, each column was eluted with 110 μl of 10 mM Tris, 1 μl EDTA, pH 8. The annealed and cleaned-up template (220 μl) was then filled in by adding 1 μl 100 mM ATP, 10 μl 25 mM dNTPs (25 mM each of dATP, dCTP, dGTP and dTTP), 15 μl 100 mM DTT, 25 μl 10×TM buffer (0.5 M Tris pH 7.5, 0.1 M MgCl₂), 2400 U T4 ligase, and 30 U T7 polymerase for 3 h at room temperature.

The filled in product was analyzed on Tris-Acetate-EDTA/agarose gels (Sidhu et al., Methods in Enzymology 328:333-363 (2000)). Three bands are usually visible: the bottom band is correctly filled and ligated product, the middle band is filled but unligated and the top band is strand displaced. The top band is produced by an intrinsic side activity of T7 polymerase and is difficult to avoid (Lechner et al., J. Biol. Chem. 258:11174-11184 (1983)); however, this band transforms 30-fold less efficiently than the top band and usually contributes little to the library. The middle band is due to the absence of a 5′ phosphate for the final ligation reaction; this band transforms efficiently and unfortunately, gives mainly wild type sequence.

The filled in product was then cleaned-up and electroporated into SS320 cells and propagated in the presence of M13/KO7 helper phage as described by Sidhu et al., Methods in Enzymology 328:333-363 (2000). Library sizes ranged from 1-2×10⁹ independent clones. Random clones from the initial libraries were sequenced to assess library quality.

Phage Selection—The human BR3ecd or variant BR3-Fc fusion (vBR3-Fc) was used as the target for phage selection (Kayagaki et al. Immunity 17:515-524 (2002) and Pelletier et al. J. Biol. Chem. 278:33127-33133 (2003)). BR3ecd or vBR3-Fc was coated on MaxiSorp microtiter plates (Nunc) at 10 μg/ml in PBS. For the first round of selection 8 wells of target were used; a single well of target was used for successive rounds of selection. Wells were blocked for 1 h using Casein Blocker (Pierce). Phage were harvested from the culture supernatant and suspended in PBS containing 1% BSA and 0.05% Tween 20 (PBSBT). After binding to the wells for 2 h, unbound phage were removed by extensive washing with PBS containing 0.05% Tween 20 (PBST). Bound phage were eluted by incubating the wells with 50 mM HCl, 0.5 M KCl for 30 min. Phage were amplified using Top 10 cells and M13/KO7 helper phage and grown overnight at 37° C. in 2YT, 50 μg/ml carbenacillin. The titers of phage eluted from a target coated well were compared to titers of phage recovered from a non-target coated well to assess enrichment.

Phage libraries were also sorted using a solution sorting method (Lee, C. V., et al. (2004) J. Mol. Biol. 340(5):1073-93). vBR3-Fc was biotinylated using Sulfo-NHS-LC-biotin (Pierce) (b-vBR3-Fc). Microtiter wells were coated with 10 μg/ml neutravidin in PBS overnight at 4 C and then blocked for 1 h using Casein Blocker (Pierce). The first round of panning was performed using the standard plate sorting method with immobilized vBR3-Fc. For the second round of selection, 200 μl phage suspended in PBS containing 0.05% Tween 20 (PBST) and 1% BSA were mixed with 100 nM b-vBR3-Fc for 2 hr. Phage bound to b-vBR3-Fc were captured on neutravidin coated wells for 5 min and unbound phage were washed away with PBST. Phage were eluted using 100 mM HCl for 30 m, neutralized, and propagated in XL1 blue cells (Strategene) in the presence of KO07 helper phage (New England Biolabs). The next rounds of selection were performed similarly with the following exceptions: in round 3 the final b-vBR3-Fc concentration was 20 nM, in rounds 4 and 5 the final b-vBR3-Fc concentration was 1 nM. After phage binding was established for 1 h in round 5, 1 ∥M unbiotinylated vBR3-Fc was added to the mixture for 64 h prior to capture on neutravidin.

Phage ELISA—MaxiSorp microtiter plates were coated with human vBR3-Fc at 10 μg/ml in PBS over night and then blocked with Casein Blocker. Phage from culture supernatants were incubated with serially diluted vBR3-Fc in PBST containing 1% BSA in a tissue culture microtiter plate for 1 h after which 80 μl of the mixture was transferred to the target coated wells for 15 min to capture unbound phage. The plate was washed with PBST and HRP conjugated anti-M13 (Amersham Pharmacia Biotech) was added (1:5000 in PBST containing 1% BSA) for 40 min. The plate was washed with PBST and developed by adding Tetramethylbenzidine substrate (Kirkegaard and Perry Laboratories, Gaithersburg, Md.). The absorbance at 405 nm was plotted as a function of target concentration in solution to determine an IC₅₀. This was used as an affinity estimate for the Fab clone displayed on the surface of the phage.

Fab Production and Affinity Determination

To express Fab protein for affinity measurements, a stop codon was introduced between the heavy chain and g3 in the phage display vector. Clones were transformed into E. coli 34B8 cells and grown in AP5 media at 30° C. (Presta et al. Cancer Res. 57: 4593-4599 (1997)). Cells were harvested by centrifugation, suspended in 10 mM Tris, 1 mM EDTA pH 8 and broken open using a microfluidizer. Fab was purified with Protein G affinity chromatography. Affinity determinations were performed by surface plasmon resonance using a BIAcore™-2000. vBR3-Fc or hBR3ecd were immobilized in 10 mM Acetate pH4.5 (220 or 100 response units (RU), respectively) on a CM5 sensor chip and 2-fold dilutions of Fab (6.25 to 100 nM) in PBST were injected. Each sample was analysed with 2-minute association and 20-minute dissociation. After each injection the chip was regenerated using 10 mM Glycine pH 1.5. Binding response was corrected by subtracting the RU from a blank flow cell. A 1:1 Languir model of simultaneous fitting of kₒₙ and k_{off} was used for kinetics analysis.

(b) Results and Discussion

Humanization of 2.1, 11G9 and 9.1—The human acceptor framework used for humanization is based on the framework used for the Herceptin® antibody and consists of the consensus human kappa I VL domain and a variant of the human subgroup III consensus VH domain. The variant VH domain has 3 changes from the human consensus: R71A, N73T and L78A. The VL and VH domains of murine 2.1, 11G9 and 9.1 were each aligned with the human kappa I and subgroup III domains; each complementarity region (CDR) was identified and grafted into the human acceptor framework to generate a CDR graft that could be displayed as a Fab on phage. When phage displaying the 2.1, 11G9 or 9.1 CDR grafts were tested for binding to immobilized vBR3-Fc, low binding affinity was observed.

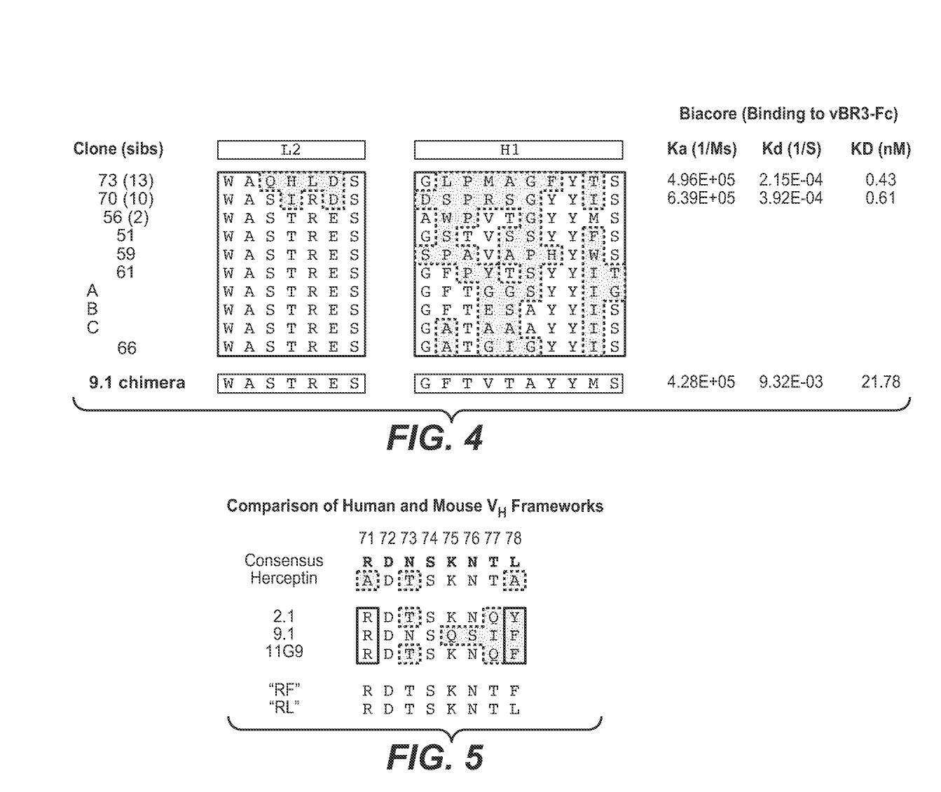

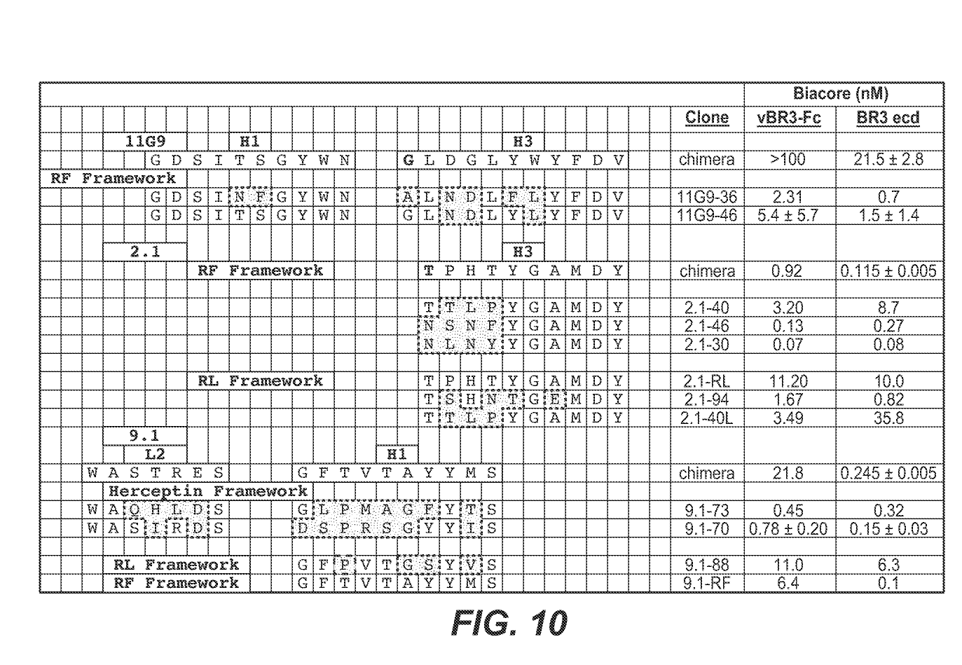

A CDR repair library was generated for each antibody in which the CDR regions of each CDR graft were soft randomized. Each CDR graft library was panned against immobilized vBR3-Fc for 4 rounds of selection. Enrichment was only observed for the CDR graft corresponding to 9.1. Clones were picked for DNA sequence analysis and revealed sequence changes targeted at CDR-L2 and CDR-H1 (FIG. 4). Clones were screened using the vBR3-Fc phage ELISA and select clones were further analyzed by Biacore using expressed Fab protein. Two clones, 9.1-70 and 9.1-73 showed improved binding to vBR3-Fc relative to the chimeric 9.1 Fab (FIG. 10).

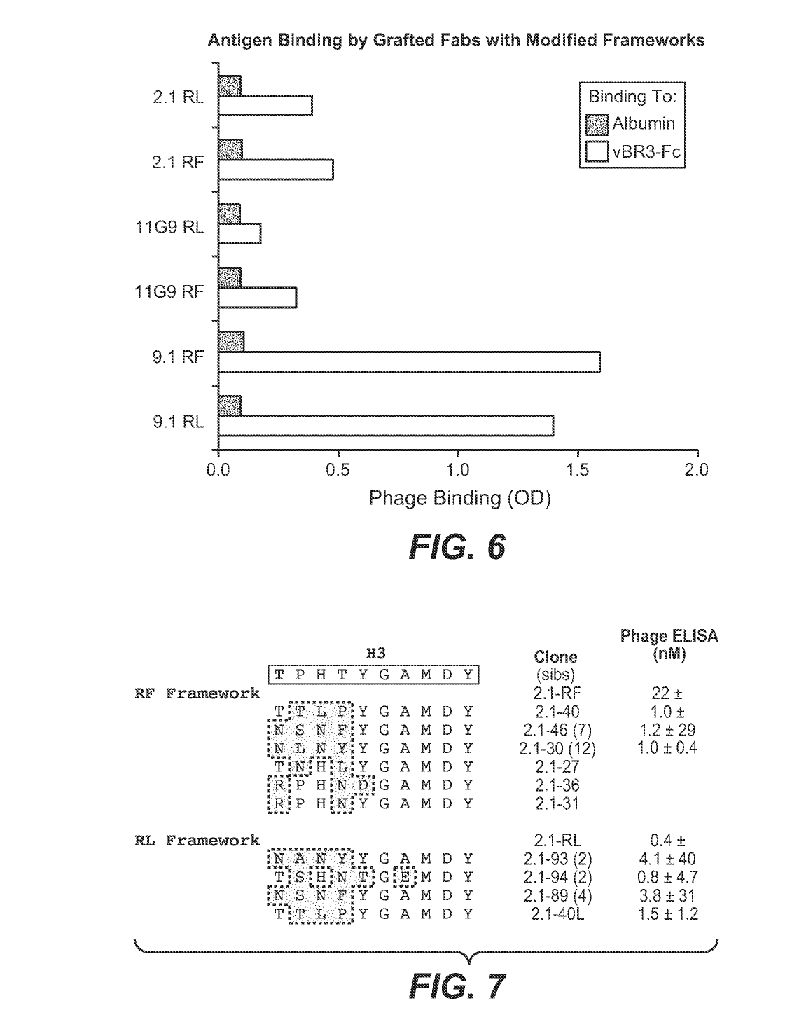

Since binding had not been recruited in the 2.1-graft and 11G9-graft using CDR repair, we inspected differences between the murine and acceptor frameworks. Interestingly 2.1 and 11G9 as well as 9.1 more closely resembled the human consensus subgroup III sequence at positions 71 and 78 than the acceptor framework we initially employed (FIG. 5). This prompted us to investigate CDR repair using 2 new frameworks, “RL” and “RF.” These frameworks differ from the acceptor framework in that R71, present in the consensus, is restored and position 78 is either changed to the consensus as a Leucine (RL) or modified to resemble the murine framework at this position by introducing a Phenylalanine (RF). These framework changes led to modest improvements in 2.1 and 11G9 phage binding to vBR3-Fc. The binding of 9.1 CDRs grafted onto either the RL or RF frameworks (9.1-RL or 9.1-RF) was greatly improved (FIG. 6).

CDR repair libraries were generated as before using a soft randomization strategy simultaneously at each of the 6 CDRs for each of the antibody/framework grafts: 2.1-RL, 2.1-RF, 11G9-RL, 11G9-RF, 9.1-RL and 9.1-RF. For these selections a solution sorting method was used to enhance the efficiency of the affinity-based phage selection process. By manipulating the biotinylated target concentration, reducing the phage capture time to lower backgrounds and the addition of unbiotinylated target to eliminate clones with faster off rates, high affinity clones can be proficiently selected (Lee, C. V., et al. J. Mol. Biol. (2004) 340(5):1073-93). The 12 libraries were sorted independently utilizing b-vBR3-Fc as described above in Methods.

Following 5 rounds of selection, DNA sequence of individual clones from each of the libraries was analyzed. Clones were screened using the vBR3-Fc phage ELISA and select clones were analyzed further by BIAcore Surface Plasmon Resonance (SPR) using expressed Fab protein. Several clones were identified that have BR3 binding affinities that met or exceeded the monomeric affinity of the chimeric antibody.

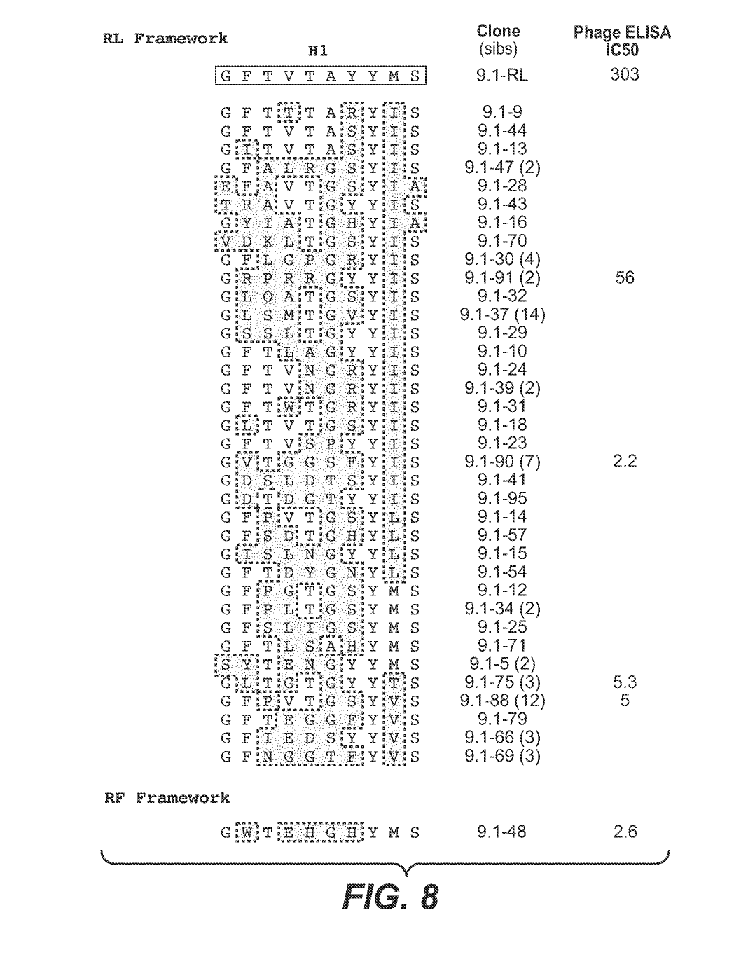

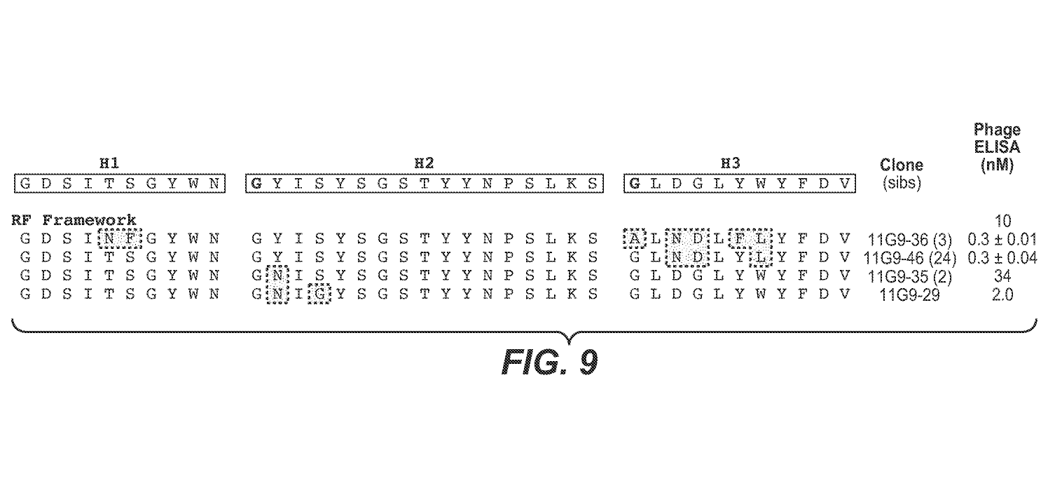

For the 9.1-RL and 9.1-RF libraries sequence changes were again concentrated in CDR-H1 suggesting that the redesign of this CDR was important to the restoration of antigen binding (FIG. 8). In particular, the mutation M341 was frequently included among the various clones. Other frequently found changes in CDR-H1 include A31G and T28P, although numerous other substitutions throughout CDR-H1 appear to be well tolerated. From these results it is clear that there are multiple sequence changes that can repair the affinity of 9.1 grafted onto a human framework and that this antibody can be humanized by either framework changes (e.g. 9.1-RF) or by CDR-repair (e.g. 9.1-70 and 9.1-73) to generate affinities that exceed that of the initial murine antibody.

For the 11G9 libraries, enrichment was only observed when using the 11G9-RF as a template for the CDR repair library where sequence changes were observed in CDR-H1, CDR-H2 and CDR-H3 (FIG. 8). The 2 highest affinity clones however, each had similar changes to CDR-H3; both clones included the changes D96N, G97D and W100L. The affinities of these clones exceeded that of the monomeric murine 11G9 affinity by >10-fold.

Enrichment was observed for both the 2.1-RL and 2.1-RF libraries (FIG. 7). Interestingly similar sequence changes, targeting CDR-H3, were observed in both libraries. In fact in 2 cases the changes to CDR-H3 were identical between the libraries (94-97_{NSNF} and 95-97_{TLP}). This is amazing given the potential sequence diversity that was introduced due to the library design. A common class of sequences observed in both libraries contained T94N and H96N in combination with other changes at positions 95 and 97 (e.g. 94-97_{NSNF, 94}-97_{NLNY}, and 94-97_{NANY}). These variants tended to have the highest affinity for vBR3-Fc or hBR3ecd. In fact, the affinity of clone 2.1-30 (94-97_{NLNY}) exceeded that of the monomeric murine 2.1 affinity.

Summary of Changes for Humanization

Starting from a graft of the 6 murine 9.1 CDRs (defined as positions 24-34 (L1), 50-56 (L2), 89-97 (L3), 26-35 (H1), 49-65 (H2) and 94-102 (H3)) into the human consensus Kappa I VL and subgroup III VH domains, 2 routes to the humanization of this antibody have been identified. The first utilized the 3 framework changes present in the Herceptin® antibody (R71A, N73T and L78A) in addition to the selection of a new CDR-H1 sequence and 2 changes in CDR-L2. This led to a humanized variant (9.1-70) with a nearly 2-fold higher affinity than the affinity of the chimeric 9.1 Fab. The second route utilized the addition of 2 changes in the framework (N73T and L78F) and no changes to the CDRs (9.1-RF), again leading to a nearly 2-fold higher affinity than the affinity of the chimeric 9.1 Fab.

Starting from a graft of the 6 murine 11G9 CDRs (defined as positions 24-34 (L1), 50-56 (L2), 89-97 (L3), 26-35 (H1), 49-65 (H2) and 94-102 (H3)) into the human consensus Kappa I VL and subgroup III VH domains, the addition of 2 changes in the framework (N73T and L78F) and 3 changes in CDR-H3 (D96N, G97D and W100L) leads to a fully human 11G9 antibody (11G9-46) with a >10-fold improved affinity relative to the chimeric 11G9 Fab affinity.

Starting from a graft of the 6 murine 2.1 CDRs (defined as positions 24-34 (L1), 50-56 (L2), 89-97 (L3), 26-35 (H1), 49-65 (H2) and 94-102 (H3)) into the human consensus Kappa I VL and subgroup III VH domains, the addition of a single change in the framework (N73T) and 4 changes in CDR-H3 (T94N, P95L, H96N and T97Y) leads to a fully human 2.1 antibody (2.1-30) with an improved affinity relative to the chimeric 2.1 Fab affinity.

Results of biacore binding assays with selected clones are shown in FIG. 10.

### Example 4

### Anti-BR3Antibodies Derived from Naive Phage Libraries

Additional antibodies that bind BR3 were initially selected from phage-displayed synthetic antibody libraries that were built on a single human framework by introducing synthetic diversity at solvent-exposed positions within the heavy chain complementarity-determining regions (CDRs) as described below.

(a) Phagemid Vectors for Library Construction

Phagemids pV0350-2b and pV0350-4, were designed to display a Fab template monovalently or bivalently, respectively, on the surfaces of M13 phage particles.

The Fab template is based on the h4D5 antibody, which antibody is a humanized antibody that specifically recognizes a cancer-associated antigen known as Her-2 (erbB2). The h4D5 sequence was obtained by polymerase chain reaction using the humAb4D5 version 8 (“humAb4D5-8”) sequence (Carter et al., (1992) PNAS 89:4285-4289). The h4D5 nucleic sequence encodes modified CDR regions from a mouse monoclonal antibody specific for Her-2 in a human consensus sequence Fab framework. Specifically, the sequence contains a kappa light chain (LC region) upstream of VH and CH1 domains (HC region). The method of making the anti-Her-2 antibody and the identity of the variable domain sequences are provided in U.S. Pat. Nos. 5,821,337 and 6,054,297.

The vector pV0350-2b was constructed by modifying a previously described phagemid (pHGHam-gIII) that has been used for the phage display of human growth hormone (hGH) under the control of a phoA promoter. An open reading frame in phGHam-gIII that encodes for the stII secretion signal sequence and hGH fused to the C-terminal domain of the M13 minor coat protein P3 (cP3) was replaced with a DNA fragment containing two open reading frames. The first open reading frame encoded for the h4D5 light chain (version 8) and the second encoded for the variable (VH) and first constant (CH1) domains of the h4D5 heavy chain fused to cP3; each protein was directed for secretion by an N-terminal stII signal sequence. The amber stop codon between the heavy chain fragment and cP3 was deleted, as this modification has been shown to increase the levels of Fab displayed on phage. An epitope tag was added to the C terminus of the h4D5 light chain (gD tag). The vector for bivalent display (pV0350-4) was identical with pV0350-2b, except for the insertion of a DNA fragment encoding for a GCN4 leucine zipper between the heavy chain CH1 domain and cP3 as described. The light chain gene was further modified in both phagemids at three positions to encode for amino acids most commonly found in the Kabat database of natural antibody sequences; specifically, Arg66 was changed to Gly and Asn30 and His91 were changed to Ser. These changes were found to increase Fab expression and display on phage. Site-directed mutagenesis was performed using the method of Kunkel et al. (Kunkel, J. D., et al., (1987) Methods Enzymol 154:367-82).

(b) Library Construction

Phage-displayed libraries were generated using oligonucleotide-directed mutagenesis and “stop template” versions of pV0350-2b or pV0350-4 as described (Lee, C. V., et al., (2004) J. Immunol. Methods 284:119-132; Lee, C. V., et al., (2004) JMB 340:1073-1093). Stop codons (TAA) were embedded in all three heavy-chain CDRs. These were repaired during the mutagenesis reaction by a mixture of degenerate oligonucleotides that annealed over the sequences encoding for CDR-H1, -H2 and -H3 and replaced codons at the positions chosen for randomization with tailored degenerate codons. Mutagenesis reactions were electroporated into E. coli SS320 cells, and the cultures were grown overnight at 30° C. in 2YT broth supplemented with KO7 helper phage, 50 g/ml of carbenicillin and 50 g/ml of kanamycin. Phage were harvested from the culture medium by precipitation with PEG/NaCl as described (Sidhu, S. S. et al., (2000), Methods Enzymol. 328:333-363). Each electroporation reaction used ˜10¹¹ E. coli cells and ˜10 ug of DNA and resulted in 1×10⁹-5×10⁹ transformants.

A distinct library was made with degenerate oligonucleotides tailored to mimic the natural diversity of CDR-H1 and CDR-H2 (Table 1 in Lee, C. V, et al., (2004), JMB, supra): library 3 (Lib-3) with Fab.zip template. See Lib-3 described in Lee, C. V, et al., (2004), supra. Two to four oligonucleotides for CDR-H1 and CDR-H2 were combined to increase the coverage of natural diversity. Lib-3 used oligonucleotides H1a and H1b (ratio 2:1) and H2a-c (ratio 1:2:0.1) for CDR-H1 and CDR-H2, respectively (see Table 1 of Lee, C. V. et al. (2004), JMB, supra, for a description of the oligonucleotides).

For positions 95-100 in CDR-H3, Lib-3 consists of a set of libraries with expanded CDR-H3 lengths containing either NNS codons (or NNK codons) or a modified version of the NNS codon (the XYZ codon) that contained unequal nucleotide ratios at each position of the codon triplet. The NNS codon encompasses 32 codons and encodes for all 20 amino acids. X contained 38% G, 19% A, 26% T and 17% C; Y contained 31% G, 34% A, 17% T and 18% C; and Z contained 24% G and 76% C. The CDR-H3 design for Lib-3 is described in Table 5 of Lee, C. V. et al., (2004), supra. Separate mutagenesis reactions were performed and electroporated for each CDR-H3 length, except for lengths seven and eight residues, which were electroporated together.

Phage display levels of complete Fabs in each library was examined by measuring the binding of 48 randomly picked clones to anti-gD antibody. For Lib-3, similar levels of display were observed for the different CDR-H3 lengths, except that libraries incorporating the longest CDR-H3s (from 15-19 residues) had a reduced percentage of Fab displaying clones (15-30%). This may reflect the reduced mutagenesis efficiency when using very long synthetic oligonucleotides.

### Phage Sorting

A F(ab)′2 (CDR-H1/H2/H3 randomized) synthetic phage antibody library was used to sort against mouse extracellular domain of BR3 (mBR3-ECD), mouse BR3 extracellular domain fused to an Fc region of IgG1 (mBR3-Fc), human BR3 extracellular domain (hBR3-ECD) and extracellular domain of human BR3 fused to an Fc region of IgG1 (vBR3-Fc) on the plate. 96-well Nunc Maxisorp plates were coated with 100 ul/well of target antigen (mBR3-ECD, mBR3-Fc, hBR3-ECD and vBR3-Fc) (5 ug/ml) in coating buffer (0.05M sodium carbonate buffer, pH9.6) at 4° C. overnight or room temperature for 2 hours. The plates were blocked with 65 ul 1% blocking protein for 30 min and 40 ul 1% Tween20 for another 30 min (blocking protein: 1^{st} round—bovine serum albumin (BSA), 2^{nd} round—ovalbumin, 3^{rd} round—milk, 4th round—BSA. Next, the phage library was diluted to 3˜5 O.D/ml with 1% BSA with 0.1% Tween 20 (1 O.D.=1.13×10¹³ phage/ml). In general, the phage input was 1^{st} round 3-5 O.D./ml, 2^{nd} round 3 O.D./ml, 3^{rd} round 0.5˜1 O.D/ml and 4^{th} round input 0.1˜0.5 O.D/ml. The diluted phage were incubated for 30 minutes at room temperature. The wells were washed at least five times continuously with PBS and 0.05% Tween 20. The blocked phage library was added 100 ul/well to 8 target antigen-coated wells and 2 uncoated wells at room temperature for 1 hour. The plates were washed continuously at least 10 times with PBS and 0.05% Tween 20. The phage were eluted with 100 ul/well of 100 mM HCl at room temperature for 20 minutes. The eluted phage (from coated wells) and background phage (from uncoated wells) were collected in separate tubes. The eluted collections were neutralized by adding 1/10 volume 1M Tris pH 11.0 to both tubes. BSA was added to a final 0.1% into the tube of eluted phage. The eluted phage were heated at 62° C. for 20 minutes. To titer the phage, 90 ul of log phase XL-1 (OD 600 nm˜0.1-0.3) was infected with 10 ul eluted phage or background phage at 37° C. for 30 minutes. Next, the infected cells were serially diluted in 10 fold increments with 90 ul 2YT. 10 ul aliquots of the infected cells were plated per carbenicillin plate.

To propagate the phage, approximately 400 ul of eluted phage was used to infect ˜4 ml log phase XL-1 soup (OD 600 nm˜0.1-0.3) at 37° C. for 30-45 minutes. Helper phage, KO7, and carbenicillin were added to the infection at a final concentration of 1×10¹⁰ pfu/ml KO7 and 50 ug/ml cabenicillin at 37 C for another hour. The culture was grown 2YT media with carbenicillin 50 ug/ml and 50 ug/ml kanamycin to final volumes of 20˜25 ml at 37° C. overnight (or at least 17 hours). The next day, the culture was grown at 30° C. for another 2 hours to increase the phage yield.

The phage were purified by spinning down the cells at 8000 rpm for 10 minutes. The supernatant was collected. 20% PEG/2.5M NaCl was added at ⅕ of the supernatant volume, mixed and allowed to sit on ice for 5 minutes. The phage were spun down into a pellet at 12000 rpm for 15 minutes. The supernatant was collected and spun again for 5 minutes at 5000 rpm. The pellets were resuspended in 1 ml PBS and spun down at 12000 rpm for 15 minutes to clear debris. The steps starting with the PEG/NaCl addition were repeated on the resuspended pellet. The OD of the resuspended phage pellet was read at 270 nm. The second, third and fourth rounds of phage sorting were completed by repeating the phage sorting steps as described above.

### ELISA Screening Assay

Clones from third and fourth rounds were screened for specificity and affinity by ELISA assay. Positive clones (binders) were clones that had binding above background to the target antigens (mBR3-ECD and hBR3-ECD) and not to the blocking protein such as bovine serum albumin.

First, the wells of a 384-well microtiter plate were coated with mBR3-ECD, hBR3-ECD and anti-gD at 20 ul per well (1 ug/ml in coating buffer) at 4° C. overnight or room temperature for 2 hours.

| | |
|---|---|
| BSA | mBR3-ECD |
| Anti-gD | hBR3-ECD |

In another 96 well plate, colonies from third and fourth round were grown overnight at 37° C. in 150 ul 2YT media with 50 ug/ml carbenicillin and helper phage KO7. The plate was spun down at 2500 rpm for 20 minutes. 50 ul of the supernatant was added to 120 ul of ELISA buffer (PBS with 0.5% BSA and 0.05% Tween20) in the coated well plate. 30 ul of mixture was added to each quadrant of 384-well coating plate and incubated at room temperature for 1 hour. Binding was quantified by adding 75 ul/well of horse radish peroxidase (HRP)-conjugated anti-M13 antibody in PBS plus 0.5% BSA and 0.05% Tween20 at room temperature for 30 minutes (Sidhu et al., supra). The wells were washed with PBS—0.05% Tween20 at least five times. Next, 100 ul/well of a 1:1 ratio of 3,3′,5,5′-tetramethylbenzidine (TMB) Peroxidase substrate and Peroxidase Solution B (H₂O₂) ((Kirkegaard-Perry Laboratories (Gaithersburg, Md.)) was added to the well and incubated for 5 minutes at room temperature. The reaction was stopped by adding 100 ul 1M Phosphoric Acid (H₃PO₄) to each well and allowed to incubate for 5 minutes at room temperature. The OD of the yellow color in each well was determined using a standard ELISA plate reader at 450 nm. The clones that bound both mBR3-ECD and hBR3-ECD three fold better than binding to BSA were selected (FIG. 11).

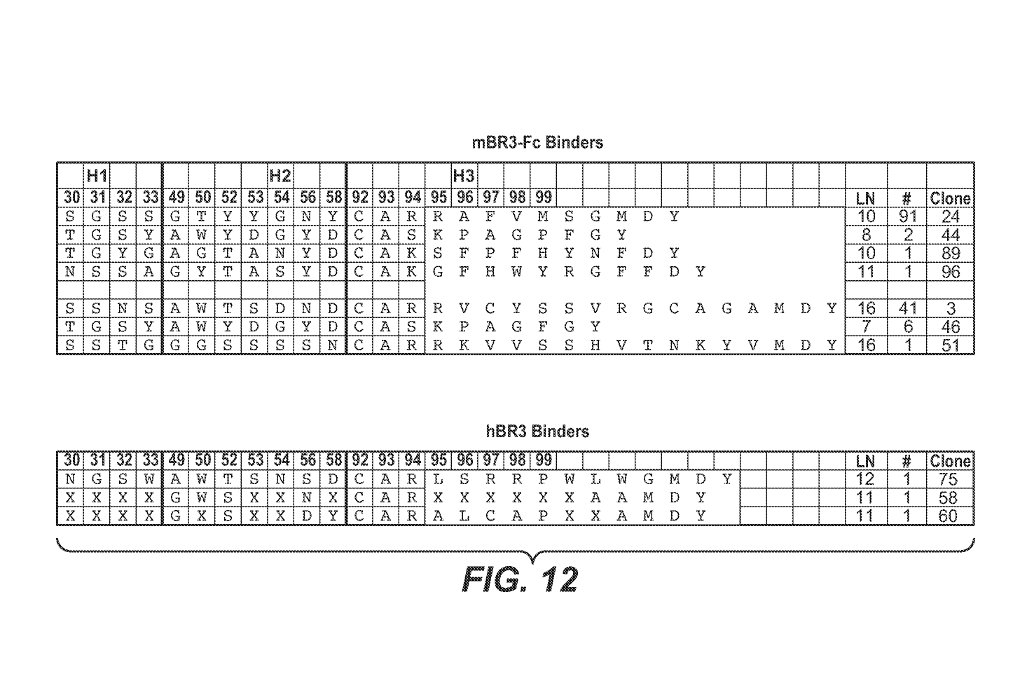

The selected binders were sequenced. Fifteen unique clones were found (one clone from sorting mBR3-ECD, six clones from sorting mBR3-Fc, 8 clones from sorting hBR3-ECD and no clones from sorting hBR3-Fc) (FIG. 12).

### Solution Binding Competition ELISA

To determine the binding affinity for the selected F(ab)′2 phage, competition ELISAs were performed.

First, the phage were propagated and purified. Ten uls of XL-1 bacteria infected with a clone for 30 minutes at 37° C. was plated on a carbenicillin plate. A colony was picked and grown in 2 mls (2YT and 50 ug/ml carbenicillin) at 37 C for 3-4 hours. Helper phage, KO7, were added to the culture at a final concentration of 10¹⁰ pfu/ml for another 1 hour at 37° C. Twenty mls of media (2YT with 50 ug/ml carbenicillin and 50 ug/ml kanamycin were added to the culture for growth overnight at 37° C. The phage were purified as described above.

Second, the concentration of purified phage that would be optimal for use in the following competition ELISA assay was determined (i.e., approximately 90% of maximal binding capacity on the coated plate). 96-well Nunc Maxisorp plates were coated with 2 ug/ml mBR3-ECD or mBR3-Fc in coating buffer at 4° C. overnight or at room temperature for 2 hours. The wells were blocked by adding 65 ul 1% BSA for 30 minutes followed by 40 ul 1% Tween20 for another 30 minutes. Next, the wells were washed five times with PBS—0.05% Tween20. F(ab)′2 phage were diluted to 0.1 O.D./ml in ELISA buffer (PBS—0.5% BSA and 0.05% Tween20) and, then, were added to the wells for 15 minutes at room temperature. The wells were then washed with PBS—0.05% Tween20 at least three times. 75 ul of HRP-conjugated anti-M13 antibody (Amersham, 1/5000 dilution with ELISA buffer) per well was added and incubated at room temperature for 30 minutes. The wells were washed again with PBS—0.05% Tween20 at least five times. Next, 100 ul/well of a 1:1 ratio of 3,3′,5,5′-tetramethylbenzidine (TMB) Peroxidase substrate and Peroxidase Solution B (H2O2) ((Kirkegaard-Perry Laboratories (Gaithersburg, Md.)) was added to the well and incubated for 5 minutes at room temperature. The optical density of the color in each well was determined using a standard ELISA plate reader at 450 nm. The dilutions of phage were plotted against the O.D. readings.

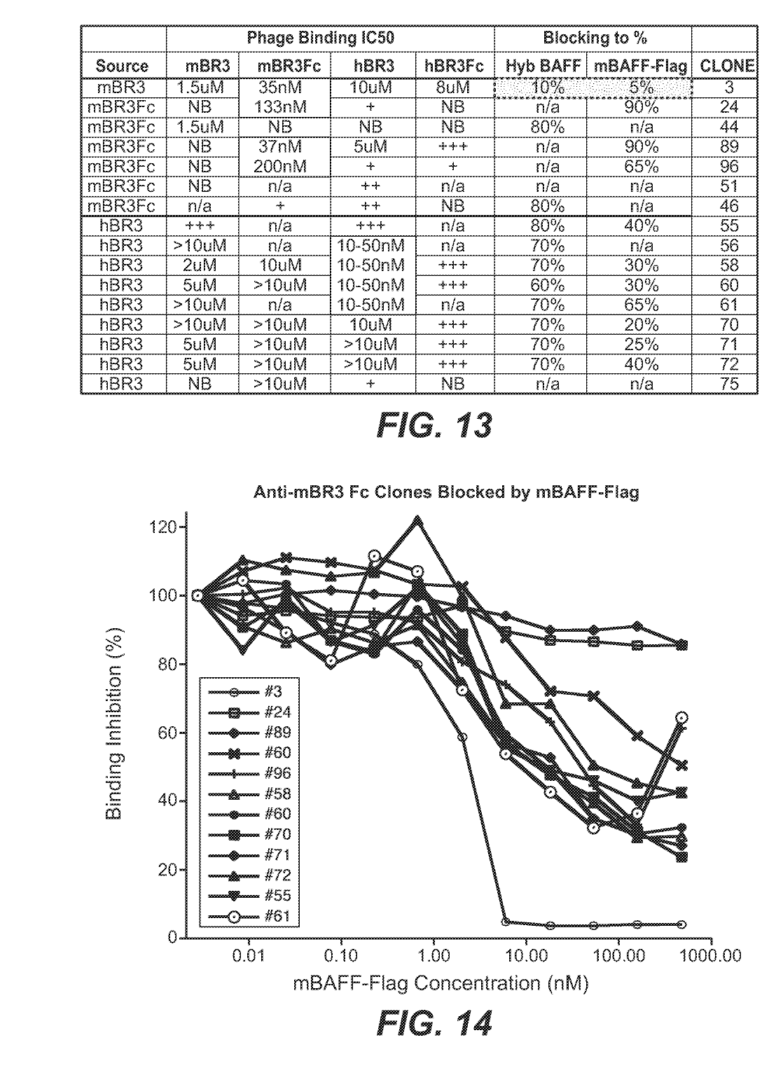

Third, a competition ELISA was performed. 96-well Nunc Maxisorp plates were coated with 2 ug/ml mBR3-ECD or mBR3-Fc in coating buffer at 4° C. overnight or at room temperature for 2 hours. The wells were blocked by adding 65 ul 1% BSA for 30 minutes followed by 40 ul 1% Tween20 for another 30 minutes. The wells were washed with PBS—0.05% Tween20 5 times. Based on the binding assay above, 50 ul of the dilution of phage that resulted in about 90% of maximum binding to the coated plate was incubated with 50 ul of various concentrations of mBR3-ECD or mBR3-Fc or hBR3-ECD or hBR3-Fc (0.1 to 1000 nM) in ELISA buffer solution for 2 hour at room temperature in a well. The unbound phage was assayed by transferring 75 ul of the well mixture to second 96-well plate pre-coated with mBR3-ECD or mBR3-Fc and incubating at room temperature for 15 minutes. The wells of the second plate were washed with PBS—0.5% Tween20 at least three times. 75 ul of HRP-conjugated anti-M13 antibody (1/5000 dilution with ELISA buffer) per well was added and incubated at room temperature for 30 minutes. The wells were washed again with PBS—0.05% Tween20 at least five times. Next, 100 ul/well of a 1:1 ratio of 3,3′,5,5′-tetramethylbenzidine (TMB) Peroxidase substrate and Peroxidase Solution B (H2O2) ((Kirkegaard-Perry Laboratories (Gaithersburg, Md.)) was added to the well and incubated for 5 minutes at room temperature. The reaction was stopped by adding 100 ul 1M Phosphoric Acid (H3PO4) to each well and allowed to incubate for 5 minutes at room temperature. The optical density of the color in each well was determined using a standard ELISA plate reader at 450 nm. The concentrations of competitor mBR3-ECD or mBR3-Fc or hBR3-ECD or hBR3-Fc were plotted against the O.D. readings. The IC50, the concentration of mBR3-ECD or mBR3-Fc or hBR3-ECD or hBR3-Fc that inhibits 50% of the F(ab)′2-phage, represents the affinity (FIG. 13). The V3 clone binds with high affinity to both mouse and human BR3.

mBAFF Blocking ELISA

To find out if these unique clones have similar binding epitope as the ligand (BAFF), mBAFF blocking ELISA was conducted as follows: 96-well Nunc Maxisorp plates were coated with 2 ug/ml mBR3-Fc in coating buffer at 4° C. overnight or at room temperature for 2 hours. The wells were blocked by adding 65 ul 1% BSA for 30 minutes followed by 40 ul 1% Tween20 for another 30 minutes. Next, the wells were washed five times with PBS—0.05% Tween20. Various concentrations of mBAFF-Flag protein in ELISA buffer were incubated in the wells for 30 minutes at room temperature. Then, F(ab)′₂ phages with unique sequences were added to each well for 10 minutes at a concentration that would normally produce 90% binding capacity in the absence of mBAFF-Flag protein. The wells were washed five times with PBS—0.05% Tween20.

Binding was quantified by adding 75 ul/well of horse radish peroxidase (HRP)-conjugated anti-M13 antibody in PBS plus 0.5% BSA and 0.05% Tween20 at room temperature for 30 minutes (Sidhu et al., supra). The wells were washed with PBS—0.05% Tween20 at least five times. Next, 100 ul/well of a 1:1 ratio of 3,3′,5,5′-tetramethylbenzidine (TMB) Peroxidase substrate and Peroxidase Solution B (H₂O₂) ((Kirkegaard-Perry Laboratories (Gaithersburg, Md.)) was added to the well and incubated for 5 minutes at room temperature. The reaction was stopped by adding 100 ul 1M Phosphoric Acid (H₃PO₄) to each well and allowed to incubate for 5 minutes at room temperature. The OD of the solution in each well was determined using a standard ELISA plate reader at 450 nm. Results shown in FIG. 13 and FIG. 14.

Another monovalent format of BAFF blocking ELISA was performed as well. By using mBR3-ECD coated plate, various concentrations of hybrid BAFF protein in ELISA buffer were incubated in the wells for 30 minutes at room temperature. Then, F(ab)′₂ phages with unique sequences were added to each well for 10 minutes at a concentration that would normally produce 90% binding capacity in the absence of hybrid BAFF protein. The following steps were as described above. Results shown in FIG. 13.

FIG. 14 shows that clone 3 (V3) readily blocks BAFF-BR3 binding. Variable region sequences of V3 are depicted in FIG. 15.

### Change F(ab)′₂ Format of V3 Backbone to Fab Format

Since V3 has the best blocking activity by BAFF and also has cross-species binding activity to both mBR3 and hBR3, V3 is the antibody candidate for further affinity improvement. In order to ensure monovalent affinity for future affinity improvement, the leucine zipper was removed by Kunkel mutagenesis with F220 oligo (5′-TCT TGT GAC AAA ACT CAC AGT GGC GGT GGC TCT GGT-3′) (SEQ ID NO:154). In addition, to ensure the incorporation of CDR-L3 in the randomization scheme, a stop codon (TAA) was incorporated in the positions that intend to be diversified in CDR-L3. F9 oligo (5′-TAT TAC TGT CAG CAA CAT TAA TAA AGG CCT TAA CCT CCC ACG TTC GGA-3′) (SEQ ID NO: 155) was used to add stop codon in CDR-L3 region.

### Construct Libraries on V3 Backbone for Affinity Improvement

Hard and soft randomization design was used for affinity improvement. Hard randomization means limited positions were randomized to all 20 amino acids. Soft randomization means that at certain positions the randomization retained 50% parental amino acid and 50% 19 other amino acids or a stop codon. Four libraries have been constructed based on V3 backbone by Kunkel mutagenesis.

V0902-1: CDR-L1(F111+F202=1:1)/L2(F201+F203=1:1)/L3 (F133a:133b:133c:133d=1:1:1:1)
V0902-2: CDR-L3 soft (F232)/H1 soft (F226)/L2 (F201+F203=1:1)
V0902-3: CDR-H3 soft(F228+F229+F230+F231=1:1:0.5:0.5)/L3 soft (F232)
V0902-4: CDR-L3 soft(F232)/H1 soft (F226)/H2 soft (F227)

### Oligos:

| | | |
|---|---|---|
| L1 | F111 (5′-ACC TGC CGT GCC AGT CAG RDT RKT RVW ANW THT GTA | (SEQ ID NO: 156) |
| | GCC TGG TAT CAA CAG AAA C-3′) | |
| | F202 (5′-ACC TGC CGT GCC AGT CAG RDT RKT RVW ANW THT CTG | (SEQ ID NO: 157) |
| | GCC TGG TAT CAA CAG AAA C-3′) | |
| L2 | F201 (5′-CCG AAG CCT CTG ATT TAC KBG GCA TCC AVC CTC TAC TCT | (SEQ ID NO: 158) |
| | GGA GTC CCT-3′) | |
| | F203 (5′-CCG AAG CTT CTG ATT TAC KBG GCA TCC AVC CTC GMA | (SEQ ID NO: 159) |
| | TCT GGA GTC CCT TCT CGC-3′) | |
| L3 | F133a (5′-GCA ACT TAT TAC TGT CAG CAA TMT DMC RVT NHT CCT | (SEQ ID NO: 160) |
| | YKG ACG TTC GGA CAG GGT ACC-3′) | |
| | F133b (5′-GCA ACT TAT TAC TGT CAG CAA TMT DMC RVT NHT CCT | (SEQ ID NO: 161) |
| | TWT ACG TTC GGA CAG GGT ACC-3′) | |
| | F133c (5′-GCA ACT TAT TAC TGT CAG CAA SRT DMC RVT NHT CCT | (SEQ ID NO: 162) |
| | YKG ACG TTC GGA CAG GGT ACC-3′) | |
| | F133d (5′-GCA ACT TAT TAC TGT CAG CAA SRT DMC RVT NHT CCT | (SEQ ID NO: 163) |
| | TWT ACG TTC GGA CAG GGT ACC-3′) | |

### Soft Randomized Oligos Symbol:

5 (70% A, 10% G, 10% C, 10% T)
6 (70% G, 10% A, 10% C, 10% T)
7 (70% C, 10% A, 10% G, 10% T)
8 (70% T, 10% A, 10% G, 10% C)

| | | |
|---|---|---|
| L3 soft | F232 (5′-GCA ACT TAT TAC TGT CAG CAA 567 857 577 577 CCG 776 | (SEQ ID NO: 164) |
| | ACG TTC GGA CAG GGT ACC-3′) | |
| H1 soft | F226 (5′-TGT GCA GCT TCT GGC TTC WCC NTT 567 567 557 567 587 | (SEQ ID NO: 165) |
| | 757 TGG GTG CGT CAG GCC-3′) | |
| H2 soft | F227 (5′-AAG GGC CTG GAA TGG GTT GST 866 ATC 577 776 567 658 | (SEQ ID NO: 166) |
| | 668 557 577 658 TAT GCC GAT AGC GTC AAG-3′) | |
| H3 soft | F228 (5′-GCC GTC TAT TAT TGT GCT CGT 768 686 TGC 857 567 567 | (SEQ ID NO: 167) |
| | 686 768 668 TGC 676 668 676 ATG GAC TAC TGG GGT CAA G-3′) | |
| | F229 (5′-GCC GTC TAT TAT TGT GCT CGT 768 686 867 857 567 567 | (SEQ ID NO: 168) |
| | 686 768 668 867 676 668 676 ATG GAC TAC TGG GGT CAA G-3′) | |
| | F230 (5′-GCC GTC TAT TAT TGT GCT 768 768 686 TGC 857 567 567 | (SEQ ID NO: 169) |
| | 686 768 GGC TGC GCG GGG GCA ATG-3′) | |
| | F231 (5′-GCT CGT CGG GTC TGC TAC 567 567 686 768 668 TGC 676 | (SEQ ID NO: 170) |
| | 668 676 ATG GAC TAC TGG GGT CAA G-3′) | |

### Expression of Phage

E. coli strain SS320/KO7 (KO7 infected) was transformed with the mutagenized DNA described above by electroporation. Transformed bacterial cells were grown up in 2YT media with 50 ug/ml carbenicillin and 50 ug/ml kanamycin for 20 hours at 30° C. Phage were harvested as described (Sidhu et al., Methods Enzymol. (2000), 328:333-363). Briefly, phage were purified by first precipitating them from the overnight culture media with polyethylene glycol, and resuspended in PBS. Phage were quantitated by spectrophotometer with its reading at 268 nm (1 OD=1.13×10¹³/ml).

### Phage Sorting Strategy to Generate Affinity Improvement Over V3

For affinity improvement selection, phage libraries were subjected to plate sorting for the first round and followed by three rounds of solution sorting. At the first round of plate sorting, four libraries were sorted against mBR3-ECD and hBR3-ECD coated plate (NUNC Maxisorp plate) separately. Phage input was approximately 3 O.D/ml in 1% BSA and 0.1% Tween 20. The following steps are as described above in phage sorting section. The elution phage from Library V0902-2, V0902-3 and V902-4 against mBR3-ECD or hBR3-ECD were pooled for propagation.

After the first round of plate sorting, three rounds of solution sorting were performed to increase the stringency of selection.

A) Biotinylation of mBR3-ECD and hBR3-ECD

Before biotinylation, the target protein was placed in amine free buffer, ideally at pH higher than 7.0 and in >0.5 mg/ml concentration. First, the buffer containing mBR3-ECD and hBR3-ECD was exchanged into PBS by using an Amicon Ultra 5K tube. Second, a fresh stock of NHS-Biotin reagent in PBS (100×) was made. An approximate 3:1 molar ratio of NHS-Biotin reagent to target protein was incubate at room temperature for 30 min to 1 h. Then, 0.1M Tris pH7.5 was added to quench the unreacted NHS for 30 min. at room temperature.

B) 96-well Nunc Maxisorp plates were coated with 100 ul/well of neutravidin (5 ug/ml) in PBS at 4° C. overnight or room temperature for 2 hours. The plate were blocked with 65 ul Superblock (Pierce) for 30 min and 40 ul 1% Tween20 for another 30 min.

C) 1 O.D./ml phage propagated from first round of plate sorting were incubated with 100 nM of biotinylated mBR3-ECD or hBR3-ECD in 150-200 ul buffer containing Superblock 0.5% and 0.1% Tween20 for at least 1 hour at room temperature. The mixture was further diluted 5-10× with Superblock 0.5% and applied 100 ul/well to neutravidin coated wells for 5 min at room temperature with gentle shaking so that biotinylated target could bind phage. The wells were washed with PBS-0.05% Tween20 eight times. To determine background binding, control wells containing phage with targets that were not biotinylated were captured on neutravidin-coated plates. As another control (the neutravidin binding control), the biotinylated target was mixed with phage and incubated in wells not coated with neutravidin. Bound phage were eluted with 0.1N HCl for 20 min, neutralized by 1/10 volume of 1M Tris pH11 and titered and propagated for the next round. Next, two more rounds of solution sorting were carried out with decreasing biotinylated mBR3-ECD or hBR3-ECD concentration to 25 nM and 1 nM to increase the stringency. Also, the phage input was decreased to 0.5 O.D/ml and 0.1 O.D/ml to lower the background phage binding.

### High Throughput Affinity Screening ELISA (Single Spot Competition)

Colonies were picked from the third and fourth round screens and grown overnight at 37° C. in 150 ul/well of 2YT media with 50 ug/ml carbenicillin and 1e 10/ml KO7 in 96-well plate (Falcon). From the same plate, a colony of XL-1 infected V3 phage was picked as control.

96-well Nunc Maxisorp plates were coated with 100 ul/well of mBR3-ECD (2 ug/ml) in coating buffer at 4° C. overnight or room temperature for 2 hours. The plates were blocked with 65 ul of 1% BSA for 30 min and 40 ul of 1% Tween 20 for another 30 min.

The phage supernatant was diluted 1:10 in ELISA buffer (PBS with 0.5% BSA, 0.05% Tween20) with or without 100 nM mBR3-ECD or hBR3-ECD in 100 ul total volume and incubated at least 1 hour at room temperature (RT) in a F plate (NUNC). 75 ul of mixture were transferred without or with mBR3-ECD or with hBR3-ECD side by side to the mBR3-ECD coated plates. The plate was gently shook for 10-15 minutes to allow the capture of unbound phage to the mBR3-ECD coated plate. The plate was washed at least five times with PBS-0.05% Tween 20. The binding was quantified by adding horse radish peroxidase (HRP)-conjugated anti-M13 antibody in ELISA buffer (1:5000) and incubated for 30 min at room temperature. The plates were washed with PBS-0.05% Tween 20 at least five times. Next, 100 ul/well of a 1:1 ratio of 3,3′,5,5′-tetramethylbenzidine (TMB) Peroxidase substrate and Peroxidase Solution B (H₂O₂) ((Kirkegaard-Perry Laboratories (Gaithersburg, Md.)) was added to the well and incubated for 5 minutes at room temperature. The reaction was stopped by adding 100 ul 1M Phosphoric Acid (H₃PO₄) to each well and allowed to incubate for 5 minutes at room temperature. The OD of the yellow color in each well was determined using a standard ELISA plate reader at 450 nm. The OD reduction (%) was calculated by the following equation.

OD₄₅₀ₙₘ reduction (%)=(OD₄₅₀ₙₘ of wells with competitor)/(OD₄₅₀ₙₘ of well with no competitor)*100

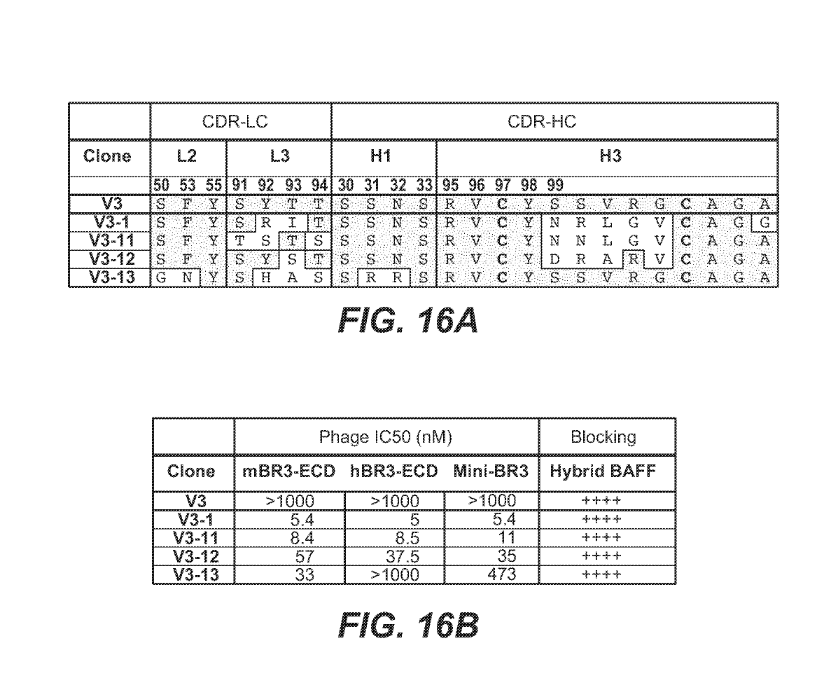

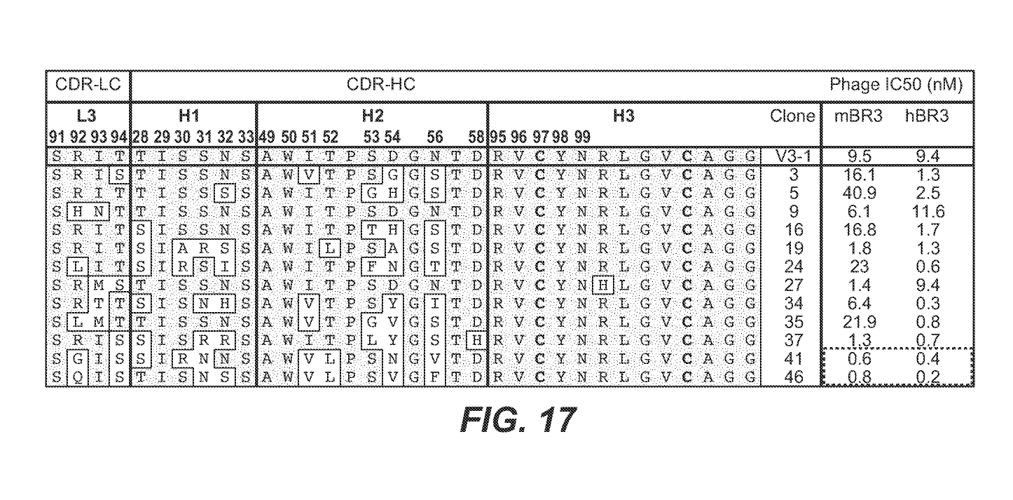

In comparison to the OD₄₅₀ₙₘ reduction (%) of the well of V3 phage (100%), clones that had the OD₄₅₀ₙₘ reduction (%) to mBR3-ECD and hBR3-ECD both lower than 50% were picked. Fourteen clones were picked only from the V0902-2,3,4 pooled library sorted against mBR3-ECD. There were no hits found either from V0902-1 LC hard randomized library sorted against mBR3-ECD or from both libraries sorted against hBR3-ECD. These fourteen clones were sequenced. In the end, there were four unique sequences (V3-1, V3-11, V3-12 and V3-13). All four unique clones have the same CDR-L1 and CDR-H2 as V3 clone, which are identical with 4D5 library template. V3-1, V3-11 and V3-12 are from library V0902-3 whereas V3-13 is from library V0902-2. FIG. 16A shows partial sequences of the L2, L3, H1 and H3 regions.

### Functional Characterization of New Clones

BAFF Blocking ELISA was performed on the V3-derived clones to test BAFF blocking activity compared to V3 clone. All four clones show complete blocking activity to hybrid BAFF. It is implied that all four clones have similar binding epitopes to BR3 as BAFF.

In addition, competition ELISAs were performed to determine the affinity of these phage clones to mBR3-ECD, hBR3-ECD and mini-BR3. Mini-BR3 is a 26 residue peptide fragment that full affinity for BAFF. The results of blocking ELISA and phage competition ELISA were summarized in FIG. 16B.

### Fab Constructs for Expression in Bacterial Cells

V3, V3-1, V3-11, V3-12 and V3-13 phagemids were modified by removing the viral cP3 sequences, replacing them with a terminator sequence containing 5′-GCTCGGTTGCCGCCGGGCGTTTTTTATG-3′ (SEQ ID NO:171) and removing the sequences encoding gD tags (pw0276-V3, pw0276-V3-1, pw0276-V3-11 and pw0276-V3-12 respectively). All constructs were transformed into E. coli 34B8 cells. Single colonies were picked and grown in complete CRAP medium with 25 ug/ml Carbenicillin at 30° C. for at least 22 hours. The expressed proteins were purified through a Protein G high trap column (Amersham Pharmacia).

Biacore measurement Surface plasmon resonance assays on a BIAcore™-2000 were used to determine the affinity of anti-BR3 Fabs. Immobilized mBR3-ECD and hBR3-ECD on CM5 chips at ˜150 response units (RU). Fab samples of increasing concentration from 3 nM to 500 nM were injected at 20 ul/min, and binding responses on mBR3-ECD or hBR3-ECD were corrected by subtracting of RU from a blank flow cell. For kinetics analysis, 1:1 Languir model of simultaneous fitting of kₒₙ and k_{off} was used. The apparent kD values are reported in Table 4.

TABLE 4

| | Clone | Kon(1/Ms) | Koff(1/s) | kD(nM) | Phage IC50 (nM) |
|---|---|---|---|---|---|
| mBR3-ECD | V3 | 7.80E+03 | 5.50E−03 | 700 | >1000 |
| | V3-1 | 7.71E+04 | 1.95E−04 | 2.5 | 5.4 |
| | V3-11 | 4.36E+04 | 8.88E−04 | 20.4 | 8.4 |
| | V3-12 | 3.60E+04 | 1.30E−03 | 36 | 57 |
| | V3-13 | 1.00E+04 | 4.10E−03 | 40 | 33 |
| hBR3-ECD | V3 | 2.10E+03 | 2.60E−03 | 1300 | >1000 |
| | V3-1 | 3.73E+04 | 2.93E−04 | 7.9 | 5 |
| | V3-11 | 2.18E+04 | 1.13E−03 | 60.1 | 8.5 |
| | V3-12 | 1.30E+04 | 9.10E−04 | 72 | 37.5 |
| | V3-13 | 2.30E+03 | 2.80E−03 | 1200 | >1000 |

### Construct Libraries Using V3-1 for Further Affinity Improvement

Soft and softer randomization has been used to further affinity improvement. Soft randomization means at certain positions 50% was retained as the parental amino acid and the other 50% were the other 19 amino acids or a stop codon. Softer randomization means at certain positions retain 75% as parental amino acid and other 25% as other 19 amino acids or stop codon. Four libraries have been constructed based on V3-1 backbone by Kunkel mutagenesis.

### V1008-1: L3 (F279+F280+F293=1:1:0.2)/H3 (F285+F286=1:1)

### V1008-2: L3 (F279)/H3 (F283+F284=1:1)

### V1008-3: H1(F281)/H2 (F282)/L3 (F279)

### V1008-4: L3(F280+F293=1:4)/H3 (F283+F284+F266+F267=1:1:1:1)

### Oligos:

| | | |
|---|---|---|
| L3 soft | F279 (5′-ACT TAT TAC TGT CAG CAA 568 767 587 577 CCG 777 ACG | (SEQ ID NO: 172) |
| | TTC GGA CAG GGT-3′) | |
| | F280 (5′-ACT TAT TAC TGT CAG CAA 568 767 587 577 568 CCG 777 ACG | (SEQ ID NO: 173) |
| | TTC GGA CAG GGT-3′) | |
| | F293 (5′-ACT TAT TAC TGT CAG CAA 878 NNK NNK NNK 878 CCG CCC | (SEQ ID NO: 174) |
| | ACG TTC GGA CAG GGT-3′) | |
| H1 soft | F281 (5′-GCA GCT TCT GGC TTC WCC ATT 568 568 568 878 ATA CAC | (SEQ ID NO: 175) |
| | TGG GTG CGT C-3′) | |
| H2 soft | F282 (5′-CTG GAA TGG GTT GCT TGG RTT 578 CCT 878 657 GGT 878 | (SEQ ID NO: 176) |
| | ACT 657 TAT GCC GAT AGC GTC AAG-3′) | |
| H3 soft | F283 (5′-GTC TAT TAT TGT GCT CGT 766 687 TGC 857 557 767 788 668 | (SEQ ID NO: 177) |
| | 688 TGC GCT GGT GGG ATG-3′) | |
| | F284 (5′-GTC TAT TAT TGT GCT CGT 766 687 TGC 857 557 767 CTT GGT | (SEQ ID NO: 178) |
| | GTT TGC 678 668 668 ATG GAC TAC TGG GGT CAA-3′) | |
| | F285 (5′-GTC TAT TAT TGT GCT CGT 766 687 RST 857 557 767 788 668 | (SEQ ID NO: 179) |
| | 688 RST GST GST GSG ATG GAC TAC TGG GGT-3′) | |
| | F286 (5′-TAT TAT TGT GCT CGT CGG 687 RST 857 557 767 788 668 | (SEQ ID NO: 180) |
| | 688 RST 678 668 668 ATG GAC TAC TGG GGT C-3′) | |
| H3 softer | F266 (5′-GTC TAT TAT TGT GCT CGT 766 687 TGC 857 557 767 788 668 | (SEQ ID NO: 181) |
| | 688 TGC GCT GGT GGG ATG-3′) | |
| | F267 (5′-GTC TAT TAT TGT GCT CGT 766 687 TGC 857 557 767 CTT | (SEQ ID NO: 182) |
| | GGT GTT TGC 678 688 668 ATG GAC TAC TGG GGT CAA-3′) | |

### Softer Randomized Oligos Symbol:

5 (85% A, 5% G, 5% C, 5% T)
6(85% G, 5% A, 5% C, 5% T)
7 (85% C, 5% A, 5% G, 5% T)
8 (85% T, 5% A, 5% G, 5% C)

### Phage Sorting Strategy to Generate Affinity Improvement Over V3-1

Four rounds of solution sorting were performed in four libraries (V1008-1, V1008-2, V1008-3 and V1008-4) by decreasing biotinylated mBR3-ECD and hBR3-ECD concentration. Phage input was 3 O.D/ml at first round and 1, 0.5, 0.1 for the following three rounds. For library V1008-1, 100 nM biotinylated targets were used for the first round. Then 10 nM, 10 nM and 2 nM biotinylated targets were used for the following three rounds. As for the other three libraries (V1008-2, V1008-3 and V1008-4), 20 nM of biotinylated targets were used for the first round. Then 1 nM, 1 nM and 0.5 nM biotinylated targets were used in the following three rounds. The sorting method used was as described above. To increase the stringency, at the fourth round, the biotinylated targets and phage libraries were incubated at 37° C. for 3 hour. Next, 1000 fold excess of unbiotinylated target was added, and the mixture was incubated at room temperature for 30 minutes before the biotinylated material was captured on the neutravidin plate competing off high off-rate binders.

### High Throughput Affinity Screening ELISA (Single Spot Competition)

The method was as performed as described above. 10 nM mBR3-ECD and hBR3-ECD were used for the single spot competition.

In comparison to the OD₄₅₀ₙₘ reduction (%) of the well of V3-1 phage (80%), clones that had the OD₄₅₀ₙₘ reduction (%) to mBR3-ECD and hBR3-ECD both lower than 50% were picked. Twelve clones were picked, sequenced and assayed (FIG. 17). The results are summarized in FIG. 17.

Clone 41 and clone 46 were the best two V3-1 affinity improved variants. Because clone 41 had more asparagines residues (N), clone 46 was been chosen for further characterization. There is a potential glycosylation site (N—S—S/T) in the CDR-H1 region of clone 46. In order to eliminate this potential glycosylation site, three single mutants of CDR-H1 at position 31 (N31A, N31S and N31Q) were made to test their binding activity to mBR3-ECD and hBR3-ECD. Competition ELISAs were performed to determine their affinity to mBR3-ECD and hBR3-ECD. The results are shown below. Among these three mutants, the affinity of N31S is the closest to the V3-46 parental clone (Table 5).

TABLE 5

Phage ID50 (nM)

| Clone | mBR3-ECD | hBR3-ECD |
|---|---|---|
| V3-46 WT | 1.42 | 0.35 |
| N31A | 2.89 | 0.26 |
| N31S | 1.53 | 0.10 |
| N31Q | 2.44 | 0.27 |

The N31S mutant of V3-46 was renamed as V3-46s. A Fab of V3-46s was made by the method described above. Surface plasmon resonance assays on a BIAcore™-2000 were used to determine the affinity of the V3-46s Fab. The results are summarized in the tables below (Table 6 and Table 7). In comparison to the V3-1 Fab, the on-rate of the V3-46s Fab to mBR3-ECD has been improved. Further, the on-rate and off-rate of V3-46s Fab for hBR3-ECD improved significantly over V3-1.
mBR3-ECD

TABLE 6

| Clone | Kon (1/Ms) | Koff (1/s) | kD (nM) | Phage IC50 (nM) |
|---|---|---|---|---|
| V3-1 | 7.71E+04 | 1.95E−04 | 2.5 | 5.4 |
| V3-46s | 2.70E+05 | 2.70−04 | 1.0 | 1.53 |

hBR3-ECD

TABLE 7

| Clone | Kon (1/Ms) | Koff (1/s) | kD (nM) | Phage IC50 (nM) |
|---|---|---|---|---|
| V3-1 | 3.73E+04 | 2.93E−04 | 7.87 | 5 |
| V3-46s | 1.40E+05 | 8.60E−04 | 0.6 | 0.1 |

### Construction of Homolog Shotgun Library on V3-46s Backbone for Further Affinity Improvement.

For further affinity improvement, the V3-46s phagmid was used as the template to make homolog shotgun libraries. The stop template was constructed by introducing TAA codons within all three light chain CDRs. The mutagenic oligonucleotides were designed to use the binomial codons that encoded only the wide-type and a similar amino acid at the desired positions (JMB 2002: 320 [415-418]). By Kunkel mutagenesis method, the stop codons were repaired and mutations were introduced at the desired sites. (Kunkel et al 1987).

Library 1109-3 was made by mixing all six CDR homolog shotgun oligos as described below. For CDR-H1, H2 and H3, in addition to the original homolog shotgun oligos, we also included the oligos (a and b) mutagenizing every other position to ensure the initial binding activity to BR3 was not disrupted.

| V1109-3: | |
|---|---|
| L1:L2:L3:H1:H2:H3 = 1:1:1:0.5:1:1.5 | |
| L1(F349)/L2(F350)/L3(F351)/H1(F352 + F352a + F352b = 1:1:1)/H2(F355 + F355a + | |
| F355b = 1:1:1)/H3(F356 + F356a + F356b = 1:1:1) | |
| Oligos | |
| <CDR-L1> | |
| F349 (5′-ACC TGC CGT GCC AGT SAA GAM RTT KCC ASC KCT GTA GCC TGG TAT | (SEQ ID NO: 181) |
| CAA CAG AAA C-3′) | |
| <CDR-L2> | |
| F350 (5′-CCG AAG CTT CTG ATT TWC KCC GCA TCC TWC CTC TWC TCT GGA GTC | (SEQ ID NO: 182) |
| CCT TCT CGC-3′) | |
| <CDR-L3> | |
| F351 (5′-GCA ACT TAT TAC TGT CAG CAS KCC SAA RTT KCC CCG SCA ACG TTC | (SEQ ID NO: 183) |
| GGA CAG GGT ACC-3′) | |
| CAS codon encodes Gln and His. | |
| <CDR-H1> | |
| F352 (5′-GCA GCT TCT GGC TTC ACC ATT KCC KCC KCC KCC ATA CAC TGG GTG | (SEQ ID NO: 184) |
| CGT CAG-3′) | |
| F352a (5′-GCA GCT TCT GGC TTC ACC ATT AGT KCC AGC KCC ATA CAC TGG GTG | (SEQ ID NO: 185) |
| CGT CAG-3′) | |
| F352b (5′-GCA GCT TCT GGC TTC ACC ATT KCC AGC KCC TCT ATA CAC TGG GTG | (SEQ ID NO: 186) |
| CGT CAG-3′) | |
| <CDR-H2> | |
| F355 (5′-AAG GGC CTG GAA TGG GTT GCA TKG RTT MTC SCA KCC RTT GST TWC | (SEQ ID NO: 187) |
| ASC GAM TAT GCC GAT AGC GTC AAG GGC-3′) | |
| F355a (5′-AAG GGC CTG GAA TGG GTT GCT TGG RTT CTT SCA TCT RTT GGT TWC | (SEQ ID NO: 188) |
| ACT GAM TAT GCC GAT AGC GTC AAG GGC-3′) | |
| F355b (5′-AAG GGC CTG GAA TGG GTT GCT TKG GTT MTC CCT KCC GTG GST TTT | (SEQ ID NO: 189) |
| ASC GAC TAT GCC GAT AGC GTC AAG GGC-3′) | |
| <CDR-H3> | |
| F356 (5′-ACT GCC GTC TAT TAT TGT GCA ARA ARA RTT TGC TWC RAC ARA MTC | (SEQ ID NO: 190) |
| GST RTT TGC KCT GST GST ATG GAC TAC TGG GGT CAA-3′) | |
| F356a (5′-ACT GCC GTC TAT TAT TGT GCT CGT ARAGTC TGC TWC AAC ARA CTT | (SEQ ID NO: 191) |
| GST GTT TGC KCT GGT GST ATG GAC TAC TGG GGT CAA-3′) | |
| F356b (5′-ACT GCC GTC TAT TAT TGT GCT ARA CGG RTT TGC TAC RAC CGC | (SEQ ID NO: 192) |
| MTC GGT RTT TGC GCT GST GGT ATG GAC TAC TGG GGT CAA-3′) | |

See Table 1 of Vajdos, et al., (2002) J. Mol. Biol. 320:415-418 for an illustration of the codon usage to encode both wt residue and its homolog residue.

### Phage Sorting for Affinity Selection of V3-46s

Three rounds of solution sorting were performed in V1109-3 by decreasing biotinylated mBR3-ECD and hBR3-ECD concentration. The phage input was 2 O.D/ml at first round and 0.5, 0.1 O.D/ml for the following two rounds. 1 nM biotinylated target was used for the first round. Then 0.2 and 0.1 nM biotinylated targets were used in the following two rounds. The sorting method has been described above. To increase the stringency, at the third round, biotinylated targets were incubated with phage libraries at 37° C. for 3 hour. Then, 1000 fold excess of unbiotinylated target was added and the mixture was incubated at room temperature for 30 minute before capture on the neutravidin plate to compete off high off-rate binders.

### High Throughput Affinity Screening ELISA (Single Spot Competition)

1 nM mBR3-ECD and hBR3-ECD were used to do the single spot competition as described above. The OD₄₅₀ₙₘ reduction (%) in the test wells were compared to the well of the V3-46s phage (95%). Clones that had 50% OD₄₅₀ₙₘ reduction (%) in the presence of both mBR3-ECD and hBR3-ECD were picked. Fourteen clones were picked, sequenced and assayed.

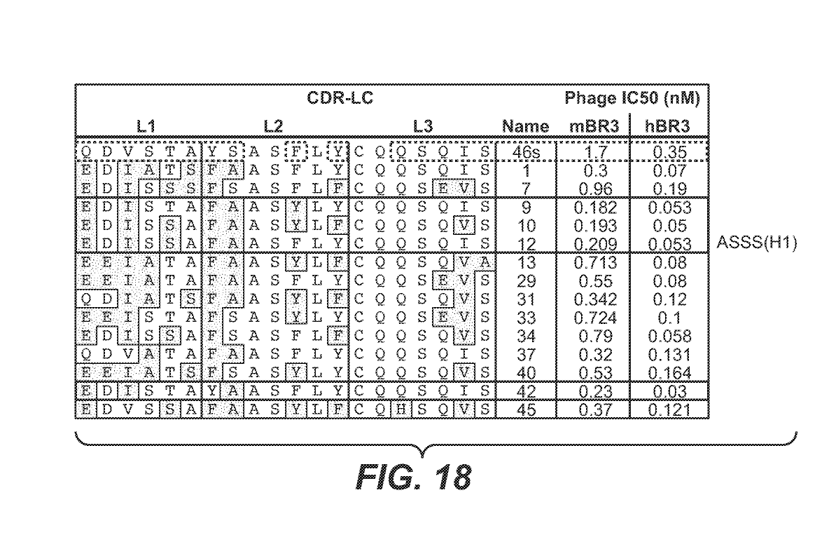

FIG. 18 shows the phage IC50 for affinity selected V3-46s clones for mBR3-ECD or hBR3-ECD compared with WT V3-46s. All fourteen clones appear to be better binders than V3-46s (WT) to mBR3-ECD and hBR3-ECD. Most of the clones have the same CDR-HC sequence as WT V3-46s except for V3-46s-12, which clone differs from WT by having a change in its CDR-H1. See FIG. 18 and SEQ ID NO:193. All the clones have changes in CDR-L1, CDR-L2 and CDR-L3 as indicated in FIG. 18. Most of the affinity-improved variants are two to five fold affinity improved compared to the V3-46s parental clone. V3-46s-42 binding to mBR3-ECD and hBR3-ECD is six to eight-fold increased to a pM range.

To confirm the protein affinity of affinity improved clones, V3-46s-9 and V3-46s-42 Fab were made by the method described above. Surface plasmon resonance assays on a BIAcore™-3000 were used to determine the affinity of the Fabs. The result is summarized in the table below. Compared to the V3-46s Fab, the on-rate of V3-46s-42 Fab to mBR3-ECD and hBR3-ECD has been improved. The Kds have good agreement with phage IC50 values. See below.

| | Kon | | | |
|---|---|---|---|---|
| | (1e5/Ms) | Koff (1e−4/S) | kD (nM) | Phage50 (nM) |
| mBR-ECD | | | | |
| V46s-9 | 4.70 | 1.50 | 0.32 | 0.18 |
| V46s-42 | 7.40 | 2.90 | 0.39 | 0.23 |
| V46s | 2.70 | 2.70 | 1.00 | 1.7 |
| hBR-ECD | | | | |
| V46s-9 | 1.60 | 0.16 | 0.09 | 0.05 |
| V46s-42 | 6.17 | 0.14 | 0.026 | 0.03 |
| V46s | 1.40 | 0.86 | 0.60 | 0.35 |

### Example 5

### BJAB Cell Binding Assay

BJAB cells, a human Burkitt lymphoma cell line, were cultured in RPMI media supplemented with 10% FBS, penicillin (100 U/ml, Gibco-Invitrogen, Carlsbad, Calif.), streptomycin (100 μg/ml, Gibco), and L-glutamine (10 mM). Analysis of receptor expression by flow cytometry demonstrated that BJAB cells express high levels of BR3 and undetectable levels of BCMA and TACI. For binding assays, cells were washed with cold assay buffer (phosphate buffered saline (PBS), pH 7.4) containing 1% fetal bovine serum (FBS)). The cell density was adjusted to 1.25×10⁶/ml, and 200 μl of cell suspension was aliquoted into the wells of 96 well round-bottom polypropylene plates (NUNC, Neptune, N.J.; 250,000 cells/well). The plates containing the cells were centrifuged at 1200 rpm for 5 min at 4° C., and the supernatant was carefully aspirated away from the cell pellets. V3-1m (or mV3-1) and V3-1 h refers to the variable region of the V3-1 antibody fused to the constant regions of mouse IgG2a or human IgG1, respectively. The term chimeric 11G9, chimeric 2.1 or chimeric 9.1 refers to the fusion of the variable regions of 11G9, 2.1 or 9.1, respectively, to the constant regions of a human IgG1. For these experiments, full length antibodies (IgG) were used.

Direct and competitive binding assays were performed as follows. For the direct binding assay, IgG antibody samples were serially diluted in cold assay buffer to concentrations ranging between 300-0.02 nM. Samples (100 μl) were added to the pelleted cells, and the plates were incubated for 45 min on ice. An additional 100 μl assay buffer was then added to each well, and the plates were centrifuged at 1200 rpm for 5 min at 4° C. After carefully aspirating the supernatant, the cells were washed two additional times with 200 μl assay buffer. An anti-mouse IgG Fc-HRP or goat anti-human IgG Fc-HRP, as appropriate, was diluted 1/10,000 in cold assay buffer was added (100 μl/well, Jackson ImmunoResearch, West Grove, Pa.), and the plates were incubated on ice for 45 min. Following two washes with 200 μl cold assay buffer, tetramethyl benzidine (TMB, Kirkegaard & Perry Laboratories, Gaithersburg, Md.) was added, and color was allowed to develop for 10 min. One hundred microliters 1 M H₃PO₄ was added to stop the reaction. The plates were then read on a microplate reader at 450 nm with a 620 nm reference. In the direct binding assay, the indicated concentrations of mAbs were added to BJAB cells and bound mAb was detected.

In the competitive binding assay, the anti-BR3 mAbs compete with biotinylated BAFF for binding to cell surface BR3. Human BAFF expressed and purified at Genentech was biotinylated using NHS-X-biotin (Research Organics, Cleveland, Ohio) as previously described (Rodriguez, C. F., et al., (1998) J. Immunol. Methods 219:45-55). The anti-BR3 antibodies were serially diluted and combined with an equal volume of biotin-BAFF to give final concentrations of 333-0.15 nM mAb and 10 ng/ml biotin-BAFF. The diluted samples were added to the pelleted BJAB cells in 96 well plates as described above. After 45 min incubation on ice, the cells were washed twice with 200 μl cold assay buffer, and streptavidin-HRP (AMDEX, Amersham Biosciences, Piscataway, N.J.) diluted 1/5,000 in assay buffer was added (100 μl/well). The plates were incubated for a final 45 min on ice. After washing twice with cold assay buffer, color was developed using TMB, the reaction was stopped with H₃PO₄, and the plates were read as described above.

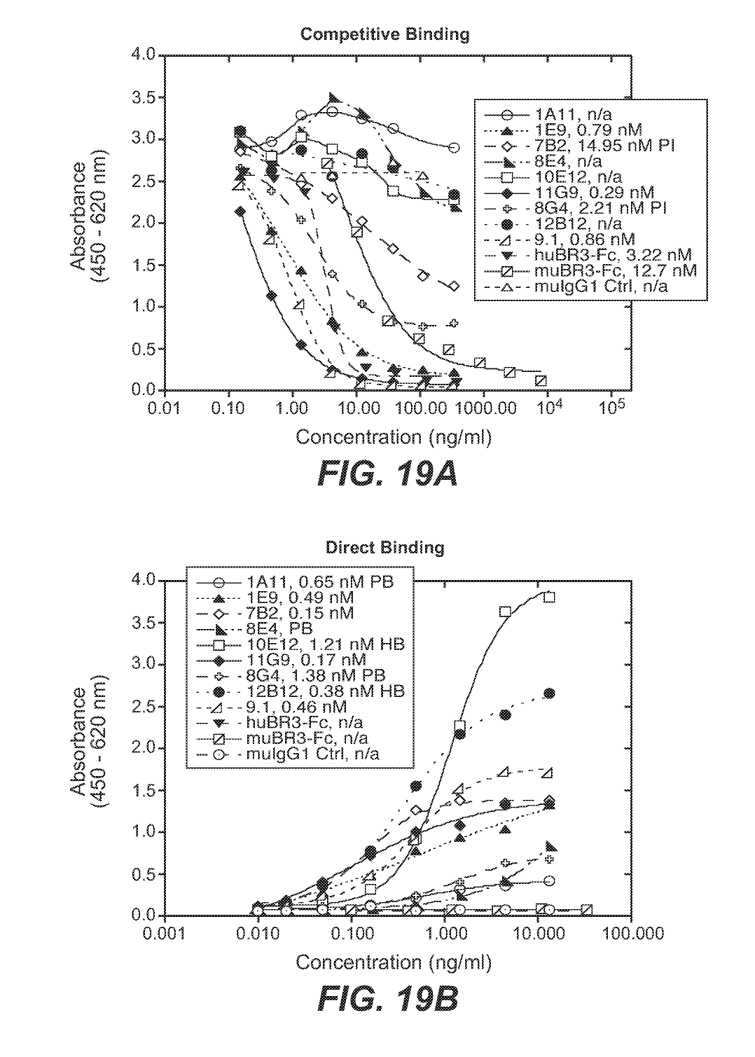

FIG. 19 shows that the antibodies bind BR3 on BJAB cells. FIG. 20 shows that while V3-1m was able to competitively displace binding of BAFF to the human BR3 expressed on BJAB cells (panel A) as well as directly bind to BJABs (panel B), B9C11 showed no ability to bind to human BR3 in either format of the assay (panels A and B, respectively). In contrast, both V3-1m and B9C11 fully blocked BAFF binding to the murine BR3 expressed on BHK cells (panel C) and were able to bind directly to the cells (panel D). Different detection antibodies were required for the direct binding assays with V3-1m (mouse IgG) and B9C11 (hamster IgG).

Based the results of the BJAB binding assays, the antibodies could be classified as either blocking or non-blocking. In the competitive assay, four mAbs (11G9, 2.1, 9.1, and V3-1) fully blocked binding of biotin-BAFF while three others (1E9, 7B2, and 8G4) resulted in partial inhibition (FIGS. 19 and 20, Table 8). MAbs 1A11, 8E4, 10E2, 12B12 and 3.1 were found to be non-blocking (FIG. 19). Of these nonblocking antibodies, 1A11 and 8E4 bound relatively poorly to the BJABs in the direct binding assay, while binding of 10E12 and 12B12 gave somewhat higher maximum signal than the other mAbs. Mouse IgG1, IgG2a, and IgG2b isotype controls showed no detectable binding to BJABs, and the HRP-conjugated anti-mouse IgG Fc detection antibody was shown to bind equally to these isotypes. MAbs V3-1m and B9C11 were evaluated in both the BJAB and BHK binding assays (FIG. 20). While both of these blocking antibodies bind to murine BR3, only V3-1m binds to human BR3. Results with V3-1 h were similar to those observed for V3-1m.

### Example 6

### Epitope Mapping ELISAS

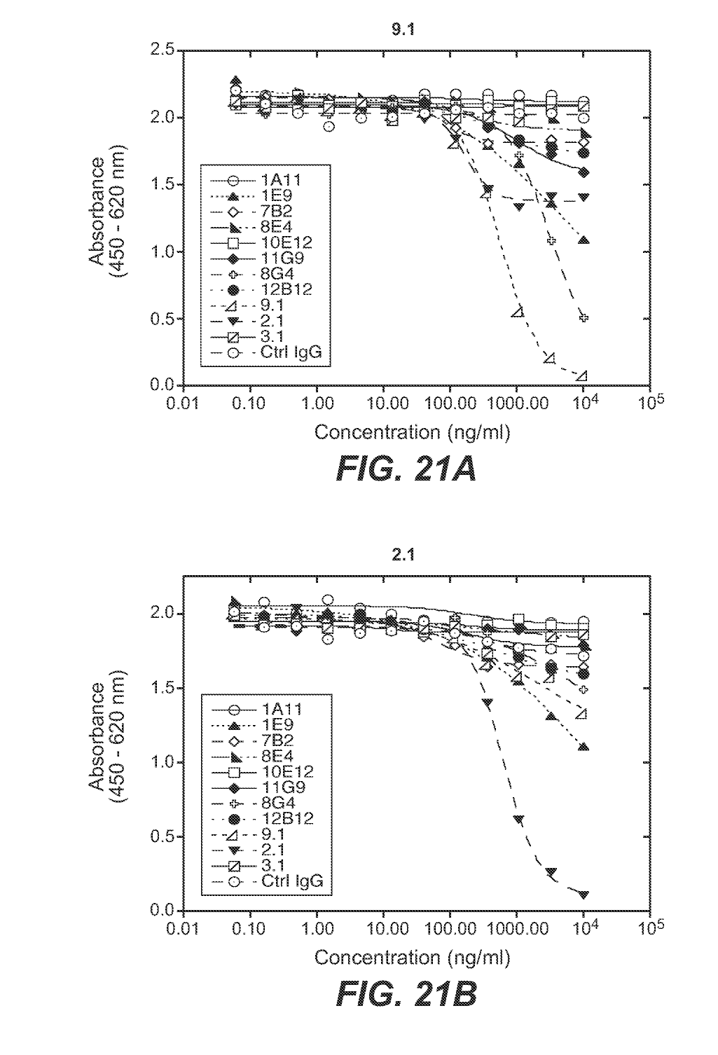

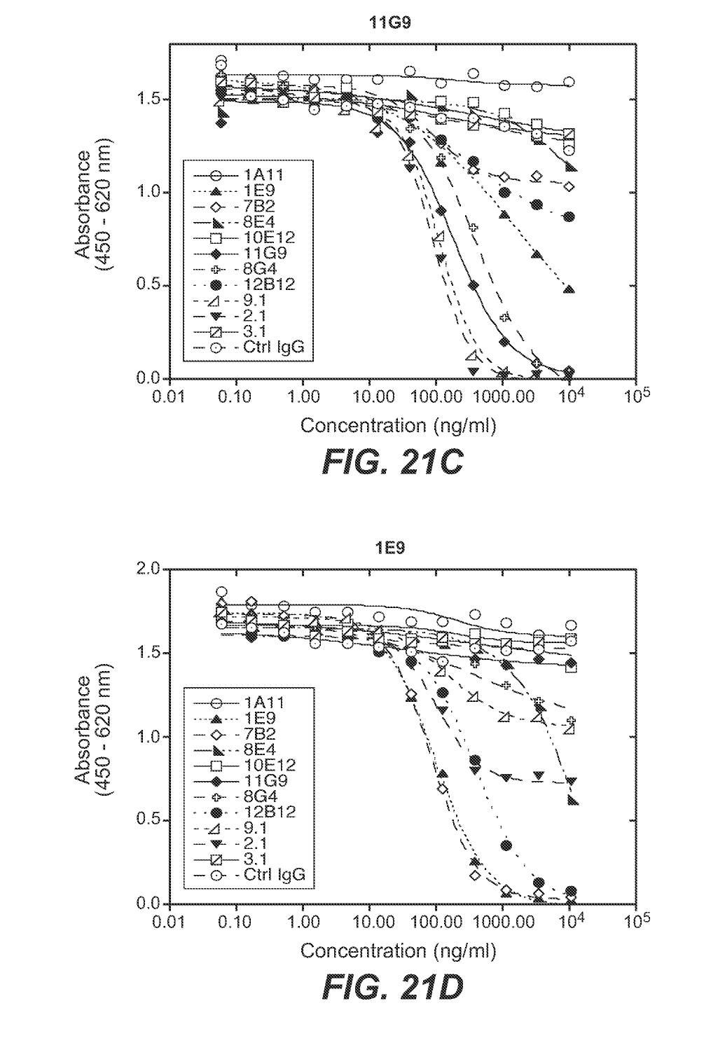

Epitope mapping studies were performed by ELISAs in which dilution curves of unlabeled mAbs competed with biotinylated 2.1, 9.1, 11G9, or 1E9 for binding to vhBR3-Fc (FIG. 21). The results for the fully blocking mAbs (11G9, 2.1, and 9.1) suggested that the epitope for 11G9 binding was spatially located between the epitopes for mAbs 2.1 and 9.1 given that both 2.1 and 9.1 effectively displaced binding of biotinylated 11G9 but showed only a marginal ability to displace each other. Three mAbs (1E9, 7B2, and 8G4) were characterized as partial blockers in the competitive BJAB binding assay. In the epitope mapping ELISA, these mAbs appeared to bind more peripherally to the central BAFF blocking site given that they only partially inhibited the binding of the 11G9, 2.1, and 9.1. Finally, the non-blocking mAb, 12B12, appeared to bind still further away from the region of the blocking antibodies given that it could be displaced by only 1E9, a partial blocker.

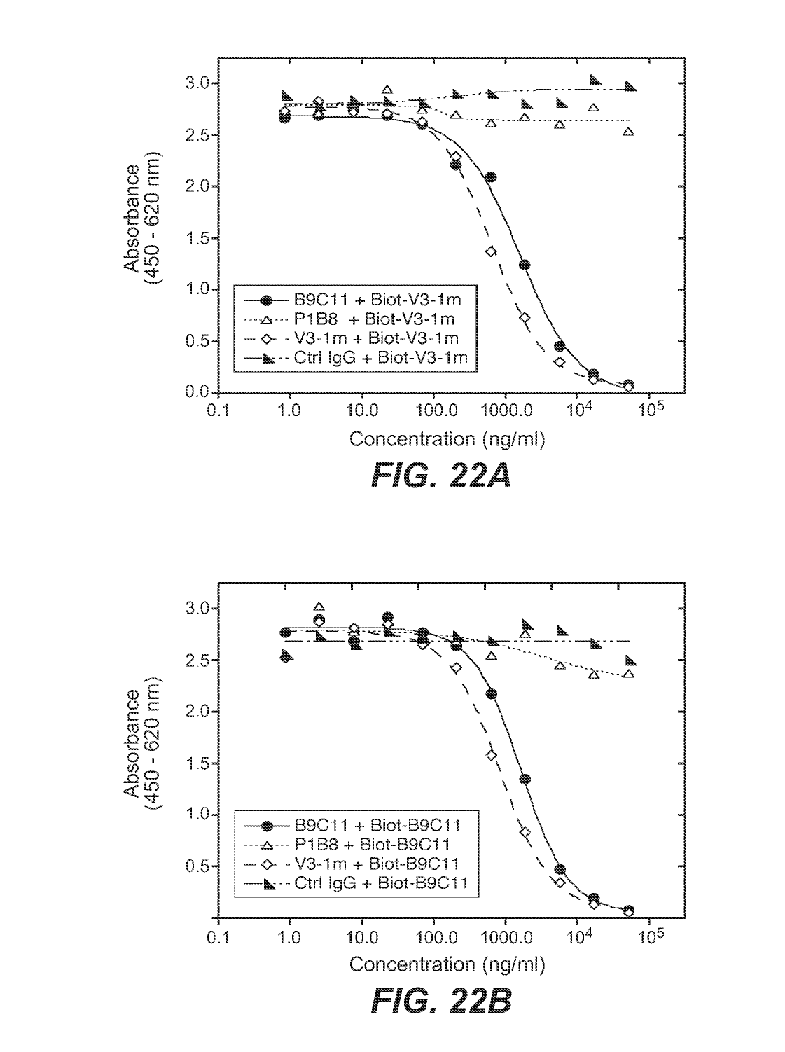

Mapping studies were also performed to evaluate the binding of V3-1m, B9C11, and P1B8 to mouse BR3. The results demonstrated that while the two blocking mAbs (V3-1m and B9C11) were able to cross-compete for binding to mouse BR3, the non-blocking mAb P1B8 appeared to bind to a separate epitope (FIG. 22).

The following table is a summary of the results of the competitive BJAB cell binding assay (Table 8). The results of assays run over a period of several months were compiled. The mean IC50 was calculated from the indicated number “n” of experiments.

TABLE 8

| mAb/Br3 | Blocking | Mean IC50 (nM) | SD | n |
|---|---|---|---|---|
| vBR3-Fc | + | 2.15 | | |
| 1A11 | − | n/a | | 2 |
| 1E9 | + | 2.75 | 4.09 | 4 |
| 7B2 | +/− | 8.03 | | 2 |
| 8E4 | − | n/a | | 2 |
| 8G4 | +/− | 2.07 | 0.24 | 3 |
| 10E12 | − | n/a | | 2 |
| 12B12 | − | n/a | | 2 |
| 11G9 | + | 0.38 | 0.11 | 4 |
| 9.1 | + | 1.30 | 0.44 | 4 |
| 2.1 | + | 0.25 | | 2 |
| Chimeric | + | 0.45 | | 3 |
| 11G9 | | | | |
| Chimeric | + | 0.96 | 0.13 | 3 |
| 9.1 | | | | |
| Chimeric | + | 0.23 | 0.37 | 3 |
| 2.1 | | | | |
| V3-1m | + | 2.47 | 0.08 | 1 |
| V3-1h | + | 5.97 | | 1 |

n/a = no inhibition was detected or it was not possible to calculated IC50
+/− = antibodies partially inhibited biotinylated BAFF binding

The following table is a summary of the results of the direct BJAB cell binding assay (Table 9). The results of assays run over a period of several months were compiled. While most antibodies gave an appreciable dose-dependent signal, three mAbs appeared to yield only partial binding and two mAbs reproducibly gave a higher maximum signal than the others. The mean EC50 was calculated from the indicated number “n” of experiments.

TABLE 9

| mAb/Br3 | Binding | Mean EC50 (nM) | SD | n |
|---|---|---|---|---|
| vBR3-Fc | − | n/a | | 2 |
| 1A11 | −/+ | 1.17 | | 2 |
| 1E9 | + | 0.66 | 0.61 | 3 |
| 7B2 | + | 0.16 | | 2 |
| 8E4 | −/+ | n/a | | 2 |
| 8G4 | −/+ | 1.78 | 0.37 | 3 |
| 10E12 | High | 1.47 | | 2 |
| 12B12 | High | 0.7 | | 2 |
| 11G9 | + | 0.19 | 0.05 | 3 |
| 9.1 | + | 0.54 | 0.10 | 3 |
| 2.1 | + | 0.16 | | 1 |
| V3-1m | + | 3.37 | | 1 |
| B9C11 | n/a | n/a | | 1 |

n/a = either no binding was detectable or it was not possible to calculate EC50.
+/− = partial binding

Humanized anti-BR3 antibodies (IgG) also blocked BAFF binding to BR3 on BJAB cells and bound BR3 on BJAB cells. See Table 10 below.

TABLE 10

| | |
|---|---|
| | BAFF |
| mAb direct | Competitive |
| binding | Assay |
| EC50 (nM) | IC50 (nM) |

| mAb anti-BR3 | Mean | SD | mean | SD |
|---|---|---|---|---|
| V3-1m | 4.3 | 0.8 | 8.9 | 3.0 |
| hV3-46S | 1.8 | 0.7 | 1.9 | 2.5 |
| ch 9.1 | 0.36 | 0.08 | 1.0 | 0.3 |
| h9.1-88 | 0.43 | 0.09 | 0.60 | 0.47 |
| h9.1-70 | 0.33 | | 0.82 | |
| h9.1-73 | 0.79 | | 1.78 | |
| h9.1-RF | 0.46 | 0.11 | 0.68 | 0.80 |
| ch 2.1 | 0.14 | 0.05 | 0.20 | 0.07 |
| h2.1-30 | 0.11 | 0.02 | 0.17 | 0.07 |
| h2.1-46 | 0.11 | 0.04 | 0.19 | 0.09 |
| h2.1-94 | Partial | | 5.1 | 3.0 |
| vhBR3-Fc | | | 1.4 | 0.4 |

*“h” indicates humanized;
“ch” indicates chimera.

### Example 7

### Antagonistic and Agonistic Effects of Anti-BR3Antibodies on B Cell Proliferation

(a) 2.1, 9.1 and 11G9 Inhibit Human B Cell Proliferation

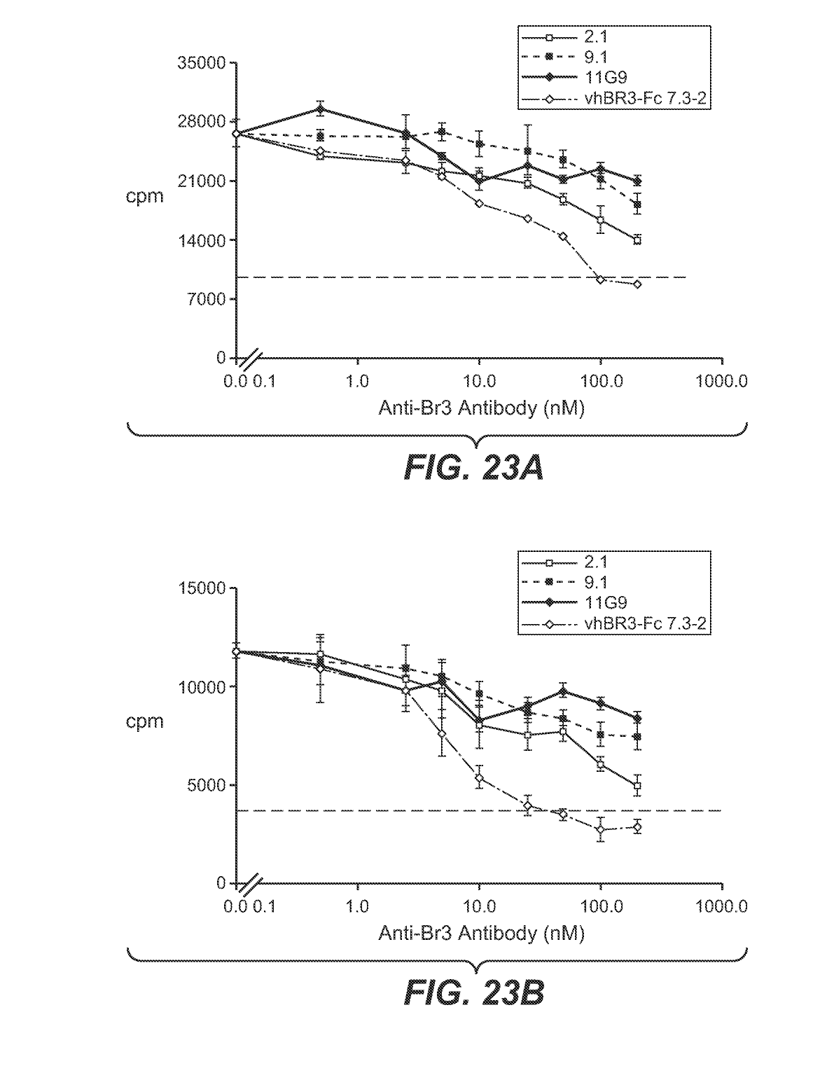

B cells were isolated from peripheral blood mononuclear cells by positive selection using CD19 MACS beads (Miltenyi Biotec). For proliferation assays, B cells were setup cells at 2×10⁵ c/well in flat-bottom 96-well plate in triplicate. Cells were cultured cells for 5 days with anti-IgM (10 mg/ml) (Jackson Immunoresearch), mBAFF (5 ug/ml) and the indicated anti-BR3 antibodies or proteins for 5 days. Antibodies used were chimeric antibodies in an hIgG1 background and purified from tissue culture. The cells were then pulsed with 1 mCi/well tritiated-thymidine for the last 6 hours of culture, harvested onto a filter and counted. The results are shown in FIG. 23.

(b) V3-1 Inhibits Murine B Cell Proliferation

Splenic B cells were prepared from C57BL/6 mice or from anti-HEL BCR transgenic mice at the age of 2-4 months, using B cell isolation kit from Miltenyi, according to the manufacture's instruction. We consistently obtained B cells with more than 95% purity. The B cells were cultured in the RPMI-1640 medium containing 10% heat-inactivated FCS, penicillin/streptomycin, 2 mM L-glutamine and 5×10⁻² uM beta-Mercaptoethanol.

The purified B cells (10⁵ B cells at final volume of 200 ul) were cultured with anti-mouse IgM Ab 5 ug/ml (IgG, F(ab′)2 fragment) (Jackson ImmunoResearch Laboratories) or Hen Egg Lysozyme (Sigma), with or without BAFF (2 ng/ml or 10 ng/ml), in the absence or presence of various concentration of anti-BR3 mabs. Proliferation was measured by ³H-thymidine uptake (1 uCi/well) for the last 8 hours of 48 hour stimulation. In some experiments, anti-BR3 mAbs as well as BR3-Fc fusion protein were pre-boiled for 5 min using PCR machine to inactivate them (controls).

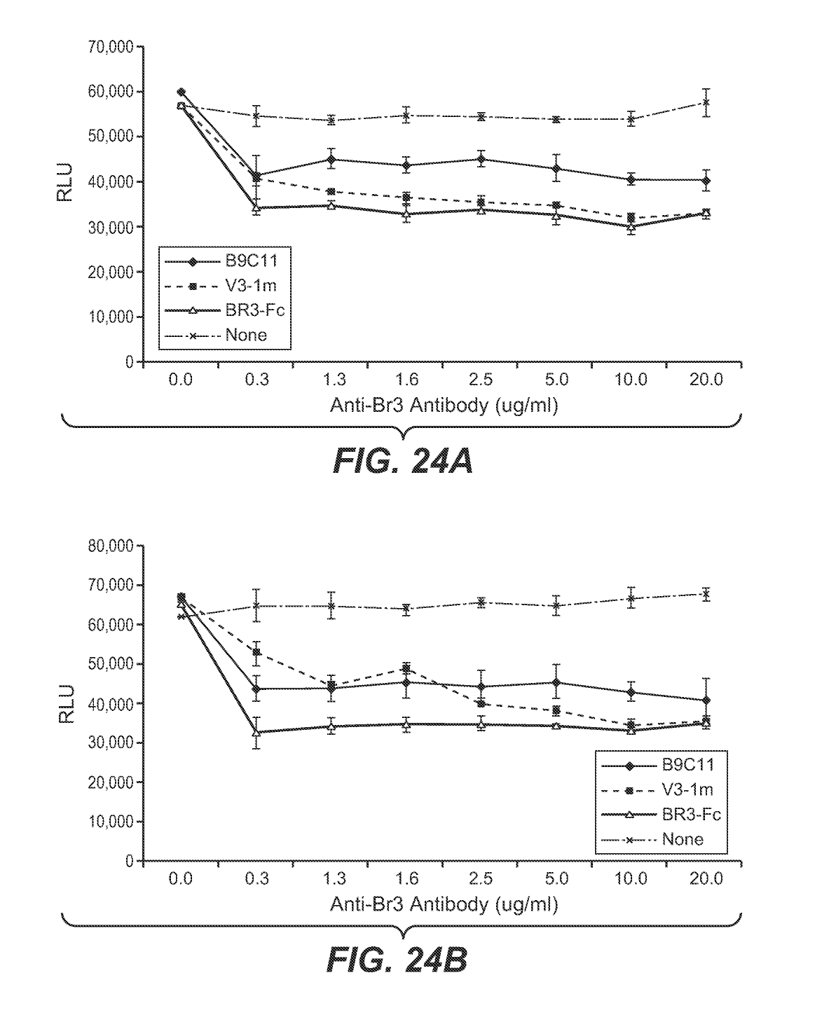

FIG. 24 shows that, like BR3-Fc, both B9C1 and V3-1m can inhibit the BAFF costimulatory activity during anti-IgM mediated primary murine B cell proliferation. Neither B9C11 nor V3-1m showed any direct effect on B cell proliferation in the absence or presence of various doses of anti-IgM antibody (data not shown). Inhibition of proliferation of B cells from anti-HEL BCR transgenic mice with V3-1m and B9C11 (not boiled V3-1m or B9C11) was also observed (data not shown). Both antibodies are not agonistic in that they do not trigger normal murine B cells proliferation on their own.

(b) Other Antibodies

Human B cells were isolated from peripheral blood mononuclear cells by positive selection using CD19 MACS magnetic beads according to the manufacturer's protocol (Miltenyi Biotec, Auburn, Calif.). Cells were either used immediately after isolation or were frozen in liquid nitrogen for later use; fresh and frozen cells performed equivalently in the assay. B cells were cultured at 1×10⁵ cells/well in black 96-well plates with clear, flat-bottomed wells (PE Biosystems, Foster City, Calif.).

For evaluating antagonistic effects of anti-BR3 antibodies, the cells were incubated with soluble recombinant BAFF (10 ng/ml) and a F(ab′)2 goat anti-human IgM (Fc specific) antibody (4 μg/ml) (Jackson ImmunoResearch, West Grove, Pa.) in the presence and absence of various concentrations of anti-BR3 antibody ranging from 100 nM to 1.3 pM (15 μg/ml-1 ng/ml). B cell proliferation was assessed at day 6 by adding Celltiter Glo (Promega, Madison, Wis., reconstituted according the manufacturer's instructions) to each assay well. The plates were then read in a luminometer after incubation for 10 minutes at room temperature.

The potential for anti-BR3 antibody agonism to stimulate B cell proliferation was assessed by incubating anti-BR3 antibody (100 nM to 1.3 pM) in the presence of the anti-IgM antibody alone (4 μg/ml) or in the presence of anti-IgM plus a ‘cross-linking’ F(ab′)2 goat anti-human IgG Fc antibody (Pierce, Rockford, Ill., 30 μg/ml) and in the absence of BAFF. Proliferation was assessed at day 6 using Celltiter Glo as described above.

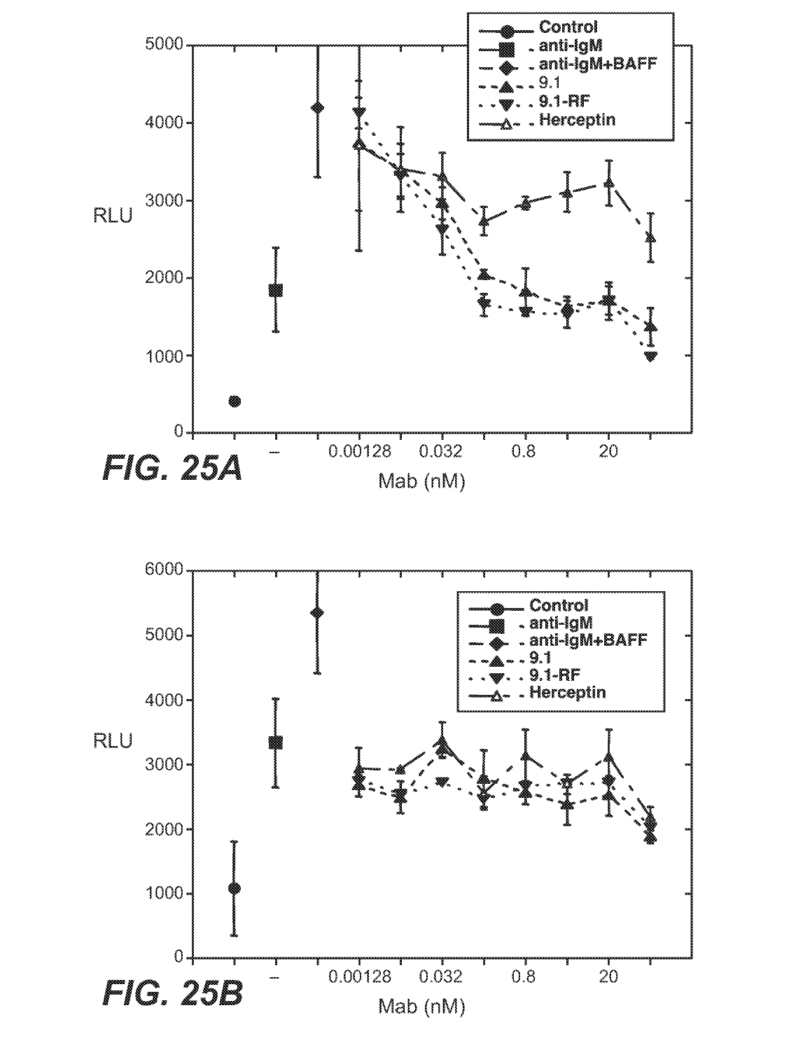

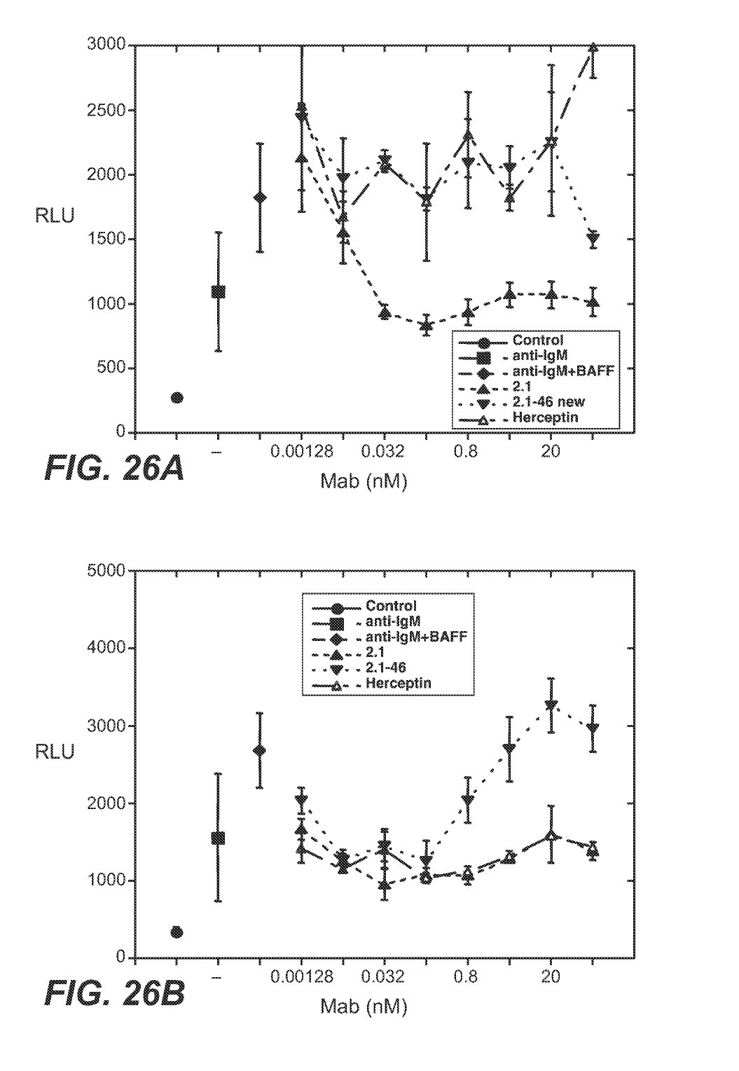

FIG. 25 shows that 9.1-RF blocks BAFF-dependent B cell proliferation and does not agonize. FIG. 26 shows that 2.1-46 stimulates B cell proliferation in the presence of anti-IgM, showing it acts as an agonist.

### Example 8

### Affinity Measurements Using Biacore

### Materials and Methods

Real-time biospecific interactions were measured by surface plasmon resonance using Pharmacia BIAcore® 3000 (BIAcore AB, Uppsala, Sweden) at room temperature (Karlsson, R., et al. (1994) Methods 6:97-108; Morton, T. A. and Myszka, D. G. (1998) Methods in Enzymology 295: 268-294). Human BR3 ECD or vBR3-Fc were immobilized to the sensor chip (CM5) through primary amine groups. The carboxymethylated sensor chip surface matrix was activated by injecting 201 of a mixture of 0.025 M N-hydroxysuccinimide and 0.1 M N-ethyl-N′(dimethylaminopropyl) carbodiimide at 5 μl/min. 5-10 μof 5 μg/ml solution of BR3 ECD or vBR3-Fc in 10 mM sodium acetate, pH 4.5, were injected at 5 μl/min. After coupling, unoccupied sites on the chip were blocked by injecting 20 μl of 1M ethanolamine, pH 8.5. The running buffer was PBS containing 0.05% polysorbate 20. For kinetic measurements, two-fold serial dilutions of anti-BR3 antibodies (6.2-100 nM or 12.5-200 nM) in running buffer were injected over the flow cells for 2 minutes at a flow rate of 30 μl/min and the bound anti-BR3 antibody was allow to dissociate for 20 minutes. The binding surface was regenerated by injecting 20-30 μl of 10 mM glycine.HCl (pH 1.5). Flow cell one, which was activated but did not have BR3 ECD or BR3-Fc immobilized, was used as a reference cell. There was no significant non-specific binding of anti-BR3 antibodies to flow cell one. Data were analyzed using a 1:1 binding model using global fitting. The association and dissociation rate constants were fitted simultaneously (BIAevaluation software). Similar results were obtained whether samples were run in the order of increasing or decreasing concentrations for selected antibodies tested.

Binding kinetics of anti-BR3 antibodies to BR3 ECD or BR3-Fc were measured by BIAcore. BR3 ECD or vBR3-Fc was immobilized on sensor chips, and serial dilutions of antibodies were injected over the flow cells (Tables 11 and 12). Alternatively, anti-BR3 antibodies were immobilized on sensor chips, and serial dilutions of BR3 ECD were injected over the flow cells (Table 13). A high flow rate was used in order to minimize mass transport effects. Results of humanized Fab and humanized IgG antibodies compared side by side. The apparent binding affinities obtained using IgG in solution are higher than those obtained using Fab in solution, likely due to the avidity effects since IgG is bivalent. The apparent kinetic parameters of anti-BR3 antibodies from the 9.1, 2.1, 11G9 and the V3-1 series of antibodies are shown in Tables 11-13.

A. BR3 ECD on Chip

TABLE 11

| | Amount | | | | | |
|---|---|---|---|---|---|---|
| | immobilized | | | | | |
| Anti-BR3 | (RU) | Kₐ (10⁵/Ms) | K_{d} (10⁻⁵/s) | K_{D} (nM) | Rₘₐₓ (RU) | Comments |
| 9.1 IgG | 150 | 4.5 | 5.6 | 0.12 | 108 | |
| 2.1 IgG | | 9.6 | 5.2 | 0.05 | 53 | |
| Chimeric 2.1 IgG | | 16.8 ± 2.4 | 6.8 ± 1.0 | 0.04 ± 0.01 | 60 ± 1 | 6.25-100 nM, |
| | | | | | | n = 3 |
| Chimeric 9.1 IgG | | 14.9 | 9.2 | 0.06 | 55 | 6.25-100 nM |
| Chimeric 11G9 | | 16.4 | 54.9 | 0.34 | 32 | 6.25-100 nM |
| IgG | | | | | | |
| Ch 9.1 Fab | 150 | 14.2 ± 0.1 | 34.6 ± 0.5 | 0.24 ± 0.01 | 29 ± 1 | n = 2 |
| Ch 11G9 Fab | | 12.0 | 2330.0 | 19.50 | 19 | |
| Ch 2.1 Fab | | 22.5 | 27.1 | 0.12 | 28 | |
| Ch 9.1 Fab | 110 | 14.4 | 33.9 | 0.24 | 171 | |
| Hu9.1_73 Fab | | 5.6 | 17.4 | 0.31 | 183 | |
| Hu9.1_ 73 | | 5.5 | 17.5 | 0.32 | 184 | |
| Fab | | | | | | |
| Hu 9.1_ RF | | 20.2 | 29.4 | 0.14 | 183 | |
| Fab | | | | | | |
| Hu9.1_ 70 | | 10.8 | 13.7 | 0.13 | 184 | |
| Fab | | | | | | |
| Hu9.1_70 | 100 | 9.5 | 16.3 | 0.17 | 137 | |
| Fab | | | | | | |
| Ch 2.1 Fab | | 26.5 | 29.4 | 0.11 | 129 | |
| Hu2.1_40Fab | | 1.1 | 92.3 | 8.67 | 46 | |
| Hu2.1_40LFab | | 0.4 | 156.0 | 35.80 | 52 | |
| Hu2.1_RLFab | | 1.8 | 176.0 | 10.00 | 67 | |
| Hu2.1_94Fab | | 13.9 | 114.0 | 0.82 | 106 | |
| Hu2.1_46Fab | | 25.5 | 69.3 | 0.27 | 118 | |
| Hu2.1_30Fab | | 38.4 | 31.1 | 0.08 | 139 | |
| Ch 11G9 Fab | | 11.2 | 2630.0 | 23.50 | 105 | |
| Hu11G9_46 Fab | | 17.6 | 80.8 | 0.46 | 125 | |
| Hu11G9_36 Fab | | 14.9 | 105.0 | 0.70 | 118 | |
| Hu11G9_46 IgG | | 16.6 | 4.2 | 0.025 | 372 | |
| Hu11G9_36 IgG | | 17.1 | 4.1 | 0.024 | 371 | |
| Hu9.1-88 IgG | | 19.4 ± 0.6 | 4.9 ± 0.02 | 0.025 ± 0.001 | 370 ± 6 | N = 2 |
| Hu2.1-30 Fab | | 39.4 | 23.2 | 0.059 | 262 | |
| Hu2.1-30 IgG | | 24.1 | 4.0 | 0.017 | 275 | |
| Hu11G9-36 Fab | | 14.7 | 95.4 | .650 | 232 | |
| Hu11G9-46 Fab | | 13.3 | 86.7 | .652 | 232 | |
| Hu9.1-88 Fab | | 13.7 | 101.0 | .736 | 215 | |
| Hu2.1-46 IgG | | 22.0 | 4.5 | 0.02 | 346 | |
| Hu2.1-94 IgG | | 17.7 | 6.6 | 0.037 | 331 | |

### BR3-Fc on Chip

TABLE 12

| | Amount | | | | | |
|---|---|---|---|---|---|---|
| | immobilized | | | | | |
| Anti-BR3 IgG | (RU) | Kₐ (10⁵/Ms) | K_{d} (10⁻⁵/s) | K_{D} (nM) | Rₘₐₓ (RU) | Comments |
| 9.1 IgG | 100 | 5.1 | 31.3 | 0.61 | 152 | |
| 2.1 IgG | | 4.4 | 3.4 | 0.08 | 208 | |
| 2.1 IgG | | 4.7 | 3.3 | 0.07 | 217 | 6.25-100 nM |
| Ch 2.1 IgG | | 9.8 ± 0.9 | 3.8 ± 0.6 | 0.04 ± 0.01 | 241 ± 3 | 6.25-100 nM, |
| | | | | | | n = 3 |
| Ch 9.1 IgG | | 13.2 | 23.2 | 0.17 | 222 | 6.25-100 nM |
| Ch 11G9 IgG | | 7.8 | 218.0 | 2.78 | 127 | 6.25-100 Nm |
| Ch 9.1 Fab | 140 | 4.4 ± 0.4 | 868.5 ± 21.9 | 20.00 ± 2.12 | 49 ± 1 | n = 2 |
| Ch 11G9 Fab | | | | | | No significant |
| | | | | | | binding |
| Ch 2.1 Fab | | 13.2 | 148.0 | 1.12 | 126 | |
| Ch 9.1 Fab | 380 | 4.3 | 932.0 | 21.80 | 137 | |
| Hu9.1_73 Fab | | 5.0 | 21.5 | 0.43 | 427 | |
| Hu9.1_ 73 | | 4.7 | 22.5 | 0.48 | 424 | |
| Fab | | | | | | |
| Hu9.1_ RF | | 2.9 | 186.0 | 6.40 | 255 | |
| Fab | | | | | | |
| Hu9.1_ 70 | | 6.4 | 39.2 | 0.61 | 357 | |
| Fab | | | | | | |
| Hu9.1_70 | 220 | 7.2 | 68.0 | 0.95 | 174 | |
| Fab | | | | | | |
| Ch 2.1 Fab | | 15.8 | 145.0 | 0.92 | 183 | |
| Hu2.1_40Fab | | 3.8 | 123.0 | 3.20 | 162 | |
| Hu2.1_40LFab | | 3.5 | 121.0 | 3.49 | 163 | |
| Hu2.1_RLFab | | 1.2 | 139.0 | 11.20 | 119 | |
| Hu2.1_94Fab | | 4.8 | 80.4 | 1.67 | 153 | |
| Hu2.1_46Fab | | 19.6 | 25.7 | 0.13 | 229 | |
| Hu2.1_30Fab | | 21.8 | 15.7 | 0.07 | 241 | |
| Ch 11G9 Fab | | | | | | No significant |
| | | | | | | binding |
| Hu11G9_46 | | 6.6 | 90.2 | 1.38 | 88 | |
| Fab | | | | | | |
| Hu11G9_36 | | 4.5 | 104.0 | 2.31 | 70 | |
| Fab | | | | | | |
| Hu11G9_36 | | 5.76 | 23.10 | 0.400 | 116 | |
| IgG | | | | | | |
| Hu11G9_46 | | 6.48 | 18.60 | 0.288 | 119 | |
| IgG | | | | | | |
| Hu9.1-88 IgG | | 13.05 ± 0.64 | 26.15 ± 0.07 | 0.2 ± 0.008 | 240 ± 2 | N = 2 |
| Hu2.1-30 Fab | | 24.10 | 22.20 | 0.092 | 184 | |
| Hu11G9-36 | | 4.66 | 96.80 | 2.080 | 46 | |
| Fab | | | | | | |
| Hu11G9-46 | | 5.00 | 80.80 | 1.62 | 51 | |
| Fab | | | | | | |
| Hu9.1-88 Fab | | 5.41 | 74.20 | 1.370 | 114 | |
| Hu2.1-46 IgG | | 9.78 | 3.96 | 0.041 | 243 | |
| Hu2.1-94 IgG | | 4.77 | 12.10 | 0.253 | 182 | |

B. Antibody on the Chip (ECD in Solution)

TABLE 13

| | Amount | | | | |
|---|---|---|---|---|---|
| Anti-Br3 | immobilized | Ka | | | |
| IgG | (RU) | (10^{17.10}/Ms) | K_{d} (10⁻⁵/s) | K_{D} (nM) | Rₘₐₓ (RU) |
| 2.1 | 1800 | 1.1 | 1.7 | 0.15 | 423 |
| 9.1 | 2600 | 2.1 | 6.4 | 0.30 | 337 |
| 2.1-46 | 1900 | 22.4 | 43.2 | 0.193 | 153 |
| 2.1-30 | 840 | 35.7 ± 9.3 | 7.6 ± 5.8 | 0.020 ± 0.011 | 69 ± 6 |
| 9.1-88 | 3900 | 10.2 | 92.6 | 0.911 | 207 |
| 9.1-RF | 1500 | 18.0 ± 6.5 | 30.9 ± 13.6 | 0.169 ± 0.016 | 111 ± 16 |
| mV3-1 to H | 2500 | 2.67 | 17.10 | 0.64 | 47 |
| mV3-46 to | 2700 | 3.00 | 7.31 | 0.24 | 49 |
| H | | | | | |
| mV3-46s to | 4500 | 15.70 | 3.18 | 0.02 | 22 |
| H | | | | | |
| mV3-1 to | 2500 | 0.84 | 13.10 | 1.56 | 51 |
| M | | | | | |
| mV3-46 to | 2700 | 1.19 | 14.00 | 1.17 | 55 |
| M | | | | | |
| mV3-46s to | 4500 | 2.98 | 9.51 | 0.32 | 33 |
| M | | | | | |

### Example 9

### Functional Epitope Mapping

The following assays were used to functionally map the epitopes on BR3 important for anti-R3 antibody binding.

Library Construction for miniBR3 Shotgun Scanning. Libraries displaying epitope-tagged miniBR3 on M13 bacteriophage were constructed by successive mutagenesis of phagemid pW1205a as previously described (Weiss, G. A., et al., (2000) Proc Natl Acad Sci USA 97:8950-4; Gordon, N. et al., (2003) Biochemistry 42:5977-83). This phagemid encodes a peptide epitope tag (MADPNRFRGKDLGG) fused to the N-terminus of human growth hormone followed by M13 gene-8 major coat protein. pW1205a was used as a template for Kunkel mutagenesis (Kunkel, J. D., et al., (1987) Methods Enzymol 154:367-82) to generate appropriate templates for miniBR3 shotgun library construction. Oligonucleotides replaced the fragment of pW1205a encoding human growth hormone with DNA fragments encoding a partial sequence of miniBR3 containing TAA stop codons in place of the region to be mutated. The two new templates generated, template 1 (encoding residues 34-42) and template 2 (encoding residues 17-25), were each used to construct a miniBR3 library as previously described (Sidhu, H. et al., Methods Enzymol 328:333-63). Each “partial miniBR3” template was used as the template for Kunkel mutagenesis with mutagenic oligonucleotides designed to replace the template stop codons with the complementary region of miniBR3, while simultaneously introducing mutations at the desired sites. At the sites of mutation, wild-type codons were replaced with the corresponding shotgun alanine codon (Weiss, supra). Each of these two libraries allowed for mutations at 11 residues in miniBR3 with no mutated positions in common between libraries. Library 1 encoded shotgun codons at positions 17, 18, 20-23, 25, 27, 28, 30, and 33, while library 2 encoded shotgun codons at positions 26, 29, 31, 34, and 36-42. Each library contained 2×10⁹ members, allowing for complete representation of the theoretical diversity (>10⁴-fold excess).

Library Sorting and Analysis. Phage from each of the two libraries described above were subjected rounds of binding selection against the neutralizing antibodies 9.1, 2.1, 8G4, 11G9 (functional selection) and V3-1 or an anti-tag antibody (3C8:2F4, Genentech, Inc.) (display selection) immobilized on 96-well Nunc Maxisorp immunoplates. The display selection was included in order to normalize the anti-BR3 antibody-binding selection for expression differences between library members. Phage eluted from each target were propagated in E. coli XL 1-blue; amplified phage were used for selection against the same target as in the previous round. After two rounds of selection, 48 individual clones from each library and selection were grown in a 96-well format in 400 L of 2YT medium supplemented with carbenicillin and KO7 helper phage. Supernatants from these cultures were used directly in phage ELISAs to detect phage-displayed variants of miniBR3 capable of binding the antibody target they were selected against to confirm binding.

Phage ELISA can be performed generally as followed. Maxisorp immunoplates (96-well) were coated with capture target protein (anti-BR3 antibody) for two hours at room temperature (100 ul at 5 ug/ml in 50 mM carbonate buffer (pH 9.6)). The plates were then blocked for one hour with 0.2% BSA in phosphate-buffered saline (PBS) and washed eight times with PBS, 0.05% Tween 20. Phage particles were serially diluted into BSA blocking buffer and 100 ul was transferred to coated wells. After one hour, plates were washed eight times with PBS, 0.05% Tween 20, incubated with 100 ul of 1:3000 horseradish peroxidase/anti-M13 antibody conjugate in BSA blocking buffer for 30 minutes, and then washed eight times with PBS, 0.05% Tween 20 and twice with PBS. Plates were developed using an o-phenylenediamine dihydrochloride/H₂O₂ solution (100 ul), stopped with 2.5 M H₂SO₄ (50 ul), and absorbance measured at 492 nm.

All clones tested were found to be positive in their respective ELISAs and were then sequenced as previously described (Weiss, supra). Sequences of acceptable quality were translated and aligned.

Data for BAFF binding and display selection were previously measured (Gordon, supra). Data for anti-BR3 binding and display selection was similarly calculated. Generally, the occurrence of the wild-type residue (wt) and each ala mutation (mut) found amound sequenced clones following two rounds of selection for binding to anti-BR3 antibody or anti-tag antibody was tabulated. The occurrence of the wild-type residue was divided by that of the mutant to determine a wt/mut ratio for each mutation at each position (not shown).

F-values were calculated as previously described (Weiss, supra; Gordon, supra). Generally, a normalized frequency ratio (F) was calculated to quantify the effect of each BR3 mutation on BAFF or anti-BR3 antibody-binding while accounting for display efficiencies: i.e., F=[wt/mutant(BAFF or anti-BR3 antibody selection)] divided by [wt/mutant(display selection)]. Deleterious mutations have ratios >1, while advantageous mutations have ratios <1; boldface indicates a >10-fold effect. Mutations that showed a greater than 10-fold effect (i.e., F>10 or F<0.1) were considered particularly significant.

TABLE 14
F values

| Residue | 9.1 | 2.1 | 8G4 | 11G9 | V3-1 | BAFF |
|---|---|---|---|---|---|---|
| T17 | 0.6 | 0.6 | 1.5 | 0.5 | 0.5 | 0.9 |
| P18 | 0.4 | 0.5 | 1.5 | 0.5 | 0.8 | 0.9 |
| C19 | | | | | | |
| V20 | 0.6 | 3 | 2.1 | 1.1 | 0.9 | 1.4 |
| P21 | 1 | 1.9 | 62 | 40 | 0.6 | 0.5 |
| A22 | 0.3 | 3.2 | 69 | 45 | 0.7 | 0.7 |
| E23 | 4.8 | 9.6 | 11 | 6.9 | 2.4 | 5.4 |
| C24 | | | | | | |
| F25 | 81 | 49 | 58 | 38 | 21 | 46 |
| D26 | 8.7 | 6.1 | 6.4 | 8.5 | 8.7 | 17 |
| L27 | 2.1 | 0.8 | 12 | 1.1 | 1.4 | 9.5 |
| L28 | 1.5 | 0.1 | 2.5 | 0.4 | 98 | 210 |
| V29 | 0.3 | 0.5 | 0.8 | 1 | 92 | 57 |
| R30 | 10 | 10 | 11 | 1.7 | 20 | 16 |
| H31 | 0.5 | 0.6 | 3.8 | 2.8 | 0.1 | 0.3 |
| C32 | | | | | | |
| V33 | 10 | 10 | 38 | 24 | 14 | 106 |
| A34 | 14 | 62 | 41 | 32 | 13 | 28 |
| C35 | | | | | | |
| G36 | 1.9 | 14 | 1.7 | 1.8 | 0.7 | 1.3 |
| L37 | 0.7 | 0.1 | 0.8 | 0.7 | 0.7 | 5.4 |
| L38 | 89 | 0.9 | 1 | 0.9 | 1.4 | 47 |
| R39 | 63 | 0.5 | 2.2 | 3.1 | 0.4 | 4.1 |
| T40 | 0.4 | 0.2 | 0.5 | 0.5 | 0.6 | 0.5 |
| P41 | 7.2 | 0.7 | 1.7 | 1.7 | 1.6 | 1.9 |
| R42 | 2.2 | 1.8 | 0.8 | 0.9 | 0.9 | 1.5 |

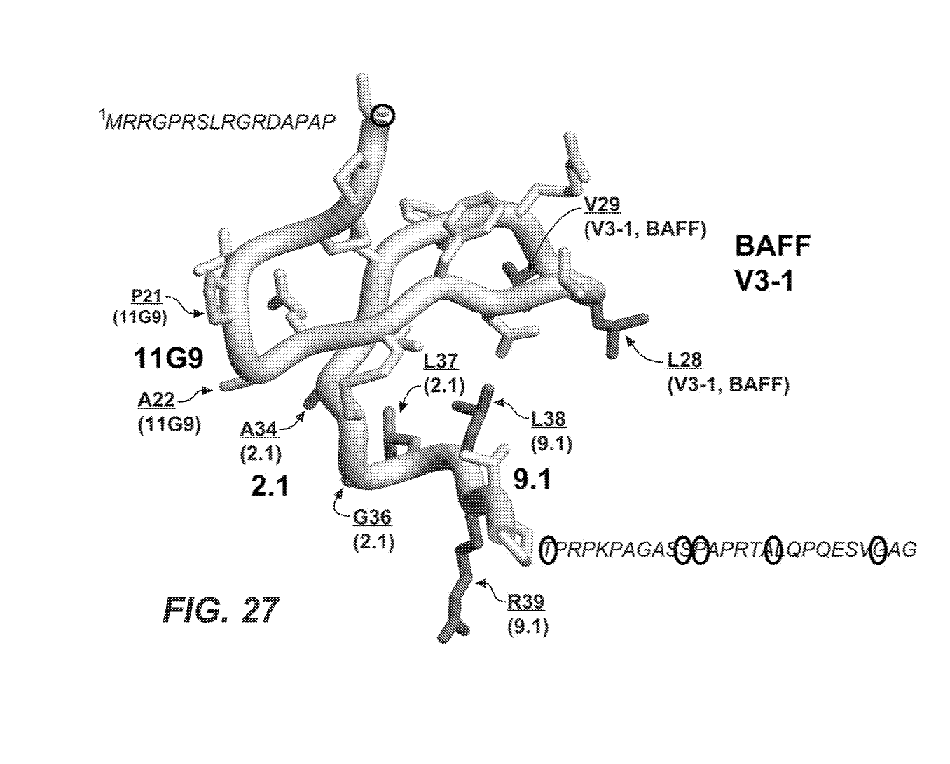

The data indicates that 11G9, 9.1 and 2.1 exploit regions of sequence variation between human and murine BR3 (Table 14). The functional epitope for V3-1 mimics the functional epitope for BAFF that is highly conserved between human and murine BR3. A schematic of this data is shown in FIG. 27. The circled residues in FIG. 27 indicate residues of potential O-linked glycoslyation outside the mini-BR3 sequence. 11G9, 2.1, 9.1 and V3-1 antibodies do not require BR3 glycosylation for binding. The functional epitope for the 9.1 antibody includes L38 and R39. The functional epitope for 2.1 includes G36. The functional epitope for V3-1 includes L28 and L29. The functional epitope for 11G9 includes P21 and A22. Alanine scanning mutation of residues A34, F25 and V33 also disrupted 9.1, 2.1, 11G9 and V3-1 binding to BR3 in this assay, which residues may be important for maintaining the structural integrity of BR3 in the phage.

### Example 10

### CLL Expression

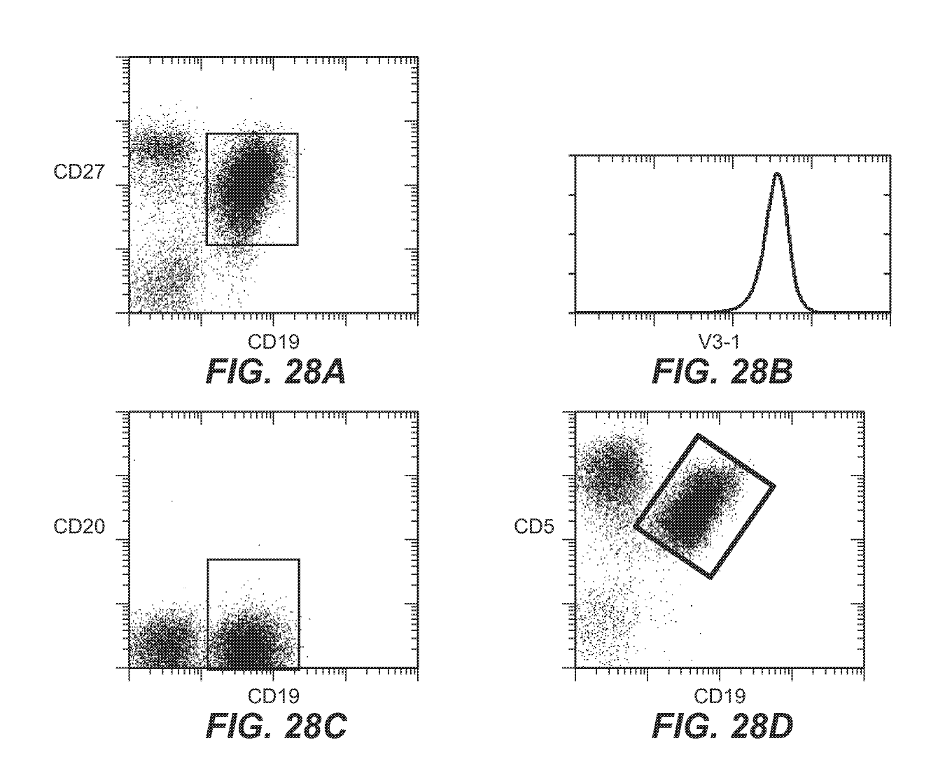

Peripheral blood cells from a chronic lymphocytic leukemia (CLL) patient were stained with antibodies against B cell markers (CD19, CD27, CD20, CD5 and BR3) (FIG. 28). V3-1 was used to stain BR3. Although this particular patient had no CD20 expression on its B cells (CD19+ bottom left), BR3 was expressed at significant levels (see peak of histogram—panel B). Twelve samples from twelve additional CLL patients were evaluated. Twelve out of the twelve samples expressed BR3. These data suggest that anti-BR3 antibodies will have therapeutic value for this indication.

### Example 11

### Antibody Dependent Cellular Cytotoxicity

Anti-BR3 chimeric monoclonal antibodies were assayed for their ability to mediate Natural-Killer cell (NK cell) lysis of BJAB cells (ADCC activity), a CD20 expressing Burkitt's lymphoma B-cell line, essentially as described (Shields et al., J. Biol. Chem. 276:6591-6604 (2001)) using a lactate dehydrogenase (LDH) readout. NK cells were prepared from 100 mL of heparinized blood from normal human donors using the RosetteSep® Human NK Cell Enrichment Cocktail (StemCell Technologies, Vancouver, B.C.) according to the manufacturer's protocol. The blood was diluted with an equal volume of phosphate buffered saline, layered over 15 mL of Ficoll-Paque™ (Amersham Biosciences, Uppsala, Sweden), and centrifuged for 20 min at 1450 RPM. White cells at the interface between layers were dispensed to 4 clean 50-mL tubes, which were filled with RPMI medium containing 15% fetal calf serum. Tubes were centrifuged for 5 min at 1450 RPM and the supernatant discarded. NK cells were diluted in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) to 2×10⁶ cells/mL.

Serial dilutions of antibody (0.05 mL) in assay medium were added to a 96-well round-bottom tissue culture plate. BJAB cells were diluted in assay buffer to a concentration of 4×10⁵/mL. BJAB cells (0.05 mL per well) were mixed with diluted antibody in the 96-well plate and incubated for 30 min at room temperature to allow binding of antibody to BR3 (opsonization).

The ADCC reaction was initiated by adding 0.05 mL of NK cells to each well. In control wells, 2% Triton X-100 was added. The plate was then incubated for 4 h at 37° C. Levels of LDH released were measured using a cytotoxicity (LDH) detection kit (Kit#1644793, Roche Diagnostics, Indianapolis, Ind.) following the manufacturers instructions. 0.1 mL of LDH developer was added to each well, followed by mixing for 10 s. The plate was then covered with aluminum foil and incubated in the dark at room temperature for 15 min. Optical density at 490 nm was then read and used to calculate % lysis by dividing by the total LDH measured in control wells. Lysis was plotted as a function of antibody concentration, and a 4-parameter curve fit (KaleidaGraph) was used to determine EC₅₀ concentrations.

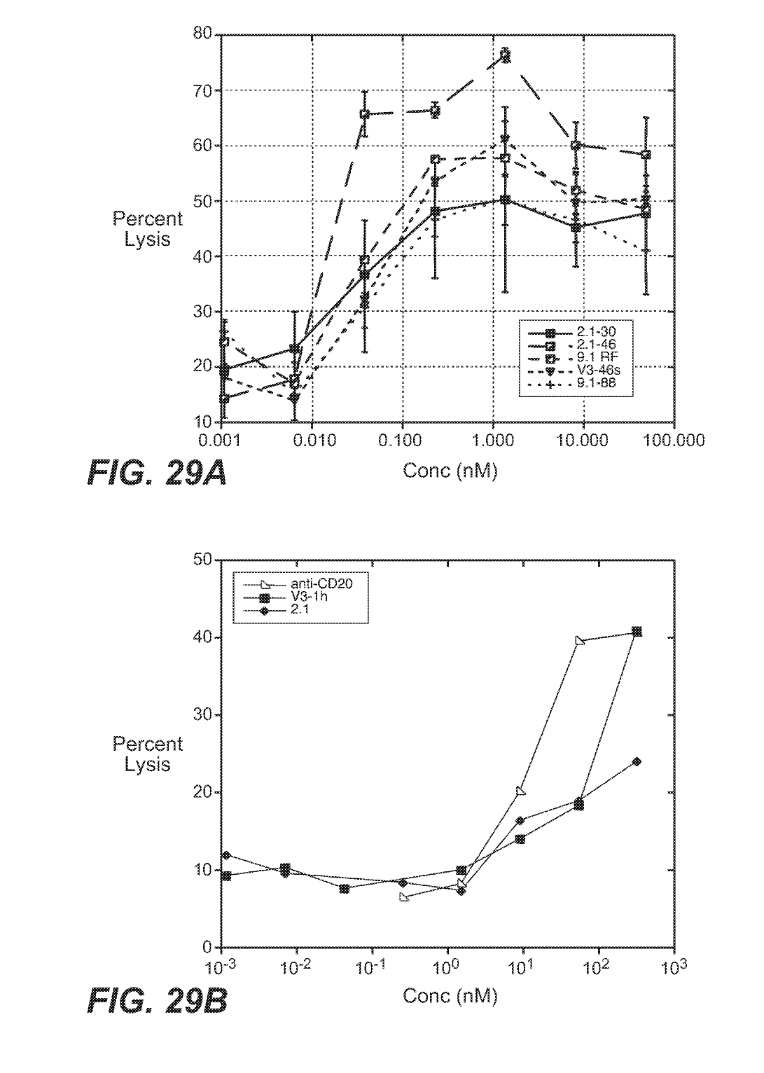

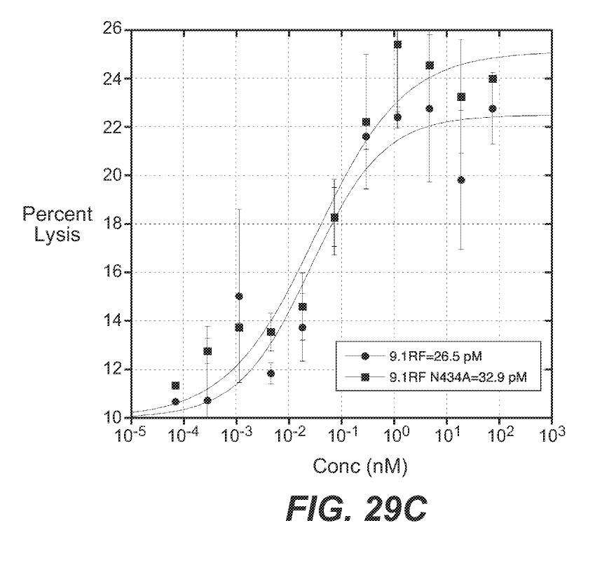

All humanized anti-BR3 antibodies were strongly active in directing NK cell mediated lysis of BJAB cells (human Burkitt's Lymphoma) with relative potencies less than 1 nM (FIG. 29). Similar assays were carried out with Ramos (human Burkitt's lymphoma) and WIL2s cells (human B-cell lymphoma) instead of BJAB cells. FIGS. 29A and B, respectively, show ADCC killing of Ramos and WIL2s cells with anti-BR3 antibodies. An anti-Her2 antibody (4D5) was used as a negative control. In general, antibodies with higher affinity for BR3 were more potent in antibody-dependent cell-killing assays.

### Example 12

### Depletion of B Cells with BR3-Fc or Anti-BR3Antibodies

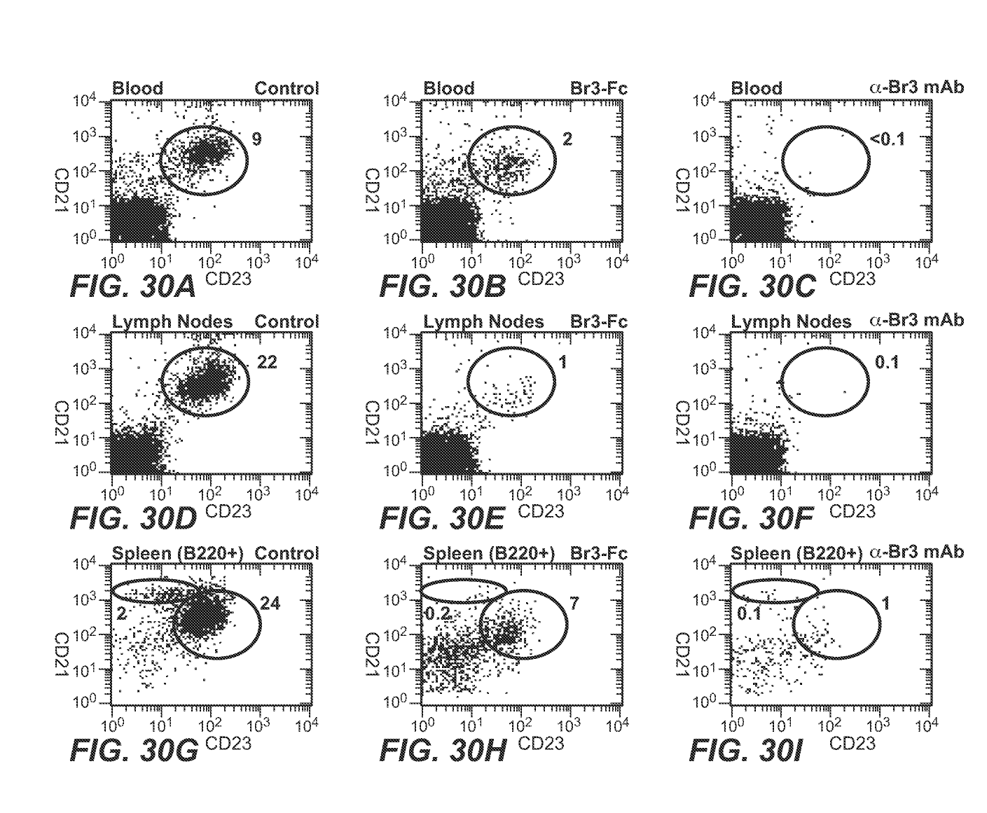

The ability of anti-BR3 antibodies to deplete B cells was compared with BR3-Fc. Six week old BALB/c mice were treated interperitoneally at day 0 with 500 ug control (mouse IgG2a), mouse BR3-Fc or anti-BR3 (V3-1) antibodies. Mice from each group were sacrificed at day 1, 3, 7 and 15. FIG. 30 shows a flowcytometry analysis of B cells in the blood, lymph nodes and spleen at day 7 of treatment. The blood, lymph nodes and spleen show fewer B cells (CD21+CD23+ and CD21highCD23low) in V3-1 treated mice than in BR3-Fc and control treated animals. BR3-Fc treatment has previously been shown to significantly reduce the number of B cells compared with control Fc treated animals. The numbers in bold next to the circles represent the percentage of lymphocytes contained in that particular region (circle).

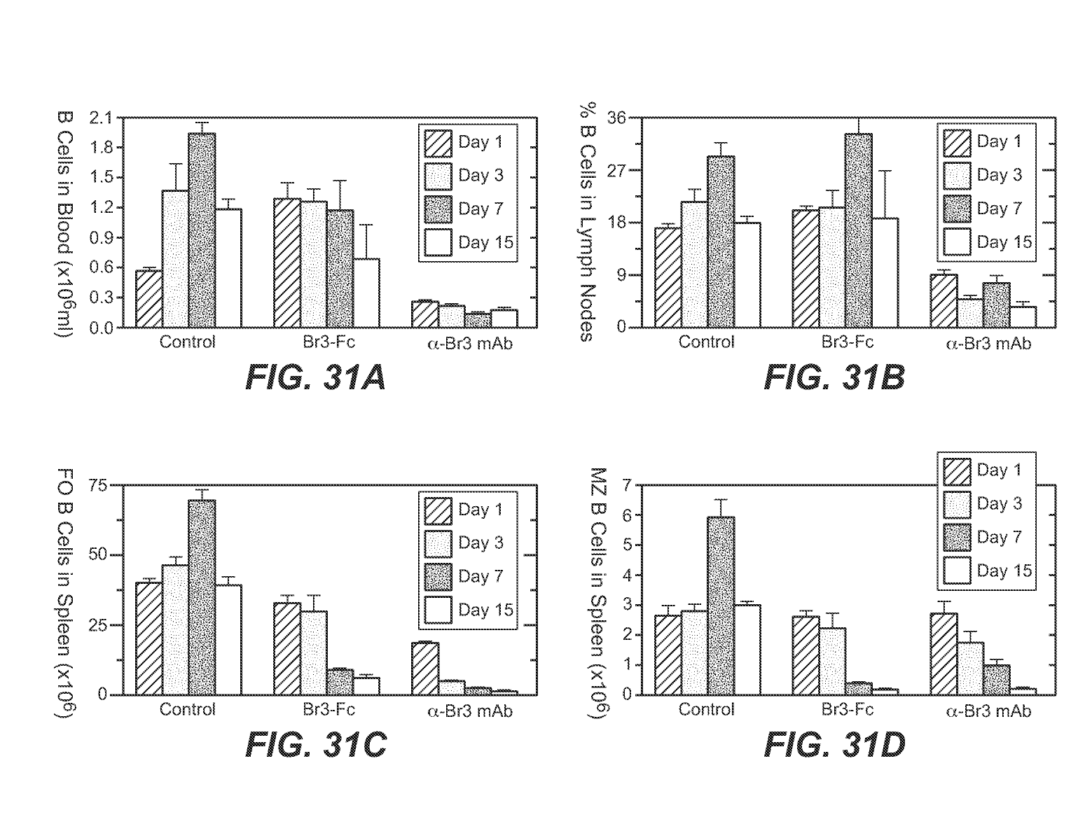

In another experiment under similar conditions, FACS analysis of blood, lymph nodes and spleen generally showed fewer B cells (CD21+CD23+ and CD21highCD23low) in V3-1 treated mice than in BR3-Fc and control treated animals (FIG. 31). BR3-Fc significantly reduced the number of B cells compared with control animals particularly at later time points. FIG. 31 shows the absolute number of B cells contained in 1 ml of blood; the % of B cells in lymph nodes and the absolute numbers of follicular (FO—CD21+CD23+) or marginal zone (MZ—CD21high CD23low) in the spleen at days 1, 3, 7 and 15. Data were expressed as the mean +/−standard error (n=4).

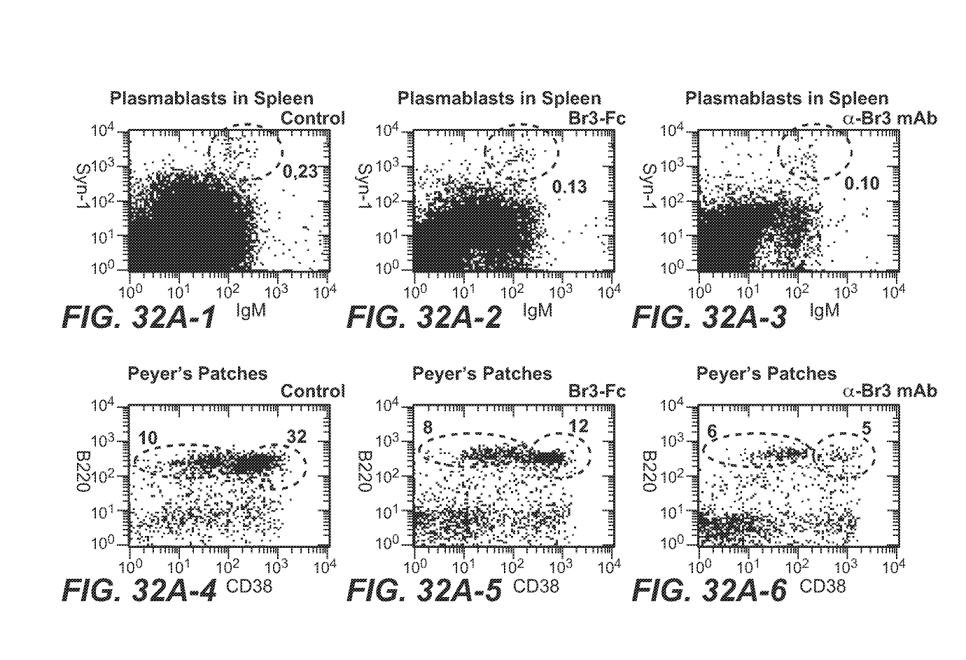

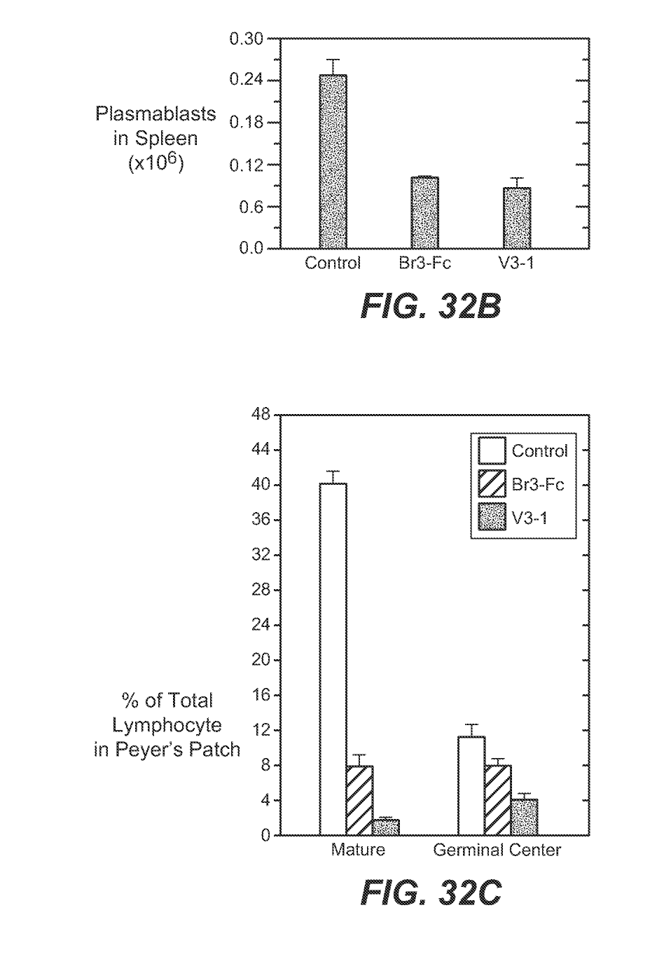

In another experiment under similar conditions, FACS analysis of plasmablasts in the spleen (top row—IgM+Syn+) and germinal center cells (middle row—B220+CD38low) show that anti-BR3 antibodies (V3-1) can deplete some plasmablasts and germinal center cells (FIG. 32). BR3-Fc significantly reduces the number of plasmablasts compared with control animals. Numbers recited in Panel A represent the percentage of lymphocytes contained in that particular region. In the graph bars data is expressed as the mean +/−standard error (n=4).

The data shows that a greater extent of B cell depletion was observed after treatment with anti-BR3 antibodies than with BR3-Fc, which fusion protein blocks BAFF binding to BR3 but does not have ADCC function.

### Example 13

### Fc-Dependent Cell Killing and BAFF Blockade for Maximal B Cell Reduction

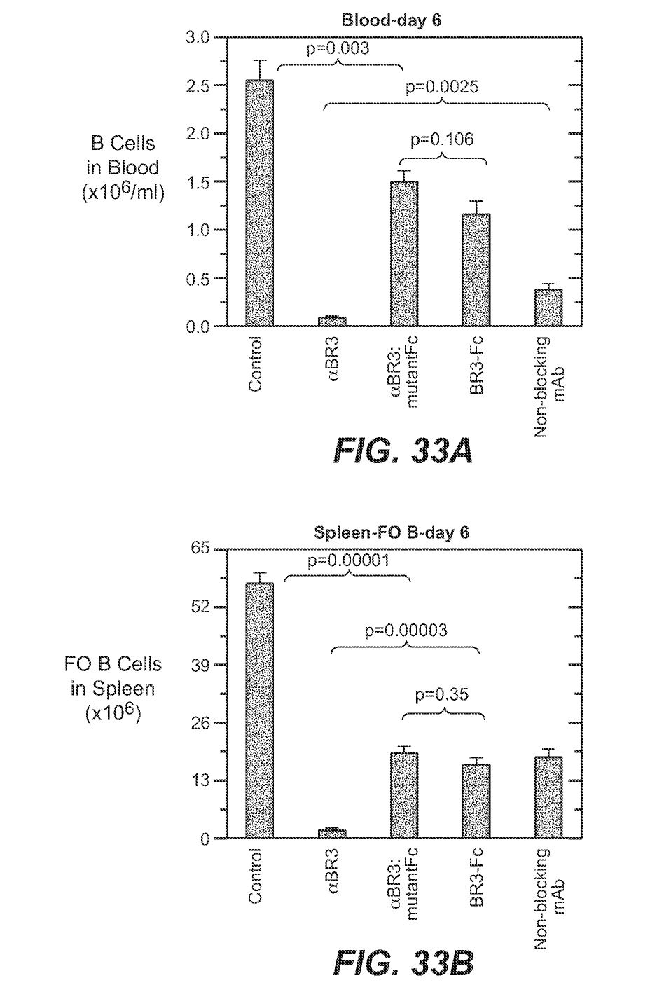

BALB/c mice were treated with a single dose 10 mg/kg of anti-BR3 antibody (mV3-1), mV3-1 with D265A/N297A mutations, a non-BAFF blocking anti-BR3 antibody PIH11 or BR3-Fc. B cells from spleen or peripheral blood were analyzed by flowcytometry at day 6 post treatment. The absolute numbers of peripheral blood B cells (B220+) and splenic follicular B cells (CD21+CD23+) after treatment are reported in FIG. 33A and FIG. 33B, respectively. Data were expressed as the mean +/− standard error (n=4). The D265A/N297A Fc mutation abolishes binding of FcgammaRIII in vitro. The results indicate that although both the non-blocking antibody, the anti-BR3 antibodies with defective Fcgamma receptor-binding, and BR3-Fc can reduce B cell populations, the anti-BR3 antibody having both Fc-dependent cell killing activity and BAFF-blocking activity can be a much more potent B cell reducing/depleting agent. This is due to combining both activities, antibody dependent cell cytotoxicity (ADCC) and B cell survival blockade, into one molecule.

### Example 14

### Lupus Mouse Model

The anti-BR3 antibodies were tested in a lupus mouse model. For these studies, approximately 8 month-old NZB/W lupus-prone positive mice were treated (ip) on day 0 and day 7 with 200 ug of mIgG2a (anti-gp120) (control), or mBR3-Fc or mV3-1 (anti-BR3 antibody). B cells in blood, lymph nodes and spleen (follicular—FO and marginal zone—MZ), were analyzed by flow cytometry. Data are expressed as individual mouse data points (n=4). Similar to BR3Fc, anti-BR3 antibodies are able to diminish the B cells in this autoimmune strain of mice (data not shown).

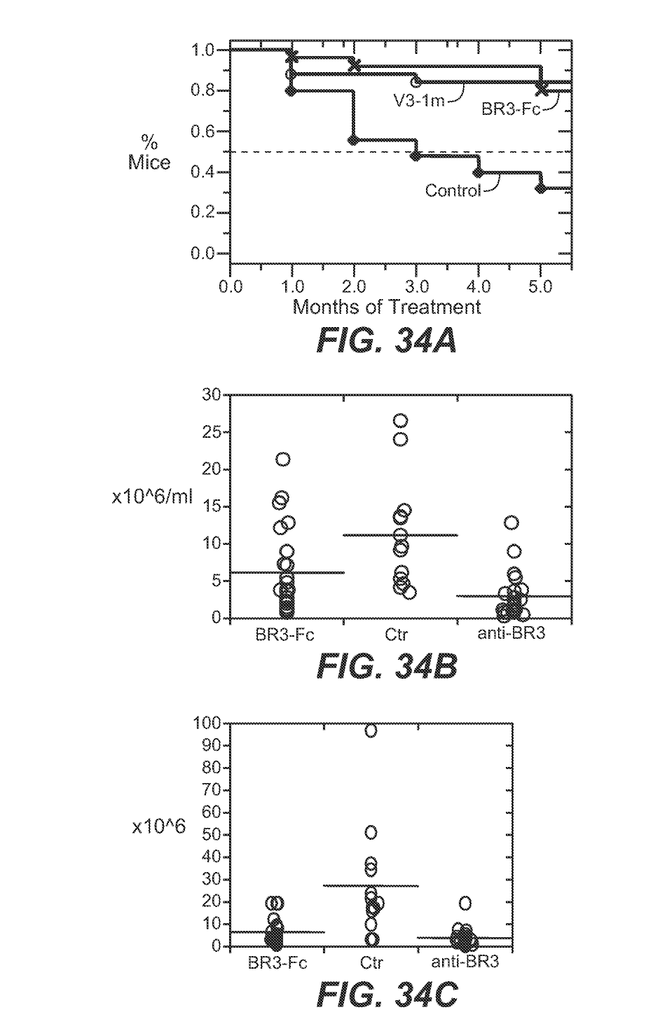

In a longer study, 7 month old NZBxW F1 mice (lupus nephritis mouse model) exhibiting approximately 100 mg/dl proteinuria were treated 2 times per week with 300 ug of mV3-1, mBR3-Fc or a control mIgG2a antibody(anti-gp120) for a period of approximately 6 months. Each treatment cohort contained 25 mice. All mice were evaluated monthly for improvement in time to progression of the disease (FIG. 34A). Time to progression was measured as the percentage of mice surviving or having less than 300 mg/dl proteinuria levels. Additionally, at approximately 6 months post-treatment, the surviving mice were sacrificed and analyzed in the FACS analysis. The median number of peripheral B cells (defined as B220+) in the anti-BR3 antibody treated mice was lower than in the BR3-Fc treated mice and the control mice (FIG. 34B). The median number of total splenic B cells (B220+) in the anti-BR3 antibody treated mice and the BR3-Fc treated mice was lower than in the control mice (FIG. 34C). The median number of activated splenic B cells (B220+CD69+) in the anti-BR3 antibody and BR3-Fc treated mice was lower than in the control mice (data not shown). The median number of splenic plasma cells/plasmablasts (CD138+) in the anti-BR3 antibody (p<0.00001) and BR3-Fc (p<0.02) treated mice was also lower than in the control mice (data not shown). The median numbers of splenic germinal center B cells (B220+CD38low) in the BR3-Fc (p<0.02) and the anti-BR3 antibody (p<0.00001) treated mice were significantly lower than in the control mice (data not shown).

### Example 15

### SCID Model

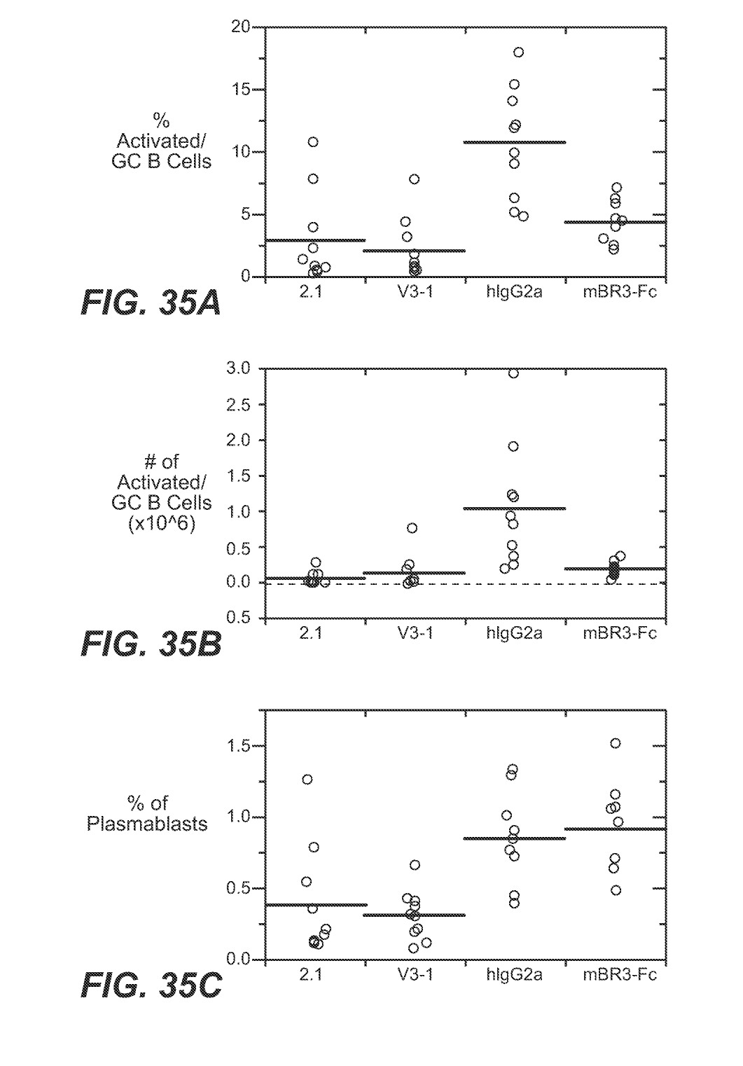

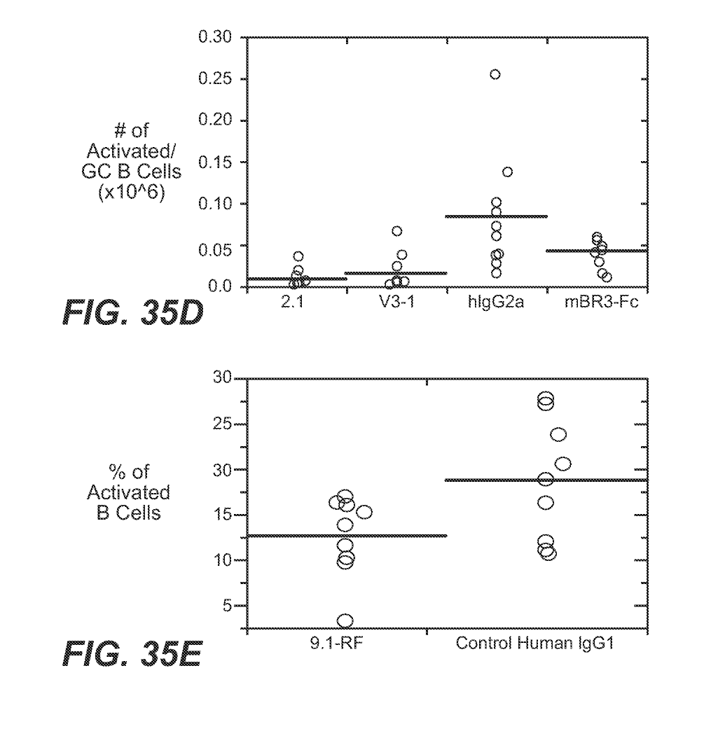

The B cell depletion activity of anti-BR3 antibodies was also tested in a severe combined immune deficient (SCID) model. 40 million human peripheral blood mononuclear cells (PBMCs), enriched magnetically in B cells and CD4 T (>90%) cells, were transferred at day 0 intrasplenically into sublethally irradiated (350rads) 6 week old scid beige mice. Mice were treated at day 0 with 500 ug anti-BR3 antibodies (2.1 or V3-1 with human IgG2a constant region), a human IgG2a isotype control or mouse BR3-Fc. Mice were sacrificed at day 4 and their spleens were analyzed by flow cytometry for human B cells. Both activated/germinal center (GC) B cells (top) and plasmablasts (bottom) were significantly reduced by anti-BR3 treatment while only the activated/GC cells were decreased significantly by BR3-Fc (FIG. 35A-D). 10 individual mice/group are depicted and the average for each group.

In another experiment, human PBMCs were depleted magnetically of CD8 T cells, CD16/CD56 NK cells and CD14 monocytes and injected intrasplenically into irradiated scid-beige mice (40×10⁶/mouse). The same day, mice were treated with 300 ug/mouse human anti-human BR3 (9.1RF) or an isotype ctrl (human IgG1). Seven days later mice were sacrificed and human B cell activation was assessed in their spleens using flowcytometry. The % of activated and germinal center B cells (CD19hiCD38+) was significantly reduced in the group treated with antiBR3 (FIG. 35E).

In yet another experiment, human PBMCs were isolated from Leukopacks from normal human donors (Blood Centers of the Pacific, San Francisco, Calif.) using standard methodologies. The PBMCs were resuspended in 40×10̂6/30 ul PBS and kept on ice during the intrasplenic injection procedure. The recipient mice were sublethally irradiated with 350 Rads using a Cesium 137 source. Four hours after irradiation, all the mice received 40×10̂6 human PBMCs in 30 ul PBS via intrasplenic (i.s.) injection. Under anesthesia, the surgical site had been shaved and prepped with Betadine and 70% alcohol. A one cm skin incision had been made in the left flank just below the costal border followed by incision of the abdominal wall and the peritoneum. The spleen had been carefully exposed and injected with 30 ul cell suspension. The incision had been closed in the muscular layer and the skin with 5-O Vicryl and surgical staples, respectively. All mice had been treated with a single 300 ug dose intravenous injection of Ab solution in 200 ul saline at day 0, four hours prior to cell transfer. Polymyxin B110 mg/liter and Neomycin 1.1 g/liter were added to the drinking water for 7 days post irradiation.

Experimental Groups:

Group 1: Excipient (n=9).

Group 2: anti-BR3 (9.1RF) (n=9).

Group 3: anti-BR3 (9.1RF N434A) (n=9).

All the mice were euthanized at day 4. The B lymphocyte subsets in their spleens were quantified by flow cytometric analysis. Serum samples (100 ul) were collected at day 4 to confirm the serum concentration of Abs at terminal time point.

The human PBMC derived B cells rapidly expanded and activated after transferred into the scid/beige mice. By day 4 after cell transfer, the major B cell population in the spleen showed an activated B cell CD19hi/CD38int phenotype (anti-CD19 and anti-CD38 antibodies). The mean percentage of activated B cells in the placebo treatment group was 10.1% whereas the mean percentage of activated B cells in the 9.1RF treatment group was 0.46%. At four days post-transfer, when the 9.1RF and 9.1RF N434A antibodies were compared in their ability to deplete the B cell precursors as well as inhibit the expansion of activated B cells by BAFF blockade, both showed a statistically significant inhibitory effect (the p values for 9.1RF and 9.1RF N434A were both <0.0001, using Dunnett's test compared to placebo control group). See below.

### Results:

Means and Standard Deviations

| Level | No. | Mean | Std Dev | Std Err Mean | Lower 95% | Upper 95% |
|---|---|---|---|---|---|---|
| 9.1RF | 9 | 1.5206 | 1.6517 | 0.5506 | 0.251 | 2.790 |
| 9.1 N434A | 9 | 1.004 | 0.7791 | 0.2597 | 0.406 | 1.603 |
| Placebo | 9 | 30.2896 | 15.3760 | 5.1253 | 18.470 | 42.109 |

Both anti-BR3 Abs (9.1RF and 9.1RF N434A) show significant depletion and inhibition of B cell survival in a human-scid in vivo model. Since this model is testing in vivo ADCC and BAFF survival blockade, both Abs have adequate properties and potential in treating human autoimmune diseases with B cell components and B cell malignancies.

### Example 16

### FcRn Binding

The 9.1RF IgG antibody (SEQ ID NOs:74 and 75) was altered at residue N434 according to the EU numbering system to increase binding to the human FcRn receptor. The IgG antibodies were produced in CHO cells.

The binding affinities of 9.1 RF and its mutants were determined using a BIAcore-3000 system (BIAcore Inc.). Using 10 mM sodium acetate, pH 4, human and cyno FcRn were immobilized on CM5 chips via amine coupling according to the manufacturer's instructions. Coupling was performed at 25° C. The final densities achieved were 700-1000 RUs.

Kinetic measurements were carried out by injecting three-fold serial dilutions of 9.1 RF or its mutants for 2 minutes in pH 6 running buffer (PBS pH 6, 0.05% Tween-20), using a flow rate of 20 μl/min at 25° C. The maximum concentration of antibody used was 1 μM. Dissociation rates were measured over 10 minutes. Surfaces were regenerated with a 20 μl injection of 10 mM Tris pH 9, 150 mM NaCl with minimal loss of binding activity. The results are presented in Table 15 below as kₐ, k_{c} and KDₐ values.

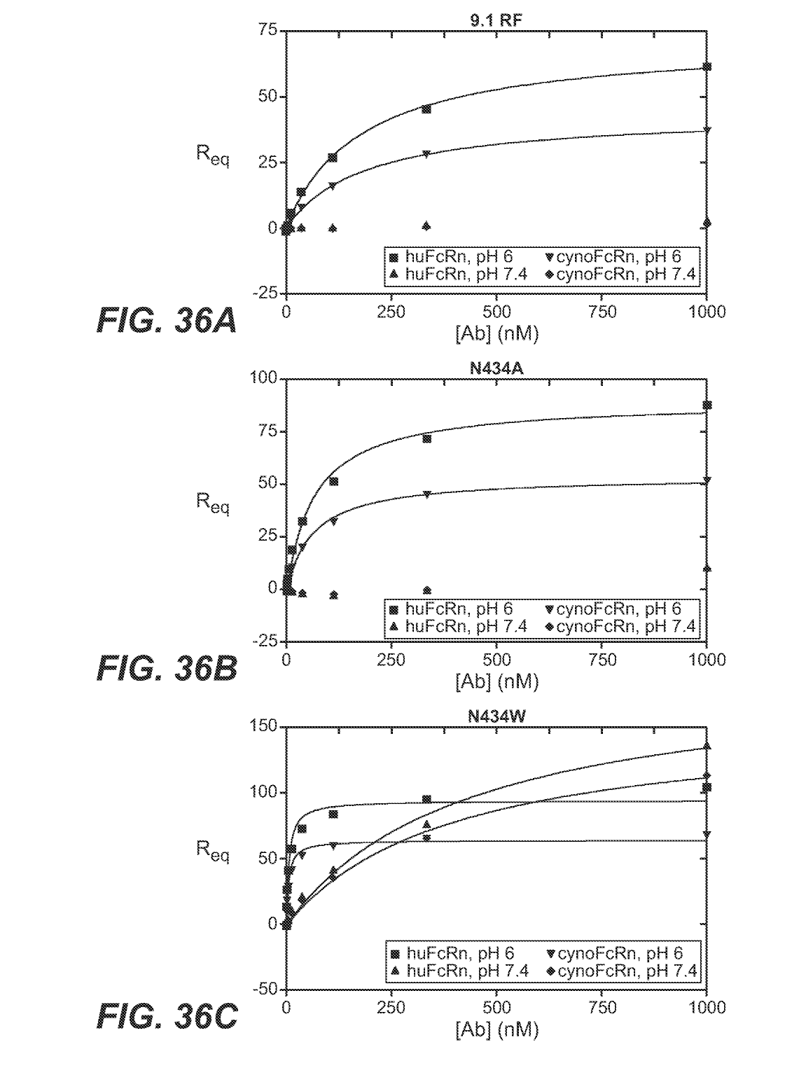

Equilibrium binding experiments were performed by injecting three-fold serial dilutions of 9.1 RF or its mutants for 6 minutes in running buffer, using a flow rate of 2 μl/min at 25° C. Dissociation was allowed to continue for 2 minutes. The maximum concentration of antibody used was 1 μM. Running buffer for the equilibrium binding experiments was either PBS pH 6, 0.05% Tween-20 or PBS pH 7.4, 0.0% Tween-20. Surfaces were regenerated with a 20 μl injection of 10 mM Tris pH 9, 150 mM NaCl. Sensorgrams were evaluated using BIAevaluation v3.2 software. The results are presented in FIG. 36 and as KD values (KD_{b}) in Table 15 below.

Overall, the results show that the N434A and the N434W mutants of 9.1RF had greater affinity for human FcRn and cyno FcRn than 9.1RF at pH 6.0 and at pH 7.4. Further, the N434W mutant had greater affinity for human FcRn and cyno FcRn than the N434A mutant at pH 6.0 and pH 7.4. This data suggests that either mutant will have increased affinity for the human and the cyno FcRn receptors and a longer half life in vivo compared to an antibody having the Fc sequence of 9.1RF.

TABLE 15

| | | | KDₐ (nM) | KD_{b} (nM) |
|---|---|---|---|---|
| Protein | kₐ (×10⁵ M⁻¹S⁻¹) | k_{c} (×10⁻²s⁻¹) | at pH 6.0 | at pH 6.0 |
| huFcRn | | | | |
| 9.1RF | 6.35 | 7.84 | 123 | 117.8 ± 14.0 |
| N434A | 8.84 | 4.67 | 52.8 | 66.6 ± 11.4 |
| N434W | 43.1 | 1.02 | 2.37 | 5.8 ± 1.4 |
| cynoFcRn | | | | |
| 9.1RF | 10.1 | 19.2 | 191 | 185.8 ± 13.7 |
| N434A | 17 | 9.62 | 56.5 | 62.7 ± 6.9 |
| N434W | 47.4 | 1.44 | 3.03 | 5.1 ± 0.9 |

### Example 17

### Fcγ Receptor Binding

Human FcγRs (also referred to as hFcgR below) lacking their transmembrane and intracellular domains and comprising His-tagged glutathione S transferase (GST) sequences at their C-terminus were prepared as described previously (Shields, R. L. et al., (2001) JBC 276:6591-6604).

MaxiSorp 96-well microwell plates (Nunc, Roskilde, Denmark) were coated with 2 ug/ml anti-GST (clone 8E2.1.1, Genentech), at 100 ul/well in 50 mM carbonate buffer, pH 9.6, at 4° C. overnight. Plates were washed with PBS containing 0.05% polysorbate, pH 7.4 (wash buffer) and blocked with PBS containing 0.5% BSA, pH 7.4, at 150 ul/well. After an hour incubation at room temperature, plates were washed with wash buffer. Human Fcγ receptor was added to the plates at 0.25 ug/ml, 100 ul/well, in PBS containing 0.5% BSA, 0.05% polysorbate 20, pH 7.4 (assay buffer). The plates were incubated for one hour and washed with wash buffer. For low affinity Fcγ receptors IIa, IIb, III (F158) and high affinity III (V158), antibodies were incubated with goat F(ab′)₂ anti-K (Cappel, ICN Pharmaceuticals, Inc., Aurora, Ohio) or anti-λ (BioSource, Camarillo, Calif.) antibody at a 1:2 (w/w) ratio for 1 hour to form antibody complexes. Eleven twofold serial dilutions of complexed IgG antibodies (1.17-50000 ng/ml in threefold serial dilution) in assay buffer were added to the plates. For the high affinity FcγRI, eleven twofold serial dilutions of uncomplexed IgG antibodies (0.017-1000 ng/ml in threefold serial dilution) in assay buffer were added to the plates. After a two-hour incubation, plates were washed with wash buffer. Bound IgG was detected by adding peroxidase labeled goat F(ab′)₂ anti-human IgG F(ab′)₂ (Jackson ImmunoResearch, West Grove, Pa.) at 100 μl/well in assay buffer. After a one-hour incubation, plates were washed with wash buffer and the substrate 3,3′,5,5′-tetramethyl benzidine (TMB) (Kirkegaard & Perry Laboratories) was added at 100 μl/well. The reaction was stopped by adding 1 M phosphoric acid at 100 μl/well. Absorbance was read at 450 nm on a multiskan Ascent reader (Thermo Labsystems, Helsinki, Finland).

The absorbance at the midpoint of the standard curve (mid-OD vs. ng/ml) was calculated. The corresponding concentrations of standard and samples at this mid-OD were determined from the titration curves using a four-parameter nonlinear regression curve-fitting program (KaleidaGraph, Synergy software, Reading, Pa.). The relative activity was calculated by dividing the mid-OD concentration of standard by that of sample. The Herceptin® Ab has previously been shown to bind Fcgamma Receptors and was used as a positive control here.

For all FcγR, binding values reported are the binding of each 9.1-RF variant relative to 9.1RF, taken as (A₄₅₀ₙₘ₍ᵥₐᵣᵢₐₙₜ₎/A_{450nm(9/IRF)}) at 0.33 or 1 μg/ml for FcγRII and FcγRIIIA and 2 μg/ml for FcγRI. A value greater than 1 denotes binding of the variant was improved compared with 9.1RF, whereas a ratio less than 1 denotes reduced binding compared with 9.1RF. The hFcγRIII(F158) and hFcγRIII(V158) refer to hFcγRIII isotypes having lower affinity and higher affinity for human IgG, respectively.

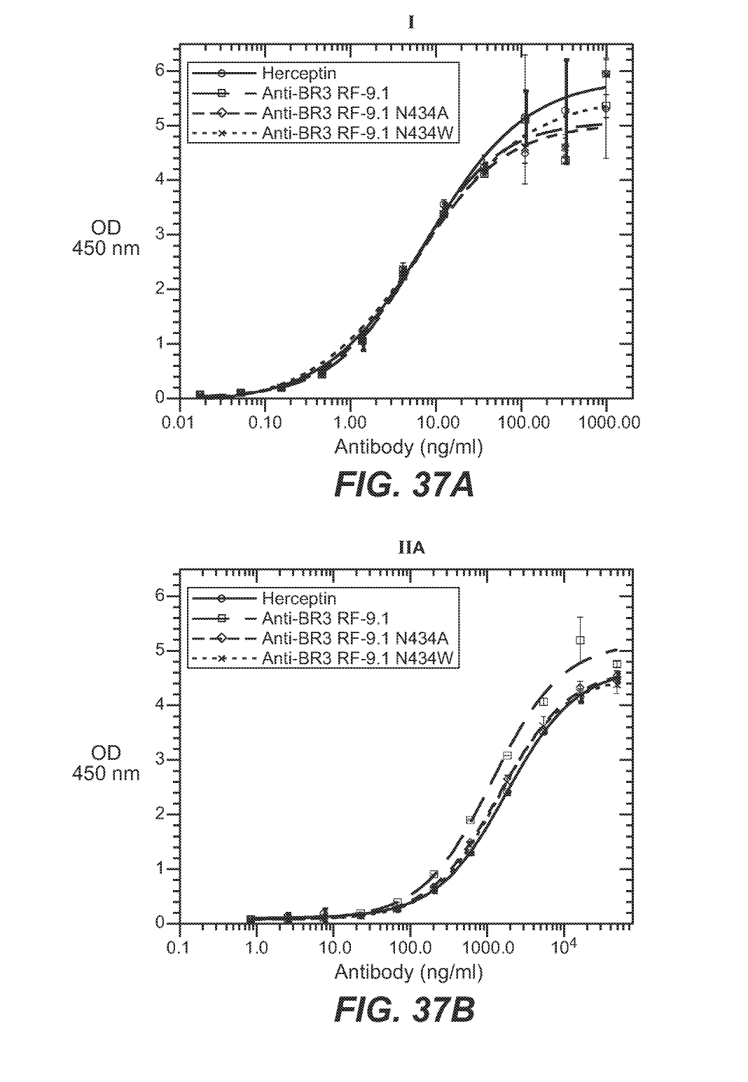

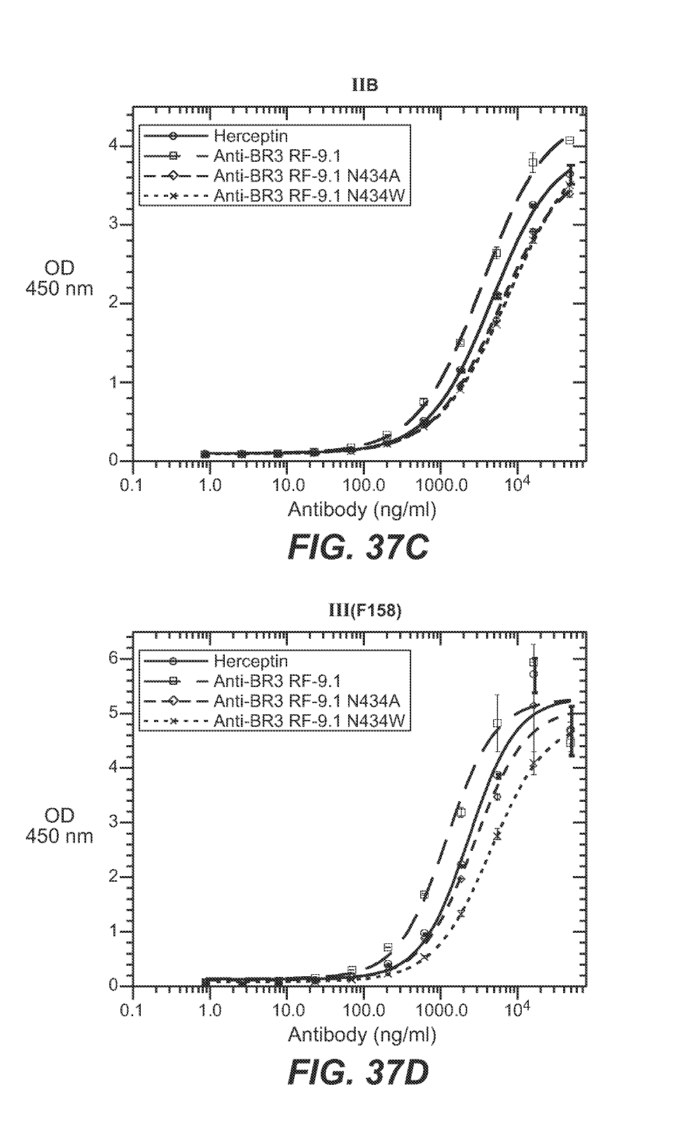

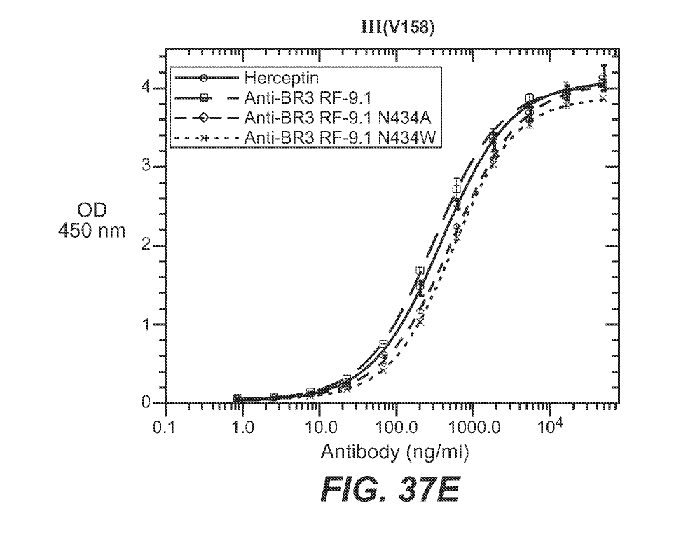

Table 16 and FIG. 37 show that the tested 9.1 anti-BR3 antibodies bind FcγRs similarly and should promote ADCC.

TABLE 16

| Antibody | hFcgRI | hFcgRIIa | hFcgRIIb | hFcgRIII(F158) | hFcgRIII(V158) |
|---|---|---|---|---|---|
| Herceptin ® Ab | 1.02 | 0.54 | 0.62 | 0.51 | 0.80 |
| 9.1-RF | 1.00 | 1.00 | 1.00 | 1.00 | 1.00 |
| 9.1-RF N434A | 0.97 | 0.66 | 0.45 | 0.42 | 0.58 |
| 9.1-RF N434W | 1.00 | 0.64 | 0.40 | 0.24 | 0.51 |

### Example 18

### B Cell Depletion with Anti-CD20 and Anti-BR3 Antibodies

Six week old human CD20 transgenic positive mice were treated (ip) with 200 ug of mIgG2a (control), or m2H7 (a murine anti-human CD20 antibody) or mV3-1. B cells in blood were analyzed one hour, 1 day, 8 days and 15 days after the antibody treatment. B cells from blood, lymph nodes, were analyzed by flowcytometry. Data were expressed as the mean +/−standard error (n=4).

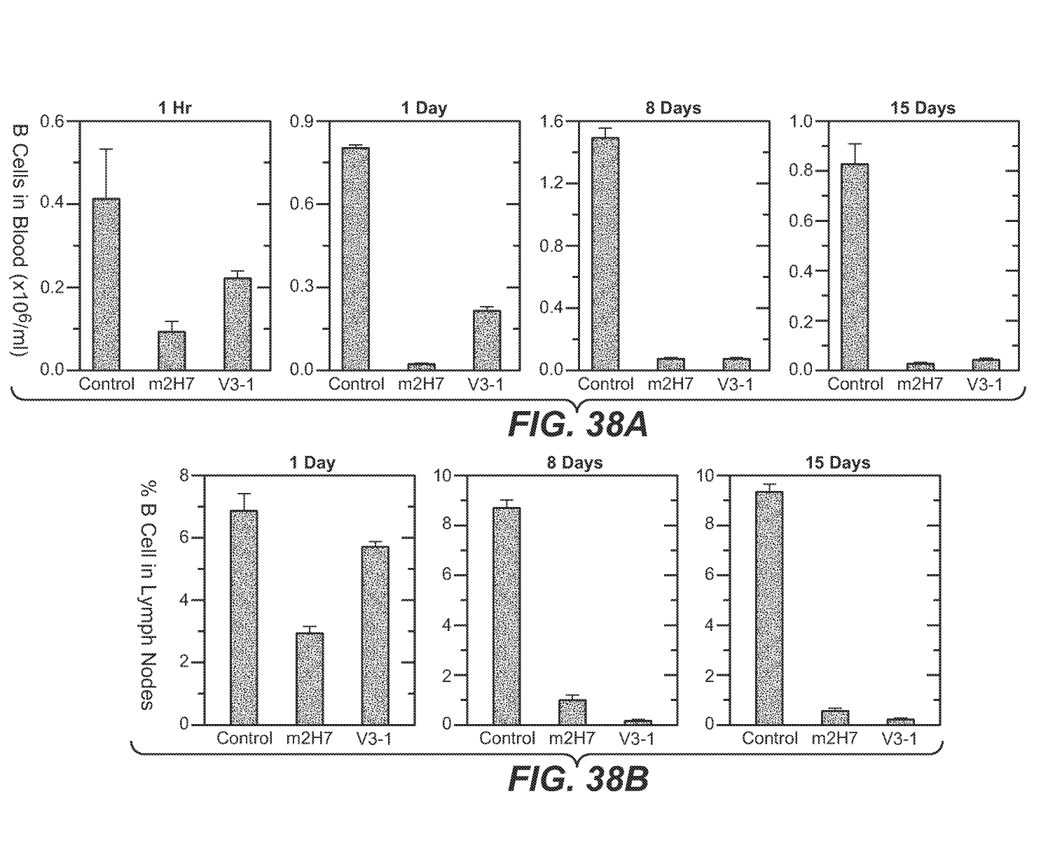

Although at early timepoints (1 hour) and 1 day the anti-CD20 antibodies depleted more cells than the antiBR3 antibodies, by day 8 and 15, the depletion by the antiBR3 antibodies surpassed the depletion by the anti-CD20 antibodies. FIG. 38 shows the post-treatment analysis of B cells levels in the blood and in the lymph nodes.

### Example 19

### Depletion of Follicular and Marginal Zone B Cells

Six week old human CD20 transgenic positive mice were treated (ip) with 200 ug of mIgG2a (control), or m2H7 (a murine anti-human CD20) or mV3-1. B cells in blood were analyzed 1 day, 8 days and 15 days after the mAb treatment. B cells from spleen, were analyzed by flowcytometry. The absolute numbers of follicular (FO—CD21+CD23+) or marginal zone (MZ—CD21high CD23low) in the spleen are compared between the three treatments. Data were expressed as the mean +/−standard error (n=4).

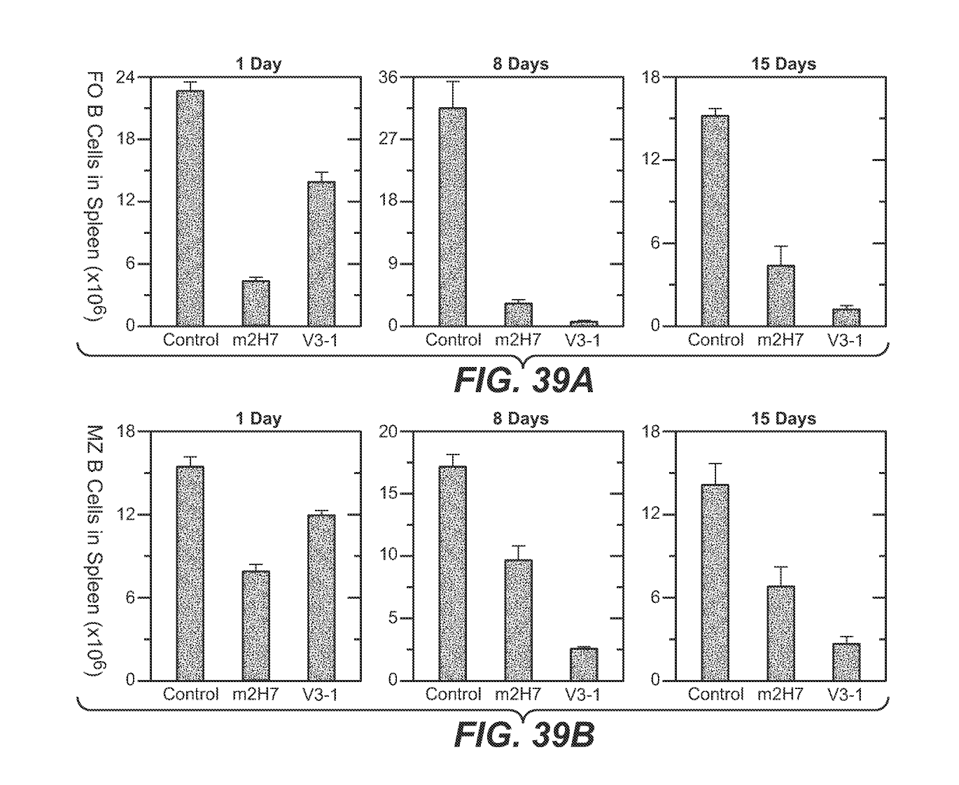

Although at 1 day the anti-CD20 antibodies depleted more cells than the antiBR3 antibodies, by day 8 and 15, the depletion by antiBR3 antibodies surpassed the depletion by antiCD20 antibodies in both follicular and marginal zone B cells (FIG. 39).

### Example 20

### Half-Life in Cyno Monkeys

The pharmacokinetics of three humanized monoclonal anti-BR3 antibodies (9.1RF, 9.1RF N434A and 9.1RF N434W) with different binding affinities to FcRn were compared in cynomolgus monkeys. Seventeen male and 17 female cynomolgus monkeys (Macaca fascicularis) 4-5 years old and weighing 2-4 kg were randomized by weight into one of three groups. Animals in Groups 1, 2, and 3 received a single IV dose of 20 mg/kg of wild type, N434A mutant, or N434W mutant, respectively. The study design is as follows.

| | | Test | | Dose Level | Dose Conc. | Dose Volume |
|---|---|---|---|---|---|---|
| Group | No./Sex | Material | Route | (mg/kg) | (mg/mL) | (mL/kg)^{a} |
| 1 | 5/M, 5/F | wild type | IV | 20 | 20 | 1 |
| 2 | 5/M, 5/F | N434A | IV | 20 | 20 | 1 |
| 3 | 5/M, 5/F | N434W | IV | 20 | 20 | 1 |

Conc. = concentration.
^{a}Total dose volume (mL) was calculated based on the most recent body weight. Dose volumes were interpolated to the nearest 0.1 mL.

Approximately 1.0 mL of blood for pharmacokinetic analysis was collected from a peripheral vein of each animal at the following timepoints:

Predose

30 minutes, and 6 hours post-dose on Study Day 1.

Once on Study Days 2, 3, 4, 5, 8, 11, 15, 18, 22, 29, 36, 43, 50, 57, 64, 71, 78, 85, 92, 99, 106, 113, 120, 127, and 134

Approximately 1.0 mL of blood for anti-therapeutic antibody analysis was collected from a peripheral vein of each animal at the following timepoints:

Predose

Once on Study Days 15, 29, 43, 57, 71, 85, 99, 113, 127 and 134

Blood samples for pharmacokinetic (PK) and anti-therapeutic antibody (ATA) analysis were collected into serum separator tubes and allowed to clot at room temperature for approximately 30-80 minutes. Serum (approximately 0.5 mL) was obtained by centrifugation (2000×g for 15 minutes at room temperature). Serum samples were transferred into prelabeled 1.5-mL Eppendorf tubes and stored in a freezer set to maintain a temperature of −60° C. to −80° C. until packed on dry ice until analysis.

The concentrations of each antibody in each serum sample were determined by using an ELISA assay. The assay lower limit of quantification (LLOQ) in serum is 0.05 ug/mL. Values below this limit were recorded as less than reportable (LTR). Anti-therapeutic antibodies in each sample were determined using a bridging ECLA assay.

Nominal dose and sample collection times with minimal deviation from the schedule were used in the data analysis. Mean and SD of serum 9.1RF, 9.1RFN434A, and 9.1RFN434W concentrations in male and female cynomolgus monkeys were calculated using Excel (version 2000, Microsoft Corporation, Redmond, Wash.,) and plotted using SigmaPlot (version 9.0; Systat Software, Inc., Point Richmond, Calif.). Serum concentrations that were less than reportable were excluded from all data analysis. The SD was not calculated when n<2. Results are presented to three significant figures.

PK parameters for each animal were estimated using a Gauss-Newton (Levenberg and Hartley) two-compartmental model with a 1 over y hat weighting scheme (WinNonlin Version 3.2; Pharsight Corporation; Mountain View, Calif.). Eight out of ten cynos in Group 1 (wild type; 9.1RF) and five out of 10 cynos in Group 3 (9.1RFN434W) developed ATA's by day 57. In general, detection of ATA's at a particular time correlated with a sharp drop in serum concentrations during or after that time, resulting in a shorter terminal half-life and decreased drug exposure. To understand the magnitude of the effect of the ATA response on PK, mean PK parameters for each group were calculated using two methods. In method 1, PK parameters (mean±standard deviation) were calculated using data from all 10 cynos in each group. In method 2, PK parameters were calculated using data only from cynos that did not develop anti-therapeutic antibodies by day 57 (n=2 for group 1, n=10 for group 2, and n=5 for group 3). For groups 1 and 3, method 1 resulted in lower estimates of terminal half-life (t_{1/2, β}) and exposure (AUC) compared to method 2. However, the overall conclusions using the two methods were similar. Therefore, the mean PK parameters reported here were calculated using method 1 (e.g., including data from all cynos).

### Results

Following a single IV bolus administration of 20 mg/kg of 9.1RF (wild type antibody), 9.1RFN434A (N434A variant), and 9.1RFN434W (N434W variant), serum concentrations exhibited biphasic disposition, with a rapid initial distribution phase followed by a slower elimination phase (FIG. 1). Estimated PK parameters for each group are shown in Table 2 and include data from all ten cynos in each group. The terminal half-life (mean±SD) of 9.1RF (wild type antibody) was 6.15±2.01 days and ranged from 4.24 to 11.0 days in ten cynos. The mean terminal half-life (t_{1/2, β}) of 9.1RF in the two cynos that did not develop ATA's by day 57 was 8.95 days. For 9.1RFN434A (N434A variant), the mean terminal half-life was 14.1±1.55 days which is 1.6-2.3 fold greater than that of 9.1RF (p<0.05). For 9.1RFN434W (N434W variant), the mean±SD terminal half-life in ten cynos was 9.55±2.49 days. This value is significantly greater than the overall mean t_{1/2, β} of 9.1RF (wild type antibody) in ten cynos (p<0.05), but it is very similar to the mean t_{1/2, β} of 9.1RF in the two cynos that did not develop detectable ATA's (8.95 days). It is likely that the observed difference in t_{1/2, β} between 9.1RF (wild type antibody) and 9.1RFN434W (N434W variant) is confounded by the ATA response in these two groups.

The area under the concentration-time curve extrapolated to infinity (AUC) of 9.1RF (wild type antibody) was 2440±398 day*ug/mL and ranged from 1740 to 3140 day*ug/mL for the ten cynos. The mean AUC of 9.1RF in the two cynos that did not develop ATA's by day 57 was 2850 day*ug/mL. For 9.1RFN434A (N434A variant), the mean AUC was 4450±685 day*ug/mL which is 1.6-1.8 fold greater than that of 9.1RF (wild type antibody) (p<0.05). There was no difference in the AUC of 9.1RF (wild type antibody) and 9.1RFN434W.

In summary, the pharmacokinetics of 9.1RF, 9.1RFN434A, and 9.1RFN434W were examined following a single IV dose of 20 mg/kg to cynomolgus monkeys. Eight out of 10 cynos developed anti-therapeutic antibodies (ATA's) to 9.1RF by day 56 while 5 out of 10 cynos developed ATA's to 9.1RFN434W by day 56. No cynos developed ATA's to 9.1RFN434A by day 56. 9.1RFN434A exhibited an increased terminal half-life and increased AUC compared to 9.1RF (wild-type antibody) (p<0.05). 9.1RFN434W exhibited a slight increase in terminal half-life compared to 9.1RF; however, it is likely that this observed difference is confounded by the anti-therapeutic antibody response to both 9.1RF and 9.1RFN434W.

| PK Parameter | | WT* | 9.1RFN434A | 9.1RFN434W |
|---|---|---|---|---|
| t_{1/2,β} (days): | Mean ± SD | 6.15 ± 2.01 | 14.1 ± 1.55** | 9.55 ± 2.49** |
| | (Range) | (4.24-11.0) | (12.3-16.5) | (6.86-15.0) |
| AUC (day * ug/mL): | Mean ± SD | 2440 ± 398 | 4450 ± 685** | 2105 ± 438 |
| | (Range) | (1740-3140) | (3390-5560) | (1500-2770) |

*Presence of anti-drug antibodies in 8/10 and 5/10 cynos in WT & 9.1RFN434W groups may confound PK parameters of WT & 9.1RFN434W (e.g., decrease AUC and decrease t_{1/2,β})
**Different from WT with p < 0.05

### Example 21

### Depletion of B Cells in Cynomolgus Monkeys

Anti-BR3 (9.1RF referred to as WT) and the FcRn variant N434A (referred to as 9.1RF N434A). Fifty-one cynomolgus monkeys were dosed with WT or 9.1RFN434A in the following study design

| | | Dose | 4 Week | 8 Week | Recovery |
|---|---|---|---|---|---|
| N | Schedule | (mg/kg) | Necropsy | Necropsy | Necropsy |
| 21 | Placebo IV × 4 weeks; | 0 × 4 | 4 Week; | 8 Weeks; | Recovery; |
| | 1 dose/week | | N = 11 | N = 6 | N = 4 |
| 5 | WT IV × 4 weeks; | 2 × 4 | 4 Weeks; | — | — |
| | 1 dose/week | | N = 5 | | |
| 16 | WT IV × 4/8 weeks; | 20 × 4 | 4 Week; | 8 Weeks; | Recovery; |
| | 1 dose/week | 20 × 8 | N = 6 | N = 6 | N = 4 |
| 9 | 9.1RFN434A IV × 4 | 20 × 4 | 4 Week; | — | Recovery |
| | weeks; | | N = 5 | | N = 4 |
| | 1 dose/week | | | | |

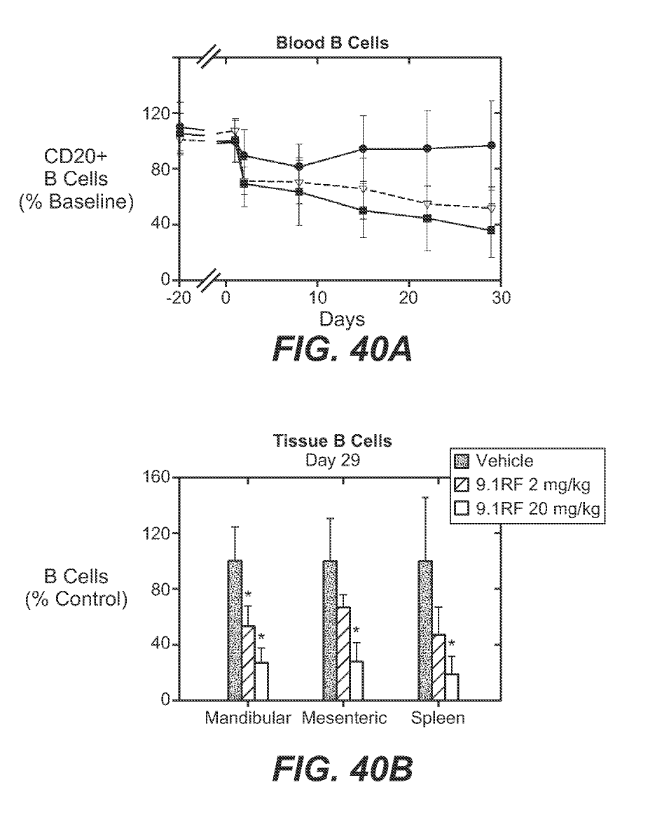

Peripheral B cell (total and B cell subsets) depletion was monitored by FACS in all groups over time and expressed as a percentage of individual animal baselines. The baseline value was a mean of 3 pre-dose sampling time points for each animal. Tissue B cell subsets were analyzed by FACS analysis at each necropsy time points. Tissues analyzed for B cell depletion included spleen, mandibular lymph node and mesenteric lymph node (FIGS. 40A and B).

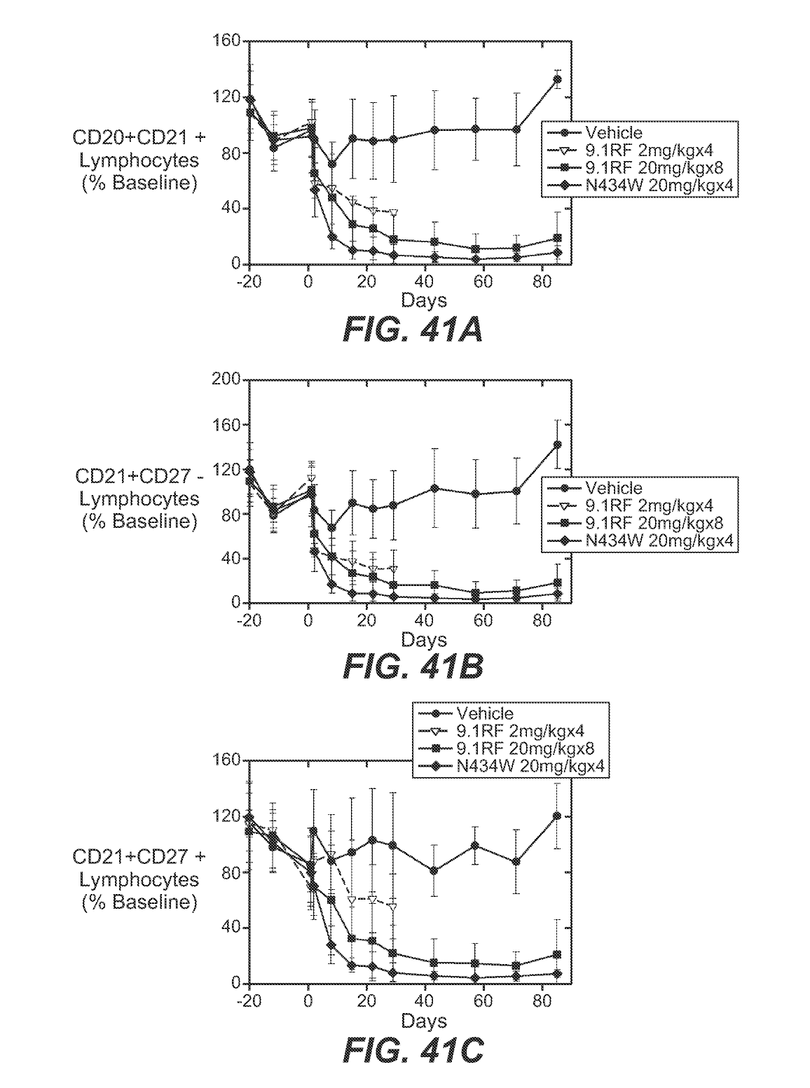

Following dosing, significant B cell depletion was observed in blood in all dose groups. Tissue B cells were depleted on day 29 (necropsy time point) in the WT and 9.1 RFN434A groups dosed at 20 mg/kg×4 doses. B cell depletion in tissue was less pronounced in the WT 2 mg/kg group. FIG. 41A-C shows subpopulations of B cells after treatment.

### Example 22

### Anti-BR3Antibodies with Increased ADCC Activity

Amino acid substitutions in the Fc portion of the anti-BR3 antibody 9.1RF were designed to enhance the ADCC activity of the molecule towards B cell tumor lines. By site-directed mutagenesis, the Fc region of the antibodies were mutated as follows: S298A/K326A/E333A/K334A or S298A/E333A/K334A (EU numbering system). Oligonucleotides specifying the amino acid substitutions were chemically synthesized and used for oligonucleotide-directed mutagenesis of plasmid encoding 9.1RF according to the protocol of Kunkel et al. (Methods in Enzymology (1987) 154, 367-382). Variant sequences were confirmed by dideoxynucleotide-based sequencing. Plasmid DNA was purified from 1 L cultures (2YT media containing 50 μg/mL carbenecillin) of E. coli XL-1 Blue (Stratagene, Inc.), transformed with the relevant plasmid and grown at 37° C. with shaking at 200 RPM, by using the gigaprep protocol described by Qiagen, Inc. Proteins were expressed by using the purified plasmid DNA for transient transfection of CHO cells. Antibodies were purified from 1 L of culture supernatant by chromatography on Protein A-Sepharose followed by cation exchange chromatography on SP-Sepharose. The identity of the purified protein was confirmed by SDS-PAGE and amino terminal sequencing. All of the purified antibodies produced a homogeneous peak upon analytical gel filtration chromatography, with a molar mass of 150,000±5000 calculated from static light scattering data, and less than 3% aggregate content. Analysis of N-linked oligosaccharides by MALDI-TOF (Table 2) indicated a carbohydrate composition typical of recombinant antibodies.

Binding of the variant antibodies to Fcγ receptors was evaluated using an ELISA-based assay. The extracellular domains of human Fcγ receptors I, IIa, IIb, IIIa(F158), IIIa(V158) and mouse Fcγ receptors I, II, and III, were expressed as His-tagged, GST fusion proteins in CHO cells and purified as described in Shields et al. (J. Biol. Chem. 276:6591-6604 (2001)). For the ELISA assay, the fusion proteins were captured on wells of microtiter plates that had been coated with an anti-GST antibody. Dilutions of the variant antibodies were added and allowed to bind followed by washing of the wells to remove unbound antibody. For the weaker binding antibodies the samples were complexed with a Fab′2 fragment of an anti-hu K-chain antibody prior to addition of the samples to the wells. Bound antibody was detected with an HRP-coupled, Fab′2 fragment of a goat anti-huFab′2 antibody. Binding curves were evaluated by using a 4-parameter equation to calculate the EC₅₀ value, the concentration of antibody that gives 50% of the signal observed at saturation. Herceptin® was used as the control antibody in these assays and the fold improvement in binding was calculated from the ratio of the EC₅₀ values (EC₅₀herceptin/EC₅₀sample).

| | Human | | | | | Mouse | | |
|---|---|---|---|---|---|---|---|---|
| Antibody | I | IIa | IIb | IIIa (F158) | IIIa (V158) | I | II | III |
| 9.1 | 1.0 | 2.3 | 0.7 | 2.3 | 1.6 | 1.2 | 0.7 | 0.8 |
| S298A/K326A/E333A/K334A | 0.6 | 0.2 | 0.7 | 25 | 9.2 | 1.9 | 1.3 | 1.2 |
| S298A/E333A/K334A | 0.7 | 0.2 | 0.4 | 18 | 6.9 | 2.5 | 0.4 | 0.6 |

These data show that all of the anti-BR3 variants have increased affinity for both the F158 and V158 allotypes of human FcγRIIa. All of the variants had insignificant changes in affinity for human FcγRI.

The anti-BR3 antibodies were assayed for their ability to mediate Natural-Killer cell (NK cell) lysis of BJAB cells (ADCC activity), a BR3 and CD20 expressing Burkitt's lymphoma B-cell line, essentially as described (Shields et al., J. Biol. Chem. 276:6591-6604 (2001)) using a lactate dehydrogenase (LDH) readout. NK cells isolated from donors heterozygous for the F/V 158 allotype of CD16 were used in the assay at an effector:target ratio of 5:1. NK cells were prepared from 100 mL of heparinized blood using the RosetteSep® Human NK Cell Enrichment Cocktail (StemCell Technologies, Vancouver, B.C.) according to the manufacturer's protocol. The blood was diluted with an equal volume of phosphate buffered saline, layered over 15 mL of Ficoll-Paque™ (Amersham Biosciences, Uppsala, Sweden), and centrifuged for 20 min at 1450 RPM. White cells at the interface between layers were dispensed to 4 clean 50-mL tubes, which were filled with RPMI medium containing 15% fetal calf serum. Tubes were centrifuged for 5 min at 1450 RPM and the supernatant discarded. NK cells were diluted in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) to 2×10⁶ cells/mL.

Serial dilutions of antibody (0.05 mL) in assay medium were added to a 96-well round-bottom tissue culture plate. BJAB cells were diluted in assay buffer to a concentration of 4×10⁵/mL. BJAB cells (0.05 mL per well) were mixed with diluted antibody in the 96-well plate and incubated for 30 min at room temperature to allow binding of antibody to BR3 (opsonization).

The ADCC reaction was initiated by adding 0.05 mL of NK cells to each well. In control wells, 2% Triton X-100 was added. The plate was then incubated for 4 h at 37° C. Levels of LDH released were measured using a cytotoxicity (LDH) detection kit (Kit#1644793, Roche Diagnostics, Indianapolis, Ind.) following the manufacturers instructions. 0.1 mL of LDH developer was added to each well, followed by mixing for 10 s. The plate was then covered with aluminum foil and incubated in the dark at room temperature for 15 min. Optical density at 490 nm was then read and used to calculate % lysis by dividing by the total LDH measured in control wells. Lysis was plotted as a function of antibody concentration, and a 4-parameter curve fit (KaleidaGraph) was used to determine EC50 concentrations.

All of the anti-BR3 variants were active in the ADCC assay giving EC₅₀ values less than 1 nM (% killing vs antibody concentration). The Fc substitutions led to an increase in potency relative to 9.1 (data not shown) by the lowering of the EC₅₀ and increase in the maximal % killing. The S298A/K326A/E333A/K334A mutant had a 3 fold higher ADCC activity in this assay relative to 9.1wt (relative EC₅₀ values). The S298A/E333A/K334A mutant had a 2.8 fold higher ADCC activity in this assay relative to 9.1 wt (relative EC₅₀ values).

1.-143. (canceled)

144. An antibody that binds to a human BR3 extracellular domain sequence, wherein the antibody has an altered Fc region compared to a wild-type IgG Fc region and wherein the antibody has antibody dependent cellular cytotoxicity (ADCC) in the presence of human effector cells or has increased ADCC in the presence of human effector cells compared to an antibody comprising a human wild-type or native sequence IgG Fc.

145. An antibody that binds to a human BR3 extracellular domain sequence, wherein the antibody has an altered Fc region compared to a wild-type IgG Fc region and wherein the antibody has an increased half-life in vivo compared to an antibody having a wild type or native sequence IgG Fc.

146. The antibody of claim 145, wherein the antibody comprises an altered Fc region with higher affinity for the human Fc neonatal receptor (FcRn) at pH 6.0 compared to an antibody comprising a wild-type IgG Fc region.

147. An antibody that binds to a human BR3 extracellular domain sequence and kills or depletes B cells in vivo by at least 20% compared to the baseline level or negative control which is not treated with the antibody, wherein the antibody comprises an altered Fc region compared to a wild-type IgG Fc region.

148. The antibody of claim 145, wherein the antibody kills or depletes B cells in the blood in vivo by at least 25% compared to the baseline level or negative control which is not treated with the antibody.

149. The antibody of claim 145, wherein the antibody kills or depletes B cells in the blood in vivo by at least 30% compared to the baseline level or negative control which is not treated with the antibody.

150. The antibody of claim 145, wherein the antibody kills or depletes B cells in the blood in vivo by at least 50% compared to the baseline level or negative control which is not treated with the antibody.

151. The antibody of claim 145, wherein the antibody can deplete at least one of the primate B cells selected from the group consisting of human, cynomologus monkey and rhesus monkey B cells.

152. The antibody of claim 145, wherein the antibody is conjugated to serum albumin, a serum albumin binding polypeptide, or a non-protein polymer.

153. The antibody of claim 145, wherein the antibody comprises an altered Fc region with higher affinity for the human Fc neonatal receptor (FcRn) at pH 6.0 compared to an antibody comprising a wild-type IgG Fc region.

154. The antibody of claim 145, wherein the antibody has an H1, H2, and H3 region with at least 70% homology to the H1, H2, and H3 region, respectively, of any one of the antibodies of Table 2.

155. The antibody of claim 145, wherein the antibody has an L1, L2, and L3 region with at least 70% homology to the L1, L2, and L3 region, respectively, of any one of the antibodies of Table 2.

156. The antibody of claim 145, wherein the antibody is conjugated to a cytotoxic agent or a chemotherapeutic agent.

157. The antibody of claim 145, wherein the antibody is a monoclonal antibody.

158. The antibody of claim 145, wherein the antibody is a humanized antibody.

159. The antibody of claim 145, wherein the antibody is a human antibody.

160. The antibody of claim 145, wherein the antibody is selected from the group consisting of a Fab, Fab′, a F(ab)′₂, single-chain Fv (scFv), an Fv fragment; a diabody and a linear antibody.

161. The antibody of claim 145, wherein the antibody is a multi-specific antibody.
